(19) United States
(12) Patent Application Publication
(10) Pub. No.: US 2025/0263490 A1
(43) Pub. Date: Aug. 21, 2025

(54) Cation-Independent Mannose-6-Phosphate Receptor Binders

(71) Applicants: VIB VZW, Gent (BE); Universiteit Gent, Gent (BE)

(72) Inventors: Nico Callewaert, Nevele (BE); Linde Van Landuyt, Gent (BE); Justine Naessens, Gent (BE); Yehudi Bloch, Gent (BE); Loes Van Schie, Gent (BE); Kenny Roose, Gent (BE); Wim Nerinckx, Astene (BE)

(21) Appl. No.: 18/547,111

(22) PCT Filed: Feb. 21, 2022
(86) PCT No.: PCT/EP2022/054278

§ 371 Date: Aug. 18, 2023

(30) Foreign Application Priority Data
Feb. 19, 2021 (EP) 21158298.6
Jul. 9, 2021 (EP) 21184788.4

(51) Int. Cl. C07K 16/28; A61K 39/00

(52) U.S. Cl. CPC C07K 16/2863; A61K 2039/505; C07K 2317/22; C07K 2317/569; C07K 2317/77; C07K 2317/92; C07K 2317/94

(57) ABSTRACT

The present invention relates to protein binding agents specifically binding the human cation-independent mannose-6-phosphate receptor, more specifically agents comprising an immunoglobulin single variable domain (ISVD) which allow internalisation upon binding to the extracellular N-terminal domains 1, 2 and/or 3 in monovalent format. More specifically said ISVD provides for means and methods for lysosomal targeting, especially when fused to further proteins such as enzymes relevant for treatment of diseases caused by a lysosomal storage phenotype or lysosomal storage diseases. Finally the binding agents of the invention provide for use in therapeutic treatments, such as in Enzyme-replacement therapy, more specifically, when fused to human acid α-glucosidase (hGAA) or human cathepsin D proteases for treatment of Pompe disease or sporadic inclusion body myositis or neuronal ceroid lipofuscinosis 10 (CLN10), respectively.

### CROSS-REFERENCE TO RELATED APPLICATIONS

This application is a national phase entry under 35 U.S.C. § 371 of International Patent Application PCT/EP2022/054278, filed Feb. 21, 2022, designating the United States of America and published in English as International Patent Publication WO 2022/175532 on Aug. 25, 2022, which claims the benefit under Article 8 of the Patent Cooperation Treaty to European Patent Application Serial No. 21158298.6, filed Feb. 19, 2021 and European Patent Application Serial No. 21184788.4, filed Jul. 9, 2021, the entireties of which are hereby incorporated by reference.

### FIELD OF THE INVENTION

The present invention relates to protein binding agents specifically binding the human cation-independent mannose-6-phosphate receptor, more specifically agents comprising an immunoglobulin single variable domain (ISVD) which allow internalisation upon binding to the extracellular N-terminal domains 1, 2 and/or 3 in monovalent format. More specifically said ISVD provides for means and methods for lysosomal targeting, especially when fused to further proteins such as enzymes relevant for treatment of diseases caused by a lysosomal storage phenotype or lysosomal storage diseases. Finally the binding agents of the invention provide for use in therapeutic treatments, such as in Enzyme-replacement therapy, more specifically, when fused to human acid α-glucosidase (hGAA) or human cathepsin D proteases for treatment of Pompe disease or sporadic inclusion body myositis or neuronal ceroid lipofuscinosis 10 (CLN10), respectively.

### BACKGROUND

Lysosomes are acidified organelles containing more than 70 hydrolytic enzymes. These enzymes are responsible for the degradation of cleavable cellular macromolecules to their original building blocks.

Macromolecules generally reach the lysosome via endocytosis, phagocytosis or endocytosis after which each elementary unit can be recycled and used for the synthesis of other macromolecules or can be further metabolized as a supply for energy. Shortly after the discovery of lytic bodies or lysosomes in the early sixties by Christian de Duve⁸⁸⁻⁸⁹, the importance of it was emphasized by Hers and colleagues as they discovered a deficiency of lysosomal acid maltase deficiency, now called Pompe disease⁹⁰. Today, more than 50 deficient lysosomal enzymes have been described and belong to the group of lysosomal storage diseases (LSD). Although clinically heterogeneous and multifactorial disorders, all share the build-up of lysosomal substrates. This leads to a number of complications such as a general lysosomal dysfunction with metabolic imbalances, cellular dysfunction through cell signalling, as well as impaired autophagy processes. Disease onset can be early or late but in any case, clinical symptoms are multifaceted, ranging from mild to severe with most pathological characteristics localised in the musculoskeletal, cardiorespiratory, renal, digestive and central nervous system.

Treatment of these metabolic diseases had been proposed in the sixties as well, but approval of the first Enzyme Replacement Therapy (ERT), in which a patient is treated with recombinant functional counterpart of the deficient enzyme, lasted until 1991⁹¹. The very first generation of therapeutic enzymes were derived from organs like human placenta but very soon, one switched to the recombinant production of lysosomal enzymes. Being decorated with terminal mannose-6-phosphate on their N-glycans, these proteins could be recognized by one of the P-type lectins, Cation-independent Manoose-6-Phosphate Receptors (CI-M6PRs), on the cell's plasma membrane. As the receptor cycles continuously through the endolysosomal pathway, therapeutic enzymes could efficiently be delivered for degradation of the stored products.

Intravenous administrations of therapeutic enzymes to LSD patients, to clear the stored substrates, generally lead to significantly improved life quality and an overall clinical benefit¹. These approved enzyme replacement therapies however, are expensive and patients require continuous treatment. Despite the orphan drug designation for ERT, accompanied by financial and regulatory advantages, ERT development remains hazardous due to the lack of appropriate pre-clinical animal models, the limited amount of patients for clinical trials and risk for immunological reactions after multiple injections. Moreover, biodistribution of ERT to difficult-to-reach sites like the central nervous system, heart, skeletal muscle, the bone, etc. remains challenging. This is mainly attributed to the a specific clearance, quick degradation and low bioavailability of ERT, which makes it not always as cost-effective as is generally accepted for expensive orphan drugs.

Today, the market size of ERT is estimated around 7,7 billion dollar and the marker is still rising with having a compound annual growth rate that is expected around 7.7% from 2019 to 2026. However, current biologics have some important drawbacks as well. Only 10-20% of the recombinantly produced ERT contain highly affine bis-mannose-6-phosphate modifications that mediate efficient receptor-mediated endocytosis⁹². Moreover, also single M6P-modified ERT are produced but have generally a 1000-fold lower affinity for the CI-M6PR. On top of that, these residues are known substrates of phosphatases in body fluids, limiting the number of CI-M6PR substrates and eliminating receptor-mediated endocytosis of enzymes. Because of this, increasing the mannose-6-phosphate content on lysosomal proteins is well investigated. Whereas some studies focus on cell-line engineering, others coupled chemically bis-M6P-containing N-glycans on native sugars or used synthetic analogues of M6P⁹³⁻⁹⁶. Other strategies entail the development of a glycosylation-independent targeting moiety e.g. Insulin-like-growth factor II (IGF-II)⁹² or receptor-associated protein (RAP) that bind the CI-M6PR and low density lipoprotein receptor, respectively. Both peptides show a high affinity for their receptor and enable efficient internalisation and delivery of lysosomal enzymes. However, this IGF-II moiety can interfere with IGF-II binding proteins present in the serum and the native IGF-II binding site on CI-M6PR (the receptor also named IGFR2). The same holds for RAP that has been described to bind other lipoprotein particles in the serum.

One of the first ERT developed was human acid α-glucosidase (hGAA). It is currently on the market as Myozyme® for the treatment of the autosomal recessive, very rare lysosomal disorder Pompe disease²′. The entire mutational scope of GAA that leads to deficient GAA activity and glycogen build-up is very heterogeneous with more than 582 mutations described⁴. These are situated throughout the entire gene and are most frequently missense mutations but can also be small deletions, splicing variants, nonsense mutations, small and large indels, duplications or complex rearrangements⁵. The functional impact of these mutations on GAA and their impact on the clinical heterogeneity of the disease is not always easy to characterise, but generally, nonsense mutations or the introduction of premature stop codons lead to more severe outcomes⁶.

Another disease with a storage phenotype, also affecting skeletal muscles, is sporadic inclusion body myositis. This is characterised, at least in part, by an excessive loading of inclusion bodies with aggregated proteins in myocytes and an impaired lysosomal protease activity due to an age-related loss of lysosomal functionality. Sporadic IBM has a prevalence of 50-150 patients per million people older than 50^{18,19} and is the most frequently occurring age-related myopathy. The disease has an unknown etiology and is characterised by a progressive weakening of the skeletal muscles due the accumulation of protein inclusion bodies. In these myocytes, the formation of autophagosomes is upregulated, as is the expression level of lysosomal hydrolases. However, the detected enzymatic activity of the main lysosomal proteases cathepsin D (hCTSD) and -B is reduced by 60% and 40% respectively²⁰. This demonstrates a molecular defect—yet of unknown nature—in the processes that lead to processing, folding and/or lysosomal targeting of these major proteases. There is currently no treatment for the disease beyond symptomatic alleviation of inflammation and pain. Novartis developed a new monoclonal antibody (Bimagrumab) that inhibits the myostatin activating II receptor, thereby stimulating muscle growth. However, the phase IIb/III clinical trial was halted because of lack of clinical improvement^{19,20}. What could be effective is a treatment that tackles the root pathogenic process, which is the intoxication of muscle cells by lysosomal protein inclusion bodies. In that regard, Orphazyme is currently developing Arimoclomol, a promising small molecule that stimulates the protein repair machinery by activating the chaperone heat shock protein 70, hence helping to keep proteins sufficiently and well-folded²¹⁻²⁵.

A further application is based on the acidic pH in the endosomes, which results in dissociation of a cargo from the CI-M6PR receptor at a pH around 5.8 in a late endosomal stage¹⁰⁷, and allows rapid recycling of the CI-M6PR receptor itself. Thus, CI-M6PR cargos are efficiently delivered to lysosomes through the endocytotic cycle, a concept that is used in design of lysosome-targeting chimaeras (LYTACs) that enable the depletion of secreted and membrane-associated proteins and are built from a small molecule or antibody fused to chemically synthesized glycopeptide ligands that are agonists of the cation-independent mannose-6-phosphate receptor (CI-M6PR)⁹⁷. LYTACs were shown to in vitro internalize and degrade a selection of both extracellular and transmembrane proteins when administered to cells. For these chimeric antibodies, a downside for in vivo applications is the large size of the construct (±150 kDa), which can hinder its biodistribution in solid tissues¹⁰⁸, and thus requires further investigation of chemical tunability, as well as pharmacokinetic and pharmacodynamic properties for therapeutic application. Moreover, when mannose-6-phosphonate (M6Pn) glycopolypeptides are used for binding the CI-M6PR, the long synthesis process to produce the ligand and subsequent conjugation to the antibody is highly complex and very expensive.

So several therapeutic applications, including ERT for treatment of lysosomal storage phenotype-related diseases, as well as translational insights leading to lysosomal targeting, would benefit from novel next-generation lysosome targeting agents, with the potential to overcome the above hurdles.

### SUMMARY OF THE INVENTION

The present invention relates to Lysosome targetable anti-CI-M6PR binding agents and is based on the identification of a panel of VHHs that specifically bind to the extracellular N-terminal portion (domain 1-3 as described herein) of human CI-M6PR, and preferably cross-reactive with mouse CI-M6PR, as present on the extracellular side of the plasma membrane, thereby enabling traffic through the endolysosomal pathway upon binding with their antigen. Moreover, a number of anti-CI-M6PR VHH families has been identified herein to adopt specific pH-dependent dissociation properties, which promote recycling with the M6PR to and from endolysosome, or rather delivery to the lysosomal compartment. Fusions of, preferably these latter type of, anti-CI-M6PR VHH moieties to therapeutic lysosomal enzymes enables to apply these binding agents in targeted ERT in a glycan-independent way to diseased cells. Moreover, linking such anti-CI-M6PR VHHs with other antigen-binding domains, for instance when synthesized as a bispecific, targeting another extracellular or cell surface molecule, may permit their application as nano-lysosomal targeting chimeras ⁹⁷.

A first aspect of the invention relates to protein binders containing at least one immunoglobulin-single-variable domain (ISVD) which specifically binding human cation-independent mannose-6-phosphate receptor (CI-M6PR; also known as IGF2R), specifically recognizing a binding site located on the extracellular N-terminal domains 1, 2 and/or 3 of human CI-M6PR, as described herein (see for instance in FIG. 22). More specifically, said CI-M6PR binding agents provide for a high affinity binding to the receptor, in vitro or in cells, with a K_{D} value in the range of 100 nM or lower. More importantly, the CI-M6PR binding agents provide for a pH-dependent dissociation binding profile that is favourable for endosomal and/or lysosomal targeting when bound on cell-expressed CI-M6PR. Surprisingly, said binding agents are capable to internalize in the cells upon binding to the CI-M6P Receptor already with just the presence of a single ISVD as described herein, so bound to the M6PR in its monovalent form, via its paratope, defined by a number of amino acid residues present in just three CDRs of the antigen binding domain. Si in a specific embodiment, the CI-M6PR binding agents described herein comprise one or more ISVDs binding the cell-expressed CI-M6PR extracellular domains 1, 2 and/or 3, wherein the binding of just a single monovalent ISVD to cell-expressed CI-M6PR is sufficient to efficiently internalize the CI-M6PR-specific ISVD or binding agent, which may further contain additional moieties. Another specific embodiment relates to said ISVD specifically binding the cell-expressed CI-M6PR extracellular domains 1, 2 and/or 3, which in monovalent form internalized the cell with an internalisation rate (expressed in voxel counts/minute, as determined herein) of at least 15 counts/min, or preferably at least 50 counts per minute, or more preferably at least 100 counts per minute.

In a specific embodiment, said binding agent comprising an ISVD specifically binding CI-M6PR, specifically recognizes a binding site positioned on N-terminal domains 2 and 3, and is defined by the epitope comprising or consisting of the amino acid residues Lys191, Gly194, Ala195, Tyr196, Leu197, Phe208, Arg219, Gln224, Leu225, Ile297, Lys357, Gly408, Asp409, Asn431, Glu433, and Phe457 as set forth in SEQ ID NO:20. A further specific embodiment provides for said binding agent comprising an ISVD which specifically binds through interaction of its residues Tyr32, Arg52, Trp53, Ser54, Ser55, Ser56, Lys57, Ile100, Asp102, Phe103 and Ser108 as set forth in SEQ ID NO:8, which are in contact with the residues depicted herein as epitope in the N-terminal domains 2 and 3 of CI-M6PR.

Another specific embodiment relates to said binding agent comprising an ISVD specifically binding CI-M6PR, specifically recognizing a binding site positioned predominantly on N-terminal domain 1, and is defined by the epitope comprising or consisting of the amino acid residues Lys59, Asn60, Met85, Asp87, Lys89, Ala146, Thr147, Glu148, and Asp118 as set forth in SEQ ID NO:20. A further specific embodiment provides for said binding agent comprising an ISVD which specifically binds through interaction of its residues Asp31, Arg33, Asp35, Ser53, Tyr54, Trp56, Lys57, Lys96, Asp104, as set forth in SEQ ID NO:7,which are in contact with the residues depicted herein as epitope in the N-terminal domain 1 of CI-M6PR.

An alternative embodiment provides for a binding agent comprising an ISVD specifically binding CI-M6PR, specifically recognizing a binding site positioned predominantly on N-terminal domain 1, and is defined by the epitope comprising or consisting of the amino acid residues Lys59, Asn60, Met85, Asp87, Lys89, Ala146, Thr147, Glu148, and Gln119 as set forth in SEQ ID NO:20, which binds through interaction of its residues Asp31, Asn32, Arg33, Asp35, Thr50, Ala52, Ser53, Tyr54, Gly55, Trp56, Lys57, Asn96, Ser97, and Gly98 as set forth in SEQ ID NO:71.

In a specific embodiment, said binding agents comprising an ISVD, according to the following formula (1): FR1-CDR1-FR2-CDR2-FR3-CDR3-FR4 (1), and comprising the CDR1, CDR2 and CDR3 regions as selected from the CDR1, CDR2 and CDR3 regions of an ISVD sequence selected from the group of SEQ ID NO: 1 to 11 and SEQ ID NO:71-82, preferably from the group of SEQ ID NO: 1, 5, 7, 8, 71-73, wherein the CDR regions are annotated according to Kabat, MacCallum, IMGT, AbM, or Chothia. In a specific embodiment, said ISVDs comprise thus CDR1, CDR2, and CDR3 from SEQ ID NO:1, or CDR1, CDR2, and CDR3 from SEQ ID NO:5, or CDR1, CDR2, and CDR3 from SEQ ID NO:7, or CDR1, CDR2, and CDR3 from SEQ ID NO:8, or CDR1, CDR2, and CDR3 from SEQ ID NO:71, or CDR1, CDR2, and CDR3 from SEQ ID NO:73, wherein said CDRs may be defined according to the annotation of Kabat, MacCallum, IMGT, AbM, or Chothia, as further defined herein. In a further embodiment, the said binding agents comprising an ISVD, according to the following formula (1): FR1-CDR1-FR2-CDR2-FR3-CDR3-FR4 (1), and comprising the CDR1 corresponding to any one of the sequences as present in SEQ ID NO:103-105, 107-109, a CDR2 sequence selected from SEQ ID NO:110-112, or 114-116, and a CDR3 sequence selected from SEQ ID NO:117-119, or 121-123.

A further embodiment relates to said CI-M6PR binding agents comprising one or more ISVDs comprising or consisting of a sequence selected from the group of SEQ ID NOs: 1-11 and 71 to 82, or a sequence with at least 85% amino acid identity thereof, containing identical CDRs, or any humanized variant thereof. In a specific embodiment, said CI-M6PR binding agents comprising one or more ISVDs comprising or consisting of a sequence selected from the group of SEQ ID NO: 1, 5, 7, 8, 71, or 73, or a sequence with at least 85% identity of any one thereof, wherein the CDRs are identical, or a humanized variant thereof, such as any one of the humanized variants disclosed in SEQ ID NO:93-102. A further specific embodiment relates to the binding agent as described herein that may be a multi-specific or multivalent binding agent. More particularly bivalent or bispecific agents are envisaged herein. Even more specific, a multi-specific binding agent is envisaged, comprising an ISVD specifically binding CI-M6PR as described herein, and a second binding moiety specifically binding a cell surface or extracellular molecule. Said second binding moiety may specifically also comprise an ISVD.

An alternative embodiment relates to fusion polypeptides comprising the binding agent comprising an ISVD specifically binding CI-M6PR, or comprising a multi-specific or multivalent binding agent comprising said CI-M6PR-specific ISVD as described herein, which is connected to or fused with another polypeptide, such as an enzyme, more specifically a lysosomal enzyme, for instance an enzyme known to be applicable in Enzyme-Replacement Therapy. In a specific embodiment, said Fusions of these anti-CI-M6PR ISVD moieties to therapeutic lysosomal enzymes enables to apply these binding agents in targeted ERT in a glycan-independent way to diseased cells.

So in a specific aspect of the invention, a method is disclosed for production of the CI-M6PR-specific binding agent comprising an ISVD as described herein, preferably linked to an enzyme involved in lysosomal storage phenotypes, comprising the steps of:
a) Transformation of the nucleic acid encoding said CI-M6PR-specific binding agent in a host cell, or providing a host cell expressing said CI-M6PR-binding agent, and
b) Purifying the CI-M6PR-specific binding agent from the cell culture.

In a specific embodiment, said host cell is a Glycodelete cell as described for instance in Reference 52 cited herein.

A further specific embodiment relates to said CI-M6PR-specific binding agent comprising an ISVD as described herein, preferably linked to an enzyme involved in lysosomal storage phenotypes, produced by said method, or obtainable by said method as described herein. Said CI-M6PR-specific binding agent comprising an ISVD as described herein, preferably linked to an enzyme involved in lysosomal storage phenotypes, preferably comprises a N-glycan structure wherein one or more glycans present on said fusion protein are selected from the group of a single GlcNac, a GalGlcNac, and a SiaGalGlcNac, as for instance described in Meuris et al. (Ref. 52).

In a further specific embodiment, the fusion protein as described herein comprises one or more CI-M6PR-specific ISVDs as described herein, linked to an enzyme for lysosomal storage function, and/or obtainable by the method as described herein, wherein said enzyme is human acid alpha-glucosidase (hGAA) or a functional homologue thereof, or is human Cathepsin D or a functional homologue thereof.

In a specific embodiment said fusion protein as described herein comprises one or more CI-M6PR-specific ISVDs as described herein, linked to an enzyme for lysosomal storage function, and/or obtainable by the method as described herein, and comprises a protein sequence selected from the group of SEQ ID NO:26-33 or a functional homologue of any one thereof, which has at least 90% identity with any of SEQ ID NO:26-33.

A further embodiment relates to said CI-M6PR-specific binding agents comprising at least one ISVD as described herein, multivalent or multispecific binding agents, or the fusion proteins, comprising an ISVD of the present invention, which is labelled for detection or labelled with a tag.

Another aspect relates to a nucleic acid encoding any of the binding agents comprising an ISVD as described herein, or the further combined multi-specific binding agents or fusion proteins. Furthermore, a vector comprising said nucleic acid molecule, for expression of said binding agents or fusion proteins is disclosed herein.

A further aspect relates to a pharmaceutical composition comprising any of the binding agents described herein, multi-specific binding agents, or fusion proteins described herein.

Another aspect relates to the application or use of the binding agent, the multi-specific binding agent, the fusion protein, the nucleic acid disclosed herein, the pharmaceutical composition, in drug discovery, in structural analysis, or in a screening assay, such as for instance in structure-based drug discovery or fragment-based screening assay.

Further embodiments relate to the application or use a multi-specific binding as described herein, for instance a bispecific agent, comprising an ISVD specifically binding CI-M6PR and a second antigen binding domain for binding a target, in a method for removing and/or degrading a target that is a cell surface molecule or extracellular molecule through lysosomal uptake of said multispecific agent in the lysosome, when bound to said target. A specific embodiment further discloses the use of said binding agent, multi-specific binding agent or fusion protein as described herein for in vitro lysosomal tracking, optionally when operably linked or chemically coupled to a label.

Another aspect of the invention relates to the medical use of the binding agent, the multi-specific binding agent, the fusion protein, or the pharmaceutical composition as described herein. More specifically said agents or proteins for use as a medicine, specifically in treatment of a lysosomal storage disease, or for use in Enzyme-replacement therapy. In a specific embodiment said ERT specifically relates to a disease caused by a lysosomal storage phenotype, preferably Pompe disease, sporadic inclusion body myositis, or neuronal ceroid lipofuscinosis 10. Another embodiment of the invention relates to the multi-specific binding agent, or the pharmaceutical composition comprising said multispecific binding agent, as described herein, for use in a disorder related to the target of the second binding moiety in said multispecific binding agent, more specifically, a target which is a cell surface or extracellular molecule.

A final aspect of the invention relates to said binding agent, multi-specific binding agent, fusion protein, or labelled form thereof, for use as a diagnostic or for in vivo imaging.

### DESCRIPTION OF THE FIGURES

The patent or application file contains at least one drawing executed in color. Copies of this patent or patent application publication with color drawing(s) will be provided by the Office upon request and payment of the necessary fee.

The drawings described are only schematic and are non-limiting. In the drawings, the size of some of the elements may be exaggerated and not drawn on scale for illustrative purposes.

FIGS. 1A-1B. Purification of recombinantly produced human Domain 1-3 of CI-M6PR (hDom₁₋₃His₆) antigen protein. (FIG. 1A) immobilized nickel affinity chromatography (IMAC) followed by (FIG. 1B) size exclusion chromatography (SEC). After IMAC elution, peak fractions containing the protein of interest were pooled (A5-A10, grey labelled) and further analyzed over a SEC column. After SEC elution, peak fractions containing highly pure protein were pooled (A6-A8, grey labelled).

FIGS. 2A-2D. Purification of recombinantly produced mouse Domain 1-3 of CI-M6PR (mDom₁₋₃His₆) antigen protein, expressed by HEK293 suspension cells. (FIG. 2A) immobilized nickel affinity chromatography (IMAC) followed by (FIG. 2B) size exclusion chromatography (SEC). After IMAC elution, peak fractions containing the protein of interest were pooled (A5-A7, grey labelled) and further analyzed over a SEC column. After SEC elution, peak fractions containing highly pure protein were pooled (A8-A11, grey labelled). (FIG. 2C) additional purification of the antigen, shown as the absorbance at 280 nm (light grey) in function of volume of mCI-M6PR_{D1-D3}, obtained after IMAC. The profiles for conductivity and percentage buffer B are shown in grey and dark grey. Eluted fractions were analyzed on SDS-PAGE with coomassie Brilliant Blue. The fractions shown in the grey bar were pooled after the IMAC run and further analyzed during SEC. (FIG. 2D) Similar as in C showing the chromatogram and SDS-PAGE of the eluted fractions after SEC (light grey) with the conductivity in dark grey.

FIGS. 3A-3B. Determination of melting temperature of the 11 purified VHHs. (FIG. 3A) Left panel, fluorescence measured (Ex/Em: 498 nm/610 nm) over an increasing temperature range during which SYPRO Orange-labelled anti-CI-M6PR VHHs denature, indicative of the melting temperature at the highest fluorescence measured. Right panel: melting temperatures (Tm) as determined for every anti-CI-M6PR VHH. (FIG. 3B) Fluorescence measurements as presented in (FIG. 3A) enlarged for the area of the VHH Tms.

FIGS. 4A-4B. Affinity of anti-CI-M6PR VHHs for recombinant human CI-M6PR Domain 1-3His₆ determined by ELISA. A & B. Measurements for each of the 11 anti-CI-M6PR VHHs in serial dilution binding to the coated human CI-M6PR Domain 1-3His₆ antigen. Detection was performed using an anti-VHH antibody coupled to HRP, and measured as the absorbance of the TMB substrate at 450 nm, correlating to the amount of bound VHH as plotted at the Y-axis. The Log₁₀ concentrations of the serially diluted VHHs were plotted at the X-axis. Corresponding EC₅₀ values are outlined in Table 2. A & B represent replicates but using a different concentration of human CI-M6PR Domain 1-3His₆ (in (FIG. 4A): 0-0.75 μM and in (FIG. 4B): 0-1 μM).

FIGS. 5A-5B. Affinity of anti-CI-M6PR VHHs for recombinant mouse CI-M6PR Domain 1-3His₆ determined by ELISA. (FIG. 5A) Measurement for each of the 11 anti-CI-M6PR VHHs in serial dilution binding to the coated mouse antigen. Detection was performed using an anti-VHH antibody coupled to HRP, and measured as the absorbance of the TMB substrate at 450 nm, correlating to the amount of bound VHH as plotted at the Y-axis. The Log₁₀ concentrations of the serially diluted VHHs were plotted at the X-axis. Corresponding EC₅₀ values are outlined in the Table on the right. (FIG. 5B) Repetition for VHH1,5,7 and 8 using a further purified antigen sample (as shown in FIGS. 2 C-D).

FIG. 6. Flow cytometry analysis of the binding of anti-CI-M6PR VHHs on HEK293 cells. Normalized cell counts were plotted against PE-signal (B575). The negative control is shown in light pink; the positive control (anti-CI-M6PR antibody-PE) is shown in green. Dark pink, orange, and blue correspond to cells incubated with 200, 100 and 50 μg/mL anti-CI-M6PR VHH, respectively.

FIG. 7. Flow cytometry analysis of the binding of anti-CI-M6PR VHHs on L-D9 cells, expressing a chimeric bovine/mouse CI-M6PR. Normalized cell counts were plotted against PE-signal (B575). The negative control is shown in grey. Green, orange, blue and pink peaks correspond to cells incubated with 200, 100, 50 and 25 μg/mL anti-CI-M6PR VHH or GFP-binding protein (GBP; negative control), respectively.

FIG. 8. Flow cytometry analysis of the binding of anti-CI-M6PR VHHs on MCF7 cells. Normalized cell counts were plotted against PE-signal (B575). The negative control (i.e. GFP-binding protein (GBP) is shown in blue. The positive control, i.e. cells incubated with anti-CI-M6PR antibody (clone 2G11), is shown in orange. Peaks in red, light green, green, pink, purple, dark blue and yellow correspond to serially (1/2) diluted VHH started from 200 μg/mL.

FIG. 9. Flow cytometry analysis of the binding and internalisation of anti-CI-M6PR VHHs on MCF7 cells. Normalized cell counts were plotted against PE-signal (B575). The negative control (i.e. GFP-binding protein (GBP)) is shown in blue. Peaks in orange, red, light green, green, pink, purple, dark blue and yellow correspond to serially (1/2) diluted VHH started from 200 μg/mL.

FIG. 10. Colocalization of anti-CI-M6PR VHHs with LysoTracker (LTR)-stained endo(lyso)somes in MCF7 cells. The % of lysosomes colocalizing with the VHH are measured by MCF7 cells containing AF488-labeled anti-CI-M6PR VHHs. Incubation (45 minutes) of MCF7 cells with AF488-labelled anti-CI-M6PR VHHs and LTR Deep Red DND-99 was performed. After fixation, imaging was performed on the LSM880 Airyscan confocal microscope (Zeiss) using the 63× objective and the percentage of VHH-positive lysosomes was determined after Airyscan processing and image analysis in Volocity. The data were further processed in Graphpad Prism 9.0.0.

FIGS. 11A-11F. Live-cell imaging to monitor endocytosis and lysosomal delivery of Alexa Fluor 488 (AF488)-labelled VHHs. Every panel corresponds to one particular VHH (i.e. VHH1, VHH5, VHH7, VHH8; anti-GFP VHH and recombinant human acid glucosidase α (rhGAA) were used as negative and positive controls, respectively). Every graph per column corresponds to a particular analysis per VHH. The first graph shows the amount of endocytosed VHH-AF488 relative to the cell volume plotted over all timepoints. The second graph shows the percentage endocytosed VHH, colocalizing with lysosomes over all measured timepoints. The third graph represents the fluorescent unit-counts of intracellular labelled protein, colocalizing with late endosomes and lysosomes (green) and the counts of intra-(endo)lysosomal labelled protein (purple).

FIGS. 12A-12B. Purification of recombinantly produced anti-CI-M6PR VHH7. (FIG. 12A) Purified VHH was obtained through immobilized nickel chromatography followed by (FIG. 12B) desalting. After the immobilized metal ion chromatography, peak fractions containing the protein of interest (blue shaded) were pooled and desalted afterwards.

FIGS. 13A-13B. Purification of recombinantly produced anti-CI-M6PR VHH8. (FIG. 13A) Purified VHH was obtained through immobilized nickel chromatography followed by (FIG. 13B) desalting. After the immobilized metal ion chromatography, peak fractions containing the protein of interest (blue shaded) were pooled and desalted afterwards.

FIGS. 14A-14B. Intact mass spectrometry of anti-CI-M6PR VHHs.

FIG. 15. Association-dissociation graphs of anti-CI-M6PR VHHs analyzed using Biolayerinterferometry (BLI). Association of anti-CI-M6PR VHH1, -5, -7 and -8 shown for eight different concentrations (0-100 nM) with their antigen, human CI-M6PR-Domain1-3His₆, in the first 100 seconds in phosphate citrate buffer of pH 7.4. Afterwards, dissociation occurred during 600 seconds in phosphate citrate buffers of six different pHs (7.4, 7.0, 6.5, 6.0, 5.5, 5.0). The degree of association and dissociation was measured in δ nm over time (seconds (s)). The anti-CI-M6PR antibody and GBP were used as positive and negative controls, respectively. Figures were made with Graphpad Prism as derived from the Octet software.

FIGS. 16A-16E. Validation of VHH7 and VHH8 binding to CI-M6PR in human and mouse cell lines. Binding was confirmed after analysis of serially diluted VHH on human HEK293T (FIG. 16A) and HepG2 (FIG. 16C) cells, while absent in the HEK 293 CIM6PR^{-/-} knock-out cells (FIG. 16B). VHH7 showed to some extent cross-reactivity towards the mouse CI-M6PR of bEND3 cells (FIG. 16D), and was shown to be internalized by mouse bEND3 cells as detected upon permeabilization (FIG. 16E), whereas the latter occurs only to a very limited extent for VHH8. IRR is an irrelevant Nb as negative control, the positive control is a non-disclosed compound known to bind a cell-surface expressed human and mouse target. Right panels shown binding profiles for anti-CI-M6PR 2G11 antibody (Abcam ab2733), used as a positive control. MFI, median fluorescence intensity.

FIGS. 17A-17B. Overview of radioactivity retention in major organs of C57BL/6J mice injected with radioactively labelled anti-CI-M6PR VHHs. The eleven anti-CI-M6PR VHHs were radioactively labelled and intravenously injected into C57BL/6J mice. Three hours after injection, the radioactivity in each of the above tissues was analyzed with a gamma counter. The measurements were normalized for the injected activity per VHH and their normalized radioactivity was plotted in injected dose (ID) per gram. (FIG. 17A) VHH1-6; (FIG. 17B) VHH7-11 and GBP negative control VHH.

FIGS. 18A-18B. Multi-angle Light Scattering of anti-CI-M6PR VHH8 and hDom1-3His6 protein complex. Both proteins were incubated (1:1) and MALLS analysis was performed after size exclusion chromatography. Eluted fractions were analysed on SDS-PAGE (samples were incubated with Laemli buffer (without) containing DTT) and western blot (anti-His DyLight800 (1/15000, Rockland)).

FIGS. 19A-19D. Size exclusion chromatography (SEC) of either anti-cation independent mannose-6-phosphate receptor (hCI-M6PR) VHH7 and VHH8 in complex with hCI-M6PR_{D1-D3} (1:1) and SDS-PAGE of crystals from each of the protein complexes. (FIG. 19A) SEC run of anti-CI-M6PR VHH7: hCI-M6PRD1-D3 (1:1) complex. (FIG. 19B) SEC run of anti-CI-M6PR VHH8:hCI-M6PR_{D1-D3} (1:1) complex. (FIG. 19C) Analysis of 1/3 diluted complexed anti-CI-M6PR VHH7:hCI-M6PR_{D1-D3} protein (+), wash fraction, which was used to remove the mother liquor from the crystal (wash) and the proteins from the washed crystal (crystal) generated in 0.1M ammonium sulphate, 0.3 M sodium formate, 0.1 M sodium cacodylate, pH 6.36, 6.5% w/v γ-PGA (Na+ form, LM) 4% w/v PEG 3350) on silver stained SDS-PAGE. (FIG. 19D) Similar analysis as in C, with 1/10 diluted complexed anti-CI-M6PR VHH8:hCI-M6PR_{D1-D3} protein from proplex D10 (2:1) crystallization condition (+) on silver stained SDS-PAGE.

FIG. 20. Cocrystal structure of VHH7 and domains 1-3 of the hCI-M6PR. Being coloured according to the rainbow spectrum from N- to C-terminus, the three CDR regions of VHH7 are shown in blue, green and orange. Residues that make up the epitope are shown as grey spheres (left). As VHH7 binds only the first domain of the CI-M6PR, a detailed figure of this and VHH7 is shown in the middle and on the right.

FIG. 21. Cocrystal structure of VHH8 and domains 1-3 of the hCI-M6PR. Being coloured according to the rainbow spectrum from N- to C-terminus, the three CDR regions of VHH8 are shown in blue, green and orange. Residues that make up the epitope are shown as grey spheres (left). The homologues residues from the bovine CI-M6PR of the epitope are shown in purple (middle). The tertiary structure of all CI-M6PR domains individually is conserved; as an example, a detailed figure of the flattened @3-barrel of domain 3 of the CI-M6PR is shown on the right.

FIG. 22. Amino acid sequence alignment of CI-M6PR domains 1-3 for human, mouse and bovine proteins and indication of the VHH7/1H11 and VHH8 epitope residues. Bovine (B/, Bos taurus), human (H/, Homo sapiens) and mouse (M/, Mus musculus) CI-M6PR Domain 1-3 sequences multiple alignment, showing the three different domains of the antigen, Domain 1 (D1; bovine residues 49-171), domain 2 (D2; bovine res. 172-325) and domain 3 (D3; bovine res. 326-476). Full circles represent the core epitope residues selected based on integrating the outputs of the 4 Angstrom distance of the VHH, PISA and FastContact analysis. Half circles define further residues within 4 Angstrom distance of the VHH.

FIGS. 23A-23B. Post-translational processing of human acid α-glucosidase (hGAA) and cathepsin D (CTSD). (FIG. 23A) hGAA processing from primary translation product to mature protein with the removed amino acids at positions 57-78, 113-122 and 781-792 shown in grey. (FIG. 23B) hCTSD processing.

FIGS. 14A-14B. Purification of cathepsin D in HEK293 and HEK293 GlycoDelete cells. (FIG. 14A) The upper chromatogram shows the immobilised metal ion chromatography (IMAC) capturing step of recombinant cathepsin D produced by HEK293 suspension cells in which the milli absorbance units (mAU) at 280 nm are shown in function of volume. His-trapped proteins were subsequently eluted by gradually increasing the imidazole concentration (% buffer B shown by the green curve) and decreasing the amount of NaCl (conductivity shown by the brown curve). The collected fractions (grey) from IMAC were subjected onto a superdex 200 pg column for size exclusion chromatography (lower chromatogram). Multiple superdex runs were performed for every IMAC, the chromatogram shown is representative for each run. During each step of the purification, the eluted fractions were analysed on SDS-PAGE, stained by Coomassie Brilliant Blue. (FIG. 14B) Similar for the purification of cathepsin D, produced by GlycoDelete cells.

FIGS. 25A-25C. Glycoforms of CTSD display little difference in specific activity. (FIG. 25A) HEK293- and HEK293 GlycoDelete-produced CTSD, digested with PNGase F and analysed on western blot. Both proteins contain a His₆-tag and are detected by anti-His antibody DeLight800. (FIG. 25B and FIG. 25C) Michaelis-Menten curve for the CTSD glycoforms' single-substrate reactions, showing the relation between the reaction rate (velocity) and substrate concentration. Values for the catalytic efficiency constant and Michaelis Menten constant (K_{M}) are outlined with standard error.

FIGS. 26A-26B. DSA-FACE analysis of recombinant Cathepsin D protein, produced in HEK293 and GlycoDelete cells. (FIG. 26A) Chromatograms showing either dextrane polymer, RNAseB Man₅₋₉GlcNAc₂ N-glycans (i.e. M5, M6, M7, M8, M9 glycans) and profiles of wild-type cathepsin D glycans, treated with several glycosidase enzymes or calf intestinal phosphatase (CIP). Mannose (green circles), N-acetylglucosamine (blue rectangles), galactose (yellow circles), sialic acid (purple diamonds) and fucose (red triangles); Pi is for phosphate. (FIG. 26B) DSA-FACE analysis of PNGaseF digestable glycans that remain on GlycoDelete produced CTSD. Mannoses are shown as green circles, Pi is for phosphate. Additional profiles show chromatograms of dextran and RNAseB Man₅₉GlcNAc₂ N-glycans (i.e. M5, M6, M7, M8, M9 glycans) are represented.

FIGS. 27A-27F. Expression of Cathepsin D (CTSD) and VHH fusion proteins. (FIG. 27A) Western blot of supernatant from transfected HEK293 with CTSD fused N-terminally to anti-CI-M6PR receptor VHHs (1-11; SEQ ID NO:1-11). Both proteins were linked by a triple Gly₄Ser linker (L) and contained a FLAG₃His₆ tag (SEQ ID NO: 22) at the C-terminus. Detection performed using anti-His antibody DyLight800. (FIG. 27B) Similar to FIG. 27A, in which VHH (1-11)-CTSD fusion proteins were detected. As negative control, GFP expression was performed (−), while pro-CTSD-His₆ was used as positive control (+). (FIG. 27C) Intracellular fraction of cells expressing VHH-CTSD proteins, detected using anti-His₆ antibody DyLight800. Controls (− and +) are similar as in FIG. 27B. (FIG. 27D) Similar to FIG. 27B: the upper western blot uses an anti-CTSD antibody detection; the lower blot uses an anti-camelid antibody-HRP. Controls (− and +) are similar as in FIG. 27B. (FIG. 27E) Investigation of different linkers in between the anti-CI-M6PR VHH7 (lane 1-5) and 8 (lane 7-11) and CTSD: (Gly₄Ser)₃ (lane 1 and 7), GlySer (lane 2 and 8) (GluAlaAlAlaLys)₃ (lane 3 and 9), (AlaPro)₇ (lane 4 and 10) and Gly8 (lane 5 and 11). Lane 6 and 12 contain CTSD-(G₄S)₃-VHH7 and -VHH8 respectively. The negative control (−) contains supernatant from cells expressing GFP, the positive (+) control is pro-CTSD-His₆. (FIG. 27F) Proteolytic activity (in relative fluorescence units (RFU)) of secreted CTSD and VHH chimeric construct, pro-CTSD-His₆ and negative controls (GFP and Freestyle293 medium) on synthetic substrate.

FIGS. 28A-28E. Purification of cathepsin D (CTSD) fused to anti-CI-M6PR VHH7, produced in HEK293 (FIG. 28A and FIG. 28B) and HEK293 GlycoDelete cells (FIG. 28C and FIG. 28D). (FIG. 28A) Results obtained from immobilised metal ion chromatography (IMAC) of recombinant CTSD-VHH7 in which the milli absorbance units (mAU) at 280 nm are shown in function of volume. His-trapped proteins were subsequently eluted by gradually increasing the imidazole concentration (% buffer B with 400 mM imidazole shown by the green curve) and decreasing the amount of NaCl (conductivity in mS/cm shown by the brown curve). (FIG. 28B) The collected fractions (grey) from IMAC were subjected onto a superdex 200 pg column for size exclusion chromatography. During each step of the purification, the eluted fractions were analysed on SDS-PAGE, stained by Coomassie Brilliant Blue. (FIG. 28C) The IMAC purification step for the capturing of CTSD-VHH7, produced by GlycoDelete cells. (FIG. 28D) Size exclusion chromatography of the IMAC-eluted fractions containing GlycoDelete CTSD-VHH7. Marker: Precision all blue protein standard. (FIG. 28E) Table giving the obtained protein yield of each CTSD-VHH.

FIGS. 29A-29E. Generation of Cathepsin D knockout HeLa and C2C12 knockout cell lines. (FIG. 29A) To generate mutations in the CTSD gene, human HeLa and mouse C2C12 cells were transfected with a single guide RNA targeting exon 2. (FIG. 29B) CTSD insertions and deletions in the HeLa cell lines were confirmed by DNA sequencing and further mapped by the Synthego Performance Analysis, ICE Analysis (2019. v2.0). The protospacer adjacent motif is underlined with a dashed red line, the sequence of the single guideRNA was underlined in black and the Cas9 cutting site was shown by a dotted black line. (FIG. 29C) similarly for the mouse C2C12 cell line. FIG. 29 (D) Intracellular activity of CTSD was analysed by measuring the fluorescence of a CTSD-cleavable peptide. The relative fluorescence units after 95 minutes of incubation for each clone was outlined and compared wild-type clones and recombinant CTSD (n=3). (FIG. 29E) For the two most relevant clones, the RFU was measured over time and compared to the recombinant CTSD (rhCathD), wild-type HeLa and C2C12 cells (n=3).

FIGS. 30A-30B. Intracellular CTSD activity, monitored by measuring the relative fluorescence units (RFU) after proteolytic cleavage of a synthetic substrate (Mca-GKPILFFRL(dinitrophenyl)DR-NH₂). (FIG. 30A) HeLa CTSD^{-/-} and (FIG. 30B) C2C12 CTSD^{-/-} cells incubated with 200 nM of HEK293 suspension (S) or GlycoDelete (GD)-produced pro-hCTSD-VHH(7/8), pro-hCTSD or culture medium for 1 h, 3 h, 6 h, 8 h and 24 h in duplicate. The measured fluorescence (λ_{Ex} Mca: 320 nm λ_{Em} Mca: 420 nm) at each time point was outlined, together with the endogenous CTSD activity in wild-type (WT) cells.

FIGS. 31A-31C. Internalisation and lysosomal targeting of recombinant human Cathepsin D (CTSD) (un)fused to anti-cation-independent mannose-6-phosphate receptor VHH7/8. AF488-chimeric proteins of the rhCTSD and C-terminally fused VHH7 and VHH8 were incubated for four hours on HeLa CTSD^{-/-} cells at 37° C. Lysosomal targeting was assessed by anti-LAMP1 staining and colocalisation analysis afterwards. The latter was performed in triplicate. (FIG. 31A) The absolute voxel counts of intracellular AF488-proteins. Replicates, i.e. specific positions in the well, are numbered (No). (FIG. 31B) The fraction of intracellular proteins colocalising with LAMP1-stained lysosomes. Replicates are numbered (No). (FIG. 31C) Images showing DAPI-stained nuclei (cyan), AF488-proteins (green) and LAMP1-lysosomes (magenta). Imaging was performed on the LSM880 Airyscan confocal microscope (Zeiss) using the 63× objective. The processed data were further processed in Graphpad Prism 9.0.0.

FIGS. 32A-32C. Pharmacokinetics of recombinant human Cathepsin D (cathD), whether or not fused to an anti-cation-independent mannose-6-phosphate receptor VHH and produced in wild-type HEK293 cells (S) or GlycoDelete cells (GD). (FIG. 32A) experimental design: 57BL/6J mice (n=5) were injected with recombinant protein (5 mg/kg) and have been micturated and blood sampled after 30 minutes, 1 h, 3 h, 6 h and 16 h. (FIG. 32B) Quantification of cathepsin D in blood serum by ELISA (duplicate); showing the absorbance (450 nm) in function of the sampling timing. (FIG. 32C) Results of the ELISA assay (duplicate) on urine samples. The graphs show the absorbance (450 nm) in function of the micturition timing. Graphs were created in Graphpad prism.

FIGS. 33A-33F. Expression and glycan analysis of acid glucosidase α (rhGAA) and expression and purification of rhGAA fused to anti-cation-independent mannose-6-phosphate receptor VHHs. (FIG. 33A) Optimisation of (non-)codon optimised ((N)CO) rhGAA expression by HEK293 cells from different expression vectors. Left: secreted fractions, Right: intracellular fractions. Green fluorescent protein (GFP) was transfected as control. Detection performed with anti-His antibody DyLight800. (FIG. 33B) Further optimisation of rhGAA expression after HEK293 transfection with pcDNA3.1-rhGAA(NCO) by administration of M6P (10 mM), sodium butyrate (NaBut, 3 mM), n-acetyl cystein (NAC, 10 mM). GFP was transfected as negative control. (FIG. 33C) Expression of hGAA, fused N- or C-terminally to anti-CI-M6PR VHHs (i.e. hGAA-VHH and VHH-hGAA). Additionally, HEK293- and GlycoDelete expression of hGAA-VHH8 every day post transfection (D1-6) was analysed and EndoT processing was monitored. (FIG. 33D) N-glycosylation profile analysis of rhGAA. The dextran panel is a malto-oligosaccharide standard with a single glucose unit corresponding to the peak-to-peak shift per carbohydrate unit. Below these two panels, the untreated glycans are shown and multiple panels of glycans treated with (combination of) exoglycosidases, indicated as such in the profile. The * indicates the peaks possibly corresponding to mannose-6-phosphorylated glycans. RNAseB represents an N-glycan standard with a typical profile consisting of Man₅₋₉GlcNAc₂ N-glycans (M5-M9). (FIG. 33E) IMAC purification of rhGAA-VHH8, expressed by HEK293 GlycoDelete cells. Fractions annotated in grey on the SDS-PAGE were pooled and subsequently subjected on a superdex 200 pg size exclusion column. (FIG. 33F) SEC of IMAC captured rhGAA-VHH8 proteins. Fractions annotated in grey were pooled and concentrated.

FIGS. 34A-34B. Amplex red and glucosidase assay to monitor the intracellular acid glucosidase α activity and degradation of its substrate glycogen inside the GAA−/− fibroblasts. (FIG. 34A) Lysates of cells treated with rhGAA-VHH7, -5, -8 and rhGAA were treated with amylase after which the obtained glucose was quantified in an Amplex red assay. (FIG. 34B) The same lysates were used in an activity assay with 4-methylumbelliferyl α-D-glucopyranoside, an artificial fluorescent substrate for GAA, to monitor the intracellular activity.

FIGS. 35A-35C. Internalisation and lysosomal targeting of recombinant human acid glucosidase α (rhGAA) N-terminally fused to anti-cation independent mannose-6-phosphate receptor VHH8 and rhGAA and VHH8 alone. AF488-labelled chimeric proteins of the rhGAA and C-terminally fused VHH8 and rhGAA and VHH8 as such were incubated for 4 h on HeLa CTSD^{-/-} cells (clone 3D5) at 37° C. for 4 h. Lysosomal targeting was assessed by anti-LAMP1 staining and colocalisation analysis afterwards. The latter was performed in triplicate. (FIG. 35A) Absolute voxel counts of intracellular AF488-labelled proteins. (FIG. 35B) The fraction of intracellular proteins colocalising with LAMP1-stained lysosomes. (FIG. 35C) Images showing DAPI-stained nuclei (cyan), AF488-labelled proteins (green) and LAMP1-stained lysosomes (magenta). Imaging was performed on the LSM880 Airysccan confocal microscope (Zeiss) using the 63× objective. The data obtained after image processing were further processed in Graphpad Prism 9.0.0.

FIGS. 36A-C. Overview of the binding affinity of the anti-CI-M6PR VHHs, whether or not fused to cathepsin D (CTSD) against human Dom₁₋₃His₆ as determined by ELISA. (FIG. 36A) ELISA assay in which each construct was serially diluted and incubated on human CI-M6PR_{D1-D3} and the detected absorbance (Abs) at 450 nm was set out in function of concentration. Curves were non-linearly fitted and EC₅₀ values were determined and outlined in the table. (FIG. 36B and FIG. 36C) Cell surface binding of CTSD-VHH fusion proteins on mouse myoblasts (C2C12 cells), analysed by flow cytometry. Graphs created in Graphpad Prism 9.0.0.

FIG. 37. Pharmacokinetics of recombinant human Cathepsin D, produced in wild-type HEK293 cells (S) or GlycoDelete cells (GD). Results of an ELISA assay on serum samples from C57BL/6J mice (n=5) injected with recombinant cathepsin D (5 mg/kg) and sacrificed for blood sampling after, 1 h, 3 h, 6 h, 16 h and 48 h. The results show the quantification of cathepsin D in blood serum by ELISA in duplicate with the absorbance (450 nm) in function of the time points of blood sampling.

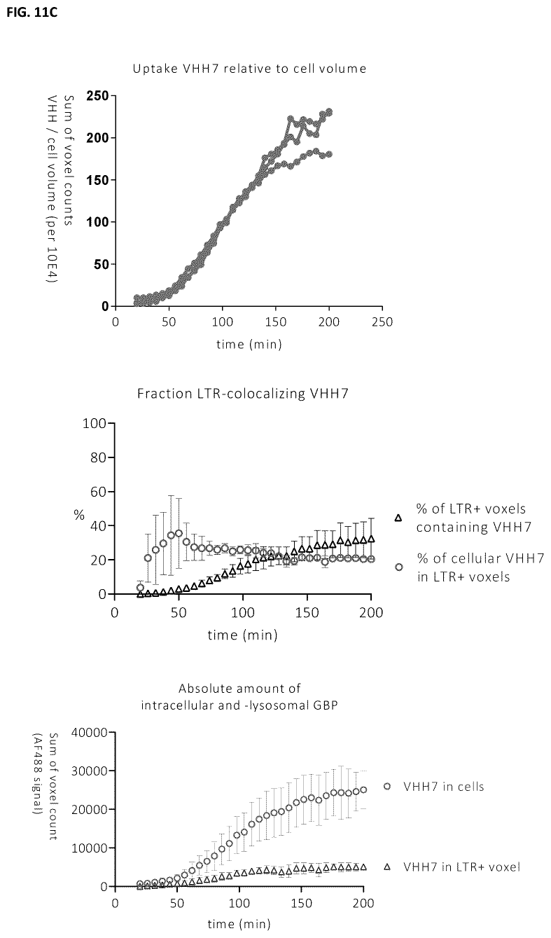

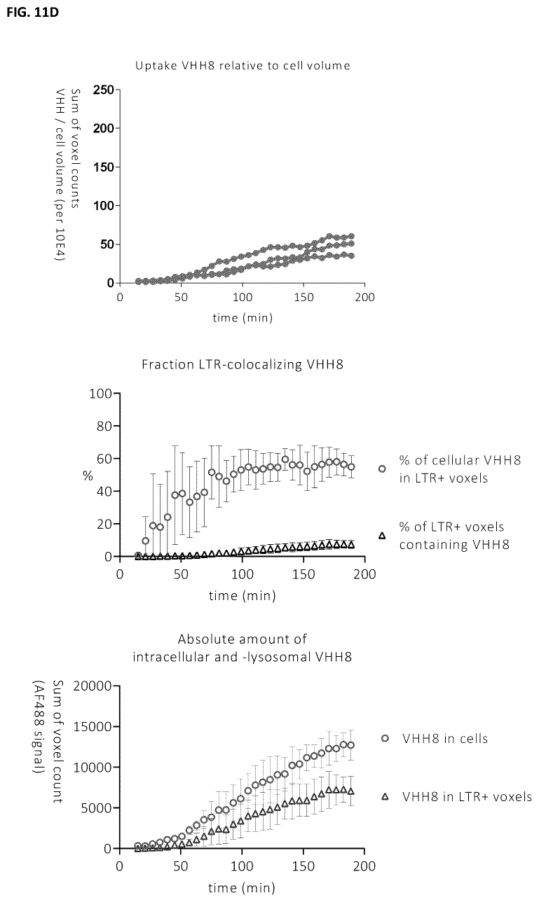

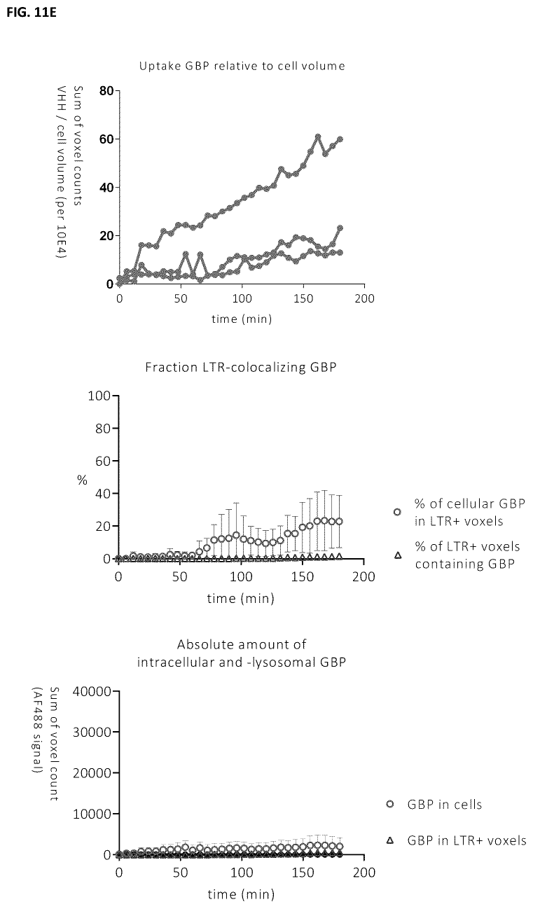

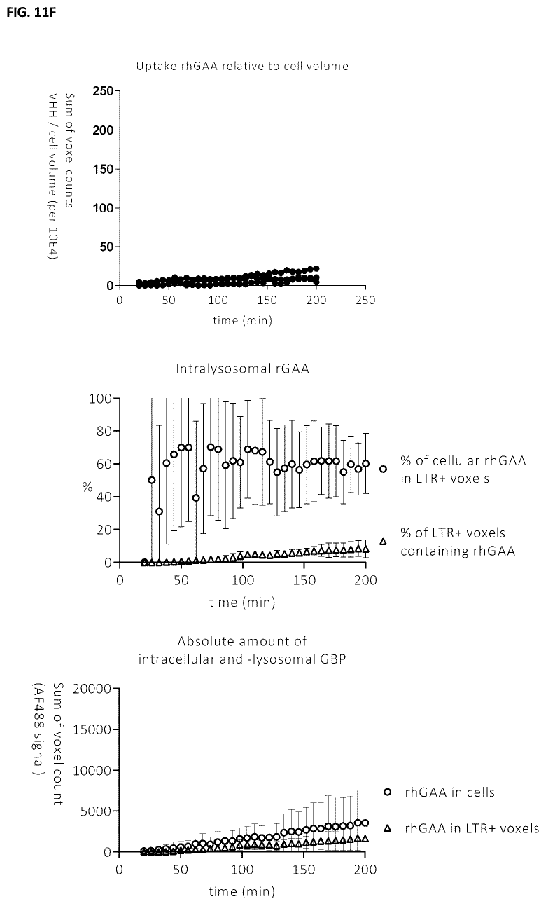

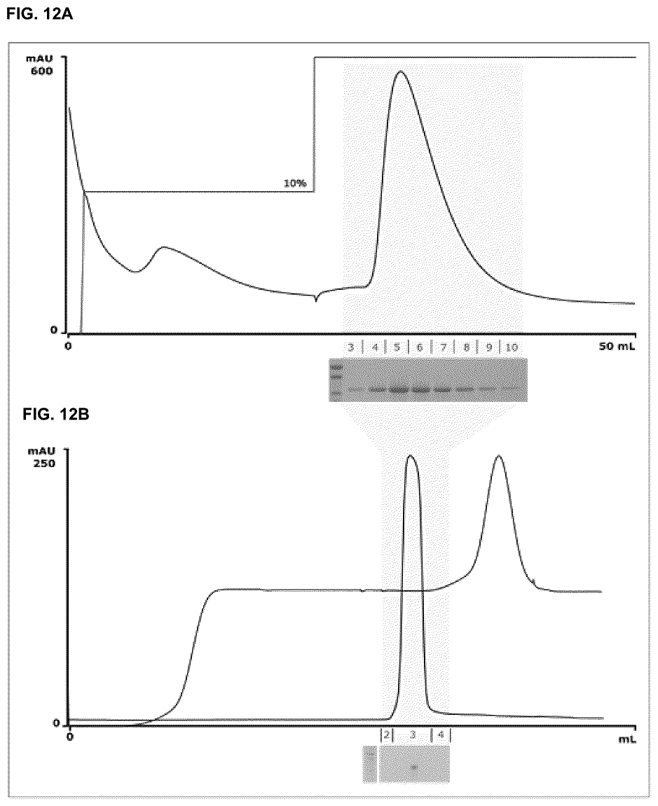

FIG. 38. Primary images corresponding to the live-cell imagingg raphs shown in FIG. 11. (A-F) Show a particular VHH (i.e. VHH7, -1, -5, -8, negative control (GBP) or recombinant human acid α-glucosidase (rhGAA), used as positive control) that were fluorescently labelled to Alexa Fluor 488. For each image, the most appropriate Z-stack was selected at 120 minutes of incubation and intracellular protein (green) was shown together with the LysoTracker (magenta) and bright-field signal. Imaging was performed on the Zeiss Spinning Disk microscope with the Plan-Apochromat 40× (1.40 oil DIC UV-Vis-IR M27) objective.

FIGS. 39A-39C. Microscopic analysis of internalized and intralysosomal anti-CI-M6PR VHH7 and VHH8. Alexa Fluor 488 (AF488)-labelled VHHs were incubated for four hours on HeLa cells (37° C.) and stained with an anti-LAMP1 antibody that was detected using a DyLight594 coupled antibody. (FIG. 39A) Percentage of endocytosed anti-CI-M6PR VHH-AF488, detected in LAMP1-positive lysosomes. (FIG. 39B) Percentage of LAMP1-stained lysosomes, containing VHH7 and VHH8. (FIG. 39C) Images corresponding to AF488-VHH7, AF488-VHH8 treated and untreated (medium) cells (green) colocalising with LAMP1(magenta). Nuclei were stained with DAPI (cyan). Imaging of three fields of view was performed for every VHH-AF488 on the LSM880 Airyscan confocal microscope (Zeiss) in SR mode using the 63× objective.

FIGS. 40A-40B. SEC-MALLS analysis of VHH8/7 and human cation-independent mannose-6-phosphate receptor domains 1-3 (hCI-M6PR_{D1-D3}) and their protein complexes. Each chromatogram shows the calculated molecular weight in function of time for the UV spectrum of each sample: (FIG. 40A) Chromatogram of hCI-M6PR_{D1-D3}:anti-hCI-M6PR VHH8 protein complex (1:1) (red). The non-complexed protein, hCI-M6PR_{D1-D3} (blue) and anti-CI-M6PR VHH8 (red) are included as well. (FIG. 40B), Similar as A but for the hCI-M6PR_{D1-D3}:anti-CI-M6PR VHH8 protein complex (1:1) (purple), hCI-M6PR_{D1-D3} (blue) and anti-hCI-M6PR VHH8 (purple). Data was obtained as described in the method section; graphs were created in Graphpad Prism 9.0.0.

FIGS. 41A-41D. Cartoon presentation of the co-crystal structure of VHH7 and domains 1-3 of the hCI-M6PR. (FIG. 41A) VHH7 is coloured in black with its paratope residues (shown as sticks), facing domain 1 (D1) of the CI-M6PR (grey). A detailed figure of the CI-M6PR epitope of VHH7 is shown in FIG. 41B and FIG. 41C. (FIG. 41B) Detailed interface of CI-M6PR D1, displayed as a surfaced cartoon, and sticked paratope residues of CDR1, -2 and -3 of VHH7. (FIG. 41C) Detailed interface of VHH7, displayed as a surfaced cartoon and the epitope residues of CI-M6PR D1 shown as sticks. (FIG. 41D) Shows the paratope residues of VHH7 (black) within less than 4 Å from the epitope region on D1 (grey).

FIGS. 42A-42D. Cartoon presentation of the co-crystal structure of VHH8 and domains 1-3 of the hCI-M6PR. (FIG. 42A) VHH8 is coloured in black with its paratope facing domain 2 (D2) and D3 of the CI-M6PR (grey). A detailed figure of the CI-M6PR epitope of VHH8 is shown in B and C. (FIG. 42B) Detailed interface of CI-M6PR D2 and D3, displayed as a surfaced cartoon (light grey), and sticked paratope residues of CDR1, -2 and -3 of VHH7 (dark grey). (FIG. 42C) Detailed interface of VHH8, displayed as a surfaced cartoon and the epitope residues of CI-M6PR D2 and D3 shown as sticks. (FIG. 42D) Shows the paratope residues of VHH8 (black) within less than 4 Å from the epitope region on D1 (grey).

FIGS. 43A-43D. Cartoon presentation of the co-crystal structure of VHH 1H11 and domains 1-3 of the hCI-M6PR. (FIG. 43A) VHH 1H11 is coloured in black with its paratope residues (shown as sticks), facing domain 1 (D1) of the CI-M6PR (grey). A detailed figure of the CI-M6PR epitope of VHH 1H11 is shown in FIG. 43B and FIG. 43C. (FIG. 43B) Detailed interface of CI-M6PR D1, displayed as a surfaced cartoon, and sticked paratope residues of CDR1, -2 and -3 of VHH 1H11. (FIG. 43C) Detailed interface of VHH 1H11, displayed as a surfaced cartoon and the epitope residues of CI-M6PR D1 shown as sticks. (FIG. 43D) Shows the paratope residues of VHH 1H11 (black) within less than 4 Å from the epitope region on D1 (grey).

FIGS. 44A-44B. Schematic presentation of the binding of anti-CI-M6PR VHHs to domains 1-3 of the hCI-M6PR. (FIG. 44A) The trefoil-shaped structure of CI-M6PR_{D1-D3} (similar to PDB: 1q25) presented schematically (white) with VHH7 and VHH8 bound to either D1 and D2-D3 respectively (grey). (FIG. 44B) Same as A but with CI-M6PR_{D1-D3} being similar to PDB: 6p8i and binding VHH 1H11 to D1 (grey).

FIG. 45. Crystal structure information of N-terminal three domains of the cation-independent mannose-6-phosphate receptor in complex with anti-CI-M6PR VHH7. Observed crystal contacts in the VHH7:hCI-M6PR_{D1-D3} structure; crystal packing enabled by Asn112-linked glycan of one protein and the M6P-binding pocket in hCI-M6PR_{D3} of another protein. Figures were created in PyMol 2.3.3.

FIG. 46. Amino acid sequence alignment of VHH7 and its humanized variants. A multiple sequence alignment of VHH7 and its humanized variants was performed using ClustalW.

FIG. 47. Amino acid sequence alignment of VHH8 and its humanized variants. A multiple sequence alignment of VHH8 and its humanized variants was performed using ClustalW.

FIGS. 48A-48E. Association-dissociation graphs of humanized VHH7 variants analyzed using Biolayer interferometry (BLI). BLI was performed on an Octet Red96 (FortéBio) instrument in kinetics buffer (0.2 M Na₂HPO₄, 0.1 M Na⁺ citrate, 0.01% bovine serum albumin, 0.002% Tween-20). Biotinylated human domain₁₋₃His₆ was immobilized on Streptavidin SA biosensors (Sartorius) to a signal of 0.6 nm. A 120 s association phase in VHH7 (FIG. 48A), VHH7 h1 (FIG. 48B), VHH7 h2 (FIG. 48C), VHH7 h3 (FIG. 48D) or VHH7hWN (FIG. 48E) serially diluted (0-200 nM) in pH 7.4 phosphate citrate buffer, was followed by 420 s of dissociation in phosphate buffer at either pH 7.4, 6.5, 6.0, 5.5 or 5.0. Between runs, biosensors were regenerated by three times 10 s exposure to regeneration buffer (10 mM glycine pH 3). The degree of association and dissociation was measured in δ nm over time (s). Black curves represent the double reference-subtracted data that were fitted according to the 1:1 binding model (grey dashed line).

FIGS. 49A-49E. Association-dissociation graphs of humanized VHH8 variants analyzed using Biolayer interferometry (BLI). BLI was performed on an Octet Red96 (FortéBio) instrument in kinetics buffer (0.2 M Na₂HPO₄, 0.1 M Na⁺ citrate, 0.01% bovine serum albumin, 0.002% Tween-20). Biotinylated human domain₁₋₃His₆ was immobilized on Streptavidin SA biosensors (Sartorius) to a signal of 0.6 nm. A 120 s association phase in VHH8 (FIG. 49A), VHH8 h1 (FIG. 49B), VHH8 h2 (FIG. 49C), VHH8 h3 (FIG. 49D) or VHH8hWN (FIG. 49E) serially diluted (0-200 nM) in pH 7.4 phosphate citrate buffer, was followed by 420 s of dissociation in phosphate buffer at either pH 7.4, 6.5, 6.0, 5.5 or 5.0. Between assays, biosensors were regenerated by three times 10 s exposure to regeneration buffer (10 mM glycine pH 3). The degree of association and dissociation was measured in δ nm over time (s). Black curves represent the double reference-subtracted data that were fitted according to the 1:1 binding model (grey dashed line).

FIGS. 50 & 51. In-tandem competitive BLI of purified anti-CI-M6PR VHHs. In-tandem competitive BLI was performed on an Octet Red96 (FortéBio) instrument in kinetics buffer (1×PBS, 1 mg/ml bovine serum albumin, 0.02% Tween-20 and 0.05% sodium azide). Human CI-M6PR domain₁₋₃His₆ (0.5 mg/mL in 50 mM MES, 150 mM NaCl, pH 6.5) was incubated for 30 minutes at room temperature with EZ-Link™ NHS-PEG4-Biotin (1 mg, Thermo Fischer A39259) and NaHCO₃ (100 mM). Biotinylated human domain₁₋₃His₆ was purified using a Zeba spin desalting Column™ (7K MWCO, 2 mL, Thermo Fischer 89890) and immobilized on Streptavidin SA biosensors (Sartorius) to a signal of 0.5 nm. In a competitive assay (left), a 60 s association phase in 400 nM purified VHH7 (top) or VHH8 (bottom) was followed by a second association phase in: 400 mM of one of a range of anti-CI-M6PR VHHs recombinantly produced in and purified from E. coli (FIG. 50), or in a periplasmic extract of E. coli expressing one of a range of anti-CI-M6PR VHHs (FIG. 51). In a second reverse assay (right), a 60 s association phase either in 400 nM anti-CI-M6PR-VHH recombinantly produced in and purified from E. coli (FIG. 50), or in a periplasmic extract of E. coli expressing one of a range of anti-CI-M6PR VHHs (FIG. 51) was followed by a second 60 s association phase in 400 nM VHH7 or VHH8. Between assays, biosensors were regenerated by three times 10 s exposure to regeneration buffer (10 mM glycine pH 3). Data were double reference-subtracted and aligned in Octet Data Analysis software v9.0 (FortéBio). Greyscale curves represent double reference-subtracted data. A competition table indicates which combinations of saturating and competing VHHs (all at 400 nM in FIG. 50) resulted in competition or non-blocking interactions.

FIG. 52. Amino acid sequence alignment of alternative anti-CI-M6PR VHHs developed by LinXis BV. A multiple sequence alignment of the VHH-sequences described by Houthoff et al. (WO2020/185069A1) was performed and sequences were clustered based on CDR3 sequence identity (wherein CDR3 is indicated in the boxed area). One representative VHH of each CDR3-family was selected for production and evaluation of binding of human CI-M6PR Dom₁₋₃His₆ and epitope competition with VHH7 or VHH8. Dots indicate identical amino acid residues as the above; ‘-’ indicates no amino acid residue is present at this position.

FIG. 53. SDS-PAGE-analysis of the produced representative for each family of the alternative anti-CI-M6PR VHHs developed by LinXis BV. One representative VHH of each CDR3-based VHH family identified from the set of VHHs developed by LinXis BV was produced in E. coli. The purified proteins were separated through SDS-PAGE and the gel was e-stained. ‘MM’=molecular weight marker.

FIG. 54. In-tandem competitive BLI of alternative anti-CI-M6PR VHHs developed by LinXis BV. In-tandem competitive BLI was performed as described in FIG. 50. In a first competitive assay (left), a 60 s association phase in 400 nM purified VHH7 (top) or VHH8 (bottom) was followed by a second association phase in a 400 nM purified VHH. In a reverse assay (right), a 60 s association phase in 400 nM purified VHH was followed by a second 60 s association phase in 400 nM VHH7 or VHH8.

FIG. 55. In-tandem competitive BLI of humanized VHH7 and humanized VHH8 variants. In-tandem competitive BLI was performed as described in FIG. 50. In a first competitive assay (left), a 60 s association phase in 400 nM purified VHH7 (top) or VHH8 (bottom) was followed by a second association phase in 400 nM of a purified humanized VHH7 or VHH8 variant. In a reverse assay (right), a 60 s association phase in 400 nM of a purified humanized VHH7 or VHH8 variant was followed by a second 60 s association phase in 400 nM VHH7 or VHH8.

FIG. 56. Gel filtration chromatograms (left) and SDS-PAGE (right) of hDom₁₋₃His₆ complexed with anti-CI-M6PR proteins VHH1, VHH5, VHH1H11 and VHH1H52. Letters A to F indicate the elution fractions analysed on SDS-PAGE, ‘inj’ indicates the injected sample. Grey rectangles on the chromatograms indicate which fractions were pooled for structural studies.

FIG. 57. Coomassie Brilliant Blue-stained SDS-PAGE of hDom₁₋₃His₆ complexed with anti-CI-M6PR proteins VHH1, VHH5, VHH 1H11 and VHH 1H52, samples used for co-crystallization.

FIGS. 58 & 59. Association-dissociation graphs of anti-CI-M6PR VHH1H11 and VHH1H52, resp. analyzed using BLI. BLI was performed on an Octet Red96 (FortéBio) instrument in kinetics buffer (0.2 M Na₂HPO₄, 0.1 M Na⁺ citrate, 0.01% bovine serum albumin, 0.002% Tween-20). Biotinylated human domain₁₋₃His₆ was immobilized on Streptavidin SA biosensors (Sartorius) to a signal of 0.6 nm. A 120 s association phase in VHH 1H11 (FIG. 58) or VHH 1H52 (FIG. 59) serially diluted (0-200 nM) in pH 7.4 phosphate citrate buffer, was followed by 420 s of dissociation in phosphate buffer of either pH 7.4, 6.5, 6.0, 5.5 or 5.0. Between assays, biosensors were regenerated by three times 10 s exposure to regeneration buffer (10 mM glycine pH 3). The degree of association and dissociation was measured in δ nm over time (s). Black curves represent the double reference-subtracted data that were fitted according to the 1:1 binding model (grey dashed line).

FIGS. 60 & 61. ELISA binding profiles of different VHH species on CI-M6PR VHH7-epitope mutants, and VHH8-epitope mutants, resp. ELISA was performed using 100 ng coated CI-M6PR mutants, a dilution series of the different indicated VHHs, with a no-VHH background control, and detection using MonoRab anti-VHH-HRP. Data were visualized and analyzed using the GraphPad Prism 9 software.

FIGS. 62 & 63. EC50 values deduced from the ELISA binding profiles for different VHH species on CI-M6PR VHH7-epitope mutants or VHH8-epitope mutants, resp. EC50 values (in nM) were deduced from the graphs shown in FIGS. 60 & 61, resp. using the GraphPad Prism 9 software. For VHH1, no EC50 could be determined for the CI-M6PR-M85E mutant (ND=not determined).

FIG. 64. Amino acid sequences of VHH7 and VHH8 with annotated CDRs. Kabat numbering is used for numbering of the amino acid residues. The Complementary-determining-regions 1, 2 and 3 (CDR1,2, 3) are indicated as grey labelled boxed, according to AbM, MacCallum, Chothia, IMGT or Kabat annotation.

### DETAILED DESCRIPTION

The present invention will be described with respect to particular embodiments and with reference to certain drawings but the invention is not limited thereto but only by the claims. Any reference signs in the claims shall not be construed as limiting the scope. Of course, it is to be understood that not necessarily all aspects or advantages may be achieved in accordance with any particular embodiment of the invention. Thus, for example those skilled in the art will recognize that the invention may be embodied or carried out in a manner that achieves or optimizes one advantage or group of advantages as taught herein without necessarily achieving other aspects or advantages as may be taught or suggested herein. The invention, both as to organization and method of operation, together with features and advantages thereof, may best be understood by reference to the following detailed description when read in conjunction with the accompanying drawings. The aspects and advantages of the invention will be apparent from and elucidated with reference to the embodiment(s) described hereinafter. Reference throughout this specification to “one embodiment” or “an embodiment” means that a particular feature, structure or characteristic described in connection with the embodiment is included in at least one embodiment of the present invention. Thus, appearances of the phrases ‘in one embodiment’ or ‘in an embodiment’ in various places throughout this specification are not necessarily all referring to the same embodiment but may.

### Definitions

Where an indefinite or definite article is used when referring to a singular noun e.g. “a” or “an”, “the”, this includes a plural of that noun unless something else is specifically stated. Where the term “comprising” is used in the present description and claims, it does not exclude other elements or steps. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments, of the invention described herein are capable of operation in other sequences than described or illustrated herein. The following terms or definitions are provided solely to aid in the understanding of the invention. Unless specifically defined herein, all terms used herein have the same meaning as they would to one skilled in the art of the present invention. Practitioners are particularly directed to Sambrook et al., Molecular Cloning: A Laboratory Manual, 4^{th} ed., Cold Spring Harbor Press, Plainsview, New York (2012); and Ausubel et al., Current Protocols in Molecular Biology (Supplement 114), John Wiley & Sons, New York (2016), for definitions and terms of the art. Unless defined otherwise, all technical and scientific terms used herein have the same meaning as commonly understood by one of ordinary skill in the art (e.g. in molecular biology, biochemistry, structural biology, and/or computational biology).

“Nucleotide sequence”, “DNA sequence” or “nucleic acid molecule(s)” as used herein refers to a polymeric form of nucleotides of any length, either ribonucleotides or deoxyribonucleotides. This term refers onlyto the primary structure of the molecule. Thus, this term includes double- and single-stranded DNA, the (reverse) complement DNA, and RNA. It also includes known types of modifications, for example, methylation, “caps” substitution of one or more of the naturally occurring nucleotides with an analog. By “nucleic acid construct” it is meant a nucleic acid sequence that has been constructed to comprise one or more functional units not found together in nature. Examples include circular, linear, double-stranded, extrachromosomal DNA molecules (plasmids), cosmids (plasmids containing COS sequences from lambda phage), viral genomes comprising non-native nucleic acid sequences, and the like. “Coding sequence” is a nucleotide sequence, which is transcribed into mRNA and/or translated into a polypeptide when placed under the control of appropriate regulatory sequences. The boundaries of the coding sequence are determined by a translation start codon at the 5′-terminus and a translation stop codon at the 3′-terminus. A coding sequence can include, but is not limited to mRNA, cDNA, recombinant nucleotide sequences or genomic DNA, while introns may be present as well under certain circumstances. The term “vector”, “vector construct,” “expression vector”, or “recombinant vector” as used herein, is intended to refer to a nucleic acid molecule capable of transporting another nucleic acid molecule to which it has been linked. More particular, said vector may include any vector known to the skilled person, including any suitable type, but not limited to, for instance, plasmid vectors, cosmid vectors, phage vectors, such as lambda phage, viral vectors, even more particular a lentiviral, adenoviral, AAV or baculoviral vectors, or artificial chromosome vectors such as bacterial artificial chromosomes (BAC), yeast artificial chromosomes (YAC), or P1 artificial chromosomes (PAC). Expression vectors comprise plasmids as well as viral vectors and generally contain a desired coding sequence and appropriate DNA sequences necessary for the expression of the operably linked coding sequence in a particular host organism (e.g., bacteria, yeast, plant, insect, or mammal) or in in vitro expression systems. Cloning vectors are generally used to engineer and amplify a certain desired DNA fragment and may lack functional sequences needed for expression of the desired DNA fragments. The construction of expression vectors for use in transfecting cells is also well known in the art, and thus can be accomplished via standard techniques (see, for example, Sambrook, Fritsch, and Maniatis, in: Molecular Cloning, A Laboratory Manual, Cold Spring Harbor Laboratory Press, 1989; Gene Transfer and Expression Protocols, pp. 109-128, ed. E. J. Murray, The Humana Press Inc., Clif ton, N.J.), and the Ambion 1998 Catalog (Ambion, Austin, Tex.).

The terms “protein”, “polypeptide”, and “peptide” are interchangeably used further herein to refer to a polymer of amino acid residues and to variants and synthetic analogues of the same. A “peptide” may also be referred to as a partial amino acid sequence derived from its original protein, for instance after tryptic digestion. Thus, these terms apply to amino acid polymers in which one or more amino acid residues is a synthetic non-naturally occurring amino acid, such as a chemical analogue of a corresponding naturally occurring amino acid, as well as to naturally-occurring amino acid polymers. This term also includes posttranslational modifications of the polypeptide, such as glycosylation, phosphorylation and acetylation. Based on the amino acid sequence and the modifications, the atomic or molecular mass or weight of a polypeptide is expressed in (kilo)dalton (kDa). Amino acids are presented herein by their 3- or 1-lettercode nomenclature as defined and provided also in the IUPAC-IUB Joint Commission on Biochemical Nomenclature (Nomenclature and Symbolism for Amino Acids and Peptides. Eur. J. Biochem. 138: 9-37 (1984)); as follows: Alanine (A or Ala), Cysteine (C or Cys), Aspartic acid (D or Asp), Glutamic acid (E or Glu), Phenylalanine (F or Phe), Glycine (G or Gly), Histidine (H or His), Isoleucine (I or lie), Lysine (K or Lys), Leucine (L or Leu), Methionine (M or Met), Asparagine (N or Asn), Proline (P or Pro), Glutamine (Q or Gln), Arginine (R or Arg), Serine (S or Ser), Threonine (T or Thr), Valine (V or Val), Tryptophan (W or Trp), and Tyrosine (Y or Tyr).

By “isolated” or “purified” is meant material that is substantially or essentially free from components that normally accompany it in its native state. For example, an “isolated polypeptide” or “purified polypeptide” refers to a polypeptide which has been purified from the molecules which flank it in a naturally-occurring state, e.g., an antibody or nanobody as identified and disclosed herein which has been removed from the molecules present in the sample or mixture, such as a production host, that are adjacent to said polypeptide. An isolated protein or peptide can be generated by amino acid chemical synthesis or can be generated by recombinant production or by purification from a complex sample.

“Homologue”, “Homologues” of a protein encompass peptides, oligopeptides, polypeptides, proteins and enzymes having amino acid substitutions, deletions and/or insertions relative to the unmodified protein in question and having similar biological and functional activity as the unmodified protein from which they are derived. The term “amino acid identity” as used herein refers to the extent that sequences are identical on an amino acid-by-amino acid basis over a window of comparison. Thus, a “percentage of sequence identity” is calculated by comparing two optimally aligned sequences over the window of comparison, determining the number of positions at which the identical amino acid residue (e.g., Ala, Pro, Ser, Thr, Gly, Val, Leu, lie, Phe, Tyr, Trp, Lys, Arg, His, Asp, Glu, Asn, Gln, Cys and Met, also indicated in one-letter code herein) occurs in both sequences to yield the number of matched positions, dividing the number of matched positions by the total number of positions in the window of comparison (i.e., the window size), and multiplying the result by 100 to yield the percentage of sequence identity. A “substitution”, or “mutation”, or “variant” as used herein, results from the replacement of one or more amino acids or nucleotides by different amino acids or nucleotides, respectively as compared to an amino acid sequence or nucleotide sequence of a parental protein or a fragment thereof. It is understood that a protein or a fragment thereof may have conservative amino acid substitutions which have substantially no effect on the protein's activity.

“Binding” means any interaction, be it direct or indirect. A direct interaction implies a contact between the binding partners. An indirect interaction means any interaction whereby the interaction partners interact in a complex of more than two molecules. The interaction can be completely indirect, with the help of one or more bridging molecules, or partly indirect, where there is still a direct contact between the partners, which is stabilized by the additional interaction of one or more molecules. By the term “specifically binds,” as used herein is meant a binding domain which recognizes a specific target, but does not substantially recognize or bind other molecules in a sample. Specific binding does not mean exclusive binding. However, specific binding does mean that proteins have a certain increased affinity or preference for one or a few of their binders. The term “affinity”, as used herein, generally refers to the degree to which a ligand, chemical, protein or peptide binds to another (target) protein or peptide so as to shift the equilibrium of single protein monomers toward the presence of a complex formed by their binding. Affinity is the strength of binding of a single molecule to its ligand. It is typically measured and reported by the equilibrium dissociation constant (K_{D}), which is used to evaluate and rank order strengths of bimolecular interactions. The binding of an antibody to its antigen is a reversible process, and the rate of the binding reaction is proportional to the concentrations of the reactants. At equilibrium, the rate of [antibody][antigen] complex formation is equal to the rate of dissociation into its components [antibody]+[antigen]. The measurement of the reaction rate constants can be used to define an equilibrium or affinity constant (1/K_{D}). In short, the smaller the K_{D} value the greater the affinity of the antibody for its target. The rate constants of both directions of the reaction are termed: the association reaction rate constant (kₐ), which is the part of the reaction used to calculate the “on-rate” (kₒₙ), a constant used to characterize how quickly the antibody binds to its target. Vice versa, the dissociation reaction rate constant (k_{d}), is the part of the reaction used to calculate the “off-rate” (k_{off}), a constant used to characterize how quickly an antibody dissociates from its target. In measurements as shown herein, the flatter the slope, the slower off-rate, or the stronger antibody binding. Vice versa, the steeper downside indicates a faster off-rate and weaker antibody binding. The ratio of the experimentally measured off- and on-rates (k_{off}/kₒₙ) is used to calculate the K_{D} value. Several determination methods are known to the skilled person to measure on and off rates and to thereof calculate the K_{D}, which is therefore, taking into account standard errors, considered as a value that is independent of the assay used. As used herein, the term “protein complex” or “complex” or “assembled protein(s)” refers to a group of two or more associated macromolecules, whereby at least one of the macromolecules is a protein. A protein complex, as used herein, typically refers to associations of macromolecules that can be formed under physiological conditions. Individual members of a protein complex are linked by non-covalent interactions.

A “binding agent” relates to a molecule that is capable of binding to another molecule, wherein said binding is preferably a specific binding, recognizing a defined binding site, pocket or epitope. The binding agent may be of any nature or type and is not dependent on its origin. The binding agent may be chemically synthesized, naturally occurring, recombinantly produced (and purified), as well as designed and synthetically produced. Said binding agent may hence be a small molecule, a chemical, a peptide, a polypeptide, an antibody, or any derivatives thereof, such as a peptidomimetic, an antibody mimetic, an active fragment, a chemical derivative, among others. The term “binding pocket” or “binding site” refers to a region of a molecule or molecular complex, that, as a result of its shape and charge, favourably associates with another chemical entity, compound, proteins, peptide, antibody or Nb. The term “pocket” includes, but is not limited to cleft, channel or site. The term “part of a binding pocket/site” refers to less than all of the amino acid residues that define the binding pocket, or binding site. For example, the portion of residues may be key residues that play a role in ligand binding, or may be residues that are spatially related and define a three-dimensional compartment of the binding pocket. The residues may be contiguous or non-contiguous in primary sequence. For antibody-related molecules, the term “epitope” is also used to describe the binding site, as used interchangeably herein.

Methods of determining the spatial conformation of amino acids are known in the art, and include, for example, X-ray crystallography and multi-dimensional nuclear magnetic resonance. A “conformational epitope”, as used herein, refers to an epitope comprising amino acids in a spatial conformation that is unique to a folded 3-dimensional conformation of a polypeptide. Generally, a conformational epitope consists of amino acids that are discontinuous in the linear sequence but that come together in the folded structure of the protein. However, a conformational epitope may also consist of a linear sequence of amino acids that adopts a conformation that is unique to a folded 3-dimensional conformation of the polypeptide (and not present in a denatured state). In protein complexes, conformational epitopes consist of amino acids that are discontinuous in the linear sequences of one or more polypeptides that come together upon folding of the different folded polypeptides and their association in a unique quaternary structure. The term “conformation” or “conformational state” of a protein refers generally to the range of structures that a protein may adopt at any instant in time. A conformational epitope may thus comprise amino acid interactions from different protein domains of the CI-M6PR protein. One of skill in the art will recognize that determinants of conformation or conformational state include a protein's primary structure as reflected in a protein's amino acid sequence (including modified amino acids) and the environment surrounding the protein. The conformation or conformational state of a protein also relates to structural features such as protein secondary structures (e.g., α-helix, β-sheet, among others), tertiary structure (e.g., the three dimensional folding of a polypeptide chain), and quaternary structure (e.g., interactions of a polypeptide chain with other protein subunits). Posttranslational and other modifications to a polypeptide chain such as ligand binding, phosphorylation, sulfation, glycosylation, or attachments of hydrophobic groups, among others, can influence the conformation of a protein. Furthermore, environmental factors, such as pH, salt concentration, ionic strength, and osmolality of the surrounding solution, and interaction with other proteins and co-factors, among others, can affect protein conformation. The conformational state of a protein may be determined by either functional assay for activity or binding to another molecule or by means of physical methods such as X-ray crystallography, NMR, or spin labeling, among other methods. For a general discussion of protein conformation and conformational states, one is referred to Cantor and Schimmel, Biophysical Chemistry, Part I: The Conformation of Biological. Macromolecules, W.H. Freeman and Company, 1980, and Creighton, Proteins: Structures and Molecular Properties, W.H. Freeman and Company, 1993.

The term “antibody”, “antibody fragment” and “active antibody fragment” as used herein refer to a protein comprising an immunoglobulin (Ig) domain or an antigen binding domain capable of specifically binding the antigen, in this case the N-terminal domains 1-3 of the (human) CI-M6PR protein. ‘Antibodies’ can further be intact immunoglobulins derived from natural sources or from recombinant sources and can be immunoreactive portions of intact immunoglobulins. Antibodies are typically tetramers of immunoglobulin molecules. The term “active antibody fragment” refers to a portion of any antibody or antibody-like structure that by itself has high affinity for an antigenic determinant, or epitope, and contains one or more complementarity-determining-regions (CDRs) accounting for such specificity. Non-limiting examples include immunoglobulin domains, Fab, F(ab)′2, scFv, heavy-light chain dimers, immunoglobulin single variable domains, Nanobodies, domain antibodies, and single chain structures, such as a complete light chain or complete heavy chain. An additional requirement for “activity” of said fragments in the light of the present invention is that said fragments are capable of binding CI-M6PR, and preferably are specifically binding and have favorable dissociation profiles at lower pH (i.e. acidic conditions as in endosomes and lysosomes below pH 7), more preferably are capable to dissociate at a pH around 5.8, and/or retain binding at said pH (depending on the application/treatment) in a subject. The term “immunoglobulin (Ig) domain”, or more specifically “immunoglobulin variable domain” (abbreviated as “IVD”) means an immunoglobulin domain essentially consisting of four “framework regions” which are referred to in the art and herein below as “framework region 1” or “FR1”; as “framework region 2” or “FR2”; as “framework region 3” or “FR3”; and as “framework region 4” or “FR4”, respectively; which framework regions are interrupted by three “complementarity determining regions” or “CDRs”, which are referred to in the art and herein below as “complementarity determining region 1” or “CDR1”; as “complementarity determining region 2” or “CDR2”; and as “complementarity determining region 3” or “CDR3”, respectively. Thus, the general structure or sequence of an immunoglobulin variable domain can be indicated as follows: FR1-CDR1-FR2-CDR2-FR3-CDR3-FR4. It is the immunoglobulin variable domain(s) (IVDs) that confer specificity to an antibody for the antigen by carrying the antigen-binding site. Typically, in conventional immunoglobulins, a heavy chain variable domain (VH) and a light chain variable domain (VL) interact to form an antigen binding site. In this case, the complementarity determining regions (CDRs) of both VH and VL will contribute to the antigen binding site, i.e. a total of 6 CDRs will be involved in antigen binding site formation. In view of the above definition, the antigen-binding domain of a conventional 4-chain antibody (such as an IgG, IgM, IgA, IgD or IgE molecule; known in the art) or of a Fab fragment, a F(ab′)2 fragment, an Fv fragment such as a disulphide linked Fv or a scFv fragment, or a diabody (all known in the art) derived from such conventional 4-chain antibody, with binding to the respective epitope of an antigen by a pair of (associated) immunoglobulin domains such as light and heavy chain variable domains, i.e., by a VH-VL pair of immunoglobulin domains, which jointly bind to an epitope of the respective antigen. An immunoglobulin single variable domain (ISVD) as used herein, refers to a protein with an amino acid sequence comprising 4 Framework regions (FR) and 3 complementary determining regions (CDR) according to the format of FR1-CDR1-FR2-CDR2-FR3-CDR3-FR4. An “immunoglobulin domain” of this invention also refers to “immunoglobulin single variable domains” (abbreviated as “ISVD”), equivalent to the term “single variable domains”, and defines molecules wherein the antigen binding site is present on, and formed by, a single immunoglobulin domain. This sets immunoglobulin single variable domains apart from “conventional” immunoglobulins or their fragments, wherein two immunoglobulin domains, in particular two variable domains, interact to form an antigen binding site. The binding site of an immunoglobulin single variable domain is formed by a single VH/VHH or VL domain. Hence, the antigen binding site of an immunoglobulin single variable domain is formed by no more than three CDR's. As such, the single variable domain may be a light chain variable domain sequence (e.g., a VL-sequence) or a suitable fragment thereof; or a heavy chain variable domain sequence (e.g., a VH-sequence or VHH sequence) or a suitable fragment thereof; as long as it is capable of forming a single antigen binding unit (i.e., a functional antigen binding unit that essentially consists of the single variable domain, such that the single antigen binding domain does not need to interact with another variable domain to form a functional antigen binding unit).

In particular, the immunoglobulin single variable domain may be a Nanobody® (as defined herein) or a suitable fragment thereof. Note: Nanobody®, Nanobodies® and Nanoclone® are registered trademarks of Ablynx N.V. (a Sanofi Company). For a general description of Nanobodies, reference is made to the further description below, as well as to the prior art cited herein, such as e.g. described in WO2008/020079. “VHH domains”, also known as VHHs, VHH domains, VHH antibody fragments, and VHH antibodies, have originally been described as the antigen binding immunoglobulin (Ig) (variable) domain of “heavy chain antibodies” (i.e., of “antibodies devoid of light chains”; Hamers-Casterman et al (1993) Nature 363: 446-448). The term “VHH domain” has been chosen to distinguish these variable domains from the heavy chain variable domains that are present in conventional 4-chain antibodies (which are referred to herein as “VH domains”) and from the light chain variable domains that are present in conventional 4-chain antibodies (which are referred to herein as “VL domains”). For a further description of VHHs and Nanobody, reference is made to the review article by Muyldermans (Reviews in Molecular Biotechnology 74: 277-302, 2001), as well as to the following patent applications, which are mentioned as general background art: WO 94/04678, WO 95/04079 and WO 96/34103 of the Vrije Universiteit Brussel; WO 94/25591, WO 99/37681, WO 00/40968, WO 00/43507, WO 00/65057, WO 01/40310, WO 01/44301, EP 1134231 and WO 02/48193 of Unilever; WO 97/49805, WO 01/21817, WO 03/035694, WO 03/054016 and WO 03/055527 of the Vlaams Instituut voor Biotechnologie (VIB); WO 03/050531 of Algonomics N.V. and Ablynx N.V.; WO 01/90190 by the National Research Council of Canada; WO 03/025020 (=EP 1433793) by the Institute of Antibodies; as well as WO 04/041867, WO 04/041862, WO 04/041865, WO 04/041863, WO 04/062551, WO 05/044858, WO 06/40153, WO 06/079372, WO 06/122786, WO 06/122787 and WO 06/122825, by Ablynx N.V. and the further published patent applications by Ablynx N.V. As described in these references, Nanobody (in particular VHH sequences and partially humanized Nanobody) can in particular be characterized by the presence of one or more “Hallmark residues” in one or more of the framework sequences. A further description of the Nanobody, including humanization and/or camelization of Nanobody, as well as other modifications, parts or fragments, derivatives or “Nanobody fusions”, multivalent or multispecific constructs (including some non-limiting examples of linker sequences) and different modifications to increase the half-life of the Nanobody and their preparations can be found e.g. in WO 08/101985 and WO 08/142164. Nanobodies form the smallest antigen binding fragment that completely retains the binding affinity and specificity of a full-length antibody. Nbs possess exceptionally long complementarity-determining region 3 (CDR3) loops and a convex paratope, which allow them to penetrate into hidden cavities of target antigens.

As used herein, the terms “determining,” “measuring,” “assessing,”, “identifying”, “screening”, and “assaying” are used interchangeably and include both quantitative and qualitative determinations.

The term “subject”, “individual” or “patient”, used interchangeably herein, relates to any organism such as a vertebrate, particularly any mammal, including both a human and another mammal, for whom diagnosis, therapy or prophylaxis is desired, e.g., an animal such as a rodent, a rabbit, a cow, a sheep, a horse, a dog, a cat, a lama, a pig, or a non-human primate (e.g., a monkey). The rodent may be a mouse, rat, hamster, guinea pig, or chinchilla. In one embodiment, the subject is a human, a rat or a non-human primate. Preferably, the subject is a human. In one embodiment, a subject is a subject with or suspected of having a disease or disorder, in particular a disease or disorder as disclosed herein, also designated “patient” herein. However, it will be understood that the aforementioned terms do not imply that symptoms are present. The term “treatment” or “treating” or “treat” can be used interchangeably and are defined by a therapeutic intervention that slows, interrupts, arrests, controls, stops, reduces, or reverts the progression or severity of a sign, symptom, disorder, condition, or disease, but does not necessarily involve a total elimination of all disease-related signs, symptoms, conditions, or disorders.

The term “medicament”, as used herein, refers to a substance/composition used in therapy, i.e., in the prevention or treatment of a disease or disorder. According to the invention, the terms “disease” or “disorder” refer to any pathological state, in particular to the diseases or disorders as defined herein.

### DETAILED DESCRIPTION

The present invention is based on the identification of CI-M6PR-specific VHHs. VHHs were chosen as binding agents to select for since they are known as highly stable and soluble, and can easily and cost-effectively be manufactured in lower organisms such as bacteria and yeast. Moreover, VHHs are unique in their great conformational stability, and high intrinsic pH and protease resistance, which all form attractive properties for cycling through the endosomal-lysosomal system. Furthermore, VHH-based formats are suitable for various routes of administration, including via intravenous injection and inhalation, thus providing for a novel approach to apply lysosomal targeting of drug products, optionally in complex with their targets. More specifically, the binders as described herein may be coupled or operably linked to further binding moieties, which may be enzymes, or which may be antigen-binding domains specific for a target protein, preferably a target present on the cell surface or extracellularly. Such bi- or multi-specific binders or ISVD-fusion polypeptides result in CI-M6PR-mediated lysosomal uptake, as cargo for delivery of specific extracellular or cell surface target(s), which will finally be recycled through the endolysosomal cycle, and/or degraded in the lysosomes, or used as vehicle for enzyme delivery.

Because the CI-M6PR constantly traffics between the late endosome and the cell membrane, the binding agents disclosed herein may dissociate at the lower pH in these subcellular organelles, or may retain binding to CI-M6PR and recycle with it. Indeed, the early endosores maintain a pH at about 6.5, while late endosomes are at about 5.5, and where endosomes fully mature into lysosomes, pH is at about 4.5.

So the pH-dependent dissociation profiles for the binding agents described herein positions them ideally to function in either of the proposed functionalities. The first option to dissociate at around pH 6-5.5 may lead to lysosomal targeting of said endosomal dissociated binder, further being degraded when delivered in the lysosome, whereas the latter (dissociation only at pH lower than 5) may contribute to an increased half-life of such binding agents in a subject through recycling. Moreover, tunability of pH dissociation of antigen-binding domains is known in the art, and may allow to generate multi-specific binders wherein for instance the CI-M6PR-specific ISVD is capable of maintaining its binding throughout the recycling process, while further antigen-domain binders may dissociate from their target at pH values corresponding to pH in the endosome and lysosome, as to release its target for degradation. This would increase their target degradation efficacy and hence potency. Though also a high protease-resistance is required for recycling of such an ISVD-based anti-CI-M6PR binders.

A first aspect of the invention thus provides for ISVD-based binding agents specifically binding the N-terminal extracellular portion of the CI-M6PR protein, more specifically binding to a conformational epitope present on domains 1, 2 and/or 3 as defined herein (see for instance FIG. 22).

The binding to the CI-M6PR protein at the extracellular surface of a cell requires a certain affinity, as to maintain its binding upon internalisation of the receptor in the endosomes. Once a threshold binding affinity is reached, which may be in the micromolar, nanomolar, or picomolar range, internalisation and uptake in the cell leads to ISVD-based binding agents being present within the cellular compartments, from early endosomes, to later endosome, to finally go to the lysosomes of the cell. For the binders of the present invention, a binding affinity in the nanomolar to picomolar range is envisaged, as determined at neutral pH, more specifically at pH 7.4, as to allow efficient uptake and or recycling with the CI-M6PR protein in the cell. In one embodiment, the CI-M6PR-specific ISVD-based binding agent binds cell-expressed CI-M6PR via a binding site on domains 1, 2 and/or 3, and is capable in its monovalent form, so through binding via its residues present in maximum 3 CDRs of one ISVD, to internalize (preferably when fused to a label or a further moiety or tag), within said cells. In a further specific embodiment, the efficiency of its internalisation is defined as the minimal internalisation rate of said CI-M6PR-specific binding agent by the voxel counts/minute in a life cell imaging experimental method (see Examples), and is herein considered as ‘internalised’ with an internalisation rate of at least 15 voxel counts/min, or at least 35, or at least 50, or at least 65, or at least 80, or at least 100, or at least 120 voxel counts/minute.

In another embodiment, the ISVDs specifically interacting with CI-M6PR, as described herein, further provide for the necessary biophysical and binding characteristics at different pH values as to retain binding to the receptor N-terminal portion upon internalisation into endosomes and/or lysosome trafficking on or in a cell.

In a specific embodiment, said binding agent provides for a retained binding to said CI-M6P receptor upon internalisation, and as shown by its pH dependent binding profile (demonstrated for the ISVDs herein by BLI), only dissociates from the receptor at a pH below the pH of the endosomal compartment, so below pH 6. Hence said ISVD-based binding agents provide for strong binders at neutral pH and in the endosomes (pH 6-5.5), but allow clear dissociation from the receptor at lower pH, which likely leads to said ISVD-binding agent to at least partially be recycled back to the outer membrane. This may lead to functional ISVD-based removal of surface- or extracellular molecules from the outside of the cell to the endosomal compartments. Such a pH-dependent dissociation profile has for instance been observed for the VHH8, VHH5, and VHH1H52 ISVDs disclosed herein. Those VHHs belong to a different VHH family, though, competition experiments revealed they compete for the same binding site on the CI-M6PR, and based on co-crystal analysis of VHH8 with the CI-M6PR dom1-3, the epitope was determined to be located on N-terminal domains 2 and 3.

More specifically, said CI-M6PR-specific binding agent its binding site (herein also defined as VHH8-epitope) may be more specifically delineated as the ISVD being in contact with the amino acid residues of CI-M6PR at position Lys191, Gly194, Ala195, Tyr196, Leu197, Phe208, Arg219, Gln224, Leu225, Ile297, Lys357, Gly408, Asp409, Asn431, Glu433, and Phe457 as set forth in SEQ ID NO:20, which presents the amino acid sequence of human CI-M6PR. The epitope is defined herein as the amino acids being in contact with each other based on an integrated analysis of a distance of 4 Angstrom or less from the VHH residues, a PISA and a FastContact analysis, as described herein.

In a further specific embodiment, said VHH8-epitope binding site may be more specifically delineated as the ISVD being in contact with the amino acid residues of CI-M6PR at position Lys191, Gly194, Ala195, Tyr196, Leu197, Phe208, Arg219, Gln224, Leu225, Ile297, Lys357, Gly408, Asp409, Asn431, Glu433, and Phe457 as set forth in SEQ ID NO:20, and contacting at least two, at least 3, at least 4, at least 5 or at least 6, or more residues out of the amino acid residues Ser193, Ser206, Asp216, Asp220, Pro221, Gly222, Ser223, Pro298, Trp312, Glu358, Thr449, Gly450, Glu451, Val452, and Thr459 as set forth in SEQ ID NO:20 wherein the epitope is defined as comprising residues that are within 4 Angstrom distance from the VHH. In a further specific embodiment, said VHH8-epitope binding site may be more specifically delineated as the ISVD being in contact with the amino acid residues of CI-M6PR at position Lys191, Ser193, Gly194, Ala195, Tyr196, Leu197, Ser206, Phe208, Asp216, Arg219, Asp220, Pro221, Gly222, Ser223, Gln224, Leu225, Ile297, Pro298, Trp312, Lys357, Glu358, Gly408, Asp409, Asn431, Glu433, Thr449, Gly450, Glu451, Val452, Phe457, and Thr459 as set forth in SEQ ID NO:20, wherein the epitope is defined as consisting of all residues that are within 4 Angstrom distance from the VHH. In a further embodiment, said CI-M6PR-specific binding agent may be defined as an agent competing for binding to said VHH8-epitope as described herein.

In a further specific embodiment said binding agent comprising an ISVD specifically binding CI-M6PR domains 2, 3, by having in contact its residues Tyr32, Arg52, Trp53, Ser54, Ser56, Lys57, Ile100, Phe103 and Ser108, as set forth in SEQ ID NO:8 (numerical order, no Kabat numbering is used here) providing for the paratope of said ISVD for binding to said epitope described above. In a further embodiment said ISVD specifically binding CI-M6PR domains 2, and 3, by having in contact its residues of CDR1, CDR2 and CDR3 of SEQ ID NO:8, as defined by Kabat, MacCallum, Chothia, AbM or IMGT. In a further specific embodiment said binding agent comprising an ISVD comprising the CDR1 of SEQ ID NO: 107, CDR2 of SEQ ID NO:114, or CDR3 of SEQ ID NO:121. In a further embodiment said ISVD specifically binding CI-M6PR domains 2, and 3, contains SEQ ID NO:8.

A further specific embodiment relates to binding agent comprising an ISVD specifically binding CI-M6PR domains 2, and 3, competing for binding to said VHH8 epitope, and comprising the CDRs as presented for the ISVDs presented by VHH5 and VHH1H52, or SEQ ID NO:5 and SEQ ID NO: 73 resp. as annotated by Kabat, MacCallum, Chothia, AbM or IMGT. In a further specific embodiment said binding agent comprising an ISVD comprising the CDR1 of SEQ ID NO: 108-109, CDR2 of SEQ ID NO:115-116, or CDR3 of SEQ ID NO:122-123. In a further embodiment said ISVD specifically binding CI-M6PR domains 2, and 3, contains SEQ ID NO: 5 or 73.

In a further alternative embodiment, said binding agent provides for a binding to said CI-M6P receptor upon internalisation, and as shown by its pH dependent binding profile (demonstrated for the ISVDs herein by BLI), which gradually dissociates from the receptor at a pH as present in the endosomal compartment, so dissociation occurs similar to the receptor's natural ligands, at a pH around 6 down to 5.5. Hence said ISVD-based binding agents provide for binders at neutral pH but with dissociation in the endosomes (pH 6-5.5), allowing the receptor to cycle back, and the ISVD-binding agent to proceed to the lysosome (and not be recycled to the outer membrane). Such a pH-dependent dissociation profile has for instance been observed for the VHH7, VHH1, and VHH1H11 ISVDs disclosed herein. Each of those VHHs belong to a different VHH family, though, competition experiments revealed they compete for the same binding site on the M6PR dom1-3, and based on co-crystal analysis of VHH7 and VHH1H11 with the CI-M6PR dom1-3, the epitope was determined to be located on N-terminal domain 1.

More specifically, said CI-M6PR-specific binding agent its binding site (herein also referred to as VHH7-epitope or VHH7/VHH1H11 epitope or VHH1H11 epitope) may be more specifically delineated as the ISVD being in contact with the amino acid residues of CI-M6PR at position Lys59, Asn60, Met85, Asp87, Lys89, Ala146, Thr147, Glu148; and Asp118 or Gln119, as set forth in SEQ ID NO:20. The epitope is defined herein as the amino acids being in contact with each other based on an integrated analysis of a distance of 4 Angstrom or less from the VHH residues, a PISA and a FastContact analysis, as described herein.

In a further specific embodiment, said VHH7-epitope binding site may be more specifically delineated as the ISVD being in contact with the amino acid residues of CI-M6PR at position Lys59, Asn60, Met85, Asp87, Lys89, Asp118, Ala146, Thr147, and Glu148, as set forth in SEQ ID NO:20, and contacting at least two, at least 3, at least 4, at least 5 or at least 6, or more residues out of the amino acid residues Asp57, Thr58, Val62, Thr90, His94, Phe143, Thr145, or His151, wherein the epitope is defined as comprising residues that are within 4 Angstrom distance from the VHH.

In a further specific embodiment, said VHH7-epitope binding site may be more specifically delineated as the ISVD being in contact with the amino acid residues of CI-M6PR at position Asp57, Thr58, Lys59, Asn60, Val62, Met85, Asp87, Lys89, Thr90, His94, Asp118, Phe143, Thr145, Ala146, Thr147, Glu148, His151, as set forth in SEQ ID NO:20, wherein the epitope is defined as consisting of all residues that are within 4 Angstrom distance from the VHH.

In a further specific embodiment said binding agent comprising an ISVD specifically binding CI-M6PR predominantly domain 1 by having in contact its residues Asp31, Arg33, Asp35, Trp53, Ser54, Ser56, Lys57, Lys96, Asp104, as set forth in SEQ ID NO:7 (numerical order, no Kabat numbering is used here) providing for the paratope of said ISVD for binding to said epitope described above. In a further embodiment said ISVD specifically binding CI-M6PR domain 1, by having in contact its residues of CDR1, CDR2 and CDR3 of SEQ ID NO:7, as defined by Kabat, MacCallum, Chothia, AbM or IMGT. In a further specific embodiment said binding agent comprising an ISVD comprising the CDR1 of SEQ ID NO: 103, CDR2 of SEQ ID NO:110, or CDR3 of SEQ ID NO:117. In a further embodiment said ISVD specifically binding CI-M6PR domain1, contains SEQ ID NO:7.

In a further specific embodiment, said VHH1H11-epitope binding site may be more specifically delineated as the ISVD being in contact with the amino acid residues of CI-M6PR at position Lys59, Asn60, Met85, Asp87, Lys89, Gln119, Ala146, Thr147, and Glu148, as set forth in SEQ ID NO:20, and contacting at least two, at least 3, at least 4, at least 5 or at least 6, or more residues out of the amino acid residues Asp57, Val62, Thr90, His94, Phe143, Thr145, His151, or Arg404 wherein the epitope is defined as comprising residues that are within 4 Angstrom distance from the VHH.

In a further specific embodiment, said VHH7-epitope binding site may be more specifically delineated as the ISVD being in contact with the amino acid residues of CI-M6PR at position Asp57, Lys59, Asn60, Met85, Asp87, Lys89, Thr90, His94, Gln119, Phe143, Thr145, Ala146, Thr147, Glu148, His151, and Arg404 as set forth in SEQ ID NO:20, wherein the epitope is defined as consisting of all residues that are within 4 Angstrom distance from the VHH.

In a further specific embodiment said binding agent comprising an ISVD specifically binding CI-M6PR predominantly domain 1 by having in contact its residues 52-57, 96-98 Asp31, Asn32, Arg33, Asp35, Thr50, Ala52, Ser53, Tyr54, Gly55, Trp56, Lys57, Asn96, Ser97, Gly98, as set forth in SEQ ID NO:71 (numerical order, no Kabat numbering is used here) providing for the paratope of said ISVD for binding to said epitope described above. In a further embodiment said ISVD specifically binding CI-M6PR domain 1, by having in contact its residues of CDR1, CDR2 and CDR3 of SEQ ID NO:71, as defined by Kabat, MacCallum, Chothia, AbM or IMGT. In a further specific embodiment said binding agent comprising an ISVD comprising the CDR1 of SEQ ID NO: 105, CDR2 of SEQ ID NO:112, or CDR3 of SEQ ID NO:119. In a further embodiment said ISVD specifically binding CI-M6PR domain1, contains SEQ ID NO:71.

A further specific embodiment relates to binding agent comprising an ISVD specifically binding (predominantly) to CI-M6PR domain 1, competing for binding to said VHH7 epitope, and comprising the CDRs as presented for the ISVDs presented by VHH1 or SEQ ID NO:1 as annotated by Kabat, MacCallum, Chothia, AbM or IMGT. In a further specific embodiment said binding agent comprising an ISVD comprising the CDR1 of SEQ ID NO:104, CDR2 of SEQ ID NO:111, or CDR3 of SEQ ID NO:118. In a further embodiment said ISVD specifically binding CI-M6PR domain 1, contains SEQ ID NO: 1.

An “epitope”, or “binding site” as used herein, refers to an antigenic determinant of a polypeptide, constituting a binding site or binding pocket on a target molecule, such as the extracellular part of the CI-M6P receptor protein, more specifically a binding pocket on the N-terminal domains (1-3) accessible for the ISVDs or VHHs. An epitope could comprise 3 amino acids in a spatial conformation, which is unique to the epitope. Generally, an epitope consists of at least 4, 5, 6, 7 such amino acids, and more usually, consists of at least 8, 9, 10, or more such amino acids. These residues are in ‘in contact’ with the binding agent. In particular, where the epitope is described as disclosed herein ‘contact’ is defined herein as closer or maximally 4 Å, from any residue (or atom) belonging to the binding agent (E.g. as identified for the VHH7 and VHH8 epitope herein in FIG. 22, half circles+full circles). Alternatively, ‘contact’ is defined herein as constituting an estimated binding free energy (as determined herein by FastContact in ΔG; see also Tables 6-8) of below 0 kcal/mol, or −1000 kcal/mol or of below −2000 kcal/mol or of below −4000 kcal/mol, between the binding agent residue and an epitope residue, wherein said binding free energy may be defined as the electrostatic binding free energy. The ‘contact’ may further be defined herein by the estimated interactions as hydrogen or salt bridges between binding agent and receptor, as determined herein by PISA analysis (see Table 6-8), or as the difference between the accessible surface versus the buried surface upon complex formation (as determined in the PISA method used herein). Finally, the core epitope or minimal epitope as defined herein (see FIG. 22, full circles) may be defined as the consensus residues which are into contact with the CI-M6P receptor based on an integrated output from the maximal 4angstrom distance and taking into account the PISA and FastContact analysis outcome. The binding agent residue specifically binding to the target, or making up the essential residues to bind the epitope of the target are defined herein as the paratope, as known in the art. Such a paratope of a binding agent for CI-M6PR may thus be described as the residues of said ISVD as disclosed herein in contact with the epitope residues on the CI-M6PR N-terminal domains 1-3. A more general approach to indicate the paratope of a VHH includes the provision of the CDR sequences, which in this case provides for a broader region of amino acids of the VHH involved in the binding site with the M6PR.

In one embodiment the ISVD-based binding agents disclosed herein comprise a CDR1, CDR2 and CDR3 region, which concern the binding residues of ISVDs, selected from the CDR1, CDR2, and CDR3, respectively of any of the sequences selected from the sequences depicted in SEQ ID NO:1-11, 71-82, and wherein said CDR regions are defined according to any one of the annotations known in the art, specifically, according to the annotation of Kabat, MacCallum, IMGT AbM or Chothia (also see FIG. 64 for an example based on VHH7 and VHH8 sequence annotation). The CDR region annotation for each VHH sequence described herein according to AbM (AbM is Oxford Molecular Ltd.'s antibody modelling package as described on http://www.bioinf.org.uk/abs/index.html) is shown in the sequence listing below (residues in bold, CDR1, 2 and 3 regions from left to right, resp.). Alternatively, slightly different CDR annotations known in the art may be applied here and relate to the IMGT (LeFranc, 2014; Frontiers in Immunology. 5 (22): 1-22), Chothia (Chothia and Lesk, 1987; J Mol Biol. 196:901-17), Kabat (Kabat et al., 1991; Sequences of Proteins of Immunological Interest. 5th edition, NIH publication 91-3242), or MacCallum et al. (J. Mol. Biol. (1996) 262, 732-745) annotation, which are all applicable to identify the CDR regions of the ISVDs as disclosed herein for SEQ ID NO: 1-11, and 71-82.

It should be noted that—as is well known in the art for VH domains and for VHH domains—the total number of amino acid residues in each of the CDRs may vary and may not correspond to the total number of amino acid residues indicated by the Kabat numbering (that is, one or more positions according to the Kabat numbering may not be occupied in the actual sequence, or the actual sequence may contain more amino acid residues than the number allowed for by the Kabat numbering, see for instance FIG. 64, where Kabat numbering is indicated for VHH7 and VHH8 sequences). This means that, generally, the numbering according to Kabat may or may not correspond to the actual numbering of the amino acid residues in the actual sequence. The total number of amino acid residues in a VH domain and a VHH domain will usually be in the range of from 110 to 120, often between 112 and 115. It should however be noted that smaller and longer sequences may also be suitable for the purposes described herein. VHHs or Nbs are often classified in different families according to amino acid sequences, or even in superfamilies, as to cluster the clonally related sequences derived from the same progenitor during B cell maturation (Deschaght et al. 2017, Front Immunol 8:420). This classification is often based on the CDR sequence of the Nbs, and wherein for instance each Nb (or VHH) family is defined as a cluster of clonally) related sequences with a sequence identity threshold of the CDR3 region. Within a single VHH family (also called CDR3-based VHH family or CDR3-family herein) defined herein, the CDR3 sequence is thus identical or very similar in amino acid composition, preferably with at least 80% identity, or at least 85% identity, or at least 90% identity in the CDR3 sequence, resulting in Nbs of the same family binding to the same binding site, and having the same effect such as functional effect.

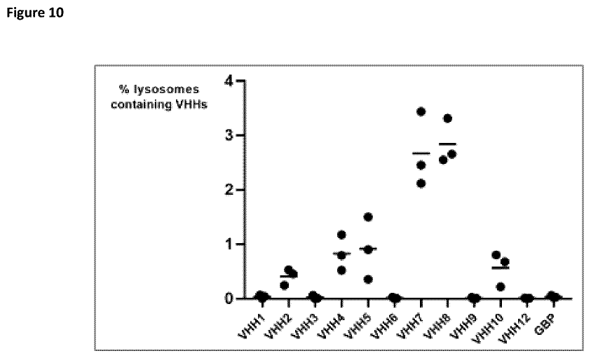

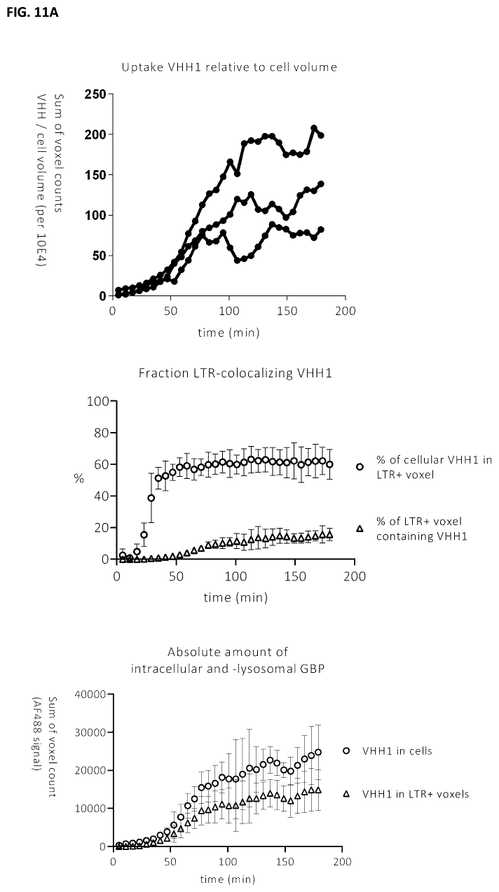

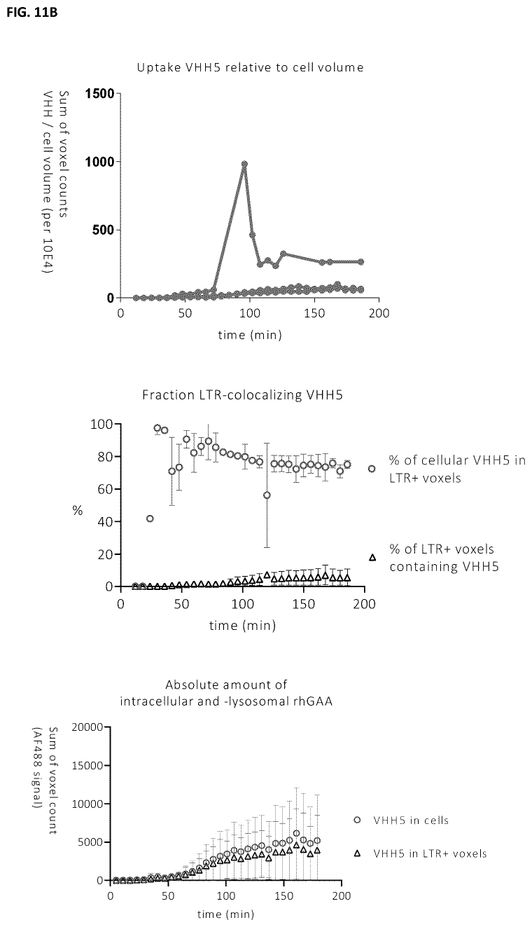

In another embodiment relates to the binding agent provided herein comprising an ISVD specifically binding the CI-M6PR extracellular N-terminal domains 1-3, wherein said ISVD contains a sequence selected from the group of sequences depicting the VHH1, 5, 7, 8, 1H11 and 1H52, and molecules exemplified herein, as shown in SEQ ID NO:1, 5, 7, 8, 71 and 73, resp., or a sequence with at least 85%, or at least 90%, or at least 95%, or at least 99% identity thereof, wherein the CDR regions are identical to the sequence selected from SEQ ID NO:1, 5, 7, 8, 71 and 73, and variation of residues is solely present for non-binding residues of the FR regions. In a further embodiment, the binding agent as described herein comprises an ISVD selected from the group of SEQ ID NO: SEQ ID NO:1, 5, 7, 8, 71 and 73, or a humanized variant of any one thereof. The term ‘humanized variant’ of an immunoglobulin single variable domain such as a domain antibody and Nanobody® (including VHH domain) refers to an amino acid sequence of said ISVD representing the outcome of being subjected to humanization, i.e. to increase the degree of sequence identity with the closest human germline sequence. In particular, humanized immunoglobulin single variable domains, such as Nanobody® (including VHH domains) may be immunoglobulin single variable domains in which at least one amino acid residue is present (and in particular, at least one framework residue) that is and/or that corresponds to a humanizing substitution (as defined further herein). Potentially useful humanizing substitutions can be ascertained by comparing the sequence of the framework regions of a naturally occurring VHH sequence with the corresponding framework sequence of one or more closely related human VH sequences, after which one or more of the potentially useful humanizing substitutions (or combinations thereof) thus determined can be introduced into said VHH sequence (in any manner known per se, as further described herein) and the resulting humanized VHH sequences can be tested for affinity for the target, for stability, for ease and level of expression, and/or for other desired properties. In this way, by means of a limited degree of trial and error, other or further suitable humanizing substitutions (or suitable combinations thereof) can be determined by the skilled person. Also, based on what is described before, (the framework regions of) an immunoglobulin single variable domain, such as a Nanobody® (including VHH domains) may be partially humanized or fully humanized. Humanized immunoglobulin single variable domains, in particular Nanobody, may have several advantages, such as a reduced immunogenicity, compared to the corresponding naturally occurring VHH domains. In summary, the humanizing substitutions should be chosen such that the resulting humanized amino acid sequence of the ISVD and/or VHH still retains the favourable properties, such as the antigen-binding capacity, and allosteric modulation capacity. The skilled person will be able to select humanizing substitutions or suitable combinations of humanizing substitutions which optimize or achieve a desired or suitable balance between the favourable properties provided by the humanizing substitutions on the one hand and the favourable properties of naturally occurring VHH domains on the other hand. Such methods are known by the skilled addressee. A human consensus sequence can be used as target sequence for humanization, but also other means are known in the art. One alternative includes a method wherein the skilled person aligns a number of human germline alleles, such as for instance but not limited to the alignment of IGHV3 alleles, to use said alignment for identification of residues suitable for humanization in the target sequence. Also, a subset of human germline alleles most homologous to the target sequence may be aligned as starting point to identify suitable humanisation residues. Alternatively, the VHH is analyzed to identify its closest homologue in the human alleles, and used for humanisation construct design. A humanisation technique applied to Camelidae VHHs may also be performed by a method comprising the replacement of specific amino acids, either alone or in combination. Said replacements may be selected based on what is known from literature, are from known humanization efforts, as well as from human consensus sequences compared to the natural VHH sequences, or the human alleles most similar to the VHH sequence of interest. As can be seen from the data on the VHH entropy and VHH variability given in Tables A-5-A-8 of WO 08/020079, some amino acid residues (i.e. hallmark residues, FIG. 10) in the framework regions are more conserved between human and Camelidae than others. Generally, although the invention in its broadest sense is not limited thereto, any substitutions, deletions or insertions are preferably made at positions that are less conserved. Also, generally, amino acid substitutions are preferred over amino acid deletions or insertions. For instance, a human-like class of Camelidae single domain antibodies contain the hydrophobic FR2 residues typically found in conventional antibodies of human origin or from other species, but compensating this loss in hydrophilicity by other substitutions at position 103 that substitutes the conserved tryptophan residue present in VH from double-chain antibodies. As such, peptides belonging to these two classes show a high amino acid sequence homology to human VH framework regions and said peptides might be administered to a human directly without expectation of an unwanted immune response therefrom, and without the burden of further humanisation. Indeed, some Camelidae VHH sequences display a high sequence homology to human VH framework regions and therefore said VHH might be administered to patients directly without expectation of an immune response therefrom, and without the additional burden of humanization. Suitable mutations, in particular substitutions, can be introduced during humanization to generate a polypeptide with reduced binding to pre-existing antibodies (reference is made for example to WO 2012/175741 and WO2015/173325), for example in at least one of the positions: 11, 13, 14, 15, 40, 41, 42, 82, 82a, 82b, 83, 84, 85, 87, 88, 89, 103, or 108. The amino acid sequences and/or VHH of the invention may be suitably humanized at any framework residue(s), such as at one or more Hallmark residues (as defined herein) or preferably at one or more other framework residues (i.e. non-Hallmark residues) or any suitable combination thereof. Depending on the host organism used to express the amino acid sequence, ISVD, VHH or polypeptide of the invention, such deletions and/or substitutions may also be designed in such a way that one or more sites for posttranslational modification (such as one or more glycosylation sites at asparagine to be replaced with G, A, or S; and/or Methionine oxidation sites) are removed, as will be within the ability of the person skilled in the art. Alternatively, substitutions or insertions may be designed so as to introduce one or more sites for attachment of functional groups, for example to allow site-specific pegylation. In some cases, at least one of the typical Camelidae hallmark residues with hydrophilic characteristics at position 37, 44, 45 and/or 47 is replaced (Kabat No; see WO2008/020079 Table A-03). Another example of humanization includes substitution of residues in FR 1, such as position 1, 5, 11, 14, 16, and/or 23, and/or 28; in FR2 such as positions 40 and/or 43; in FR3, such as positions 60-64, 73, 74, 75, 76, 78, 79, 81, 82b, 83, 84, 85, 93 and/or 94; and in FR4, such as position 103, 104, 105, 108 and/or 111 (see WO2008/020079 Tables A-05-A08; all numbering according to the Kabat).

In a specific embodiment, the binding agents comprise a CI-M6PR-specific ISVD which is a humanized variant of any one of SEQ ID NO:1, 5, 7, 8, 71 and 73. In a particular embodiment, said binding agent comprises a CI-M6PR-specific ISVD which is a humanized variant of SEQ ID NO:7, or 8, resp., which may specifically but not limited be presented as in SEQ ID No: 93-102.

Another embodiment relates to a binding agent comprising an ISVD specifically binding to CI-M6PR domain 1-3, which is a multi-specific agent, comprising a first binding agent with an ISVD-binder for CI-M6PR as described herein, and a further (second or more) binding agent directly or indirectly linked or coupled to said binding agent. When said further binding agent comprises a binding agent specific for a CI-M6PR, but with a chemical structure different from the first binding agent, this may result in a multiparatopic or multispecific binding agent. When said further binding agent comprises a binding agent specific that is the same or identical to the first binding agent, this provides for a multivalent CI-M6PR binder, which may increase the avidity for binding for instance. Furthermore, said further binding agent of said multi-specific binding agent may also comprise another form of a multi-specific CI-M6PR binding agent, including a binding agent with a different target specificity. By coupling several binders, which all may comprise an ISVD in a specific embodiment, interacting with different target, preferably targets present on the cell surface or extracellular environment, these are defined as multispecific binding agents. A “multi-specific” form for instance, is formed by bonding together two or more immunoglobulin single variable domains, of which at least one with a different specificity. Non-limiting examples of multi-specific constructs include “bi-specific” constructs, “tri-specific” constructs, “tetra-specific” constructs, and so on. To illustrate this further, any multivalent or multi-specific (as defined herein) protein binding agent of the invention may be suitably directed against two or more different epitopes on the same antigen, for example against epitope 1 on one domain and epitope 2 on another domain of CI-M6PR; or may be directed against two or more different antigens, for example against CI-M6PR and one as a half-life extension against Serum Albumin. One of the most widely used techniques for increasing the half-life and/or reducing immunogenicity of pharmaceutical proteins comprises attachment of a suitable pharmacologically acceptable polymer, such as poly(ethyleneglycol) (PEG) or derivatives thereof (such as methoxypoly(ethyleneglycol) or mPEG). Another technique for increasing the half-life of a binding domain may comprise the engineering into bifunctional or bispecific domains (for example, one or more ISVDs or active antibody fragments against CI-M6PR coupled to one ISVD or active antibody fragment against serum albumin aiding in prolonging half-life)) or into fusions of antibody fragments, in particular immunoglobulin single variable domains, with peptides (for example, a peptide against a serum protein such as albumin). The coupling to additional moieties will result in multispecific binding agent, as further disclosed herein.

Multivalent or multi-specific binding agents of the invention may also have (or be engineered and/or selected for) increased avidity and/or improved selectivity for the desired CI-M6PR interaction, and lysosome targeting function, and/or for any other desired property or combination of desired properties that may be obtained by the use of such multivalent or multi-specific binding agents. For instance, the combination of one or more ISVDs binding epitope 1, and one or more ISVDs binding epitope 2 as described herein, results in a multi-specific binding agent of the invention with the potential of cellular uptake of the full complex of binding agent and all its targets bound to it, via CI-M6PR internalisation, which may ultimately lead to degradation of said target(s) in the lysosome. Said multi-specific binding agent comprises at least said binding agents directed against epitope 1 and epitope 2, which may be coupled via a linker, spacer. Upon binding CI-M6PR, said multi-specific binding agent or multivalent ISVD may have an additive or synergistic impact on the CI-M6PR internalizing activity, or may be used to target and extract or shuffle cell-surface or extracellular molecules from the extracellular or membrane environment into the endosomes and lysosome, or alternatively, used to prolong their half-life by recycling those targets through the endosome cycling pathway. The multispecific binders of the invention may be coupled to a functional moiety, a targeting moiety, a half-life extending moiety, or to a cell penetrant carrier.

So in a specific embodiment, the invention relates to bifunctional bispecific agents which target CI-M6PR, as described herein, and as a second binding specifically target a cell surface molecule or extracellular molecule, wherein such a bispecific agent may enhance degradation of the target relative to degradation of the cell surface molecule or extracellular molecule in the presence of the CI-M6PR binding agent alone (so not coupled to said further binding agent specifically binding the target).

In a further specific embodiment, said further binding agent specifically binding a cell surface or extracellular target may comprise an ISVD, a VHH, or a Nb, or alternatively may comprise a small molecule (which may be linked via covalent chemical coupling) or may be a peptide or peptidomimetic. Further specific embodiments relate to bispecific or multispecific formats comprising said ISVD-based CI-M6PR binders as described herein, and directly or indirectly via a spacer or linker, or chemically, coupled to further binding agents. Said coupling or fusion of a CI-M6PR specific ISVD to for instance, another ISVD, antibody fragment or antibody-type of VH or VL structure as defined herein, may also occur through linking via an Fc tail as to produce bispecific ISVD-Fc antibodies.

Hence, specific embodiments envisaged herein include the those bispecific chimeras, wherein the ISVD-based against specific interacting with the N-terminal part of CI-M6PR retains its binding to the CI-M6PR during its endosomal cycle, and this has a binding affinity that is stable and resistant to dissociation down to pH ˜5.5. The anti-CI-M6PR VHHs described herein provide for a panel of highly specific and high affinity binders at neutral pH, though with different pH dissociation profiles when lowering pH (in vitro) down to pH6, 5, 4.5 or 4. This panel thus provides for a versatile toolbox to explore bispecifics with lysosomal degradation and recycling potential of different nature depending on the needs for specific targets and applications. Moreover, the high affinity of said CI-M6PR binding agents (nanomolar to picomolar KD values) at neutral pH is required as to ensure specific tight binding to the receptor on the cell surface, though subsequently a need to dissociate rapidly when internalized in endosome/lysosome may be desired as to increase the chance that the same late endosomal/lysosomal delivery route is followed as the natural cargo of the CI-M6PR. In view of optimizing binding affinity at specific pH conditions, methods are known to the skilled person as how to engineer the binding agents such as the VHHs using for instance histidine scanning method mutagenesis¹⁰⁹, which is specifically aimed at reducing the binding affinity of antibodies at acidic pH as compared to neutral pH. As the imidazole side chain of a histidine residue has a pKa^{˜}6.0, the switching of its protonation state alters binding interactions at interfaces where it occurs. Briefly, a combinatorial phage library is obtained with histidines incorporated into the VHH CDRs. This library will then be screened through biopanning with binding at pH 7.4 and elution at pH 5.5, followed by determination of the exact binding characteristics of the resulting VHHs at these pH's through BLI.

In a particular embodiment, kits are provided which contain means to detect CI-M6PR protein, including the binding agent or ISVDs as described herein, allowing to detect or modulate CI-M6PR localisation and trafficking in a system, which may be an in vitro or in vivo system. It is envisaged that these kits are provided for a particular purpose, such as for endosome/lysosome labeling, or for in vivo imaging, or for diagnosis of an altered CI-M6PR quantity, response or effect in a subject. In another embodiment, said kit is provided which contains means including a nucleic acid molecule, a vector, or a pharmaceutical composition as described herein. The means further provided by the kit will depend on the methodology used in the application, and on the purpose of the kit. For instance, detection of a labelled CI-M6PR ISVD-based binding agent, as described herein, or nucleic acid molecule as described herein, which may be desired for CI-M6PR quantification on nucleic acid or protein level. For protein-based detection, the kits typically will contain labelled or coupled CI-M6PR binding agents such as ISVDs. Likewise, for detection at the nucleic acid level, the kits may contain labels for nucleic acids such as primers or probes. Further control agents, antibodies or nucleic acids may also be provided in the kit. A standard, for reference or comparison, a CI-M6PR substrate or signaling component, a reporter gene or protein or other means for using the kit may also be included. Of course, the kit may further comprise pharmaceutically acceptable excipients, buffers, vehicles or delivery means, an instruction manual and so on.

Another aspect of the invention provides for a method for detecting the presence, absence or level of CI-M6PR protein in a sample, the method comprising: contacting the sample with the CI-M6PT binding agent or ISVD as described herein, and detecting the presence or absence or level, i.e. quantifying, the bound CI-M6PR ISVD, which is optionally a labelled, conjugated or multispecific CI-M6PR binding agent. The sample used herein may be a sample isolated from the body, such as a body fluid, including blood, serum, cerebrospinal fluid, among others, or may be an extract, such as a protein extract, a cell lysate, etc.

Furthermore, the ISVD-based binding agent, in particular comprising a CI-M6PR-specific ISVD, the nucleic acid molecule, the vector, or the pharmaceutical composition comprising said CI-M6PR-specific binding agent, as described herein, may also be used for in vivo imaging.

For the purpose of detection and/or imaging, in vitro or in vivo, the CI-M6PR binding agent, comprising a CI-M6PR-specific ISVD, as described herein may further comprise in some embodiments a detection agent, such as a tag or a label. For instance, the ISVDs, VHHs, or Nbs as exemplified herein were also tagged, by the 6-His-EPEA double tag (or for an EPEA tag: see also WO2011/147890A1). Such a tag allows affinity purification and detection of the antibody or active antibody fragments of the invention.

Some embodiments comprise the CI-M6PR binding agent, ISVD, further comprising a label or tag, or more specifically, the CI-M6PR binding agent labelled with a detectable marker. The term detectable label or tag, as used herein, refers to detectable labels or tags allowing the detection and/or quantification of the CI-M6PR binding agent as described herein, and is meant to include any labels/tags known in the art for these purposes. Particularly preferred, but not limiting, are affinity tags, such as chitin binding protein (CBP), maltose binding protein (MBP), glutathione-S-transferase (GST), poly(His) (e.g., 6×His or His6), biotin or streptavidin, such as Strep-Tag®, Strep-tag II® and Twin-Strep-Tag®; solubilizing tags, such as thioredoxin (TRX), poly(NANP) and SUMO; chromatography tags, such as a FLAG-tag; epitope tags, such as V5-tag, myc-tag and HA-tag; fluorescent labels or tags (i.e., fluorochromes/-phores), such as fluorescent proteins (e.g., GFP, YFP, RFP etc.) and fluorescent dyes (e.g., FITC, TRITC, coumarin and cyanine); luminescent labels or tags, such as luciferase, bioluminescent or chemiluminescent compounds (such as luminal, isoluminol, theromatic acridinium ester, imidazole, acridinium salts, oxalate ester, dioxetane or GFP and its analogs); phosphorescent labels; a metal chelator; and (other) enzymatic labels (e.g., peroxidase, alkaline phosphatase, beta-galactosidase, urease or glucose oxidase); radioisotopes. Also included are combinations of any of the foregoing labels or tags. Technologies for generating labelled polypeptides and proteins are well known in the art. A CI-M6PR binding agent comprising a CI-M6PR-specific ISVD of the invention, coupled to, or further comprising a label or tag allows for instance immune-based detection of said bound CI-M6PR-specific agent. Immune-based detection is well known in the art and can be achieved through the application of numerous approaches. These methods are generally based upon the detection of a label or marker, such as described above. See, for example, U.S. Pat. Nos. 3,817,837; 3,850,752; 3,939,350; 3,996,345; 4,277,437; 4,275,149 and 4,366,241. In the case where multiple antibodies are reacted with a single array, each antibody can be labelled with a distinct label or tag for simultaneous detection. Yet another embodiment may comprise the introduction of one or more detectable labels or other signal-generating groups or moieties, or tags, depending on the intended use of the labelled or tagged CI-M6PR binding agent of the present invention. Other suitable labels will be clear to the skilled person, and for example include moieties that can be detected using NMR or ESR spectroscopy. Such labelled CI-M6PR binding agents, such as CI-M6PR-specific ISVDs or Nanobodies as described herein may for example be used for in vitro, in vivo or in situ assays (including immunoassays known per se such as ELISA, RIA, EIA and other “sandwich assays”, etc.) as well as in vivo imaging purposes, depending on the choice of the specific label.

So in another aspect, an in vitro method is disclosed for detection of the localization and distribution of human CI-M6PR protein in a biological sample, comprising the steps of: reacting the sample with a CI-M6PR binding agent, comprising a CI-M6PR-specific ISVD as described herein, and detecting, the localization and distribution of said CI-M6PR binding in said biological sample. The biological sample as used herein may envisage any sample derived from a biological system, and for example comprise cells of brain tissue, or an extract or an in vitro sample, or a body fluid such as cerebrospinal fluid or blood.

Application of CI-M6PR-specific binding agents in Enzyme-replacement therapy.

The identification of CI-M6PR-specific VHHs revealed the potential of generating novel strategies to overcome the known hurdles in currently used ERT approached. So the ISVDs as described herein were tested in genetic fusions to enzymes relevant for ERT. The typical properties of VHHs, known as highly stable and soluble, conformational stability, high intrinsic pH and protease resistance, all form attractive properties for cycling through the endosomal-lysosomal system. Furthermore, VHH-based formats are suitable for various routes of administration, including via intravenous injection and inhalation, thus providing for a novel approach to apply lysosomal targeting of drug products, optionally in complex with their targets. More specifically, the binders as described herein may as fusion be further coupled or operably linked to further binding moieties, which may be additional ISVDs, or antigen-binding domains specific for a target protein, preferably a target present on the cell surface or extracellularly, or to extend the half-life (e.g. serum albumin specific binders), or alternative compounds that are providing a function. Such multispecific binders or enzyme/CI-M6PR-VHH fusion polypeptide result in CI-M6PR-mediated lysosomal uptake, as cargo for enzyme delivery.

Because the CI-M6PR constantly traffics between the late endosome and the cell membrane, the binding agents disclosed herein, preferably for use in ERT applications, may dissociate at the lower pH in these subcellular organelles, such as around pH 5.8, being the endosomal condition, where upon M6PR dissociation or release, the binding agent may continue to the lysosome, where it is finally degraded.

The invention provides for binding agents comprising fusion proteins comprising the ISVD-based binding agent specific for binding to CI-M6PR, as described herein, and an enzyme, coupled for instance by a genetic fusion. Said enzyme preferably being an enzyme known to be required or for use in Enzyme-Replacement Therapy.

### Fusion Proteins for Lysosomal Targeting of Acid α-Glucosidase (GAA)

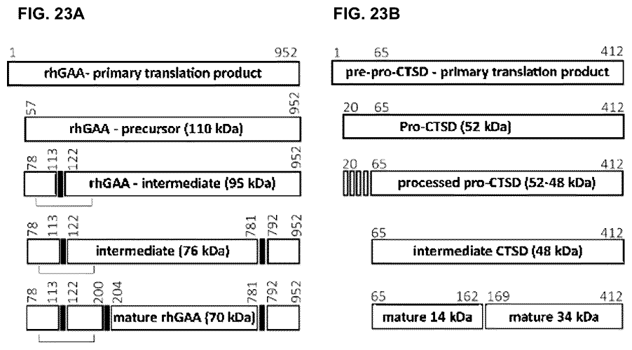

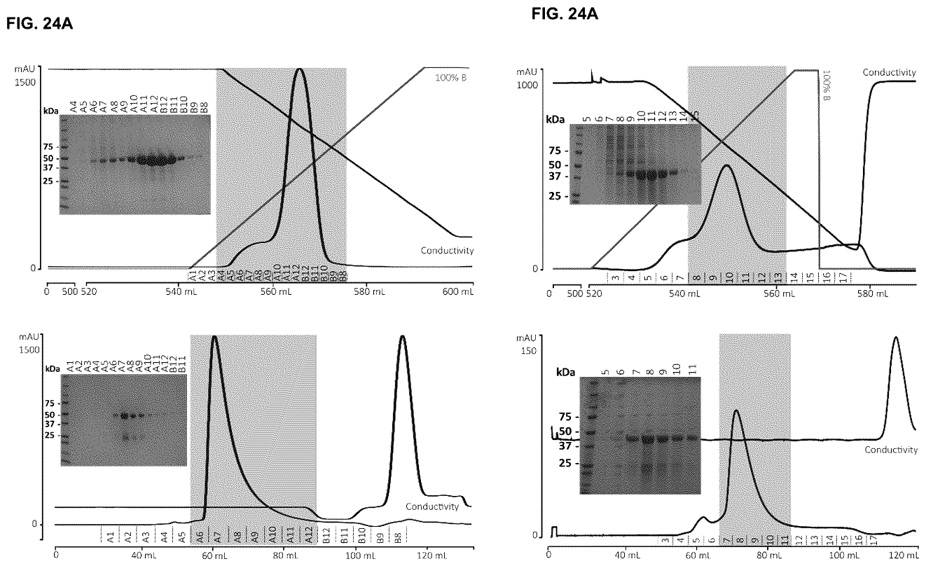

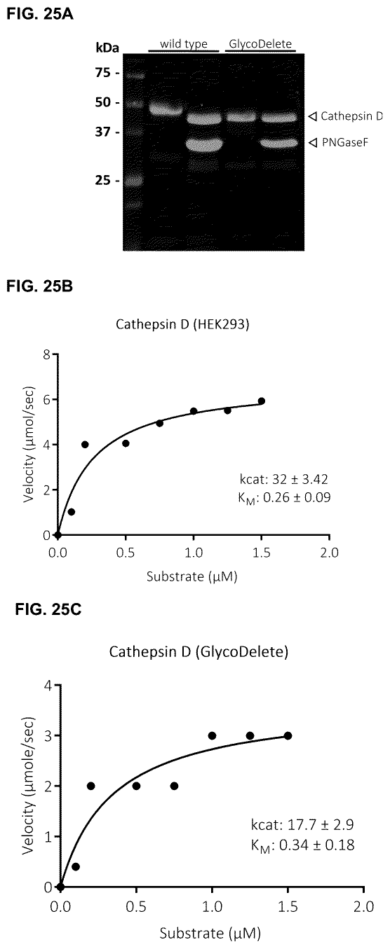

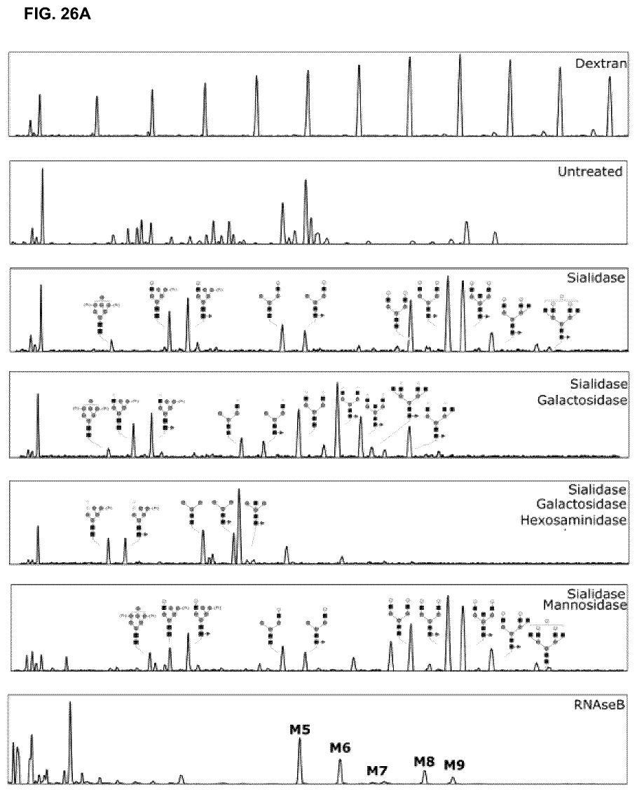

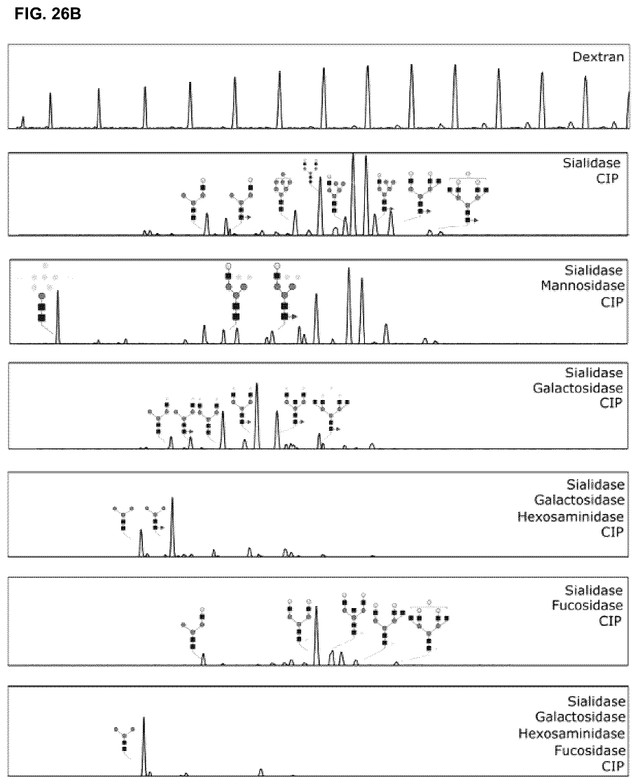

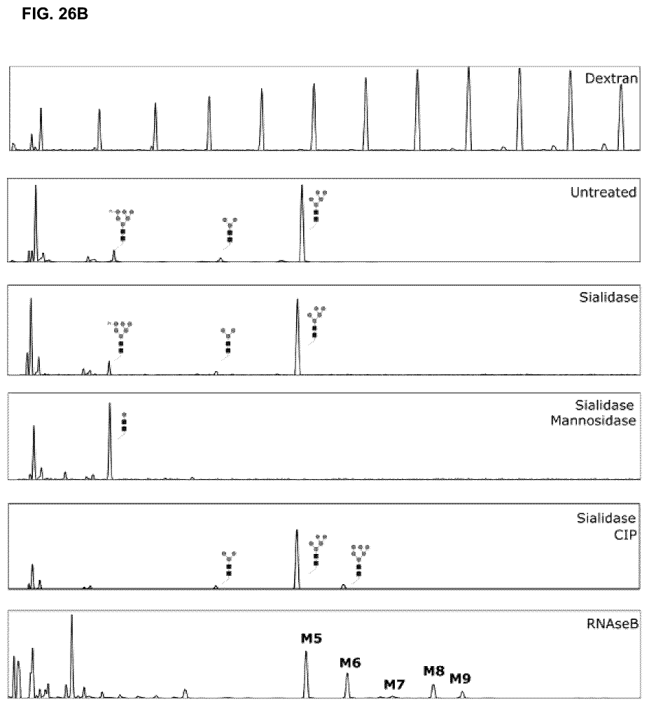

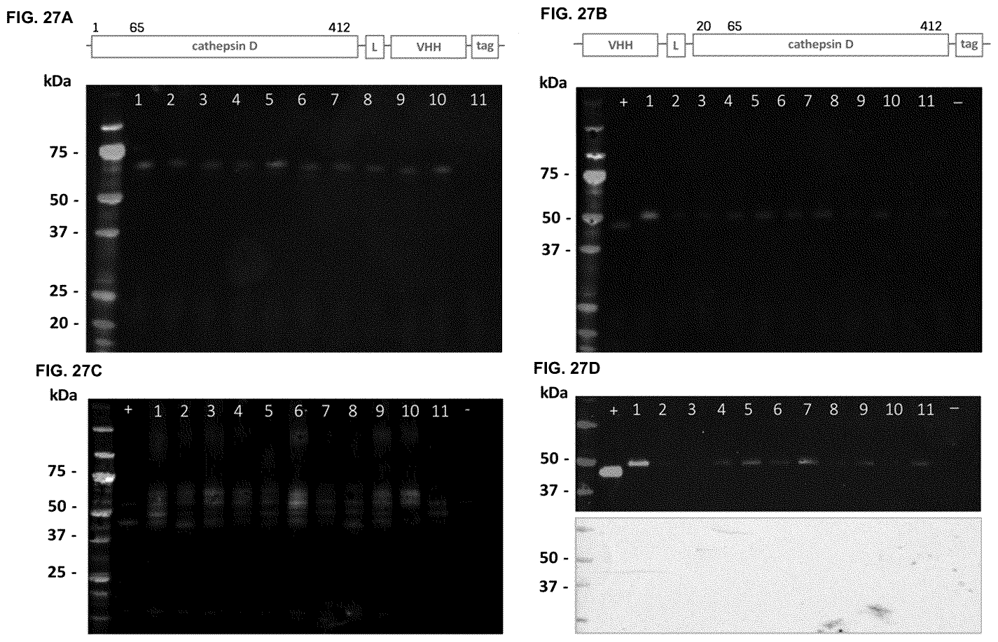

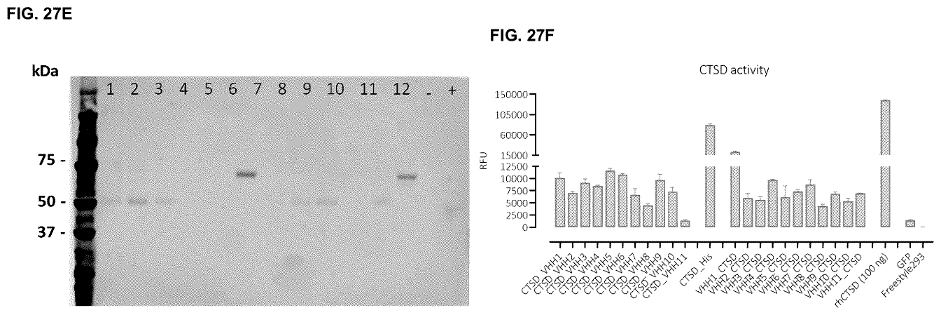

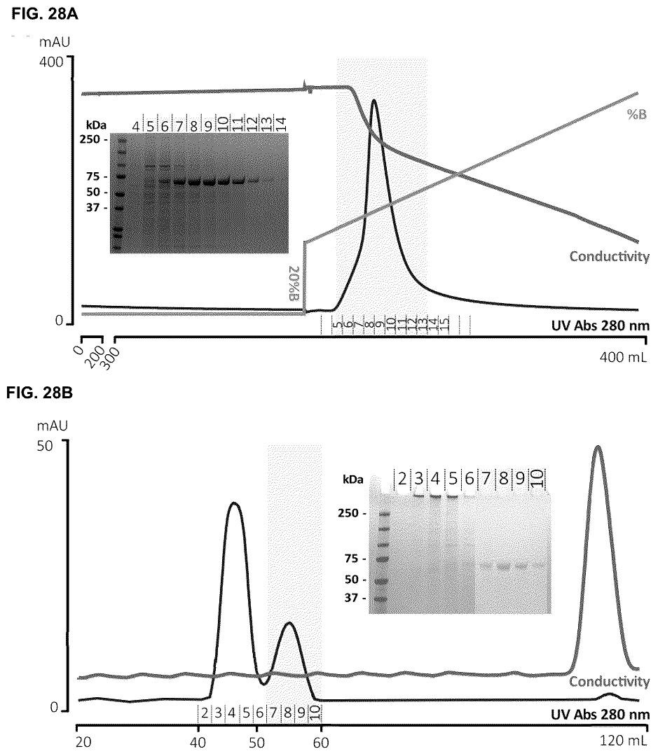

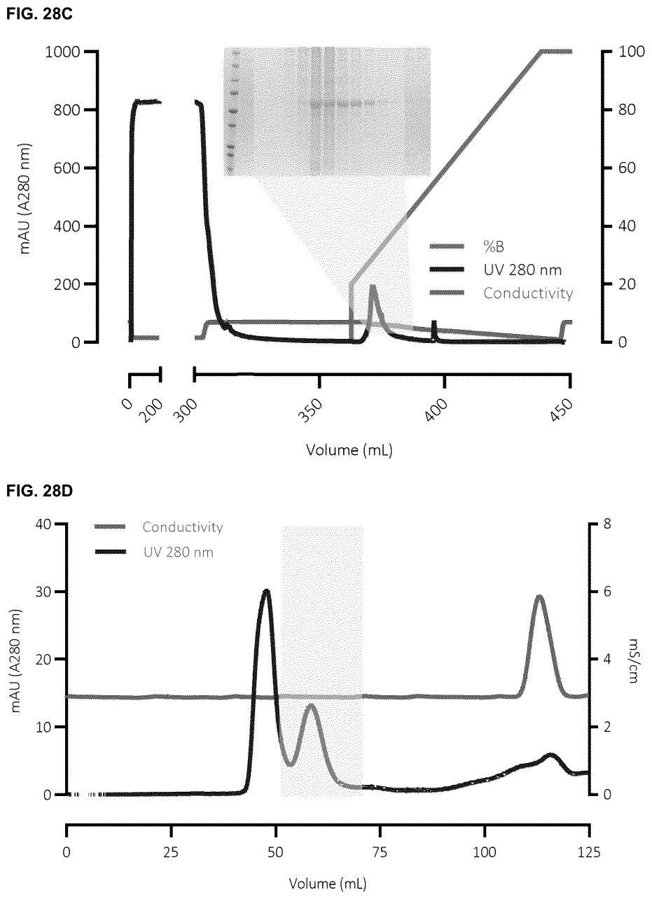

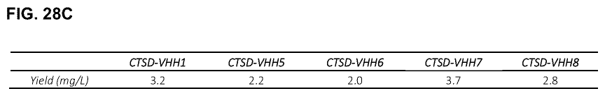

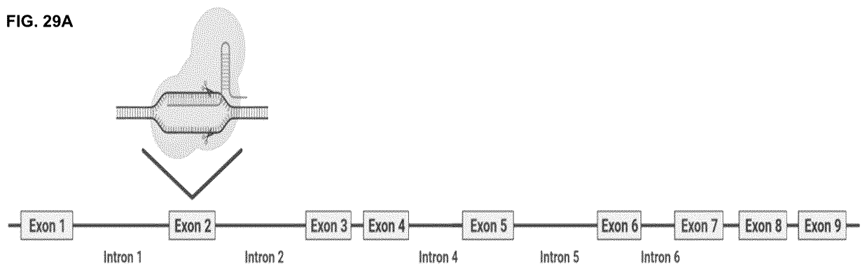

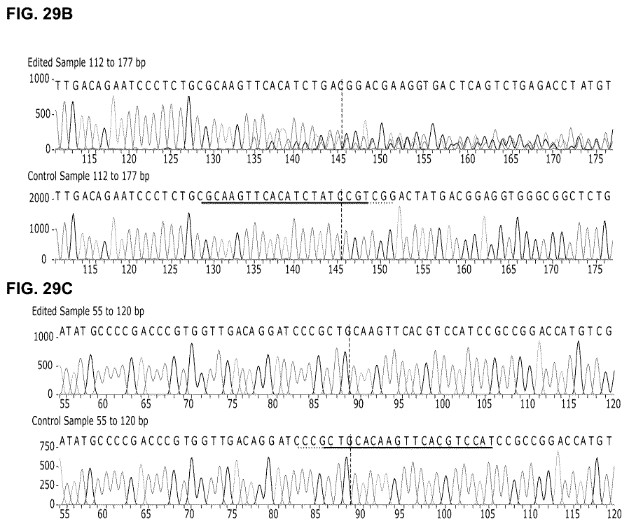

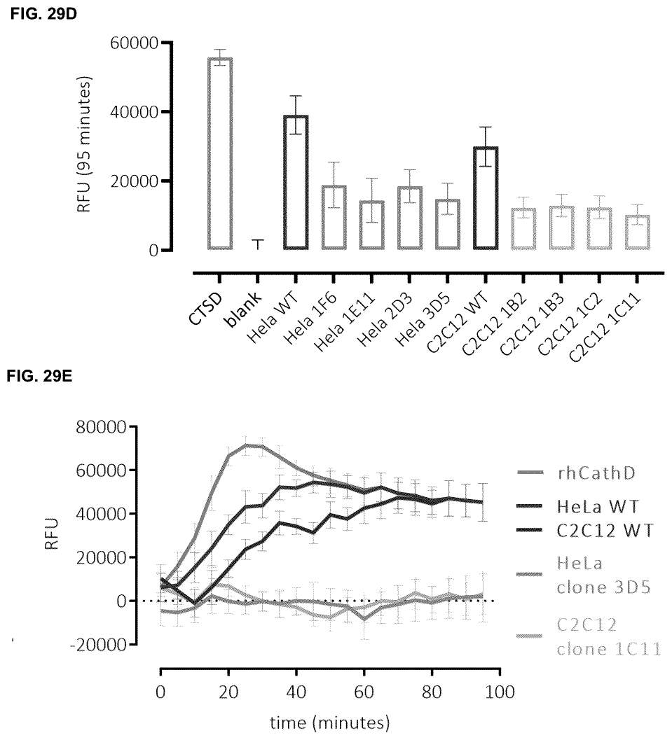

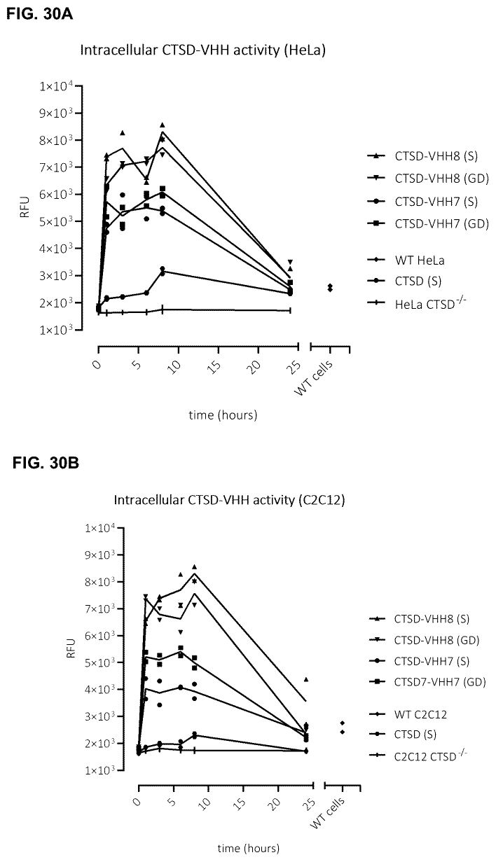

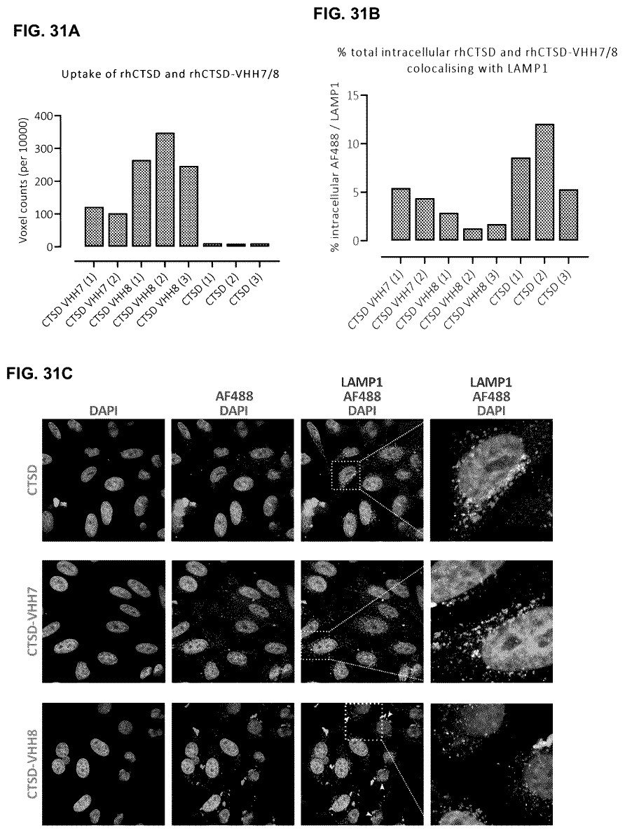

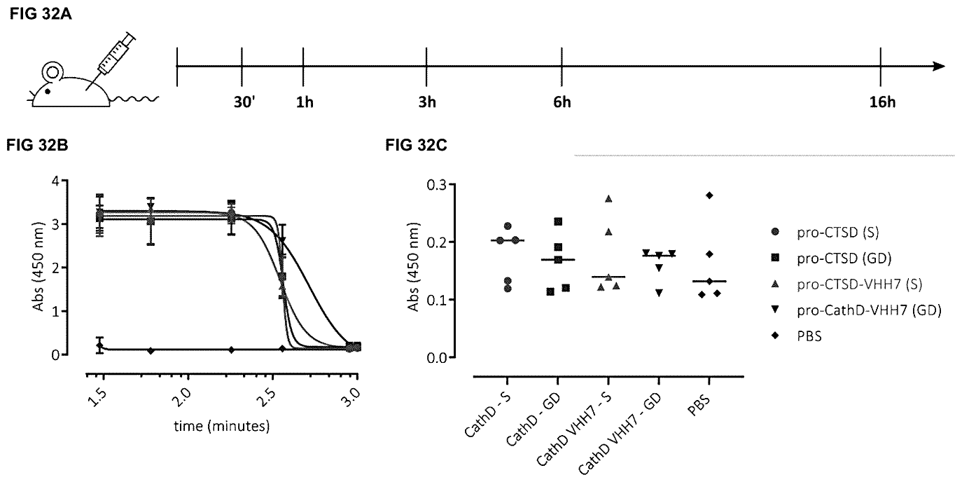

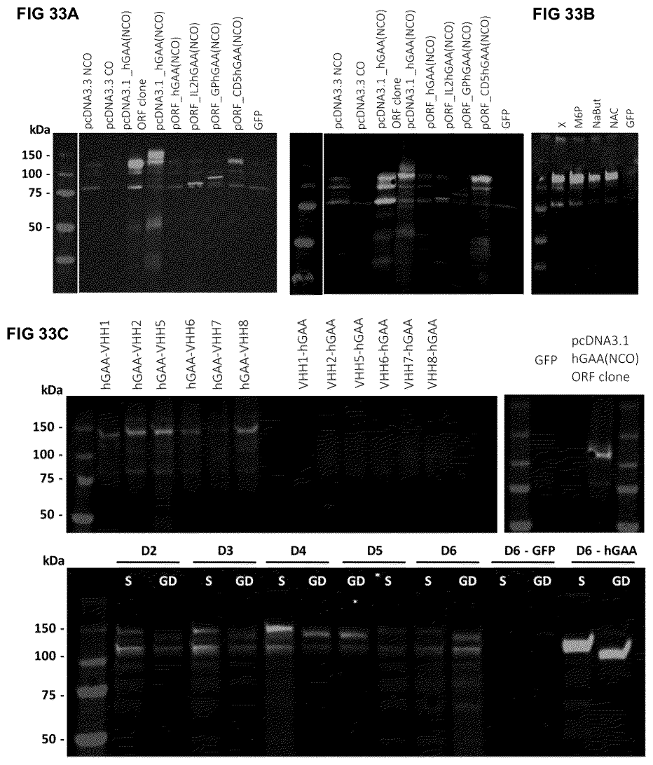

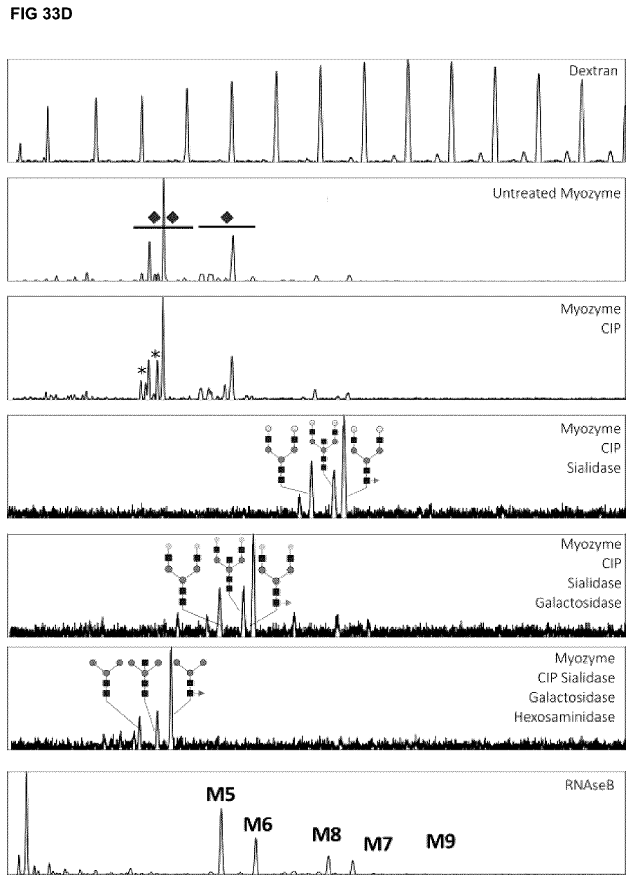

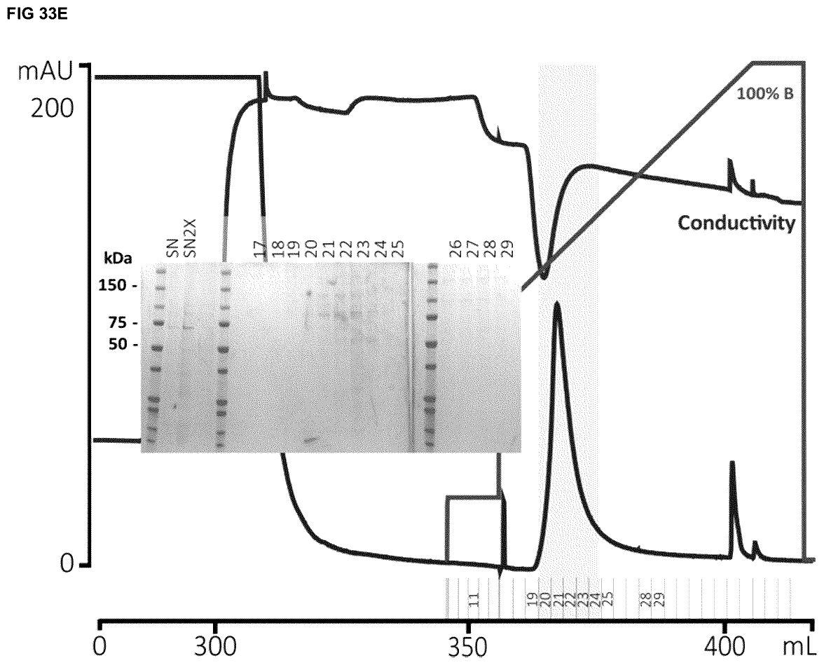

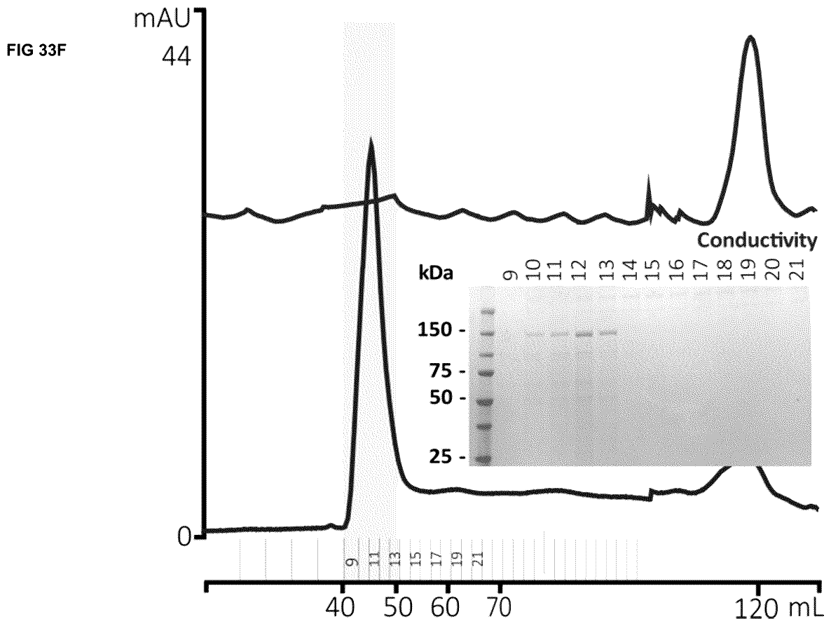

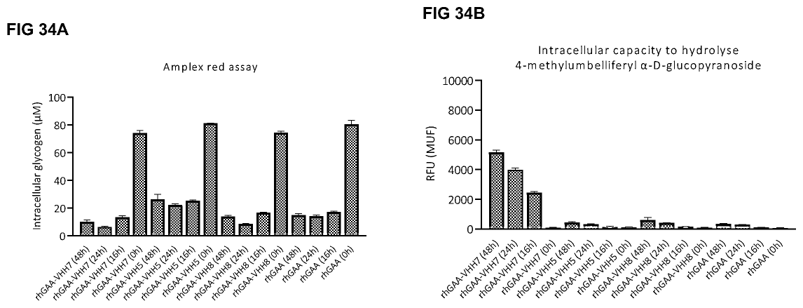

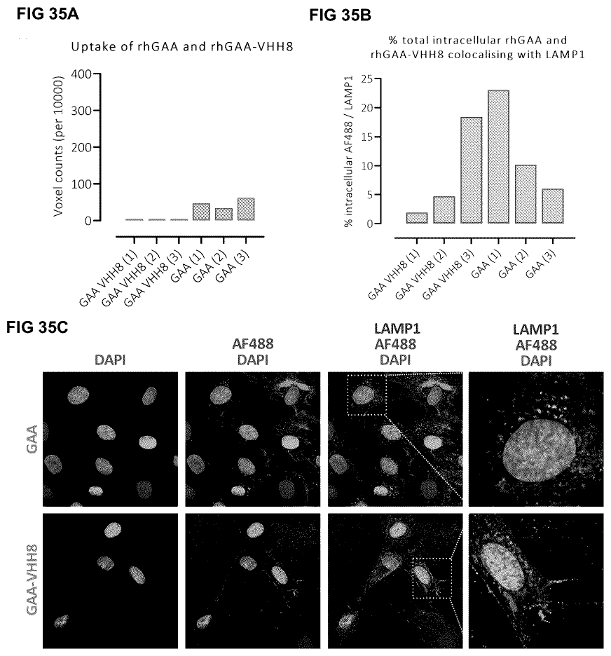

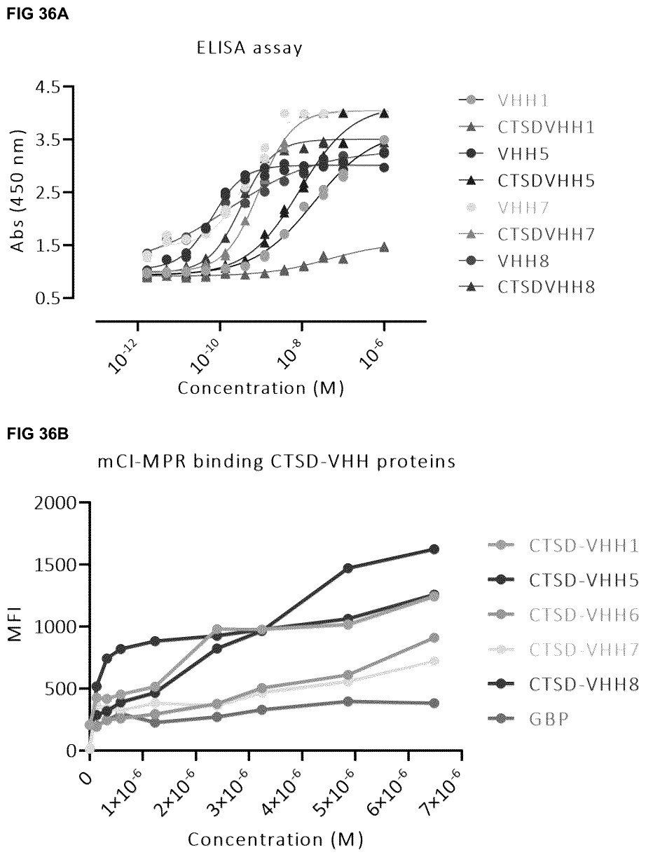

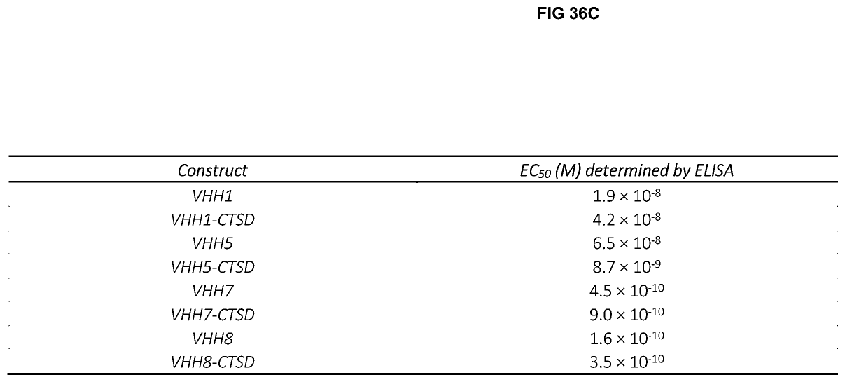

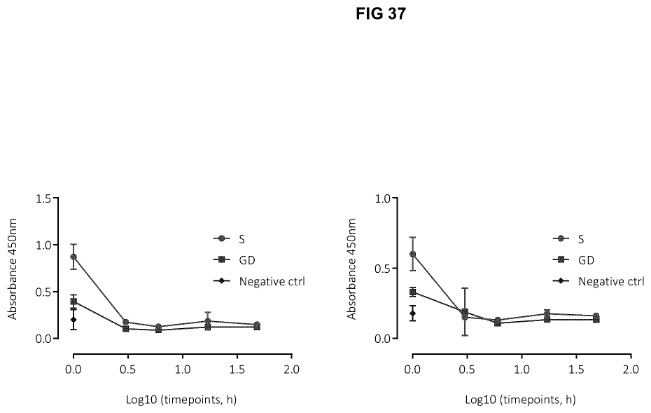

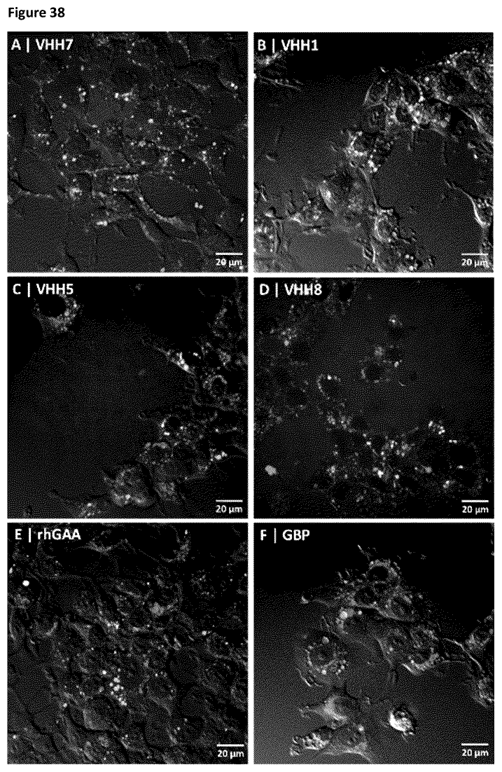

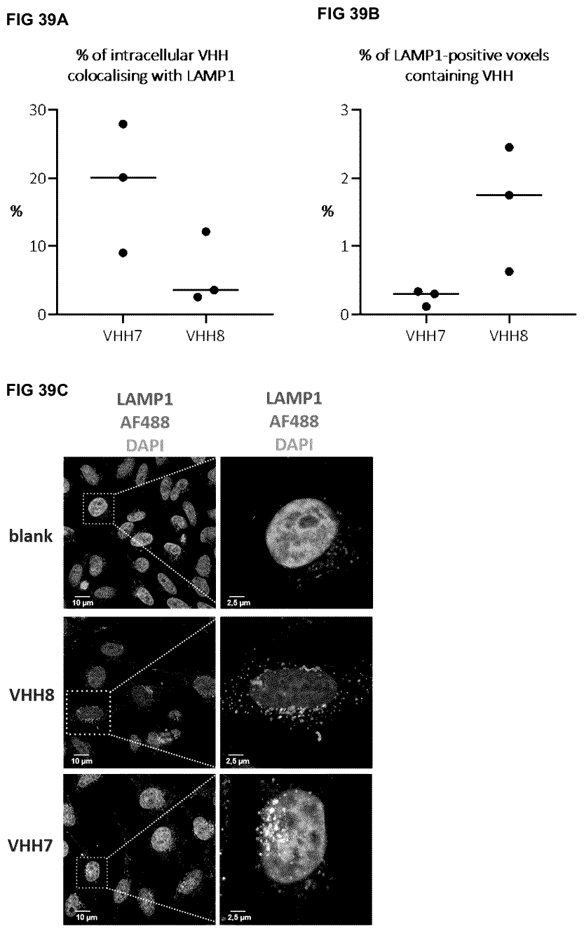

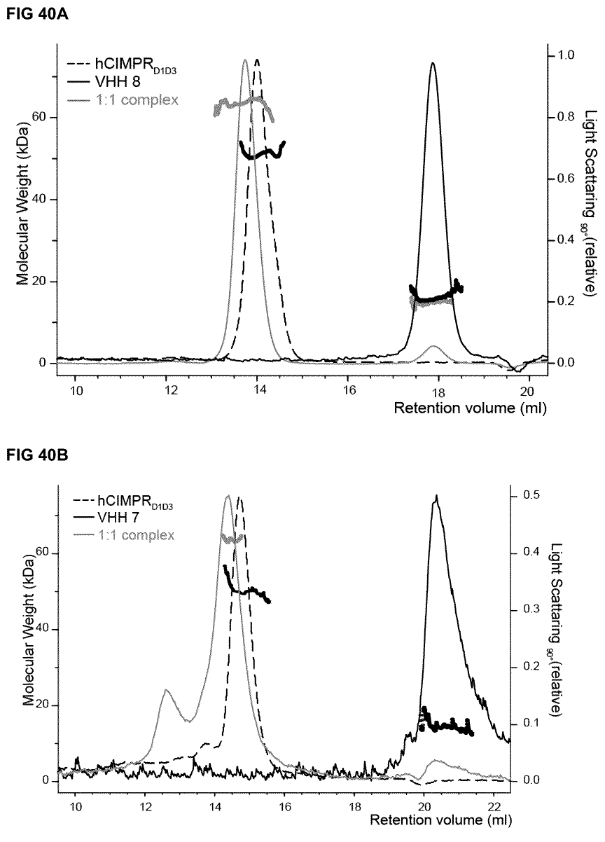

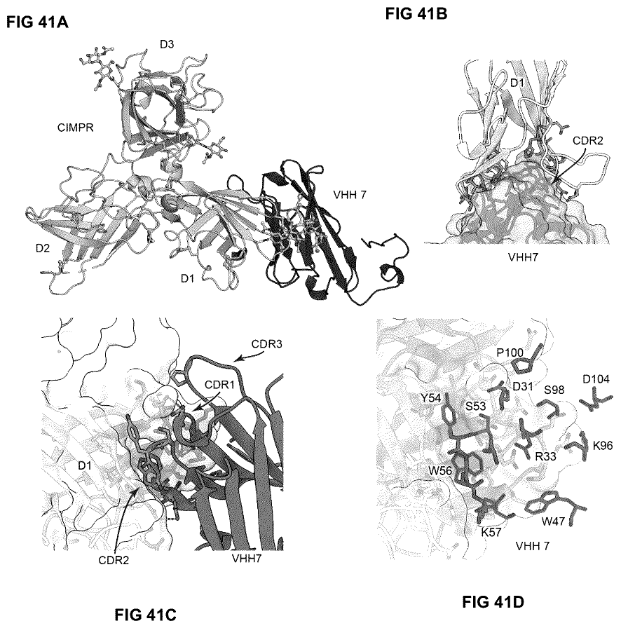

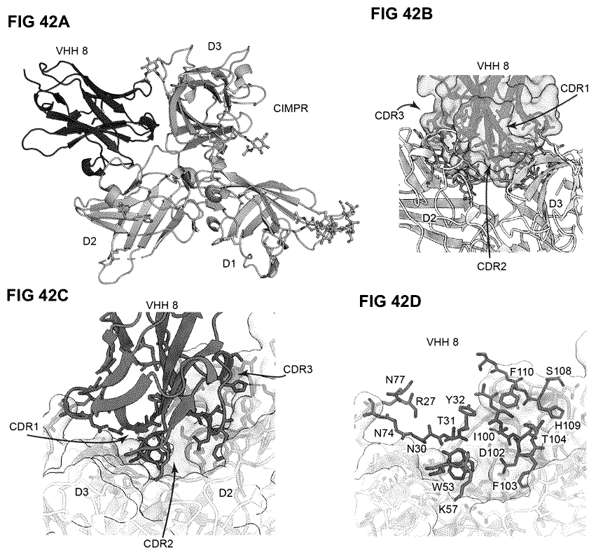

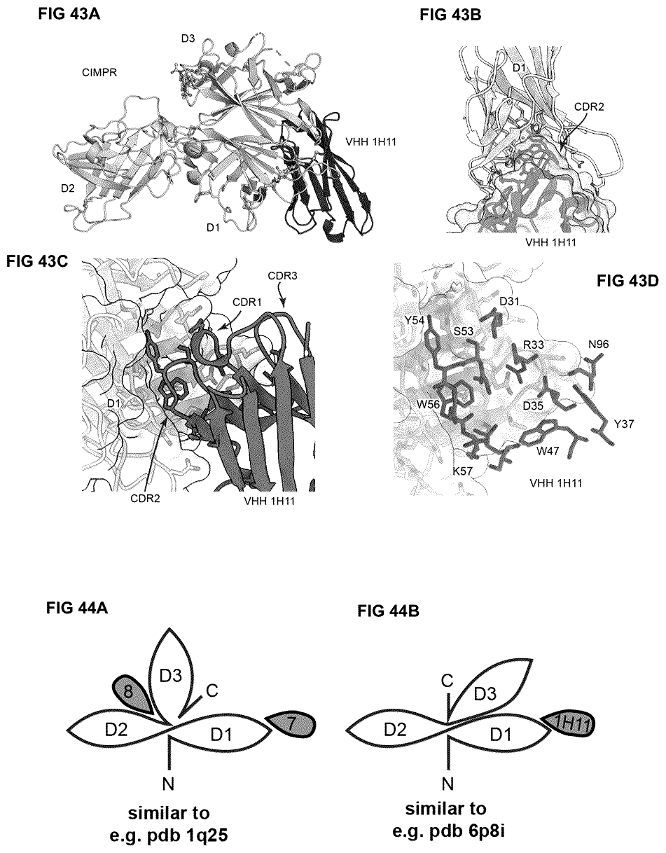

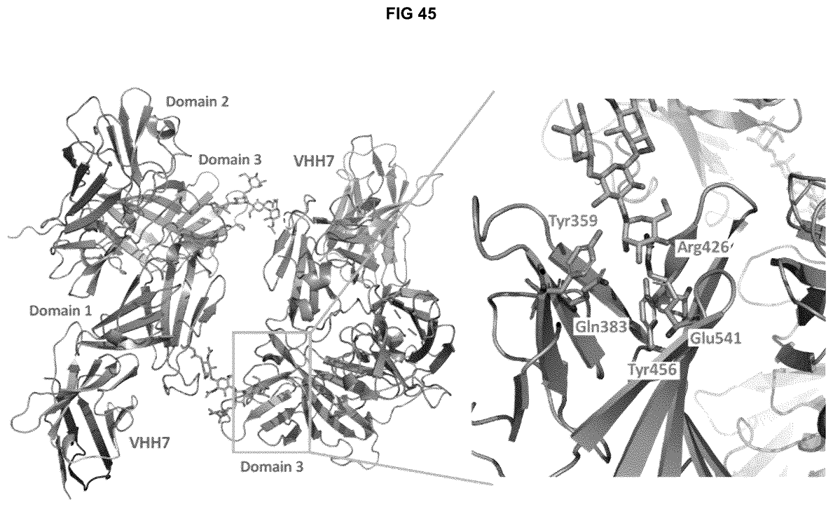

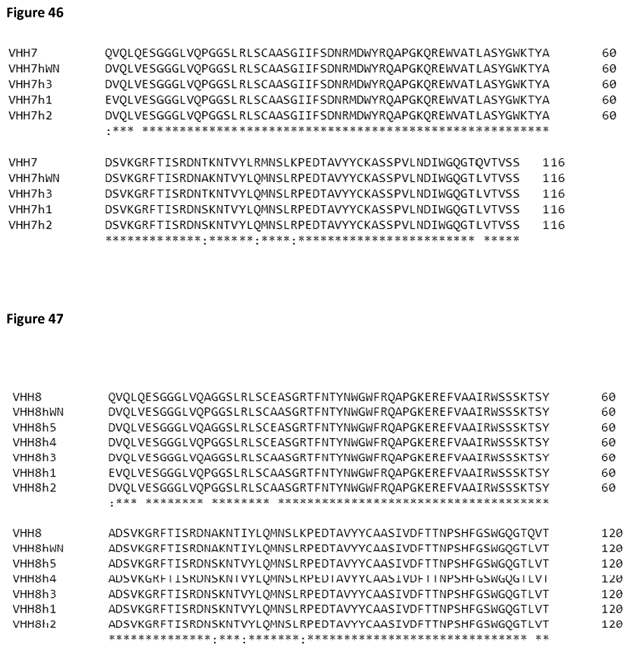

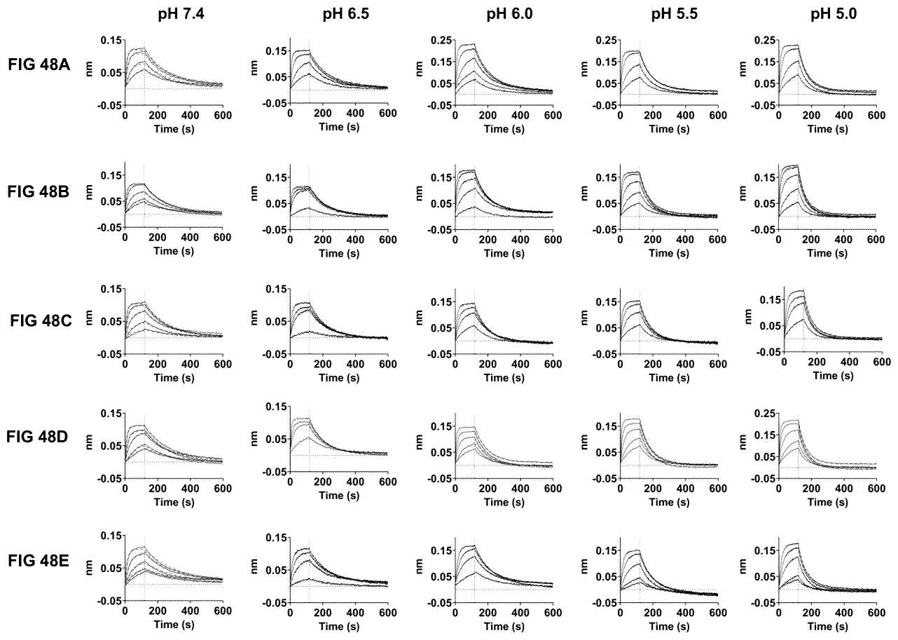

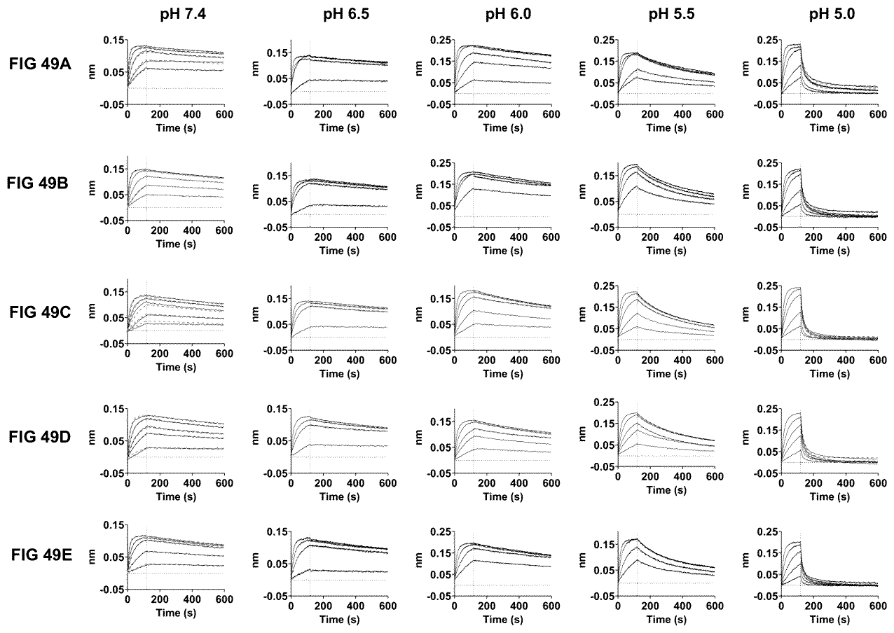

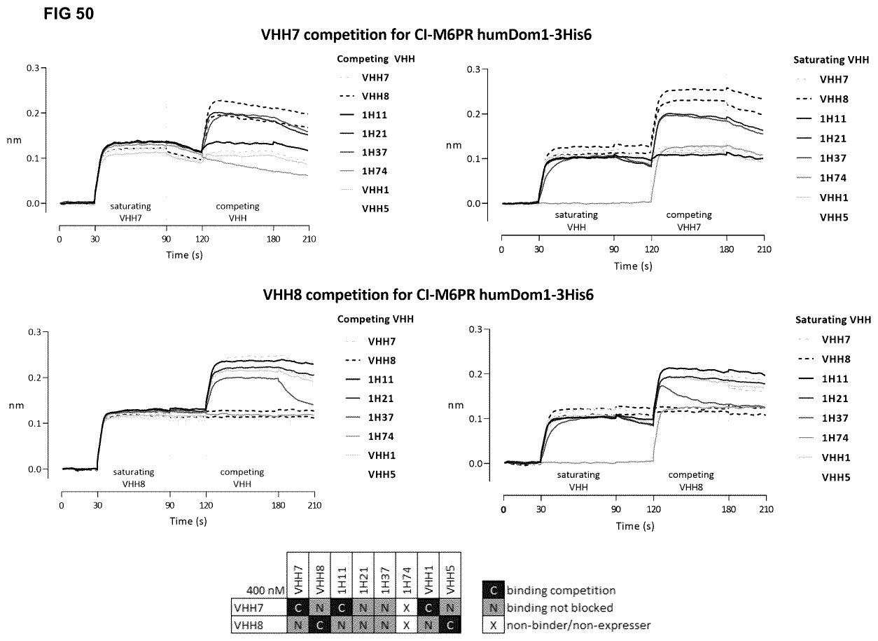

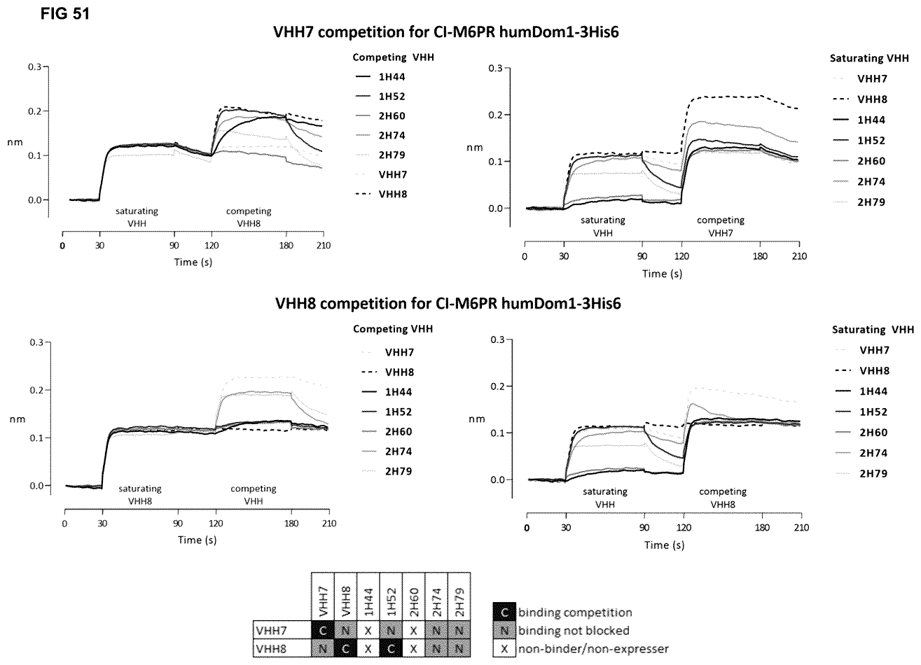

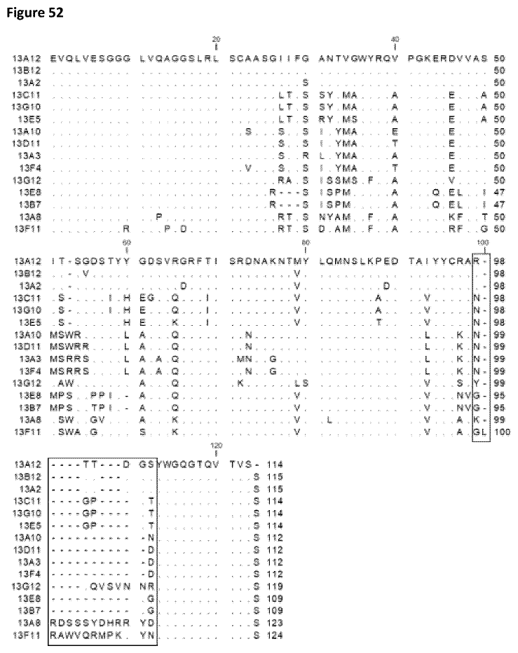

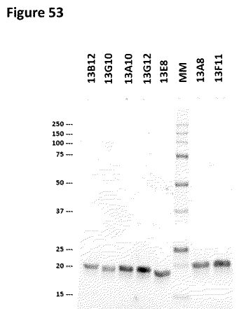

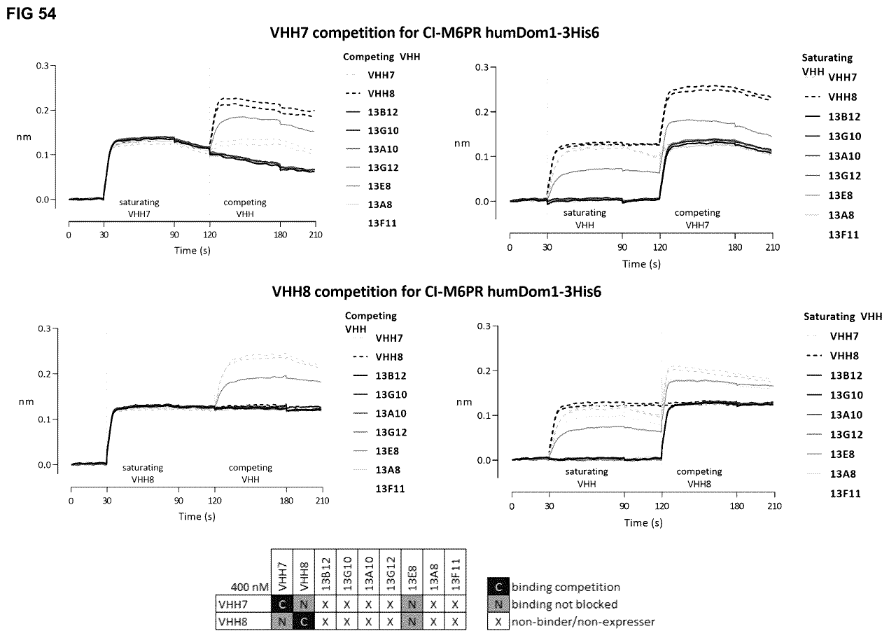

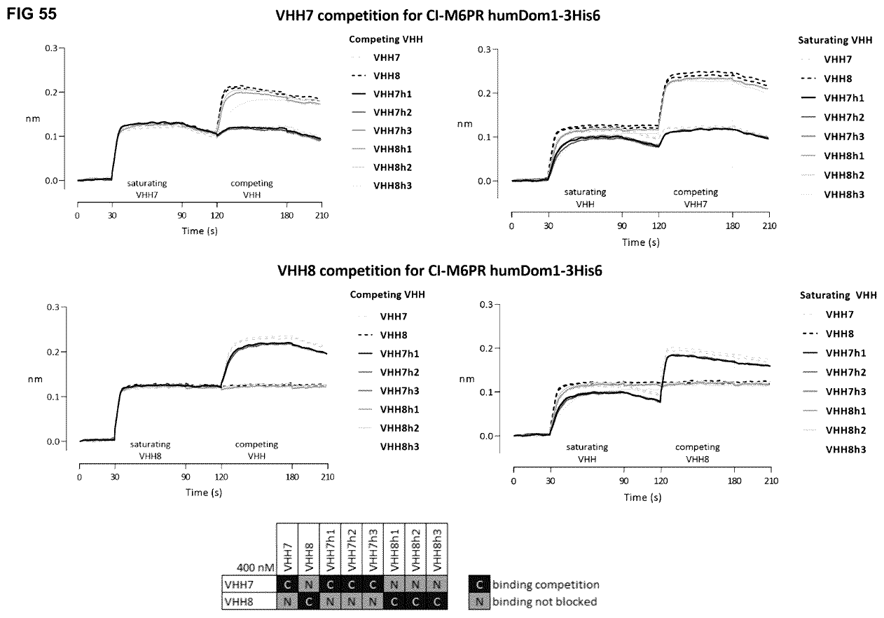

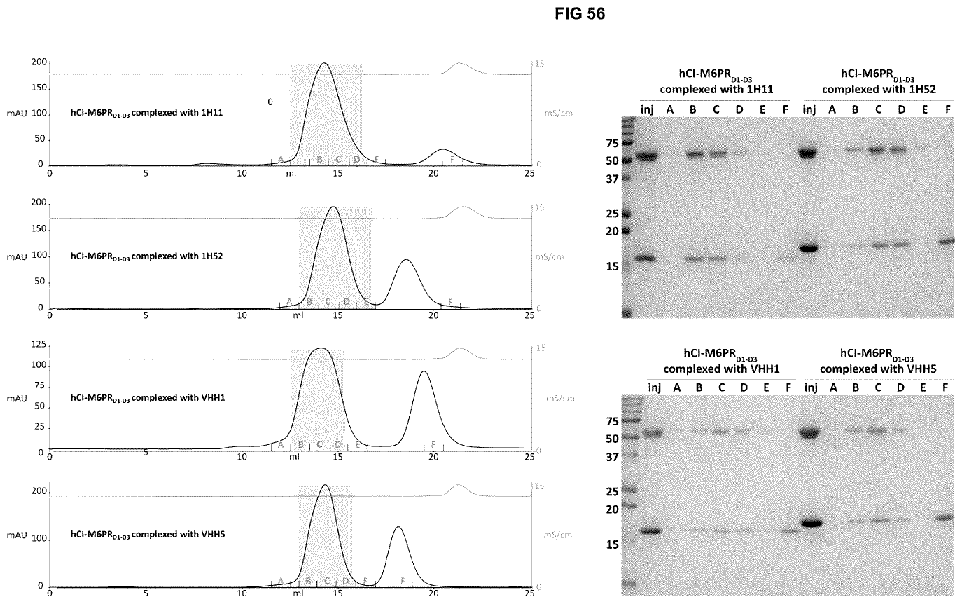

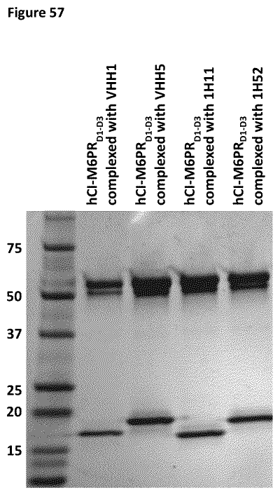

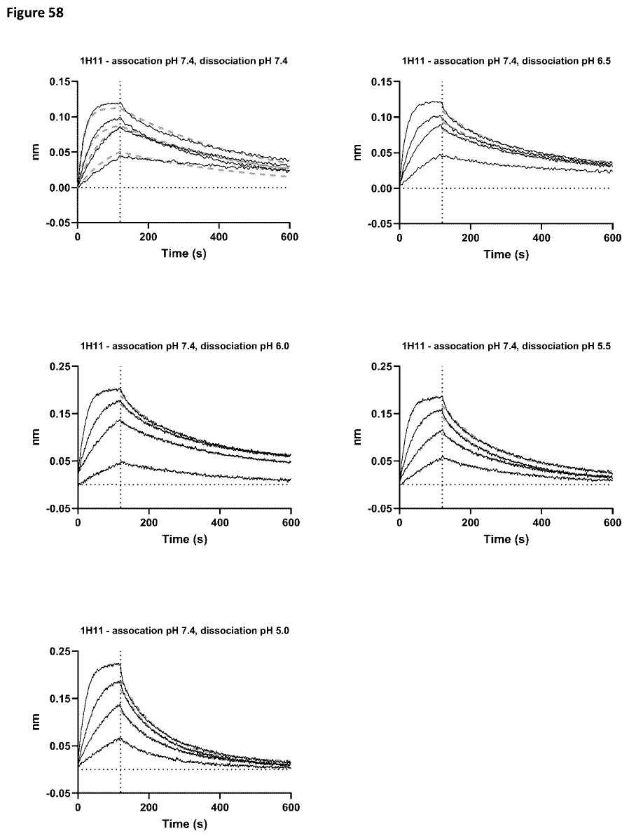

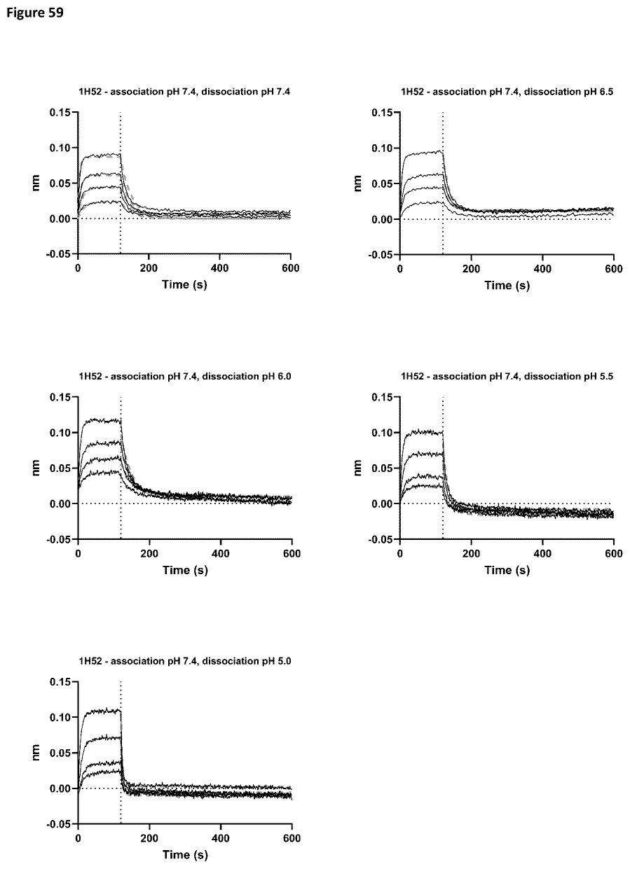

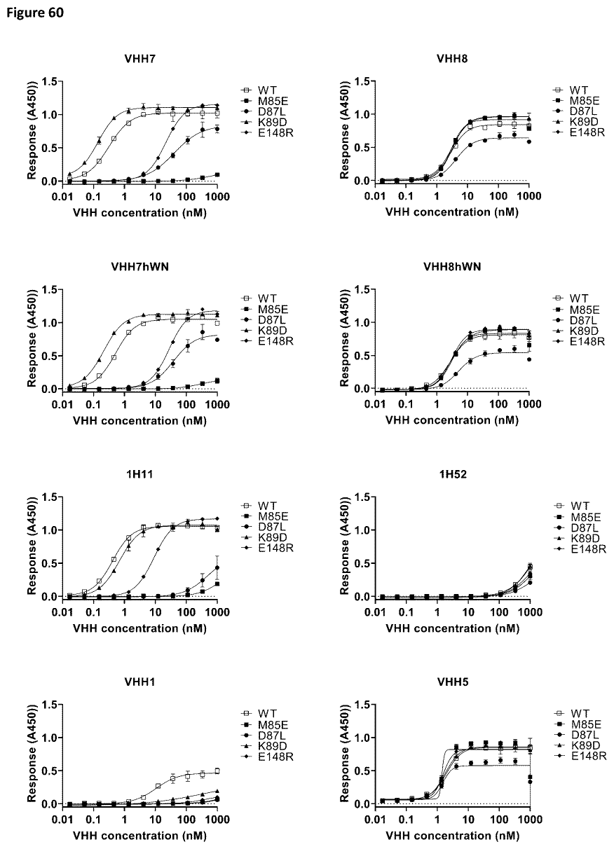

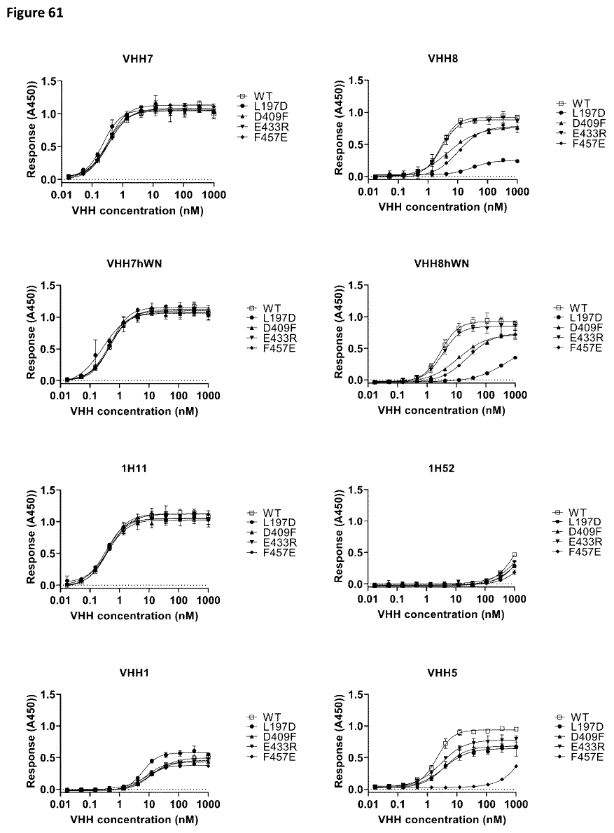

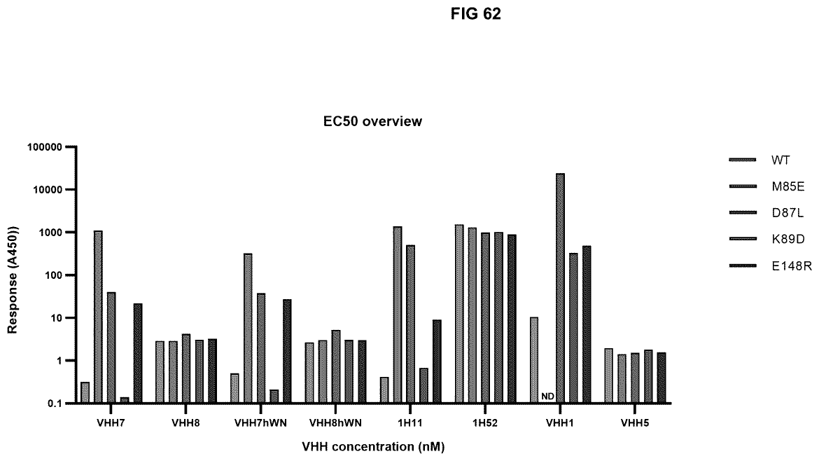

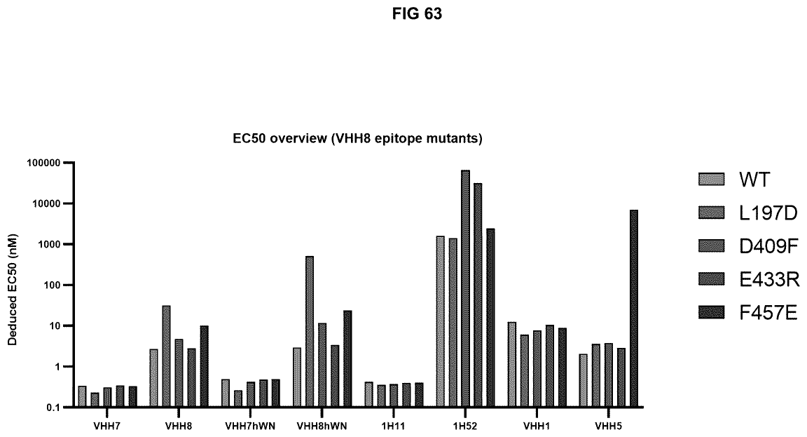

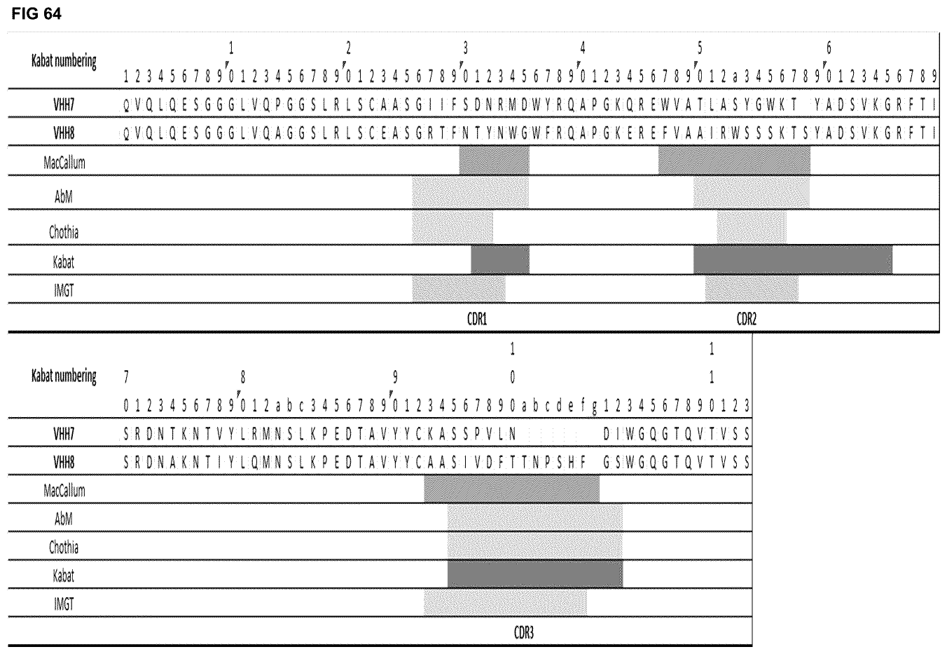

In healthy cells, human GAA (hGAA) is expressed in the ER as a large precursor protein of 952 amino acids and 105 kDa⁷. Upon co-translational transport, the 27-amino acid signal peptide is cleaved and five of the seven N-glycans may become phosphorylated⁸⁻¹⁰, resulting in a glycosylated precursor with an apparent molecular weight of 110 kDa¹¹. The enzyme is then transferred to the Golgi and recognised by CI-MPR in the trans-Golgi network¹²⁻¹⁵. This mediates its transfer to the endolysosomal system. Herein, GAA undergoes a series of post-translational processing events shown in FIG. 23¹⁶: a first protease cleaves amino acids at the N-terminus to retain a polypeptide of amino acids 57-952. Secondly, another yet unknown protease in the endolysosome generates a 3.9 kDa peptide (amino acids 78-113) that is linked via a disulphide bond to the remaining GAA chain (amino acids 122-952). This creates a 95 kDa intermediate while the sequences 57-78 and 113-122 are lost. Afterwards, a new processing event creates an intermediate GAA-variant due to a C-terminal cleavage by a third protease. This leads to the formation of a 19.4 kDa peptide (amino acids 792-952). Together with the 3.9 kDa peptide and the remaining polypeptide, the 19.4 kDa is associated to form a 76 kDa intermediate. Lastly, the 76 kDa is cleaved N-terminally releasing a 10.3 kDa peptide (amino acids 122-200) by a fourth protease. All four peptides remain associated to each other to form a 70 kDa mature GAA enzyme. The mature forms have a low pH optimum and hydrolyse glycogen in the lysosomes¹⁷.

The immature, 110 kDa GAA variant consist of multiple domains from the N- to C-terminus: a trefoil type-P domain, a β-sheet domain, the catalytic GH31 (β/α)₈ barrel and a proximal and distal β-sheet domain^{9,16}. The crystallisable polypeptide described (i.e. amino acids 81-952), missed out on four disordered loops as a diffractable crystal could only be obtained after limited protease treatment⁹.

In specific embodiments disclosed herein, fusion proteins comprising rhGAA, which is recombinant pre-processed precursor human GAA protein, fused at its C-terminus via a linker to a CI-M6PR-specific VHH, are provided for recombinant expression in mammalian cells as well as for administration to cells; tissues, and/or subjects, as ERT.

### Fusion Proteins for Lysosomal Targeting of Cathepsin D Protease (CTSD)

Human CTSD (hCTSD) is an ubiquitously expressed, broadly specific and highly abundant lysosomal protease²⁹. The enzyme belongs to a large cathepsin family containing a dozen members, which are distinguished by which proteins they cleave, their structure, or their catalytic mechanism. It is synthesised as a zymogen in the ER and contains an N-terminal pre-pro-peptide upon translation (Error! Reference source not found.B). During translocation or shortly after arriving in the ER, its 20-amino acid signal sequence (pre-peptide) is cleaved off, enabling the pro-hCTSD to move through the Golgi. There, modifications with M6P permits CI-MPR recognition and subsequent targeting to the lysosome. Upon arrival at the endolysosome, the pro-hCTSD 52 kDa protein is further processed to an intermediate form. This intermediate CTSD may still contain part of the pro-sequence—(i.e. residues 17-44 of the pro-sequence) or can be removed, resulting in a mature hCTSD (48 kDa). Final maturation of hCTSD consists of a combination of, so far unknown, enzymes³⁰ and auto-activation³¹, occurring in the lysosome. However, it includes hCTSB- and hCTSL-mediated cleavage of the 48 kDa CTSD into a N-terminal 14-kDa light chain and one C-terminal 34-kDa heavy chain bound together by non-covalent interactions³⁰. Both chains form a β-sheet domain, located at one side of the active side cleft, thereby each providing one catalytic Asp residue³². These actions combined with the acidic environment ensure proteolytic hCTSD activity in late endosomes and lysosomes only³³⁻³⁵.

Lysosomal targeting of hCTSD is, besides the M6P:CI-MPR pathway, also mediated by M6P-independent pathways such as the Lrp1/LDL receptor system^{36,37}, sortilin 1³⁸ but also SEZ6L2, a transmembrane I receptor predominantly expressed in the brain³⁹. The large amount and diversity of substrate that hCTSD can process in the lysosome⁴⁰⁻⁴² reflects the pleiotropic functions of the enzyme and its involvement in a number of physiological processes^{42,43}. hCTSD is not only important for metabolic protein degradation but it can also process hormones, inhibitors or activators of other enzymes, or activate enzymes themselves by cleavage. This way, hCTSD contributes to several metabolic processes, which reflect its importance and contribution in multiple diseases. Not only is the hCTSD activity reduced in muscles of inclusion body myositis patients²⁰, hCTSD also plays a role in neurodegenerative diseases (e.g. Alzheimer's disease)^{44,45} and tumour metastasis⁴⁶. Moreover, missense mutations in hCTSD cause the lysosomal storage disease neuronal ceroid lipofuscinosis 10^{47,48} for which recently an effective recombinant pro-hCTSD ERT has been described in vivo⁴⁹.

Considering its lysosomal storage phenotype and the effective approach of enzyme replacement therapy, a similar type of treatment for storage disorders beyond the heritable diseases is proposed to introduce hCTSD as a therapeutical degrader in proteinopathic cells or conditions such as inclusion body myositis²⁶⁻²⁸. This ERT could increase lysosomal flux and provide degradative capacity to the diseased lysosomes in order to degrade the accumulated proteins, thereby tackling the main pathological manifestation. As these diseases only cause pathology after decades of life, we hypothesise that they result from minor, yet chronic imbalances in proteastasis and hence that enhancing lysosomal proteolytic capacity by even a small amount could resolve this imbalance and treat diseases effectively.

In specific embodiments disclosed herein, fusion proteins comprising pro-hCTSD protein fused at its C-terminus via a linker to a CI-M6PR-specific VHH are provided for recombinant expression, and for administration to cells; tissues, and/or subjects, as ERT.

### Producing Binding Agents for Use in ERT

In another aspect, an in vitro method is disclosed for production the binding agents comprising the fusion protein of the invention, comprising the steps of: introducing the nucleic acid molecule encoding the binding agent or fusion protein, as described herein, or introducing a chimeric gene comprising said nucleic acid molecule in a host, incubating said host for cell-culturing and expressing the fusion protein, and extracting, isolating or purifying the fusion protein from said cell culture or growth medium.

Introduction in the host may be obtained upon transfecting a cell or recombinant expression in a cell. Preferably an eukaryotic cell is used, which may be a yeast cell, preferably a mammalian cell, but any type of cell. ‘Host cells’ can thus be either prokaryotic or eukaryotic. The cells can be transiently or stably transfected. Such transfection of expression vectors into prokaryotic and eukaryotic cells can be accomplished via any technique known in the art, including but not limited to standard bacterial transformations, calcium phosphate co-precipitation, electroporation, or liposome mediated-, DEAE dextran mediated-, polycationic mediated-, or viral mediated transfection. For all standard techniques see, for example, Sambrook et al., Molecular Cloning: A Laboratory Manual, 4^{th} ed., Cold Spring Harbor Press, Plainsview, New York (2012); and Ausubel et al., Current Protocols in Molecular Biology (Supplement 114), John Wiley & Sons, New York (2016). Recombinant host cells, in the present context, are those which have been genetically modified to contain an isolated DNA molecule, nucleic acid molecule or expression construct or vector of the invention. The DNA can be introduced by any means known to the art which are appropriate for the particular type of cell, including without limitation, transformation, lipofection, electroporation or viral mediated transduction. A DNA construct capable of enabling the expression of the chimeric protein of the invention can be easily prepared by the art-known techniques such as cloning, hybridization screening and Polymerase Chain Reaction (PCR). Standard techniques for cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described in Sambrook et al. (2012), Wu (ed.) (1993) and Ausubel et al. (2016). Representative host cells that may be used with the invention include, but are not limited to, bacterial cells, yeast cells, plant cells and animal cells. Bacterial host cells suitable for use with the invention include Escherichia spp. cells, Bacillus spp. cells, Streptomyces spp. cells, Erwinia spp. cells, Klebsiella spp. cells, Serratia spp. cells, Pseudomonas spp. cells, and Salmonella spp. cells. Animal host cells suitable for use with the invention include insect cells and mammalian cells (most particularly derived from Chinese hamster (e.g. CHO), and human cell lines, such as HeLa. Yeast host cells suitable for use with the invention include species within Saccharomyces, Schizosaccharomyces, Kluyveromyces, Pichia (e.g. Pichia pastoris), Hansenula (e.g. Hansenula polymorpha), Yarowia, Schwaniomyces, Schizosaccharomyces, Zygosaccharomyces and the like. Saccharomyces cerevisiae, S. carlsbergensis and K. lactis are the most commonly used yeast hosts, and are convenient fungal hosts. The host cells may be provided in suspension or flask cultures, tissue cultures, organ cultures and the like. Alternatively, the host cells may also be transgenic animals. The eukaryotic cell-expressed fusion protein as provided in the binding agent of the invention used in ERT applications may thus further comprise post-translational modifications including glycan modifications. In a preferred embodiment said eukaryotic host cell is a Glycodelete cell, which may be a yeast-based or mammalian-based Glycodelete-engineered host cell, as known in the state of the art (see for instance Ref. 52), as to influence the N-glycan profile of the resulting fusion protein produced in the host, and/or secreted in the cell culture medium.

In specific embodiments the binding agent of the present invention is thus the agent obtained from the method of production presented herein. And further specific embodiments provide for the use of said glycosylated products as medicament or specifically for treatment of lysosomal storage disease or ERT.

A “pharmaceutically or therapeutically effective amount” of compound or binding agent or composition is preferably that amount which produces a result or exerts an influence on the particular condition being treated. A “therapeutically active agent” is used to refer to any molecule that has or may have a therapeutic effect (i.e. curative or stabilizing effect) in the context of treatment of a disease (as described further herein). Preferably, a therapeutically active agent is a disease-modifying agent, and/or an agent with a curative effect on the disease. By “pharmaceutically acceptable” is meant a material that is not biologically or otherwise undesirable, i.e., the material may be administered to an individual along with the compound without causing any undesirable biological effects or interacting in a deleterious manner with any of the other components of the pharmaceutical composition in which it is contained. A pharmaceutically acceptable carrier is preferably a carrier that is relatively non-toxic and innocuous to a patient at concentrations consistent with effective activity of the active ingredient so that any side effects ascribable to the carrier do not vitiate the beneficial effects of the active ingredient. Suitable carriers or adjuvantia typically comprise one or more of the compounds included in the following non-exhaustive list: large slowly metabolized macromolecules such as proteins, polysaccharides, polylactic acids, polyglycolic acids, polymeric amino acids, amino acid copolymers and inactive virus particles. Such ingredients and procedures include those described in the following references, each of which is incorporated herein by reference: Powell, M. F. et al. (“Compendium of Excipients for Parenteral Formulations” PDA Journal of Pharmaceutical Science & Technology 1998, 52(5), 238-311), Strickley, R. G (“Parenteral Formulations of Small Molecule Therapeutics Marketed in the United States (1999)-Part-1” PDA Journal of Pharmaceutical Science & Technology 1999, 53(6), 324-349), and Nema, S. et al. (“Excipients and Their Use in Injectable Products” PDA Journal of Pharmaceutical Science & Technology 1997, 51 (4), 166-171). The term “excipient”, as used herein, is intended to include all substances which may be present in a pharmaceutical composition and which are not active ingredients, such as salts, binders (e.g., lactose, dextrose, sucrose, trehalose, sorbitol, mannitol), lubricants, thickeners, surface active agents, preservatives, emulsifiers, buffer substances, stabilizing agents, flavouring agents or colorants. A “diluent”, in particular a “pharmaceutically acceptable vehicle”, includes vehicles such as water, saline, physiological salt solutions, glycerol, ethanol, etc. Auxiliary substances such as wetting or emulsifying agents, pH buffering substances, preservatives may be included in such vehicles.

A further aspect relates to a nucleic acid molecule or the vector encoding said binding agents disclosed herein, or the pharmaceutical composition comprising these, as described herein.

Final aspect of the invention relate to the medical use of any one of the CI-M6PR-specific ISVDs as described herein, in monovalent form, as part of a further binding agent or compound or therapeutic molecule, including multivalent or multi-specific binding agents and fusion proteins as described herein, or formulated as a pharmaceutical composition optionally including further components. Specifically the use in treatment of lysosomal storage disease or use in ERT, involves said fusion proteins comprising one or more CI-M6PR-specific ISVDs linked to a lysosomal storage phenotype-related enzyme.

It is to be understood that although particular embodiments, specific configurations as well as materials and/or molecules, have been discussed herein for methods, samples and biomarker products according to the disclosure, various changes or modifications in form and detail may be made without departing from the scope of this invention. The following examples are provided to better illustrate particular embodiments, and they should not be considered limiting the application. The application is limited only by the claims.

### ASPECTS OF THE DISCLOSURE

The current application describes a binding agent comprising an immunoglobulin-single-variable domain (ISVD) specifically binding human cation-independent mannose-6-phosphate receptor (CI-M6PR), wherein said ISVD specifically binds residues located on the extracellular N-terminal CI-M6PR domains 1, 2 and/or 3.

Said binding agent, wherein the K_{D} value for binding human CI-M6PR is in the range of 100 nM or lower.

Said binding agent wherein said ISVD comprises 4 framework regions (FR) and 3 complementarity-determining regions (CDR) according to the following formula (1): FR1-CDR1-FR2-CDR2-FR3-CDR3-FR4 (1), and the CDR1, CDR2 and CDR3 regions are selected from those CDR1, CDR2 and CDR3 regions of a sequence selected from the group of sequences of SEQ ID NO: 1 to 11, wherein the CDR regions are annotated according to Kabat, MacCallum, IMGT, AbM, or Chothia.

Said binding agent, wherein said ISVD specifically binds an epitope comprising the amino acid residues 191, 194-196, 208, 219, 220, 224, 225, 407-409, 431, and 433 as set forth in SEQ ID NO:20, or comprising the amino acid residues 57-64, 83, 85, 87, 89, 90, 93, 98, 118, 143, and 145-151 as set forth in SEQ ID NO:20.

Said binding agent wherein said ISVD specifically binds to said CI-M6PR epitope, via the paratope comprising residues 52-55, 57, 100-103, and 108 as set forth in SEQ ID NO:8.

Said binding agent wherein said ISVD specifically binds to said CI-M6PR epitope, via the paratope comprising residues 31-35,47, 53-57, 71, 72, 100, 101, 104, 116, and 118 as set forth in SEQ ID NO:7.

Said binding agent wherein said ISVD comprises a sequence selected from the group of sequences of SEQ ID NO:1-11, or a sequence with at least 85% amino acid identity thereof, or a humanized variant thereof.

Said binding agent which is a multi-specific or multivalent binding agent.

Said multi-specific binding agent comprising said binding agent of the above, and a further binding agent specifically binding a cell surface or extracellular molecule.

A fusion protein comprising said binding agent of the above aspects, and preferably an enzyme.

Said binding agent of the above aspects, the multi-specific binding agent of the above aspects, or the fusion protein of the above aspects which comprises a detectable label or a tag.

A nucleic acid molecule encoding said binding agent of the above aspects, the multi-specific binding agent of the above aspects, or the fusion protein of the above aspects.

Use of said binding agent of the above aspects, the multi-specific binding agent of the above aspects, or the fusion protein of the above aspects in drug discovery, in structural analysis, or in a screening assay.

Use of the multi-specific binding agent of the above aspects, in a method for degrading a cell surface molecule or extracellular molecule in the lysosome.

Use of said binding agent of the above aspects, the multi-specific binding agent of the above aspects, or the fusion protein of the above aspects for in vitro lysosomal tracking.

A pharmaceutical composition comprising said binding agent of the above aspects, the multi-specific binding agent of the above aspects, or the fusion protein of the above aspects.

Said binding agent of the above aspects, the multi-specific binding agent of the above aspects, or the fusion protein of the above aspects, or the pharmaceutical composition of the above aspect, for use as a medicament.

Said binding agent of the above aspects, the multi-specific binding agent of the above aspects, or the fusion protein of the above aspects, or the pharmaceutical composition of the above aspect, for use in treatment of a lysosomal storage disease.

Said binding agent of the above aspects, the multi-specific binding agent of the above aspects, or the fusion protein of the above aspects, or the pharmaceutical composition of the above aspect, for use as a diagnostic or for in vivo imaging.

In a further aspect of the disclosure a binding agent is described comprising a fusion protein comprising an immunoglobulin-single-variable domain (ISVD) specifically binding human cation-independent mannose-6-phosphate receptor (CI-M6PR), and an enzyme, wherein said ISVD is fused directly or via a linker at the C-terminus of the enzyme, and wherein said ISVD specifically binds residues located on the extracellular N-terminal CI-M6PR domains 1, 2 and/or 3.

Said binding agent wherein the ISVD comprises 4 framework regions (FR) and 3 complementarity-determining regions (CDR) according to the following formula (1): FR1-CDR1-FR2-CDR2-FR3-CDR3-FR4 (1), and the CDR1, CDR2 and CDR3 regions are selected from those CDR1, CDR2 and CDR3 regions of a sequence selected from the group of sequences of SEQ ID NO: 1 to 11, wherein the CDR regions are annotated according to Kabat, MacCallum, IMGT, AbM, or Chothia.

Said binding agent wherein said fusion protein comprises an ISVD comprising a sequence selected from the group of sequences of SEQ ID NO:1-11, or a sequence with at least 85% amino acid identity thereof, or a humanized variant thereof, wherein the CDRs are identical.

Said binding agent wherein said ISVD and enzyme are fused by a linker, preferably a glycine-serine linker, such as a triple Gly₄Ser linker.

Said binding agent wherein said enzyme is a lysosome-localized enzyme.

Said binding agent wherein said fusion protein is a multi-specific or multivalent binding agent comprising said CI-M6PR-specific ISVD, an enzyme and a further antigen-binding domain or functional moiety.

Said binding agent wherein said further antigen-binding domain or functional moiety is a half-life extension.

Said binding agent which comprises a detectable label or a tag.

Said binding agent wherein said enzyme is acid alfa-glucosidase or a functional homologue thereof, or is Cathepsin D or a functional homologue thereof.

Said binding agent wherein said fusion protein comprises a sequence selected from the group of sequences of SEQ ID NO:14-21 or a functional homologue with at least 90% identity thereof.

A nucleic acid molecule encoding the binding agent of any of the above aspects.

A method to produce the binding agent of any of the above aspects, comprising the steps of:
a. Introducing the nucleic acid molecule of the above aspect in a host cell, and
b. Isolating the binding agent from the medium.

Said method wherein the host cell is a Glycodelete cell.

Said binding agent obtainable by said method of the above aspects.

Said binding agent of the above aspects, or obtainable by the above method wherein said fusion protein comprises N-glycans comprising one or more glycans selected from the group of a single GlcNAc, a GalGlcNAc and a SiaGalGlcNAc.

A pharmaceutical composition comprising the binding agent of the above aspects or the nucleic acid molecule of the above.

Said binding agent of any of the above aspects, the nucleic acid molecule of the above aspect, or the pharmaceutical composition of the above aspect, for use as a medicament.

Said binding agent of any of the above aspects, the nucleic acid molecule of the above aspect, or the pharmaceutical composition of the above aspect, for use in treatment of a lysosomal storage disease.

Said binding agent of any of the above aspects, the nucleic acid molecule of the above aspect, or the pharmaceutical composition of the above aspect, for use in treatment of diseases caused by a lysosomal storage phenotype, preferably Pompe disease, sporadic inclusion body myositis, or neuronal ceroid lipofuscinosis 10 (CLN10).

Use of said binding agent of any of the above aspects, the nucleic acid molecule of the above aspect, or the pharmaceutical composition of the above aspect, in a method for degrading a cell surface molecule or extracellular molecule in the lysosome.

Use of Said binding agent of any of the above aspects, the nucleic acid molecule of the above aspect, or the pharmaceutical composition of the above aspect, in a method for in vitro lysosomal tracking.

### EXAMPLES

### Example 1. Recombinant Production of Human and Mouse Dom1-3His₆ Antigen, Llama Immunization and Panning

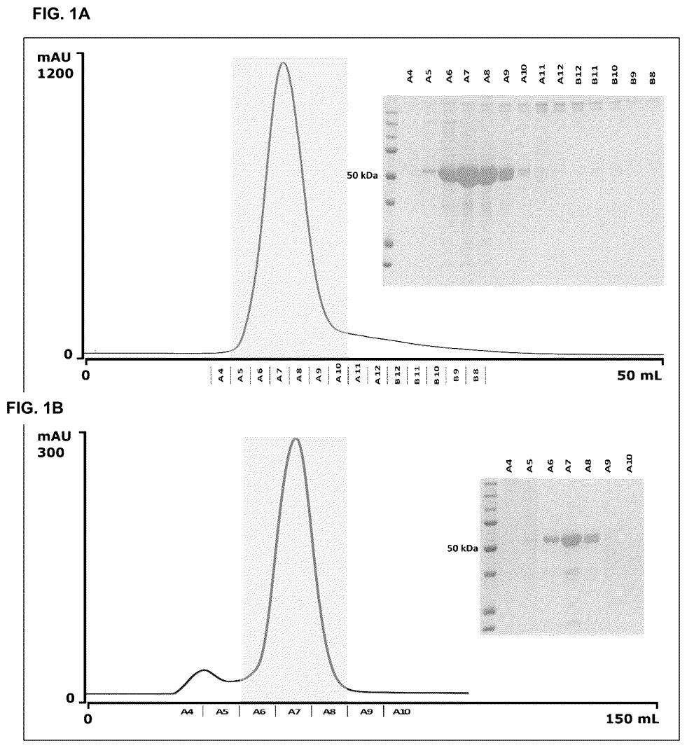

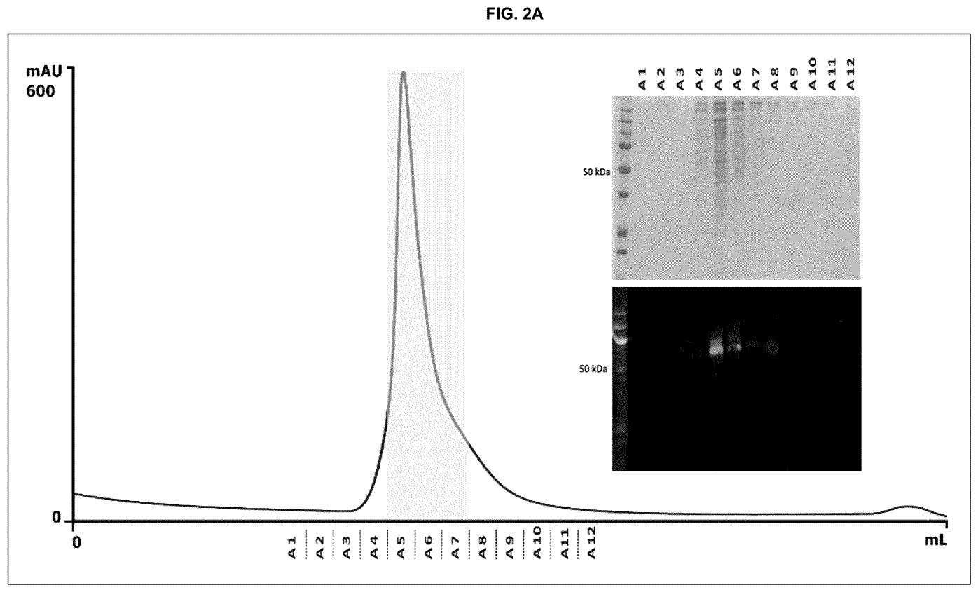

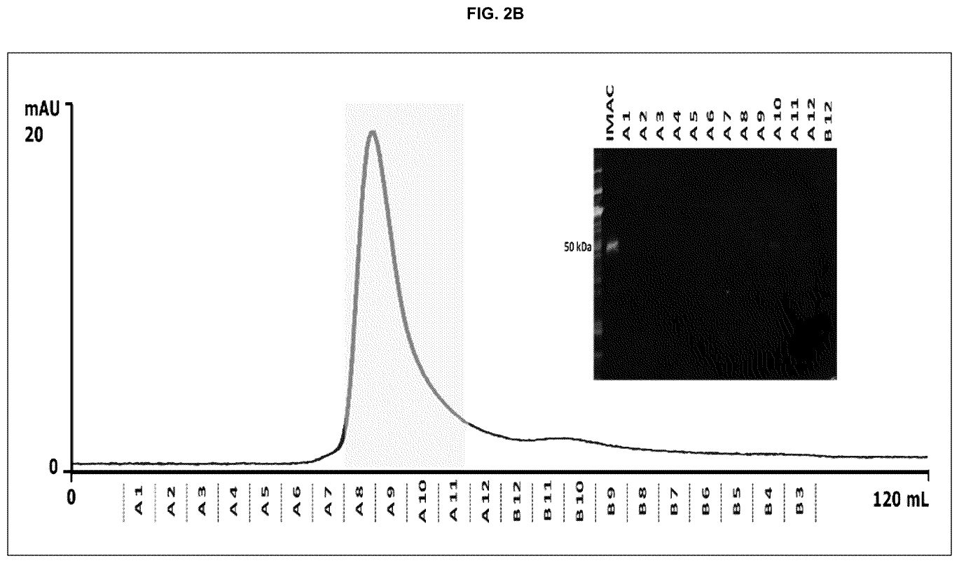

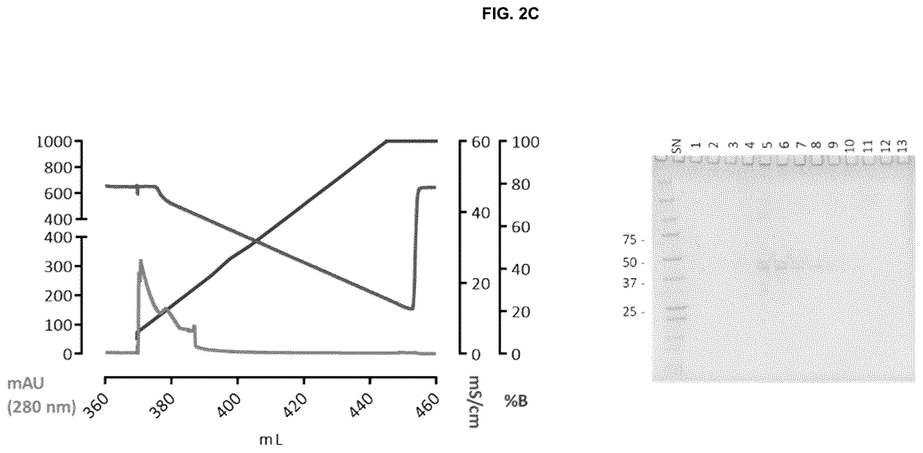

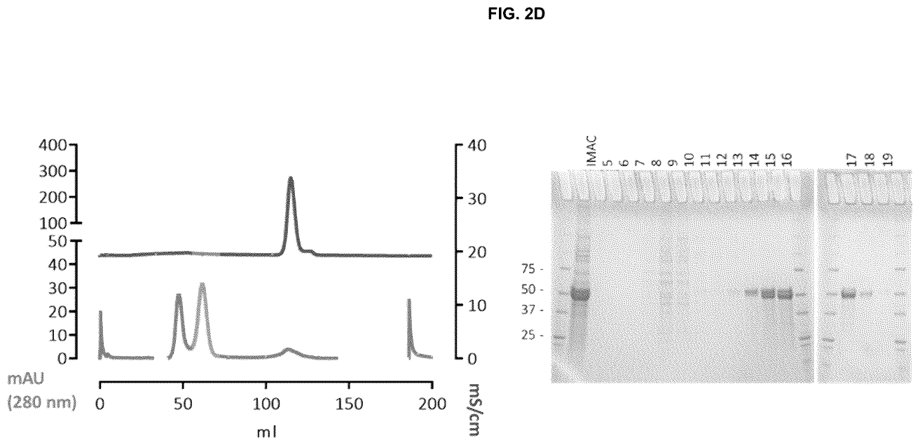

The recombinant human domain 1-3His₆ (hDom₁-3His₆) of the Cation Independent-Mannose-6-Phosphate-Receptor (CI-M6PR) was expressed and produced in HEK293S suspension cells and purified using Immobilized Metal Ion Affinity Chromatography (IMAC) (HisTrap HP 5 mL) (FIG. 1) and Size Exclusion Chromatography (SEC) (HiLoad 16/600 Superdex 200 pg) (FIG. 2) to a final yield of 21 mg protein/L in 50 mM MES, 150 mM NaCl of pH 6.5. In contrast, only 150 μg/L (FIG. 2A, B; using expression of amino acids 1-418 of SEQ ID NO: 21) and 3.1 mg/L (FIGS. 2 C,D; using expression of amino acids 1-462 of SEQ ID NO: 21) of the domains 1-3 of the mouse CI-M6PR (mDom₁₋₃His₆) could be produced and purified.

Immunization of two llama's using hDom₁-3His₆ antigen led to two different VHH libraries that were panned onto the coated antigen. For one library, no positive enrichment was observed in any of the panning rounds. Still, 95 colonies from round 1 and 95 colonies from round 3 were randomly selected and analyzed by ELISA for the presence of antigen-specific VHHs in their periplasmic extracts (ELISA using crude periplasmic extracts including soluble VHHs). Out of these 190 colonies, none scored positive in this assay. The phage population of the second VHH library however, was enriched for antigen-specific phages about 10-fold, 2000-fold and 3000-fold each round, respectively. In total, 285 colonies from round 2 were randomly selected and analysed by ELISA for the presence of antigen-specific VHHs in their periplasmic extracts. Out of these 285 colonies, 219 colonies scored positive in this assay of which 55 different full length VHHs were distinguished after sequence analysis. Via the Cluster Database at High Identity with Tolerance¹⁰³, highly similar VHHs were grouped into the same CDR3 family. Their amino acid sequences suggest that they are from clonally-related B-cells resulting from somatic hypermutation or from the same B-cell but diversified due to RT and/or PCR error during library construction. The high frequency of identical clones for the panning of the second library resulted in a limited number of different CDR3 groups. So the antigen does not seem to be very immunogenic, with one llama generating only a limited number of 11 different CDR3 families and a second llama not showing any response.

### Example 2. Production and Purification of Anti-CI-M6PR VHHs

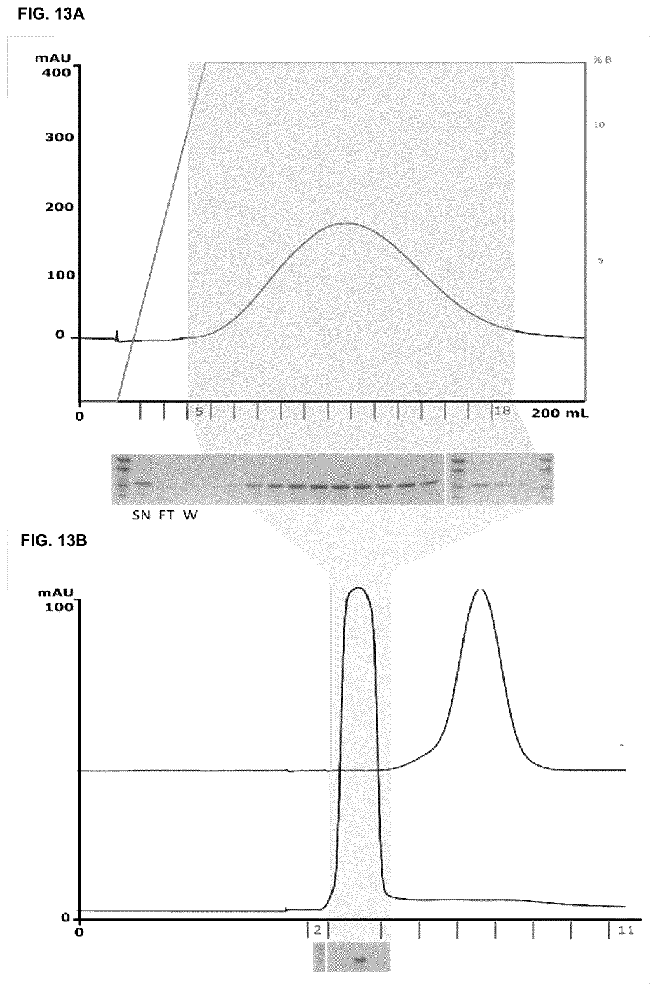

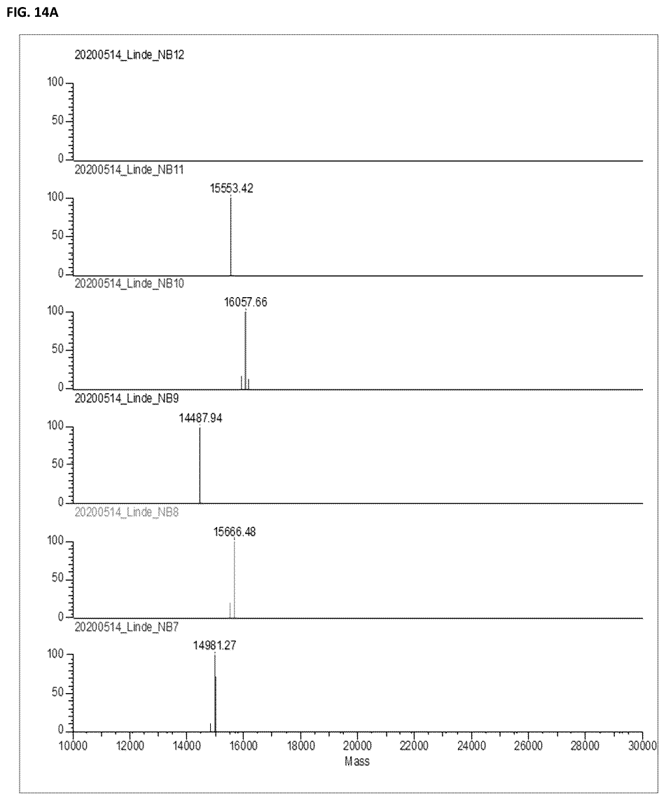

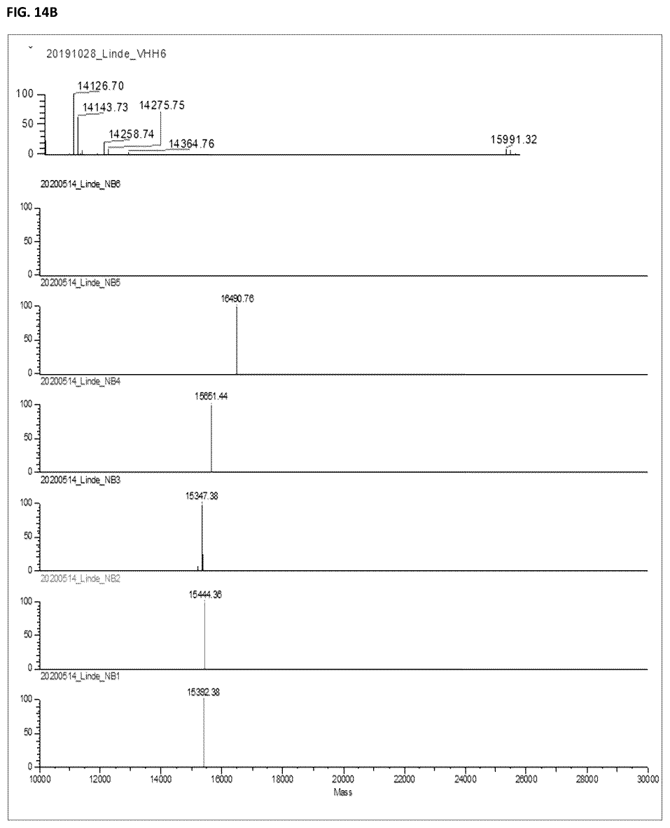

Out of the 11 anti-CI-M6PR VHH families, 10 VHHs were produced in P. pastoris and one VHH clone (i.e. VHH6) was expressed in E. coli. After recombinant production, the VHHs were purified using IMAC and finally desalted. Results for VHH7 and VHH8 are outlined in FIG. 12 and FIG. 13. Verification was obtained by Intact mass analysis for all 11 VHHs (FIG. 14). An overview of their expression levels per 100 mL is shown in Table 1. The best expression yields were obtained for VHH10 and VHH11 with a total yield of 20 mg and 19 mg per 100 mL culture, respectively (Table 1). VHH2 and VHH4 also showed high production levels with respectively 7 mg and 13 mg total yield per 100 mL culture. On the other hand, VHH1, VHH3, VHH6 and VHH8 showed the lowest production yield with values around 2-3 mg per 100 mL culture.

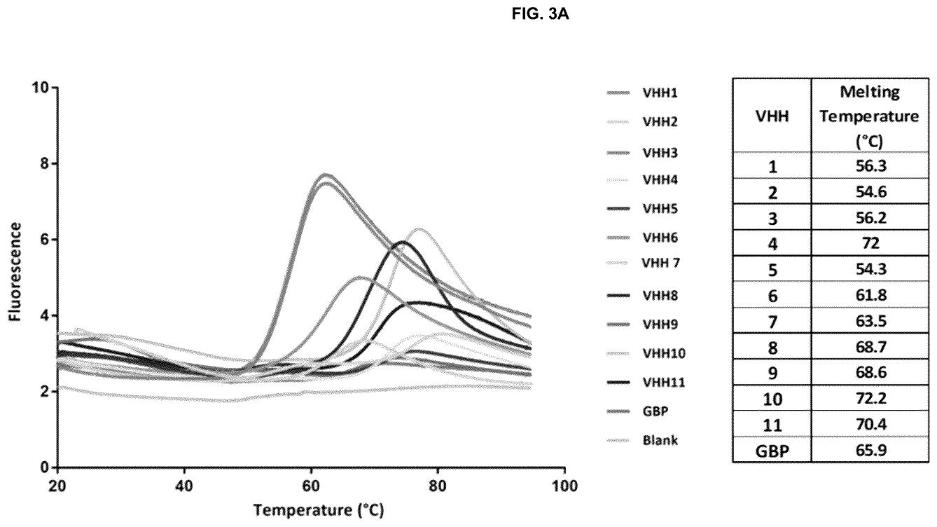

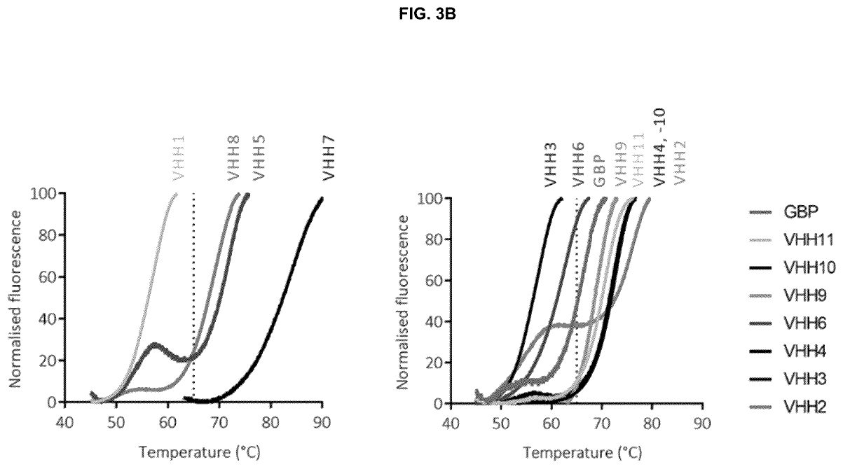

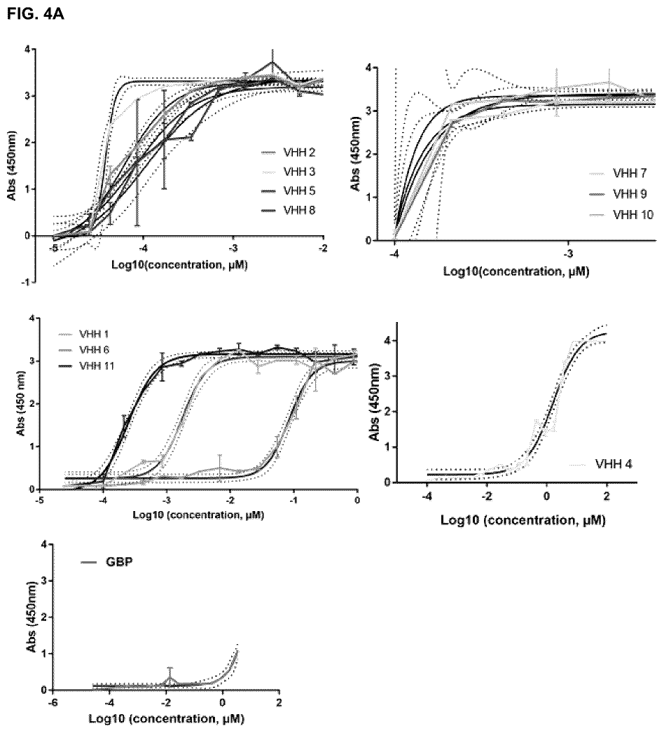

In addition, the thermal stability of each anti-CI-M6PR VHH was measured using SYPRO Orange. Melting temperature curves and data are shown in FIG. 3 and are in the range of 57° C. to 75° C.

TABLE 1
The expression yield of anti-CI-M6PR VHHs from Pichia pastoris.

| | Total yield (mg) |
|---|---|
| VHH | (100 ml culture) |
| 1 | 3.60 |
| 2 | 7.10 |
| 3 | 3.78 |
| 4 | 12.97 |
| 5 | 5.68 |
| 6 | 2.13 |
| 7 | 5.94 |
| 8 | 3.21 |
| 9 | 4.03 |
| 10 | 20.07 |
| 11 | 19.11 |

### Example 3. Affinity of Anti-CI-M6PR VHHs and Cross-Reactive Binding to Mouse Dom₁₋₃His₆

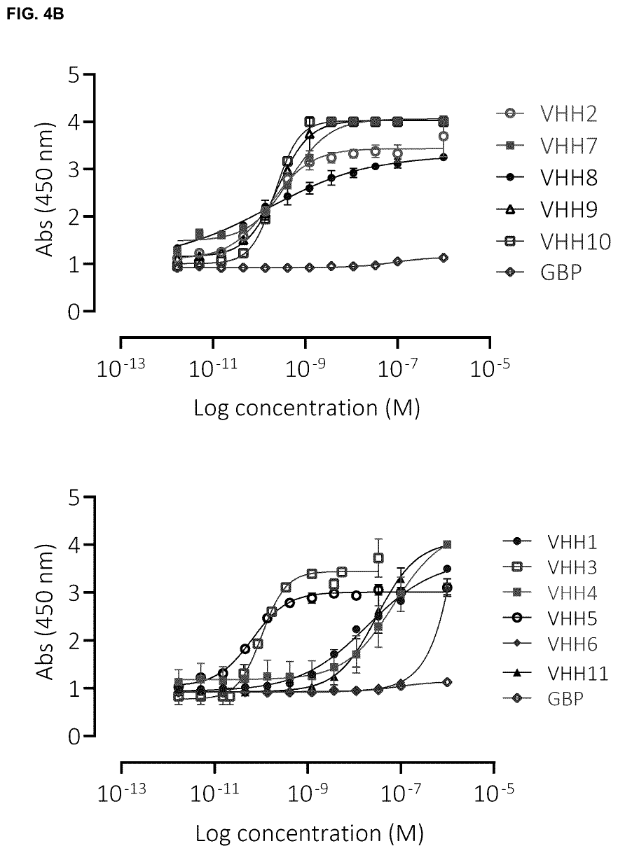

We evaluated the affinity of the VHHs against the hDom₁₋₃His₆ using ELISA. Coated hDom₁₋₃His₆ was incubated with serially diluted VHH that was afterwards detected using an anti-VHH antibody, coupled to HRP. According to the results from this assay, VHH2, VHH3, VHH5, VHH7, VHH9, and VHH11 bind the recombinant hDom₁₋₃His₆ with high affinity while a lower affinity was observed for VHH1 and VHH6 (FIG. 4, Table 2). For VHH4 and the GFP-binding VHH (GBP) only α-specific signal could be detected.

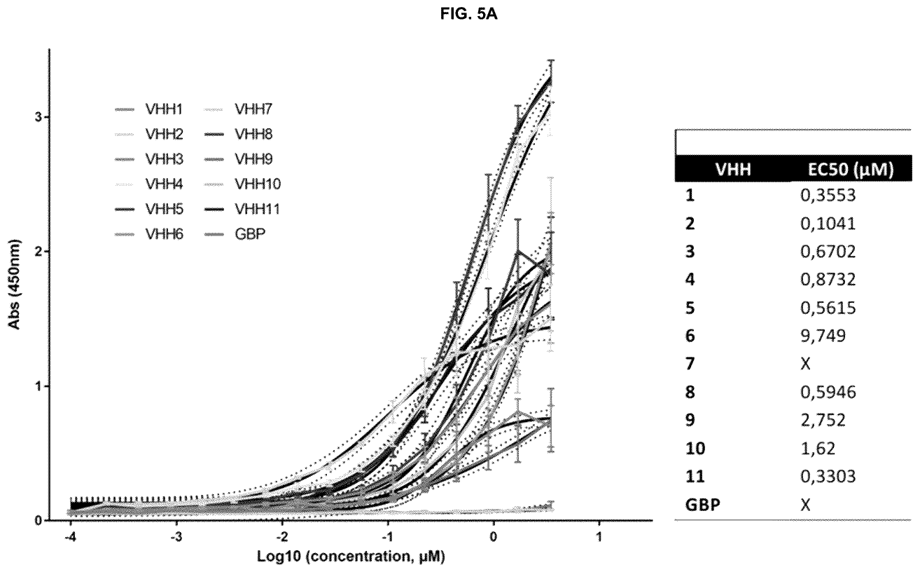

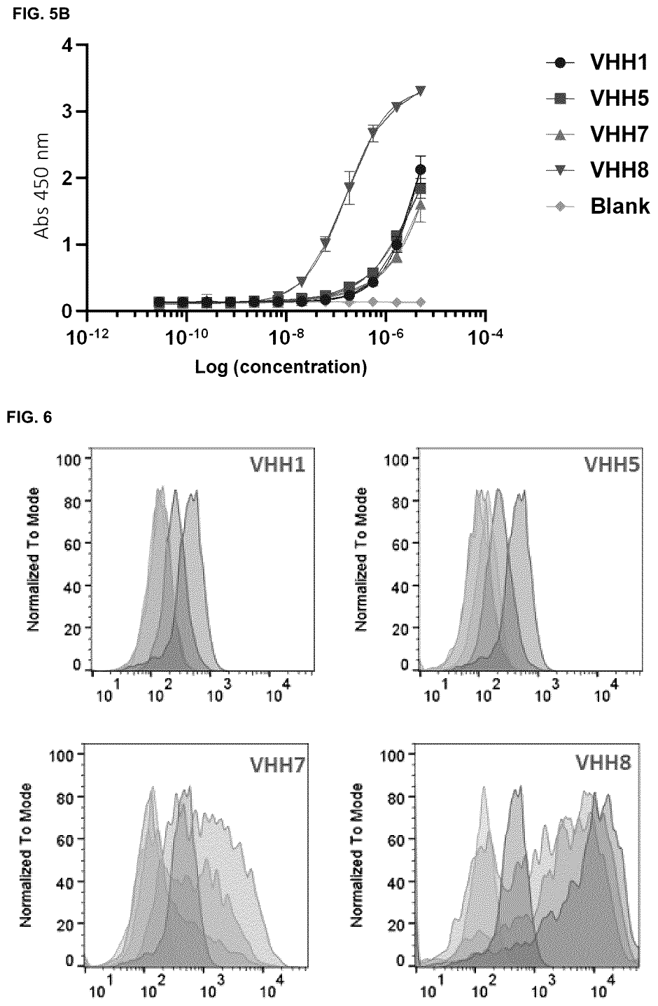

To evaluate the cross-reactivity of the anti-CI-M6PR nanobodies between the human and mouse CI-M6PR domain 1-3His₆ antigen, ELISA was performed with the eleven serially diluted anti-CI-M6PR nanobodies. We aimed for the saturation of the coated antigen at the highest VHH concentrations, resulting in sigmoidal absorbance graphs. However, saturation was not reached for VHH 3, 4, 6, 8, 9 and 10, which complicates the interpretation (FIG. 5). By performing sigmoidal regression on the derived absorbance curves, half-maximal effective concentration (EC₅₀) values of each VHH were acquired, representing the required concentration to occupy 50% of the coated antigen. As a negative control, GBP was included in the assay. The largest EC₅₀ values were detected for VHH 6, 7, 9 and 10, while the smallest for VHH 1, 2 and 11 (FIG. 5). Overall, we could deduce from this assay that VHH 1, 2, 3, 4, 5, 8 and 11 may show cross-reactivity, hence bind to mouse antigen, though verification in a mouse cell-based system may be required.

### Example 4. Flow Cytometry Analysis of Anti-CI-M6PR VHHs Binding to the Native Receptor

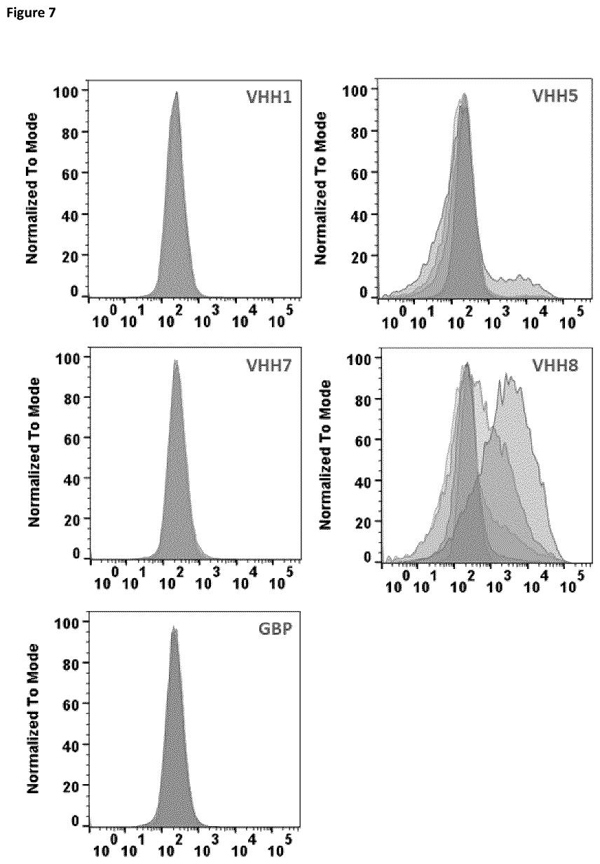

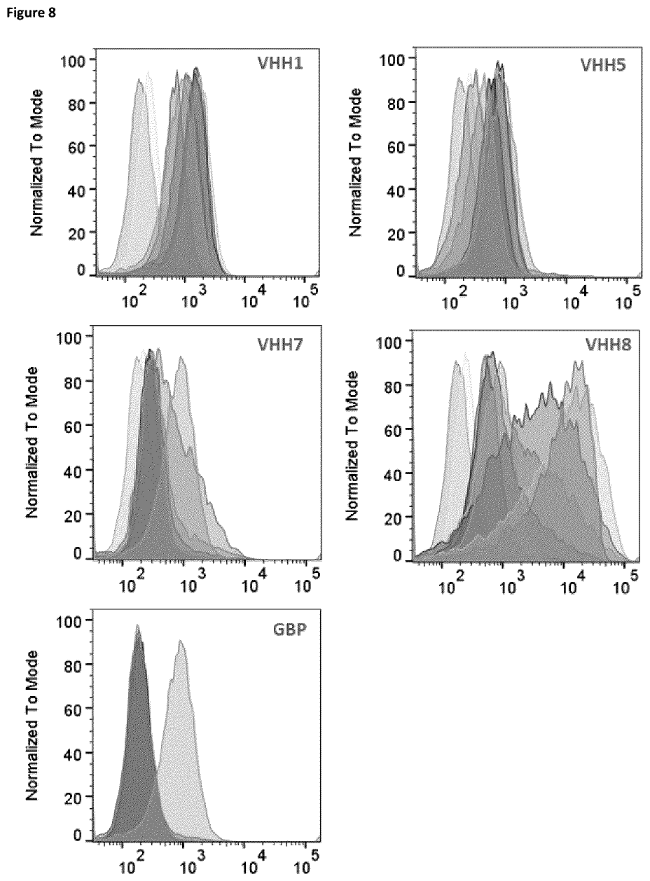

Anti-CI-M6PR VHHs were serially diluted (starting from 200 μg/mL) and incubated for 2 h at 4° C. on HEK293 cells (FIG. 6), MCF7 (FIG. 8) cells, as well as L-D9 (FIG. 7) cells which express a chimeric bovine/mouse CI-M6PR. All 11 VHHs were analyzed in each assay, and on every type of cells, and for VHH1, VHH5, VHH7, and VHH8, the results are shown in FIGS. 6 to 8. VHH7 and VHH8 showed the highest affinity for the native CI-M6PR receptor in the HEK293 and MCF7 cell lines.

The profiles for the VHHs from our panel of 11 VHH families not shown in FIG. 6 resemble those of the profiles of VHH1 and VHH5. For the MCF7 cells, for the profiles of VHHs not shown in FIG. 8, VHH10 and VHH11 profiles resemble the GBP negative control profile; VHH2, 3, 4 and 9 resemble the profile of VHH7; and VHH6 resembles the profiles of VHH5.

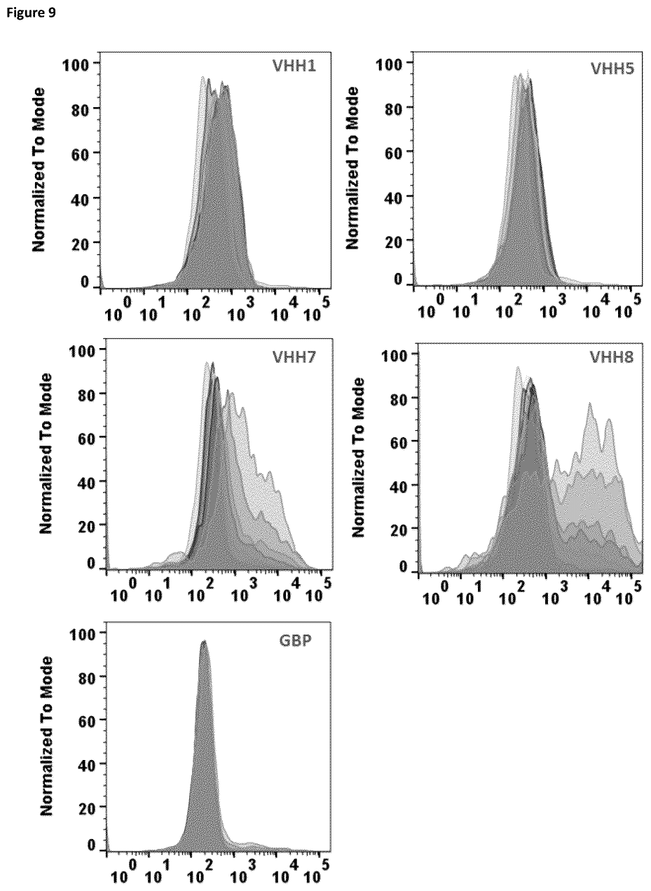

On the cell line expressing the bovine/mouse CI-M6PR chimera, only VHH8 showed clear binding on the cell surface (FIG. 7). Finally, when looking at internalisation of all 11 anti-CI-M6PR VHHs by MCF7 cells (FIG. 9), we observed similar profiles as compared to cell surface binding i.e. most VHHs showed overlapping peaks with a profile as for VHH1 and 5 in FIG. 9, while there was clearly binding to the native CI-M6PR and internalisation observed for VHH7 and VHH8.

### Example 5. Validation of VHH7 and VHH8 Binding to CI-M6PR in Human and Mouse Cell Lines

Synthetic DNA fragments encoding VHHs were ordered (IDT, Leuven, Belgium) and subcloned into an E. coli expression vector under control of an IPTG-inducible lac promoter, in frame with N-terminal PelB signal peptide (which directs the recombinant proteins to the periplasmic compartment) and C-terminal FLAG3 and His6 tags. Electrocompetent E. coli TG1 cells (Lucigen, cat. #60502) were transformed and the resulting clones were sequence verified. VHH proteins were purified from these clones by means of IMAC chromatography followed by desalting according to well established procedures¹⁰⁶. Cells used herein were recovered using cell dissociation non-enzymatic solution (Sigma Aldrich, cat. #C5914-100ML) and resuspended to a final concentration of 1,000,000 cells/mL in FACS buffer. Cells were incubated with different concentrations of purified FLAG3-tagged VHHs for 30 minutes on ice with shaking, followed by washing and incubation with mouse M2 anti-Flag mAb (Sigma Aldrich, cat. #F-1804) in FACS buffer for 30 min on ice with shaking, followed again by washing. VHH binding to cells was detected with R-Phycoerythrin AffiniPure F(ab′)₂ Fragment Goat Anti-Mouse IgG (Jackson ImmunoResearch, cat. #115-116-071) in FACS buffer, incubated for 30 minutes on ice with shaking and protection from light. Flow cytometry and analysis were done on an Attune NxT Flow Cytometer (Thermo Fisher Scientific).

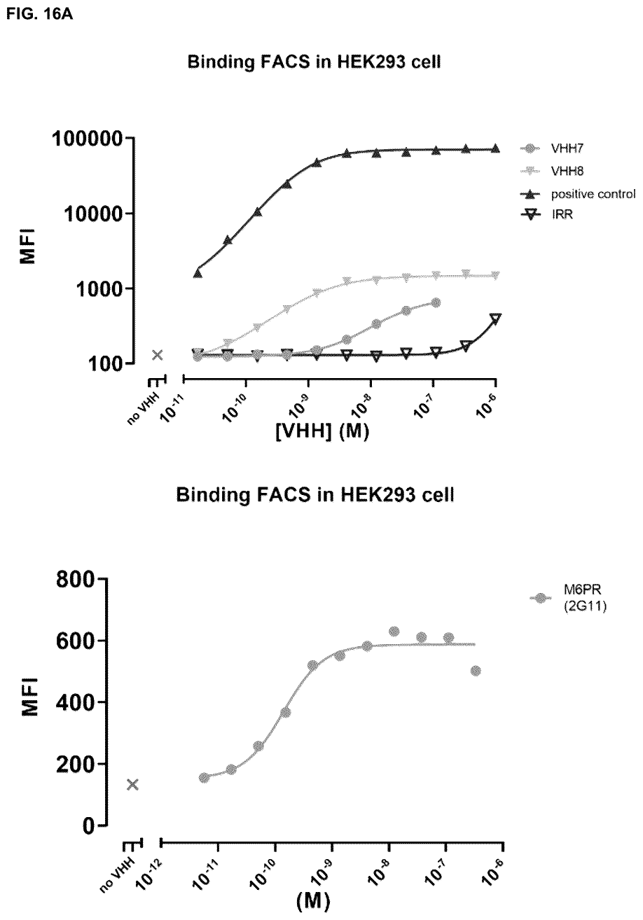

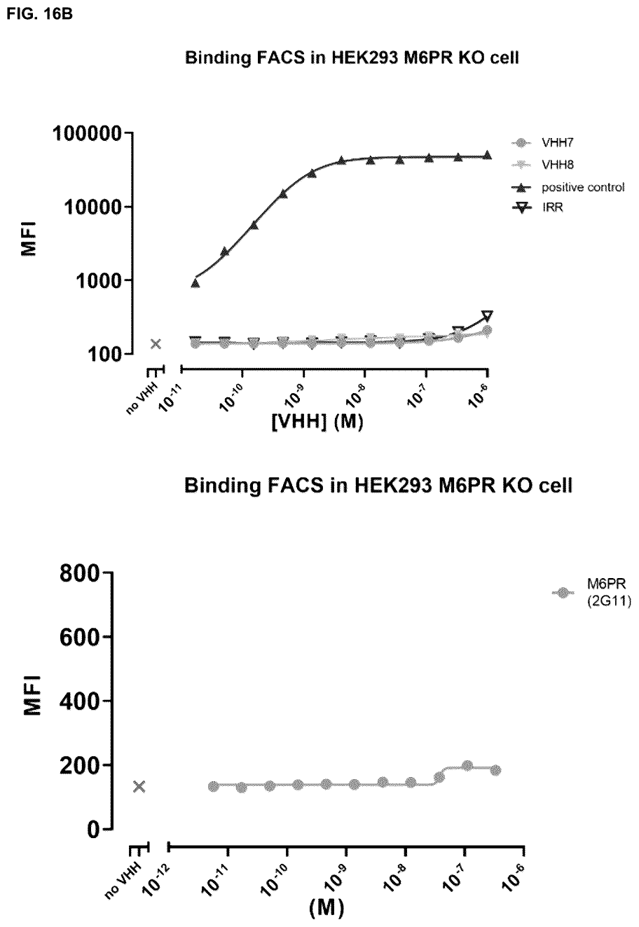

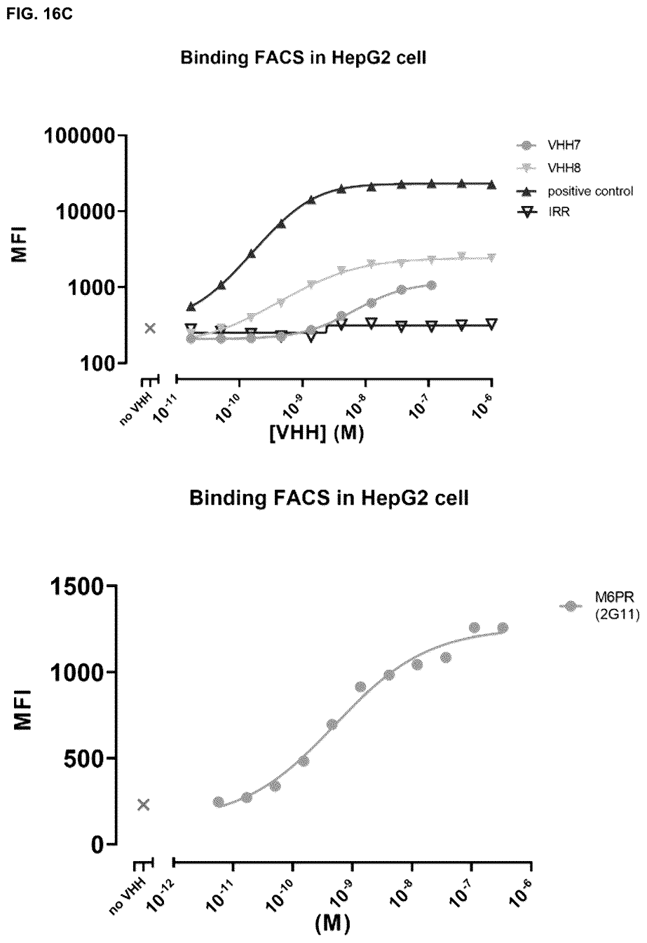

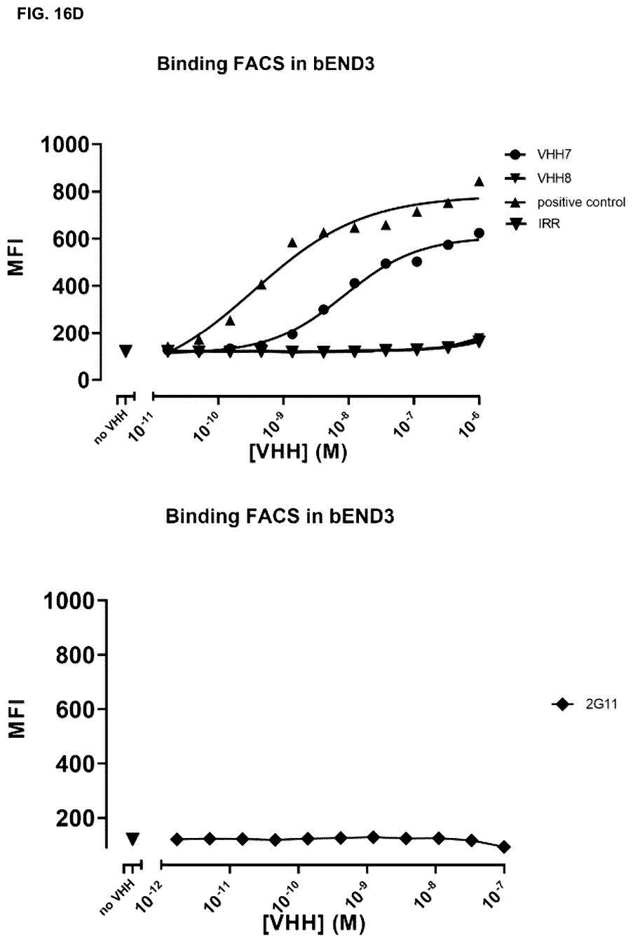

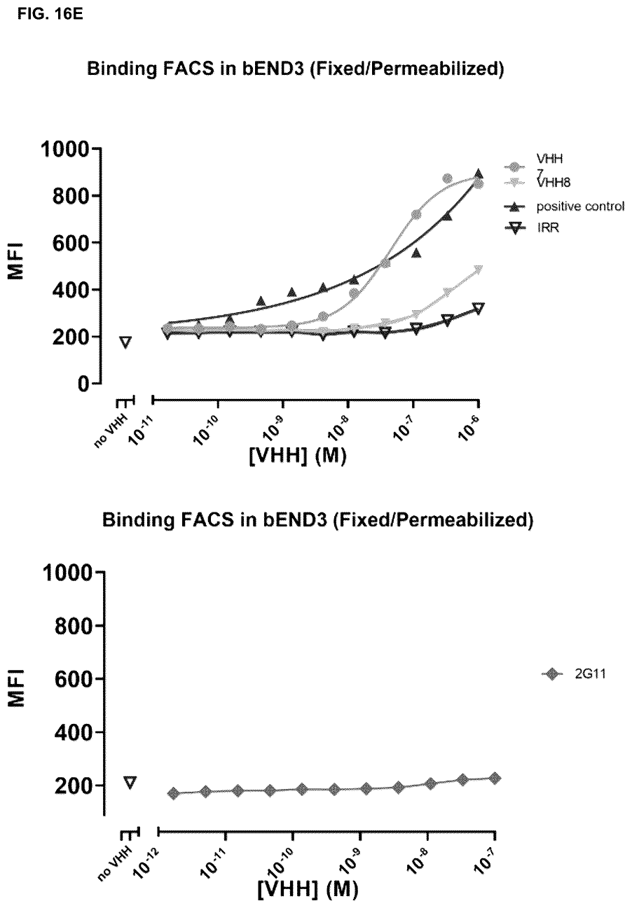

The specific binding of VHH7 and VHH8 to the human cell-expressed CI-M6PR was confirmed after analysis of serially diluted VHH on human HEK293T and HepG2 cells (FIGS. 16A and C), and absent on HEK 293 CIMPR^{-/-} cells (FIG. 16B). An EC₅₀ of 20 nM and 0.8 nM for binding to human CI-M6PR from HEK cells was obtained for VHH7 and VHH8, respectively. Moreover, cross-reactivity to mouse CI-M6PR was analyzed, in view of the amino acid homology between mouse and human being overall about 80% identity, but for domain 1-3 of CI-M6PR just about 75% identity. VHH7 showed to some extent cross-reactivity towards mouse CI-M6PR of bEND3 cells, with an EC₅₀ around 8.5 nM (FIG. 16D), and VHH7 showed to be internalized by mice cells (FIG. 16E), whereas this was only seen to a very limited extent for VHH8, in the currently tested system.

### Example 6. pH-Dependent Dissociation of Anti-CI-M6PR VHHs from the CI-M6P-Receptor

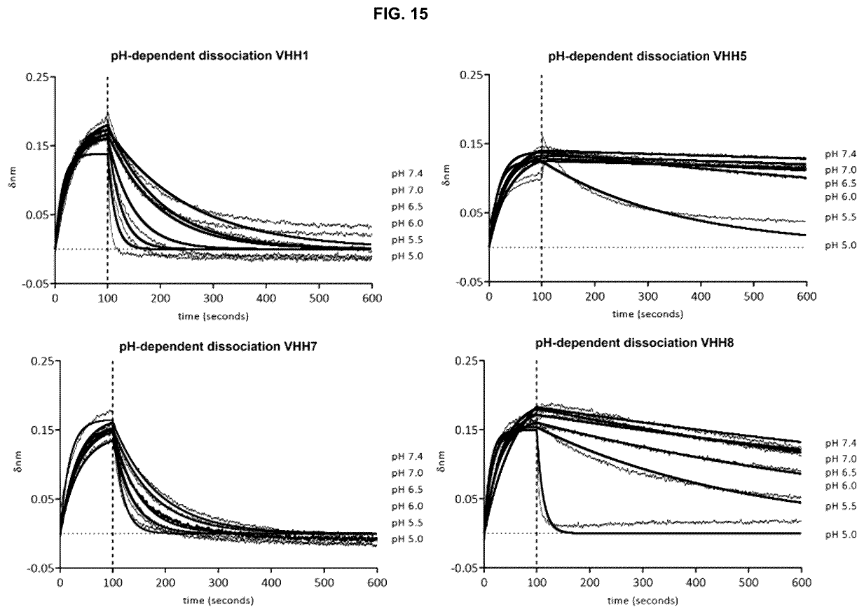

For each of the 11 anti-CI-M6PR VHHs, a biolayer interferometry (BLI) experiment was performed in which the hDom₁₋₃His₆ was biotinylated and coupled to streptavidin biosensor tips. Afterwards, the tips were incubated with serially diluted VHHs during the association phase and dissociation was performed at pH 7.4. Similarly, dissociation was performed at pH 7.0, pH 6.0 and pH 5.0 as well after regeneration of the biosensor tips. The K_{D}, kₒₙ (or kₐ or association rate constant) and k_{off} (or k_{d} or dissociation rate constant) values retrieved at pH 7.4 after processing and curve fitting of the BLI measurements are provided in Table 2 and Table 3; dissociation curves for VHH1, 5, 7, and 8 are shown in FIG. 15. The anti-CI-M6PR antibody (clone 2G11) and GBP were used as positive and negative controls, respectively.

TABLE 2
Overview of the binding affinity of the anti-CI-M6PR VHHs
for hDom₁₋₃his6 as determined by BLI at pH 7.4 (shown as K_{D}, Kₒₙ and
k_{off}) and by ELISA (shown as EC₅₀).

| | K_{D} (M) | k_{ON} | k_{OFF} | EC₅₀ (M) |
|---|---|---|---|---|
| VHH | (BLI) | (M⁻¹ s⁻¹ ) | (s⁻¹ ) | (ELISA) |
| VHH1 | 7.44E−09 | 8.14E+05 | 3.90E−05 | 1.78E−09 |
| | 1.12 × 10⁻⁹ | 1.0 × 10⁶ | 9.9 × 10⁻³ | 1.94 × 10⁻⁸ |
| VHH2 | 5.30E−09 | 6.79E+05 | 3.60E−03 | 6.08E−11 |
| | 4.89 × 10⁻⁹ | 4.83 × 10⁶ | 1.05 × 10⁻² | 1.77 × 10⁻¹⁰ |
| VHH3 | 1.17E−13 | 8.53E+05 | 1.00E−07 | 3.79E−11 |
| | 7.10 × 10⁻⁹ | 4.86 × 10⁴ | 3.45 × 10⁻⁴ | 1.04 × 10⁻¹⁰ |
| VHH4 | 2.33E−09 | 5.71E+05 | 1.33E−03 | 1.61E−06 |
| | 2.34 × 10⁻⁹ | 5.71 × 10⁵ | 1.33 × 10⁻³ | 7.01 × 10⁻⁸ |
| VHH5 | 7.87E−10 | 1.37E+06 | 1.24E−04 | 8.16E−11 |
| | 5.60 × 10⁻¹⁰ | 1.8 × 10⁶ | 1.8 × 10⁻⁴ | 6.47 × 10⁻¹¹ |
| VHH6 | 3.35E−09 | 1.05E+06 | 3.52E−03 | 8.42E−08 |
| | 4.74 × 10⁻⁹ | 7.43 × 10⁵ | 3.52 × 10⁻³ | ND |
| VHH7 | 1.08E−08 | 9.24E+05 | 9.99E−03 | 1.09E−11 |
| | 9.08 × 10⁻⁹ | 1.3 × 10⁶ | 1.18 × 10⁻² | 1.19 × 10⁻⁹ |
| VHH8 | 1.27E−09 | 4.65E+05 | 7.87E−04 | 1.10E−10 |
| | 2.65 × 10⁻⁹ | 1.0 × 10⁶ | 6.5 × 10⁻⁴ | 1.65 × 10⁻¹⁰ |
| VHH9 | 2.82E−10 | 1.57E+06 | 4.44E−04 | 6.42E−12 |
| | 2.82 × 10⁻¹⁰ | 1.57 × 10⁶ | 4.44 × 10⁻⁴ | 2.51 × 10⁻¹⁰ |
| VHH10 | 4.63E−08 | 3.81E+04 | 1.77E−03 | 6.57E−11 |
| | 4.65 × 10⁻⁸ | 3.81 × 10⁴ | 1.77 × 10⁻³ | 2.24 × 10⁻¹⁰ |
| VHH11 | 4.14E−08 | 7.67E+04 | 3.17E−03 | 2.49E−10 |
| | 5.10 × 10⁻⁸ | 7.67 × 10⁴ | 3.91 × 10⁻³ | 3.05 × 10⁻⁸ |
| anti-Cl- | 2.76E−06 | 2.15E+02 | 5.92E−04 | ND |
| M6PR Ab | | | | |

For each VHH the experiment was repeated twice, with the 2 values indicated below each other in this table. Indication of ‘E+/−0n’ is the same as ‘×10^{+/-n}’.

TABLE 3
Overview of the binding affinity of the anti-CI-M6PR VHHs for human hCI-M6PRD1-D3 as determined
by biolayer interferometry at pH 7.4 and additionally when dissociated at pH 7.0, 6.0 and 5.0.

| Experiment 1 | | | | | | | | | |
|---|---|---|---|---|---|---|---|---|---|
| | | K_{D} | k_{ON} | k_{OFF} | | | K_{D} | k_{ON} | k_{OFF} |
| VHH | pH | (M) | (M⁻¹s⁻¹) | (s⁻¹) | VHH | pH | (M) | (M⁻¹s⁻¹) | (⁻¹) |
| VHH1 | 7.4 | 7.44E−09 | 8.14E+05 | 3.90E−05 | VHH7 | 7.4 | 1.08E−08 | 9.24E+05 | 9.99E−03 |
| | 7 | 6.05E−09 | 1.25E+06 | 5.81E−05 | | 7 | 8.08E−09 | 8.64E+05 | 6.98E−03 |
| | 6 | 1.27E−08 | 1.30E+06 | 1.88E−04 | | 6 | 8.75E−09 | 3.67E+06 | 3.21E−02 |
| | 5 | 2.47E−08 | 4.00E+06 | 2.31E−03 | | 5 | 1.17E−08 | 4.62E+06 | 5.42E−02 |
| VHH2 | 7.4 | 5.30E−09 | 6.79E+05 | 3.60E−03 | VHH8 | 7.4 | 1.69E−09 | 4.65E+05 | 7.87E−04 |
| | 7 | 2.42E−08 | 6.65E+05 | 1.61E−02 | | 7 | 6.45E−09 | 5.70E+05 | 3.68E−03 |
| | 6 | 5.96E−09 | 8.62E+05 | 5.13E−03 | | 6 | 1.27E−08 | 2.31E+05 | 2.93E−03 |
| | 5 | 1.00E−08 | 7.45E+05 | 7.46E−03 | | 5 | 1.09E−07 | 3.02E+05 | 3.28E−02 |
| VHH3 | 7.4 | 1.17E−13 | 8.53E+05 | 1.00E−07 | VHH9 | 7.4 | 2.82E−10 | 1.57E+06 | 4.44E−04 |
| | 7 | 2.69E−10 | 2.24E+06 | 6.04E−04 | | 7 | 4.76E−10 | 1.07E+06 | 5.10E−04 |
| | 6 | 2.70E−10 | 3.16E+06 | 8.51E−04 | | 6 | 6.74E−10 | 1.81E+06 | 1.22E−03 |
| | 5 | 1.36E−09 | 1.10E+06 | 1.49E−03 | | 5 | 8.41E−08 | 5.90E+05 | 4.96E−02 |
| VHH4 | 7.4 | 2.33E−09 | 5.71E+05 | 1.33E−03 | | 7.4 | 4.63E−08 | 3.81E+04 | 1.77E−03 |
| | 7 | 2.20E−09 | 5.72E+05 | 1.26E−03 | VHH10 | 7 | 8.97E−10 | 9.93E+02 | 1.48E−05 |
| | 6 | 3.18E−09 | 3.16E+05 | 1.01E−03 | | 6 | 3.29E−09 | 3.91E+02 | 2.05E−05 |
| | 5 | 1.00E−08 | 7.29E+05 | 7.29E−03 | | 5 | 1.78E−08 | 9.11E+02 | 1.06E−04 |
| VHH5 | 7.4 | 9.03E−11 | 1.37E+06 | 1.24E−04 | | 7.4 | 4.14E−08 | 7.67E+04 | 3.17E−03 |
| | 7 | 3.46E−10 | 1.19E+06 | 4.12E−04 | VHH11 | 7 | 2.03E−08 | 1.94E+05 | 3.94E−03 |
| | 6 | 1.11E−11 | 1.38E+06 | 1.52E−05 | | 6 | 3.42E−08 | 1.95E+05 | 6.65E−03 |
| | 5 | 5.76E−09 | 1.50E+06 | 8.61E−03 | | 5 | 1.13E−07 | 2.25E+05 | 2.53E−02 |
| VHH6 | 7.4 | 3.35E−09 | 1.05E+06 | 3.52E−03 | 2G11Ab | 7.4 | 2.76E−06 | 2.15E+02 | 5.92E−04 |
| | 7 | 2.65E−09 | 8.56E+05 | 2.27E−03 | | 7 | 4.43E−06 | 1.61E+02 | 7.12E−04 |
| | 6 | 1.56E−09 | 1.55E+06 | 2.42E−03 | | 6 | 2.35E−06 | 2.04E+02 | 4.79E−04 |
| | 5 | 8.50E−09 | 1.84E+06 | 1.56E−02 | | 5 | 1.51E−06 | 2.41E+02 | 3.63E−04 |

Experiment 2:

| VHH | pH | K_{D} (M) | kₒₙ (M⁻¹s⁻¹) | k_{Off} (s⁻¹) | VHH | pH | K_{D} (M) | Kₒₙ (M⁻¹s⁻¹) | k_{Off} (s⁻¹) |
|---|---|---|---|---|---|---|---|---|---|
| VHH1 | 7.4 | 1.12 × 10⁻⁹ | 1.0 × 10⁶ | 9.9 × 10⁻³ | VHH7 | 7.4 | 9.08 × 10⁻⁹ | 1.3 × 10⁸ | 1.18 × 10⁻² |
| | 7 | | 1.5 × 10⁶ | 2.1 × 10⁻³ | | 7 | | 1.3 × 10⁵ | 1.3 × 10⁻² |
| | 6.5 | | 1.1 × 10⁶ | 1.1 × 10⁻² | | 6.5 | | 4.3 × 10⁵ | 2.0 × 10⁻² |
| | 6 | | 5.6 × 10⁵ | 2.1 × 10⁻² | | 6 | | 4.0 × 10⁴ | 2.8 × 10⁻² |
| | 5.5 | | 4.7 × 10⁴ | 4.0 × 10⁻² | | 5.5 | | 3.5 × 10⁴ | 2.7 × 10⁻² |
| | 5 | | 2.0 × 10 [symbol] | 7.2 × 10⁻² | | 5 | | 5.5 × 10⁴ | 5.5 × 10⁻² |
| VHH2 | 7.4 | 4.89 × 10⁻⁹ | 4.83 × 10⁶ | 1.05 × 10⁻² | VHH8 | 7.4 | 2.65 × 10⁻⁹ | 1.0 × 10⁶ | 6.5 × 10⁻⁴ |
| | 7 | | 1.56 × 10⁶ | 1.36 × 10⁻² | | 7 | | 4.04 × 10⁵ | 9.76 × 10⁻⁴ |
| | | | | | | 6.5 | | 1.2 × 10⁶ | 7.0 × 10⁻⁴ |
| | 6 | | 3.76 × 10⁶ | 7.48 × 10⁻² | | 6 | | 1.3 × 10⁸ | 1.2 × 10⁻³ |
| | | | | | | 5.5 | | 1.9 × 10⁸ | 2.5 × 10⁻³ |
| | 5 | | 7.45 × 10⁵ | 7.4 × 10⁻² | | 5 | | 2.4 × 10⁶ | 8.4 × 10⁻² |
| VHH3 | 7.4 | 7.09 × 10⁻⁹ | 4.86 × 10⁴ | 3.45 × 10⁻⁴ | VHH9 | 7.4 | 2.82 × 10⁻¹⁰ | 1.57 × 10⁶ | 4.44 × 10⁻⁴ |
| | 7 | | 5.30 × 10⁴ | 8.44 × 10⁻⁴ | | 7 | | 3.79 × 10⁵ | 5.10 × 10⁻⁴ |
| | 6 | | 4.06 × 10⁴ | 4.99 × 10⁻⁴ | | 6 | | 6.15 × 10⁵ | 1.22 × 10⁻⁴ |
| | 5 | | 5.56 × 10⁴ | 3.69 × 10⁻⁴ | | 5 | | 5.90 × 10⁵ | 4.96 × 10⁻² |
| VHH4 | 7.4 | 2.34 × 10⁻⁹ | 5.71 × 10⁵ | 1.33 × 10⁻³ | VHH10 | 7.4 | 4.65 × 10⁻⁸ | 3.81 × 10⁴ | 1.77 × 10⁻³ |
| | 7 | | 3.16 × 10⁵ | 1.01 × 10⁻³ | | 7 | | 9.93 × 10⁴ | 1.48 × 10⁻³ |
| | 6 | | 5.72 × 10⁵ | 1.26 × 10⁻³ | | 6 | | 3.91 × 10⁴ | 2.05 × 10⁻³ |
| | 5 | | 7.29 × 10⁵ | 7.29 × 10⁻³ | | 5 | | 9.11 × 10⁴ | 1.06 × 10⁻² |
| VHH5 | 7.4 | 5.6 × 10⁻¹⁰ | 1.8 × 10⁶ | 1.8 × 10⁻⁴ | VHH11 | 7.4 | 5.10 × 10⁻⁸ | 7.67 × 10⁴ | 3.91 × 10⁻³ |
| | 7 | | 1.5 × 10⁶ | 1.9 × 10⁻⁴ | | 7 | | 1.94 × 10⁵ | 3.94 × 10⁻³ |
| | | | 1.3 × 10⁶ | 1.7 × 10⁻⁴ | | | | | |
| | 6 | | 1.7 × 10⁶ | 2.9 × 10⁻⁴ | | 6 | | 1.95 × 10⁵ | 6.65 × 10⁻³ |
| | | | 2.1 × 10⁶ | 6.2 × 10⁻⁴ | | | | | |
| | 5 | | 2.33 × 10⁸ | 3.3 × 10⁻² | | 5 | | 2.25 × 10⁵ | 2.53 × 10⁻² |
| VHH6 | 7.4 | 4.74 × 10⁻⁹ | 7.43 × 10⁵ | 3.52 × 10⁻³ | Anti− | 7.4 | 1.31 × 10⁻⁸ | 6.07 × 10⁴ | 7.94 × 10⁻⁴ |
| | 7 | | 3.92 × 10⁵ | 2.42 × 10⁻³ | CIMPR | 7 | | 3.11 × 10⁴ | 1.03 × 10⁻³ |
| | 6 | | 7.18 × 10⁵ | 2.27 × 10⁻³ | Ab | 6 | | 4.26 × 10⁴ | 5.34 × 10⁻⁴ |
| | 5 | | 3.3 × 10⁵ | 1.56 × 10⁻² | (2G11) | 5 | | 3.59 × 10⁴ | 3.89 × 10⁻⁴ |

[symbol]

indicates data missing or illegible when filed

For each association-dissociation cycle, affinity constants, on- and off-rates are shown (K_{D}, kₒₙ and k_{off}). As negative and positive control, GBP and an anti-CI-M6PR antibody (Ab) were included respectively. Curves were fitted according to the 1:1 binding model for each of the VHHs and the bivalent model was used for the anti-CI-M6PR Ab in the Octet RED software.

### Example 7. Anti-CI-M6PR VHH1, VHH5, VHH7 and VHH8 are Endocytosed and Colocalise with Late Endosome and Lysosomes

Alexa Fluor 488-labelled anti-CI-M6PR VHHs (10 μM) were incubated for 45 minutes on MCF7, together with Lysotracker Deep Red DND-99. Lysosomal targeting was the most pronounced for VHH7 and -8, as 3% of the lysosomes contained fluorescently labelled VHH at the moment of fixation (i.e. 45 minutes). In contrast to VHH7 and VHH8, VHH1, -3, -6, -9 and GBP (i.e. negative control) did almost not colocalize with Lysotracker Deep Red (FIG. 10 and Table 4) while between 0.4-1% of the MCF7-lysosomes contained VHH2, -4, -5 and -10. Because the fixation of treated MCF7 cells leads to decreased Lysotracker Deep Red DND-99 signal, we performed live cell imaging experiments.

Subsequent targeting to the endolysosomal compartment was assessed by monitoring AAF488-VHH signal inside living cells over time (3 h). Three fields of view were imaged every 6 minutes after LTR-incubation and administration of AF488-labeled anti-CI-M6PR VHHs, GFP binding protein (GBP) and recombinant human acid glucosidase α (rhGAA). After imaging, we calculated the uptake per cell volume by dividing the sum of voxel count for each fluorescent VHH by the sum of voxel count per imaged cell at a certain time point.

Results are shown in FIG. 11 for VHH1, -5, -7 and -8, and control. The uptake of the proteins relative to cell volume is shown in the upper graphs in grey and provided the best result for VHH1, -5, -7 and -8. The highest uptake of protein relative to cell volume was observed for VHH7, following a sigmoidal trend observed over three hours and an internalisation rate of 125.5×10⁴ summed AF-voxels/minute. Similarly, calculated by dividing the sum of AF488-positive voxel counts by time, the internalisation rate for VHH1 was 138.2×10⁴ summed AF-voxels/minute Compared to VHH7 and -1, the observed intracellular fluorescence of VHH5 was lower and more variable, while for VHH8 and rhGAA, internalisation rates were 68.7, 67.3 and 17.8×10⁴ summed AF-voxels/minute The profiles of the remaining VHHs were comparable to the negative control (GBP) and confirm that these indeed do not bind cell-surface hCI-M6PR.

The graphs in the middle show the mean percentages of VHH colocalising with lysosomes and were calculated by taking the ratio of the voxel counts of intracellular AF488-signal that colocalized with LTR and of the total intracellular VHH signal (circles). Next to this also the mean percentage of the entire endolysosomal pool containing the particular VHH or rhGAA was determined by the voxel count ratio of the VHH-signal colocalising with LTR and the total LTR signal (triangles). Due to the—sometimes—low intracellular AF488 signal and variable percentages, we included graphs showing the absolute voxel counts of the intracellular VHH signal and the VHH-LTR colocalising signal (third, lowergraph in each VHH panel of FIG. 11). Primary images after 120 minutes of incubation are shown in FIG. 38.

After 60 minutes, the percentage intralysosomal VHH1, 5 and 7 reaches equilibrium whereas VHH8 is coming to a plateau at 90 minutes. LTR-positive voxels of cells treated with VHH1, -8 and rhGAA contained up to 60% of the internalized protein while the total VHH7-positive LTR-positive pool was around 20% after three hours. The triangled curves outline the monitored fraction of LTR-stained organelles that colocalize with an AF488-VHH or -rhGAA. The total LTR-pool, positive for AF488 signal was the highest for VHH7, being between 30-40% after three hours, and was around 15% for VHH1. The fraction of the LTR-pool containing VHHs was less than 10% for VHH5, VHH8 and rhGAA and even lower for the other VHHs. Overall, these results clearly indicate specific endocytosis and highest percentage of lysosomal targeting with anti-CI-M6PR VHH1, 5, 7 and 8 (when compared to the negative control (i.e. GBP); Table 5). The positive control shows only limited lysosomal colocalization (FIG. 11).

TABLE 4
Degree of labelling of anti-CI-M6PR VHHs with Alexa Fluor 488.

| VHH | Degree of labeling |
|---|---|
| 1 | 0.3 |
| 2 | 0.9 |
| 3 | 0.2 |
| 4 | 0.3 |
| 5 | 0.7 |
| 6 | 0.2 |
| 7 | 2.4 |
| 8 | 0.7 |
| 9 | 0.3 |
| 10 | 0.4 |
| 11 | 0.2 |
| GBP | 0.3 |

TABLE 5
Percentage of intralysosomal VHHs and percentage of lysosomes colocalizing
with VHH after 60 minutes incubation on MCF7 cells.

| | % cellular VHH in | |
|---|---|---|
| | lysosomes | % of lysosomes containing VHH |
| anti-GFP VHH | 2.081556425 | 0.01631969 |
| anti-CI-M6PR VHH1 | 59.04545714 | 4.085886889 |
| anti-CI-M6PR VHH2 | 19.74416491 | 0.150080973 |
| anti-CI-M6PR VHH3 | 99.45199228 | 0.248934051 |
| anti-CI-M6PR VHH4 | 78.59529604 | 0.379712423 |
| anti-CI-M6PR VHH5 | 89.43648369 | 1.550855749 |
| anti-CI-M6PR VHH6 | 4.58991723 | 0.035321469 |
| anti-CI-M6PR VHH7 | 25.9084669 | 9.472710875 |
| anti-CI-M6PR VHH8 | 62.29034866 | 6.642164726 |
| anti-CI-M6PR VHH9 | 38.80981524 | 0.259127814 |
| anti-CI-M6PR VHH10 | 74.60317461 | 0.122773461 |
| anti-CI-M6PR VHH11 | 0.279329609 | 0.002311123 |

Although labelled with a differential efficiency, the variation in endolysosomal content for these four anti-CI-M6PR VHHs upon endocytosis may indicate a differential lysosomal delivery or a variable cycling path for these molecules. This is the most pronounced for VHH7, for which we observed—compared to the others—a lower VHH7-endolysosomal pool but an increased fraction of endolysosomes containing VHH7 (FIG. 11).

To explore whether these variations in endolysosomal content were the result of true lysosomal delivery, we investigated the fraction of AF488-VHH colocalizing with LAMP1, a lysosomal membrane protein increasingly present in mature lysosomes, on fixed cells. We did this for VHH7—which is increasingly endocytosed—and also for VHH8, for which a low intracellular fraction but larger LTR-positive fractions could be observed. After their incubation for four hours on HeLa cells, an anti-LAMP1 antibody was used for staining.

As shown in FIG. 39, the intralysosomal fraction of intracellular VHH7 after 240 minutes in the same range (i.e. 19%) as to what we observed during live-cell imaging experiments after 200 minutes with LTR (i.e. 20%, FIG. 11). However, this is entirely opposite for VHH8 as we detected only 2.5-12% intracellular VHH8 colocalising with LAMP1-positive lysosomes compared to the 60% with LTR (FIG. 11). Next to the intralysosomal fraction, we also calculated the percentage of LAMP1-stained voxels containing VHH (FIG. 39 A). These values are overall low and within the range of what was previously observed for LTR-stained fixed cells (FIG. 10).

It is remarkable that the LAMP1-colocalising and LTR-colocalising fraction of AF488-VHH7, being 19% and 20% respectively, is similar while VHH8 has a much higher LTR-colocalising fraction (i.e. 60%) compared to the LAMP1-colocalising fraction (i.e. maximally 12%). Although it is difficult to compare live-cell imaging and microscopic examination after fixation, also of course because of the different cell lines used, these experiments possibly suggest that VHH7 and VHH8, may follow different endolysosomal paths. VHH7 shows a more or less equal colocalisation with LTR and LAMP1, suggesting VHH7's increased lysosomal targeting. Given VHH7's five times higher dissociation rate at pH 6.0 versus pH 7.4, it is plausible that immediate endosomal acidification upon endocytosis allows VHH7 to quickly dissociate after which it can be delivered to the lysosome together with the endosomal cargo during maturation.

In that case, the unbound receptor is recycled and may participate in a new round of binding. The low amount of VHH8 in LAMP1-stained compared to LTR-stained organelles could indicate an increased colocalisation with the earlier endolysosomal network. However, a higher amount of LAMP1-lysosomes with VHH8 was detected (FIG. 39B). Because VHH8's transition in dissociation between pH 6.0 and pH 5.0, it is plausible that it may remain bound to hCI-M6PR at the early endosomal stage (pH 5.9-6.5) instead of being delivered to the lysosome. The high LAMP1-colocalisation of VHH7 on the one hand and the peripheral localisation of VHH8, on the other hand, can be indicative of this (FIG. 39C).

It should also be noted that once the VHHs reach the mature lysosome, they would probably be denatured by lysosomal proteases. What then happens to the fluorophore in terms of localisation is unknown. However, we can assume that this behaviour will be similar across the studied VHHs.

Important throughout the interpretation of the absolute counts in these microscopic examinations is to be aware of the obtained degree of labelling of every VHH and its reduced affinity for the recombinant hCI-M6PR_{D1-D3}. While this is unavoidable and comparable to what is generally expected for these NHS-ester labels, absolute counts of VHH7 are probably overestimated due to its high labelling efficiency. It is also important to recall that the divergent affinity combined for recombinant hCI-M6PR_{D1-D3} does not directly correspond binding to the native hCI-M6PR. Moreover, as we calculated fractions of colocalising AF488 and LTR or LAMP1 signal, it was observed, with all other noted, that low-level endocytosis with variability among the replicate VHHs can quickly result in aberrantly high colocalising percentages. The other VHHs had no evidence of CI-M6PR receptor engagement at the cell surface in previous experiments; yet, most did show this low-level highly variable uptake, similar to GBP and were excluded for these reasons from the above discussion

### Example 8. In Vivo Biodistribution of VHH1-11 in Mice

The anti-CI-M6PR VHHs (50 μg) were each subjected to radiolabelling with ⁹⁹mTc on the hexahistidine tag using the ^{99m}Tc-tricarbonyl method. Radiochemical purity of all VHHs was determined by iTLC analysis and free ^{99m}Tc was retained on a NAP5 column. Additionally, unfolded and potential aggregated formats were removed by size exclusion chromatography. Three mice per VHH group were intravenously (i.v.) injected with ^{99m}Tc-radiolabeled anti-CI-M6PR VHH. SPECT acquisition was started three hours after injection, animals were scanned on a 75-pinholes stationary detector SPECT system (U-SPECT-II, MILabs) for 15 minutes total body. The SPECT scan was followed by a 2-minute CT for anatomical information and image reconstruction.

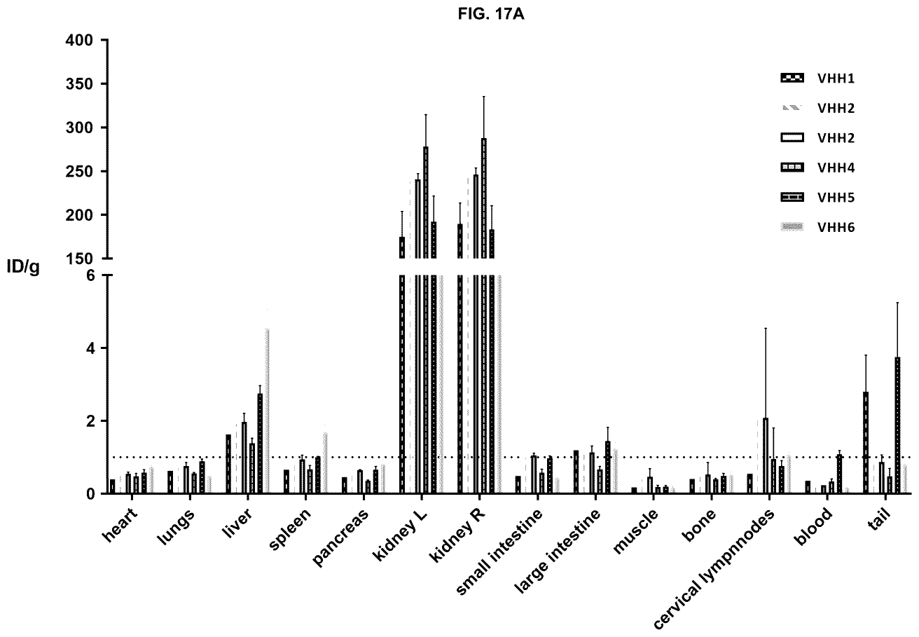

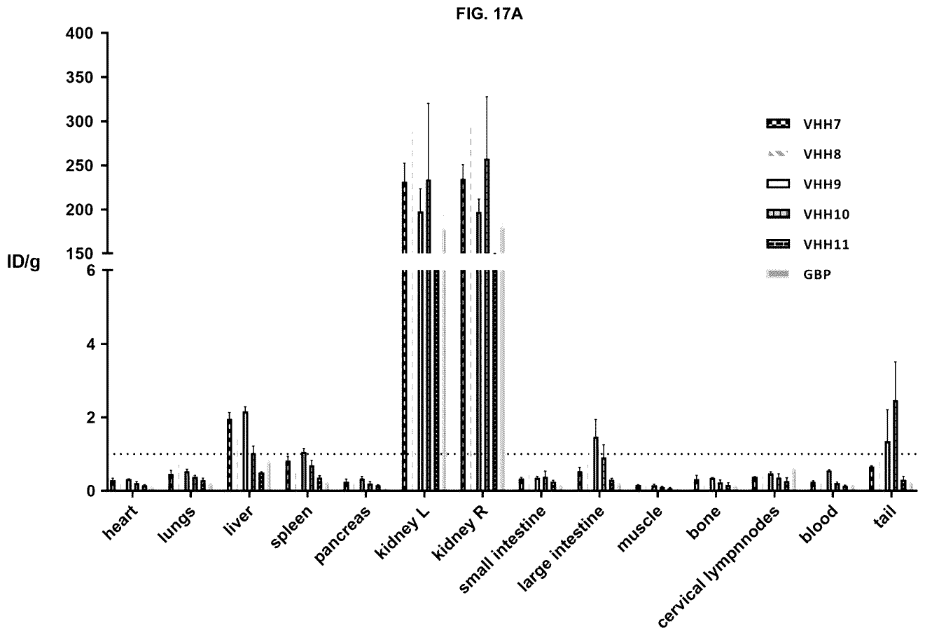

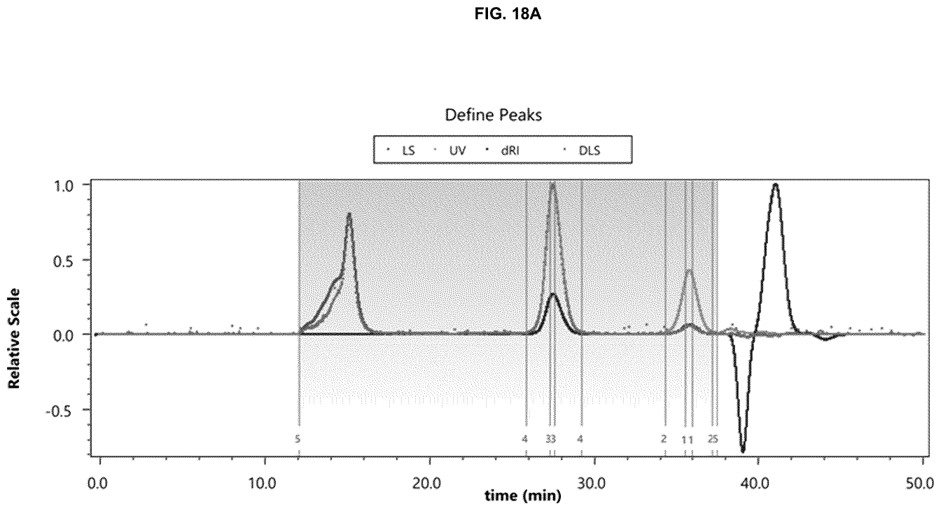

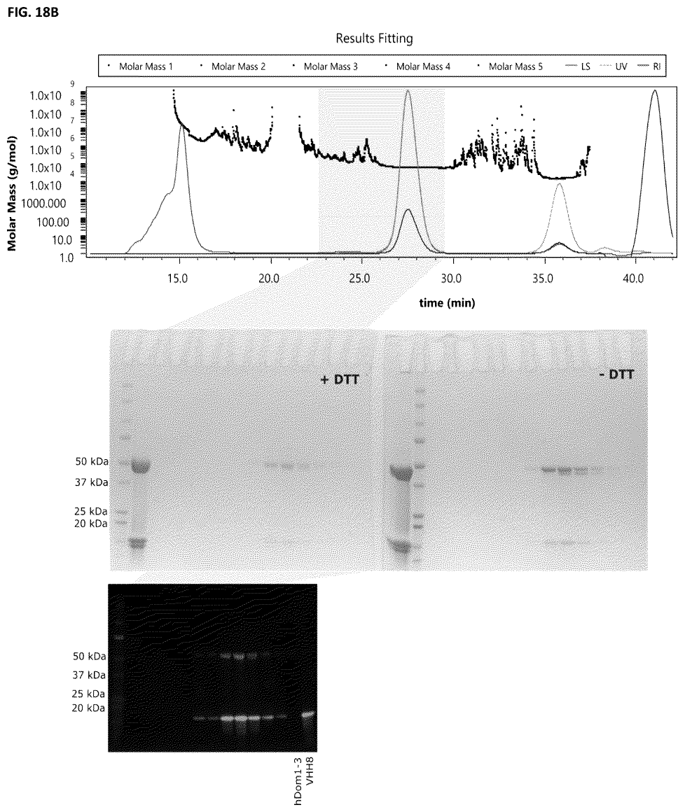

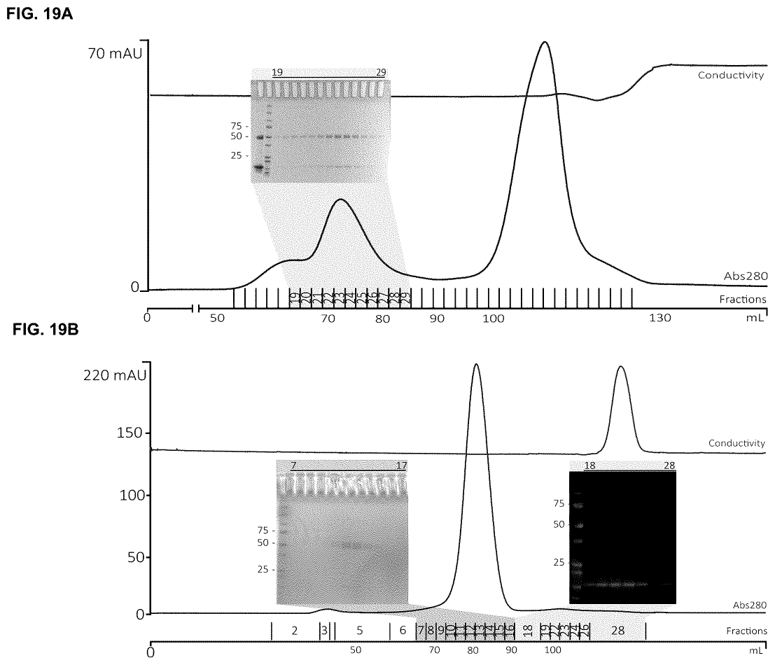

Three hours after being injected in mice, SPECT/CT imaged mice demonstrated high levels of radioactivity in the kidney and the bladder, the non-specific clearance route of VHHs in general. Additionally, ^{99m}Tc-labelled VHHs, were detected in increased amounts in the liver, the gut, the heart, lungs and the lymph nodes as compared to the GFP-binding protein (GBP). Additionally, γ-counting of each isolated tissue after SPECT/CT scanning resulted in values of isolated doses per gram (ID/g) that were normalized for the injected activity of each VHH. As partially represented by SPECT/CT images, organs showing higher anti-CI-M6PR VHH uptake than the control (GBP) were the liver, heart, lungs, gut and lymph nodes (image data not shown). Up to 3 times increased uptake in the liver was observed for VHH5, -6 and -9 while VHH 2, 5 and 6 had elevated amounts in the heart. In the lungs, VHH2, -5 and -8 were abundantly present. Small and large intestine compromised mostly VHH1, -2, -5, -6 and -9 and the lymph node generally -2 and -6, compared to the other VHHs. Finally, 3 h post injection VHH5 and -9 had the highest concentrations in the blood and VHH6 and -11 the least in the kidneys. As a final point, most VHHs (i.e. VHH 1, 2, 3, 4, 5, 6, 7, 8 and 9) showed at least a 3-fold higher uptake in the muscle than GBP (FIG. 17).

In conclusion, in this experiment we can conclude that our lead candidates VHH7 and VHH8, also show a favourably low level of aspecific tissue binding; lead candidates VHH1 (hCI-M6PR specific), is overall also acceptable, with some evidence of spleen and large intestine aspecific binding. VHH candidate 5, shows elevated likely aspecific binding to a variety of murine organs.

### Example 9. Multi-Angle Light Scattering and Crystallography of VHH-hDom1-3his Complexes

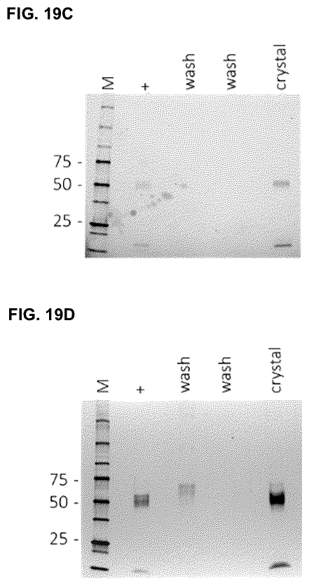

Because the three N-terminal domains of CI-M6PR are repeats of CI-M6PR type domains (Pfam PF00878), we first determined the molecular mass and oligomeric state of the hCIMPR_{D1-D3}:VHH protein complex, we analysed SEC-MALLS-eluted and fractionated samples after incubating hCIMPR_{D1-D3}:VHH8 in a 1:1 (FIGS. 18 & 40A) and 1:3 (FIG. 19) molarfashion. The calculated protein masses corresponded to what was expected for the VHH and antigen, 17 kDa (±1 kDa) and 51.3 kDa (±0.9 kDa) respectively, and 62 kDa (±2 kDa) for the complex, which complies to an equimolar binding of both proteins. Aggregated or other oligomeric structures could be detected but remain limited, also when fractionated samples were analyzed on non-reducing SDS-PAGE. The complexation of VHH and antigen proteins was also independent from hCI-M6PR_{D1-D3} N-glycans, as investigated after endoglycosidase H digest. Because we found an equimolar binding of VHH8 and hCI-M6PR_{D1-D3}, also equal concentrations of VHH7 and hCIM6PR_{D1-D3} were used during a next SEC-MALLS run (FIG. 40B). Molecular masses of 50 kDa (±2 kDa) for hCIM6PR_{D1-D3}, 15 (±1 kDa) for anti-CI-M6PR VHH7 and 63.7 (±0.5 kDa) for the complex were measured. A final preparative SEC run was therefore performed accordingly for both VHH7, VHH8 and VHH 1H11 (the latter being obtained after re-panning experiments, see Example 18, and characterized thereafter, followed by its production and complexation as described in Example 21) with (glycosylated) hCI-M6PR_{D1-D3} before the co-crystallisation screening (FIGS. 19 A & B). After crystallisation, the presence of both the antigen and the VHH was verified on SDS-PAGE (FIGS. 19 C & D).

The N-terminal first three domains of the CI-M6PR (CI-M6PR_{D1-D3}), resemble previously published conformations. In the co-crystal with VHH7 and VHH8, hCI-M6PR_{D1-D3} adopts a trefoil-shaped structure similar to a conformation observed for bovine CI-MPR_{D1-D3} (pdb 1q25).

In the co-crystal structure with VHH 1H11 the third domain has shifted towards D1 to resemble the conformation present in pdb 6p8i (FIG. 43). While present in the crystallisation mixture of VHH7 and -8-containing complexes mannose-6 phosphate was not observed in any of the structures. N-glycans at the three N-glycosylation sequons (i.e. Asn112, Asn400 and Asn435) could be identified to varying degrees from the electron density. In the co-crystal structures with VHH7 and VHH8 clear electron density could be interpreted for a Man₃GlcNAc₂ or Man₄GlcNAc₂ containing glycan at Asn112. Only partial core GlcNAc or GlcNAc₂ could be interpreted from the electron density at the other positions. Interesting however are the crystal contacts, observed in these crystal structures between the N-linked glycan on Asn112 and the M6P binding pocket in D3 of the crystallographic symmetry-related CI-M6PR copy. More specifically the α1,3-Man of the oligomannosylated glycan on Asn112 binds a cleft on CI-M6PR_{D3} interacting with residues Tyr359, Gln383, Arg426, Glu451 and Tyr456 mostly via hydrogen bonds (FIG. 45). The N-linked glycans on Asn112 and Asn435 of the VHH 1H11 co-crystal structure could be identified as core 1-6 fucosylated.

The core structure of each domain consists of a flattened @3-barrel (Pfam domain CIMR PF00878) comprising a five-stranded antiparallel β-sheet (β3-β6) with its strand running orthogonally oriented over a second five-stranded β-sheet (β8-β11), of which the fourth strand interjects between β9 and β11. Each domain should contain four disulfide bonds, as comparable to the bovine crystal structure of the N-terminal three domains of the CI-M6PR (PDB: 1sy0, 1sz0, 1q25,6p8i)¹⁰⁴. The N-terminus of human domain 2 (residues 161-313) and domain 3 (residues 314-467) each contain a linker region composed of a random coil followed by two ancillary β-strands (β0, β1 and β2) that connect the core-flattened β-barrel structures.

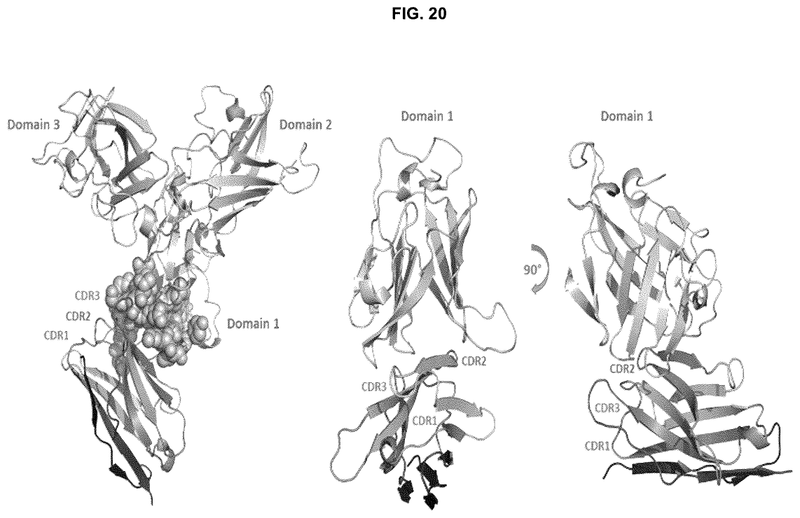

Anti-CI-M6PR VHH7, VHH8 and VHH 1H11 adopt the general immunoglobulin-like fold with a neutral, and stretched-twist turned CDR3 loop respectively. The highest resolution crystal structure of the anti-CI-M6PR VHH7 and hCI-M6PR_{D1-D3} protein complex was solved to a resolution of 2.2 Å (FIG. 41A) and was grown at pH 6.5 (FIG. 41A). The first protein complex reveals a unilateral positioned VHH7 that is packed in between the two β-sheets of hCI-M6PR_{D1}'s flattened β-barrel (FIG. 41B). While presenting one flank to its antigen, VHH7 interacts via its CDR1, 2 but also with residues in CDR3 (FIG. 41C). These make contacts with the amino acid side chains of the intradomain loops A-D of D1 (FIGS. 20 and 41). This complex is nearly identical in the other crystal form.

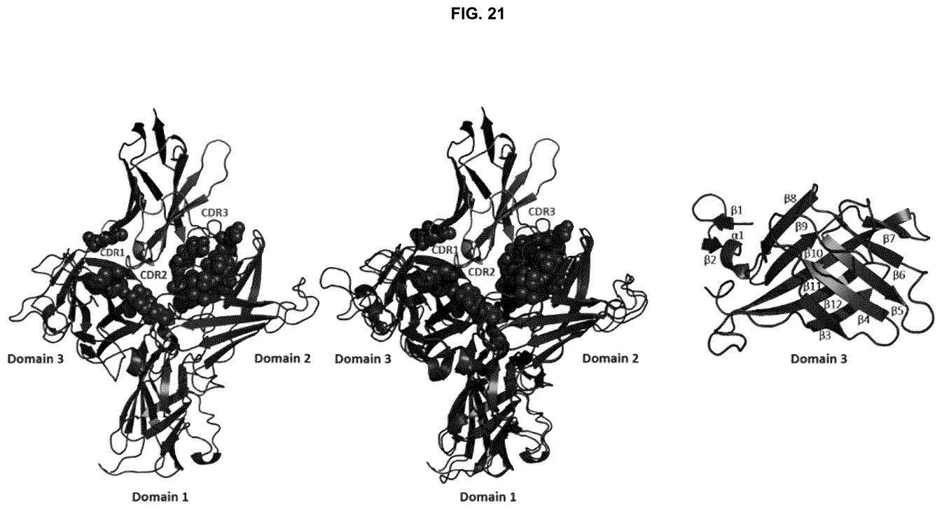

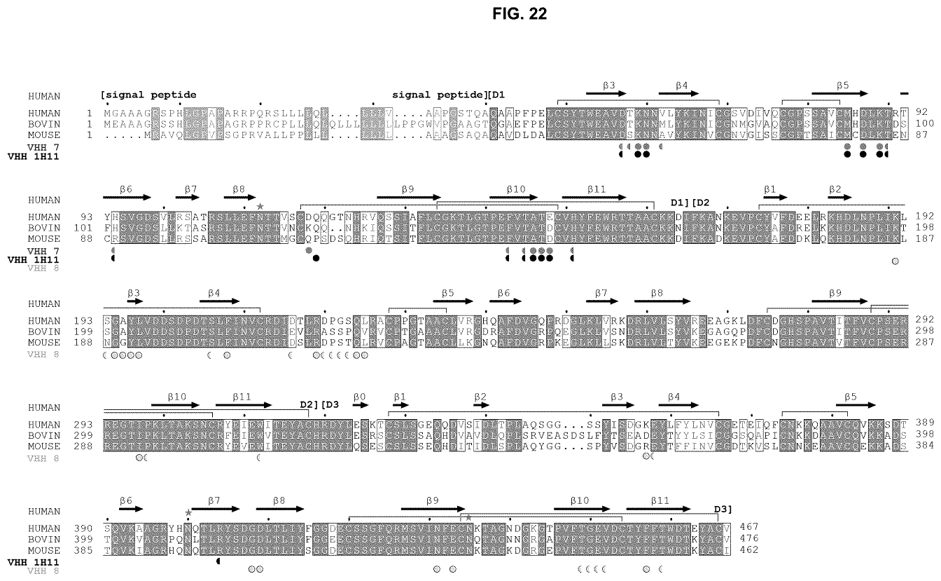

The VHH8 co-crystal structure which was solved to a resolution of 2.75 Å reveals VHH8 is situated in between hCI-M6PR_{D2} and hCI-M6PR_{D3} of the CI-M6PR (FIG. 21, 42A). These form a V-shaped surface from which the amino acids contact the variable protruding loops of VHH8 (FIG. 42B). In general, most of the residues from CDR2 interact with residues of D3, whereas the residues from CDR3 are faced towards D2. The contribution of CDR1 is, compared to the other CDRs, only limited for the overall interaction (FIG. 42C).

The crystal structure of the VHH7-competing anti-CI-M6PR VHH 1H11 and hCI-M6PR_{D1-D3} was solved to a resolution of 2.7A and thereby confirmed the results obtained from the mutational screening and competitive BLI. Comparable to VHH7, VHH 1H11 faces hCI-M6PR_{D1}'s flattened β-barrel unilaterally (FIGS. 43A-B) and interacts with residues from both β-sheets with CDR1 and CDR2 predominantly (FIG. 43C). As a general overview, a schematic representation of the binding of the lead anti-CI-M6PR VHHs is shown in FIG. 44.

The PISA²⁷ and FastContact²⁸⁻³⁰ software were consulted to roughly calculate and identify the interacting residues at the binding surface of anti-CI-M6PR VHH7, -8 and -1H11 with hCI-M6PR_{D1-D3}. Because FastContact analysis is biased towards electrostatic interactions, we combined the calculations of the desolvation free energy and electrostatic energy with distance measures, calculated in PyMol to approximate the interfacing residues of anti-CI-M6PR VHHs and its antigen. From this information, two very different para- and epitopes could be delineated for either VHH7, VHH 1H11 and VHH8. The epitope of anti-CI-M6PR VHH7 (FIG. 20, 41) mainly consists of amino acids that are part of the intradomain loops A, B, C and D of the β-sheets in CI-M6PR_{D1} (FIG. 41). In addition, hydrophobic residues (e.g. Phe143) that make up the hydrophobic core of the flattened βbarrel contribute to the VHH binding. According to current estimations, important paratope residues comprise Arg33, Lys57 and Asp104 and interact with hCI-M6PR_{D1} residues Asp 87, Glu148 and Lys89 respectively (Table 6A & B, FIG. 41D). These calculations allowed us to confirm the similarity of the epitopes of VHH 1H11 and VHH7. Generally, residues estimated to contribute to the interaction were comparable to the epi- and paratope of VHH7 (Table 6 A & B). The residues Arg33 and Lys57 for example, were estimated to be highly involved in the binding of VHH 1H11 to CI-M6PR_{D1-D3} (FIG. 43D), whereas residues of the VHH 1H11's CDR3 are probably contributing less. On top of this, the estimations here showed a high similarity between both epitopes with Asp 87, Lys 89 and Glu148 as highly contributing residues (Table 7B). The epitope of anti-CI-M6PR VHH8 is highly different (FIG. 21, 42). In contrast to VHH7, interactions of CI-M6PR_{D1-D3} with VHH8 occur with inter- and intradomain loops of hCI-M6PR_{D2} and hCI-M6PR_{D3} but also residues within the β-strands of these domains are impactful (Table 8A & B). As described, amino acids that constitute CDR2 contact D3. Of these, the Lys57 of VHH8 is estimated to form an electrostatic interaction with Glu409 and Glu433 of D3 (Table 8 A, FIG. 42D). Strong contacts between D2 and CDR3 were estimated to be Asp102 and Lys191 respectively (Table 8A, FIG. 42D).

The epitope information allows us to further discuss the (non-)cross-reactive binding of VHH7, -8 and 1H11. Despite a sequence identity of 75% between the human Domain 1-3 and either Bos taurus or Mus musculus domain 1-3 sequences, the VHH7 and VHH8 interface is rather conserved. In FIG. 22, we indicated each of the specific epitope residues in the orthologous sequences for hCI-M6PR_{D1-D3}. A higher degree of variation can be observed for VHH7 than for VHH8 when taking into account residues that contribute significantly to the total binding free energy (i.e. ΔG below −1.5 kcal/mol).

For Tables 6-8 corresponding to the information on VHH 7, VHH 1H11, and VHH8, resp., the interacting residues and their estimated type of interactions as the estimated binding free energy (ΔG) determined by the sum of the calculated electrostatic free energy, desolvation free energy and configuration entropy for the interaction between the residues of anti-CI-M6PR VHH and hCI-M6PR_{D1-D3} by FastContact and PISA are shown.

TABLE 6
Overview of the epi- and paratopes of anti-
CI-M6PR VHH7 binding the rhCI-M6PR_{D1-D3}.

| | VHH [symbol] | hCl-MPR [symbol] | Estimated type of | Estimated binding free |
|---|---|---|---|---|
| | residues | residues | interaction | energy (kcal/mol) |
| CDR1 | Asp 31 | Lys 59 | Electrostatic | ΔG < −1.0 |
| | Arg 33 | Asp 57 | Polar | ΔG < 0.0 |
| | Arg 33 | Asp 87 | Electrostatic | ΔG < [symbol] |
| | Arg 33 | Asn 60 | Polar | ΔG < −1.0 |
| | Asp 35 | Lys 89 | Electrostatic | ΔG < −1.0 |
| CDR2 | Ser [symbol] | A [symbol] 60 | Polar | ΔG < −1.0 |
| | Tyr 54 | Asp [symbol] | Polar | ΔG < 0.0 |
| | Tyr 54 | Ala 146 | Polar | ΔG < 0.0 |
| | Tyr 54 | Thr 147 | Polar | ΔG < 0.0 |
| | Tyr [symbol] | Phe [symbol] | Hydrophobic | ΔG < 0.0 |
| | Trp [symbol] | Met 85 | Polar | ΔG < 0.0 |
| | TRP 56 | Glu 148 | Polar | ΔG < 0.0 |
| | Lys 57 | Glu 148 | Electrostatic | ΔG < −4.0 |
| | Lys 57 | Asp 118 | Electrostatic | ΔG < −3.8 |
| CDR3 | Lys 96 | Asp 87 | Electrostatic | ΔG < −0.0 |
| | Asp 104 | Lys 89 | Electrostatic | ΔG < −4.0 |

[symbol]

indicates data missing or illegible when filed

TABLE 7
Overview of the epi- and paratopes of anti-
CI-M6PR VHH 1H11 binding the rhCI-M6PRD1-D3.

| | VHH [symbol] | hCL-MPR [symbol] | Estimated type of | Estimated binding free |
|---|---|---|---|---|
| | residues | residues | interaction | energy (kcal/mol) |
| CDR1 | Asp 31 | Lys [symbol] | Electrostatic | ΔG < −4.0 |
| | A [symbol] 32 | Lys 59 | Electrostatic | ΔG·< −1.8 |
| | Arg 33 | Asp 87 | Electrostatic | ΔG < −4.0 |
| | Arg 33 | Asn 60 | Polar | ΔG < −1.0 |
| | Arg 33 | Thr 90 | Polar | ΔG > 0.0 |
| | Arg 33 | Asp [symbol] | Polar | ΔG < 0.0 |
| | Asp [symbol] | Lys [symbol] | Electrostatic | ΔG < −4.0 |
| CDR2 | Thr 50 | Asp 87 | Polar | ΔG < 0.0 |
| | Ala 52 | Asn [symbol] | NA | ΔG < 0.0 |
| | Ser 53 | [symbol] | Polar | ΔG < −1.0 |
| | Tyr 54 | Ala 146 | NA | ΔG < 0.0 |
| | Tyr [symbol] | Glu 148 | Polar | ΔG: NA |
| | Tyr 54 | Arg 404 | Polar | ΔG: NA |
| | Gly 55 | Glu 148 | NA | ΔG > 0.0 |
| | Trp 56 | Ala 146 | NA | ΔG > 0.0 |
| | Trp 56 | Thr 147 | Polar | ΔG > 0.0 |
| | Trp 56 | Glu 148 | Polar | ΔG < 0.0 |
| | Trp 56 | Met 85 | NA | ΔG < 0.0 |
| | Lys 57 | Gln 119 | Polar | ΔG < 0.0 |
| | Lys 57 | Asp 118 | Electrostatic | ΔG < 0.0 |
| | Lys 57 | Glu 148 | Electrostatic | ΔG < −1.0 |
| CDR3 | Asn 96 | Lys 89 | Polar | ΔG < −1.0 |
| | Ser 97 | Lys 89 | Polar | ΔG: NA |
| | Gl y98 | Lys 88 | Polar | ΔG: NA |

[symbol]

indicates data missing or illegible when filed

TABLE 8
Overview of the epi- and paratopes of anti-
CI-M6PR VHH8 binding the rhCI-M6PRD1-D3.

| | [symbol] l | hCl-MPR− [symbol] | Estimated type of | Estimated binding free |
|---|---|---|---|---|
| | residues | residues | interaction | energy (kcal/mol) |
| CDR1 | Tyr 32 | Arg 219 | Polar | ΔG < −0.0 |
| CDR2 | Arg 52 | Asp 4019 | Polar | ΔG < −1.0 |
| | Trp 53 | Phe 457 | Hydrophobic | ΔG < −0.0 |
| | Ser 54 | Asn 431 | Polar | ΔG < −0.0 |
| | Ser 55 | Asp 409 | Polar | ΔG < −0.0 |
| | Ser 56 | Glu 433 | Polar | ΔG < −0.0 |
| | Lys 57 | Gly 408 | Electrostatic | ΔG < −1.0 |
| | Lys 57 | Asp 409 | Electrostatic | ΔG < −4.0 |
| | Lys 57 | Glu 433 | Eletrostatic | ΔG < −4.0 |
| FR2 | Arg 72 | Glu 433 | Polar | ΔG < −0.0 |
| | Asp 73 | Lys 357 | Polar | ΔG < −1.0 |
| | Asn 74 | Lys 357 | Polar | ΔG < −1.0 |
| CDR3 | Ile 100 | Gly 194 | Hydrophobic | ΔG < −0.0 |
| | Ile 100 | Ala 195 | Hydrophobic | ΔG < −0.0 |
| | Val 101 | Ala 195 | Hydrophobic | ΔG < −0.0 |
| | Val 101 | Phe 208 | Hydrophobic | ΔG < −0.0 |
| | Val 101 | Leu 225 | Hydrophobic | ΔG < −0.0 |
| | Asp 102 | Lys 191 | Electrostatic | ΔG < −4.0 |
| | Asp 102 | Ala 195 | Polar | ΔG < −0.0 |
| | Asp 102 | Leu 197 | Polar | ΔG < −0.0 |
| | Phe 103 | Tyr 196 | Hydrophobic | ΔG < −1.0 |
| | Phe 103 | Leu 197 | Hydrophobic | ΔG < −0.0 |
| | Phe 103 | Ile 297 | Hydrophobic | ΔG < −1.0 |
| | Ser 108 | Gln 224 | Polar | ΔG < −0.0 |

[symbol]

indicates data missing or illegible when filed

### Examples 10-15: Therapeutic Potential of Fusion Proteins of Anti-CI-M6PR-VHH and Cathepsin D Protease or Acid α-Glucosidase in Enzyme-Replacement Therapy (ERT)

Multiple strategies have been employed to enhance current ERT and to tackle its drawbacks. In this context, we have generated VHHs that bind in a pH-dependent way to the cation-independent mannose-6-phosphate receptor (CI-M6PR) with subsequent transport to the endolysosomal system. Here, we describe the expression of chimeric proteins containing both the lysosomal enzyme of interest and the anti-CI-M6PR VHHs. We choose for pro-hCTSD, which we envision as ERT to treat lysosomal storage disorders such as sporadic inclusion body myositis or neuronal ceroid lipofuscinosis 10⁴⁹. The inactive pro-hCTSD format was chosen as it is only limitedly active at physiological pH (e.g. in the blood) and therefore preferred over the active form to prevent potential side effects. Additionally, we explored rhGAA that could be used to treat Pompe disease^{50,51}. Besides their mammalian expression, we outline their purification and further investigate the cellular uptake, activity or effectivity in vitro. Because we aimed to create a next-generation ERT biologic with improved half-life in vivo by means of our previously developed GlycoDelete technology⁵², we describe the behaviour of CTSD-VHH chimeric constructs in vivo and outline the analysis of their glycan-based clearance when produced in GlycoDelete cells versus wild-type CTSD-VHH protein.

### Example 10. Cathepsin D can be Equally-Well Produced by GlycoDelete Cells and Wild-Type HEK293 Expression

First, we prepared pure recombinant (r) pro-hCTSD proteins from HEK293 and HEK293 GlycoDelete cells (Error! Reference source not found.). rhCTSD was first captured by its C-terminal His₆ tag, followed by a polishing step by SEC. Ultimately, pure protein was obtained, with a low level of unidentified lower molecular weight fragments or contaminants in the sample after SEC. Pro-hCTSD is represented as different glycoforms on SDS-PAGE, with variants eluting at slightly lower imidazole concentrations. With a yield around 16 mg/L for both, pro-hCTSD could be quite efficiently expressed by both HEK293 and HEK293 GlycoDelete cells in general.

Human CTSD harbours two N-glycosylation sites at positions Asn134⁵⁷ and Asn263⁵⁷⁻⁵⁹. We characterised the N-glycans by mass spectrometry and DSA-FACE to compare the profiles for wild-type and GlycoDelete pro-hCTSD. The absence of N-glycans in GlycoDelete material was observed by the lower molecular weight of CTSD on western blot and verified by PNGaseF digestion (Error! Reference source not found.A). We additionally verified that the activity of both pro-hCTSD variants is not influenced by the N-glycan removal, as we determined a K_{M} and kcat value that are situated in the same range (Error! Reference source not found.B and C).

To obtain a more detailed view on the glycan profile, we compared DSA-FACE profiles of the untreated and exoglycosidase digested sample (Error! Reference source not found.A) and identified wild-type pro-hCTSD glycans to be highly sialylated, as observed after sialidase treatment. Subsequently, peak-shifts occurring after digestions with galactosidase, hexosaminidase and mannosidase or combinations of these, revealed a heterogeneous set of glycans for pro-hCTSD. These entailed bi- to tetra-antennary complex type N-glycans but also hybrid type N-glycans. A combined treatment with sialidase and phosphatase (CIP), identified ˜20% phosphorylated sugars. These are present on high mannose sugars, proven by the peak-shifts after mannosidase digestion, but also on the hybrid-type N-glycans, shown by the combined digestions with CIP and mannosidase, galactosidase, hexosaminidase and fucosidase enzymes. Core-fucosylated N-glycans were observed when panels from sialidase and CIP were compared to the panels derived from sialidase, CIP and fucosidase treatments (Error! Reference source not found.A).

The glycan profile of GlycoDelete pro-hCTSD was very much simplified but revealed some remaining Man₃/5GlcNAc₂ and M6P-containing high mannose N-glycans, as seen after digestions with mannosidase and CIP. These were probably less accessible for EndoT and therefore insufficiently processed (Error! Reference source not found.B). By mass spectrometry the typical GlycoDelete glycan profile was identified for pro-hCTSD: single GlcNAc, GalGlcNAc and SiaGalGlcNAc glycans were observed of which at least 12% was sialylated (Error! Reference source not found.B). This also shows that the remaining high mannose-glycans are trace quantities, consistent with the more or less complete deglycosylation. However, these trace-quantities correspond to the remaining peaks observed on the mass spectrometry profile.

### Example 11. Production of VHH-Cathepsin D Chimeric Proteins

To progress in the development of our glycosylation-independent targeted enzyme replacement therapy, we created fusions of pro-hCTSD and any of the 11 anti-CI-M6PR VHHs. These VHHs were either N- (i.e. VHH-pro-hCTSD) or C-terminally fused to pro-hCTSD (i.e. pro-hCTSD-VHH), linked by a triple Gly₄Ser linker and all constructs contained a FLAG₃His₆ tag (SEQ ID NO:34) at the C-terminus. The pro-hCTSD-VHH constructs contained the endogenous CTSD signal sequene, while the VHH-pro-hCTSD proteins contained an immunoglobulin signal sequence.

A pilot expression analysis revealed successful but limited expression of a, approximately, 65 kDa protein by cells transfected with the pro-hCTSD-VHH constructs (Error! Reference source not found.A). When analysing the supernatant that should contain secreted VHH-pro-hCTSD proteins, we only observed 50 kDa protein, which is comparable to the molecular weight of the positive control, pro-hCTSD-His₆ (Error! Reference source not found.B). Therefore, we analysed the corresponding crude lysate of cells expressing VHH-pro-hCTSD (Error! Reference source not found.C) and stained for CTSD and VHH specifically on a western blot containing the secreted fraction (Error! Reference source not found.D). However, only a (partially) processed VHH-pro-hCTSD protein could be identified by either an anti-His₆ antibody (Error! Reference source not found.C) or an anti-CTSD antibody (Error! Reference source not found.D), whereas the VHH could not be identified in the secreted fraction (Error! Reference source not found.C). To explore whether linker processing along or after VHH-pro-hCTSD secretion was the culprit, we cloned and expressed VHH-pro-hCTSD proteins that contained other linkers than the triple Gly₄Ser linker. We chose two other flexible linkers (i.e. GlySer and Gly₈) and two rigid and stable linkers (i.e. AlaPro₇ and triple GluAlaAlaAlaLys) but these could not prevent the posttranslational processing of the VHH-pro-hCTSD recombinant protein (Error! Reference source not found.E). It is most plausible that VHH-pro-hCTSD constructs can only be secreted without the VHH, due to pro-peptide processing along the secretory pathway, which of course removes the N-terminal VHH.

To verify the CTSD activity after its fusion to anti-CI-M6PR VHHs, we monitored proteolytic cleavage of a synthetic fluorescent substrate in the secreted fraction oftransfected cells. In the same experiment, our proCTSD-His₆ construct and GFP were used as positive and negative control. In Error! Reference source not found.F the relative fluorescence for each non-normalised sample is outlined. Compared to the negative controls, being the supernatant of cells expressing GFP orjust Freestyle293 medium, proteolytic acitivty is clearly observed for the medium with recombinant chimeric protein. The increased activity observed for recombinant pro-hCTSD-His₆ corresponds to its high expression levels, vice versa for the chimeric proteins that have rather low expression yields. It is clear that the pro-hCTSD-VHH constructs are expressed at ˜10-folds lower levels than non-VHH fused proCTSD-His₆ in these non-optimised transient transfections. Typically this can be resolved by proper construct- and stable cell line development.

For further investigation of the effect of anti-CI-M6PR VHHs on lysosomal enzyme internalisation, in vitro efficacy and in vivo distribution, we purified the chimeric pro-hCTSD-VHH7 by subjecting the secreted fraction from transfected HEK293 and GlycoDelete cells over a HisTrap column (Error! Reference source not found.A and C) and polished the eluted sample by SEC (Error! Reference source not found.B and D). Although we were capable of obtaining pure chimeric proteins, larger and probably aggregated material eluted at about 45 mL from each run of the column (Error! Reference source not found.B and D) and was also observed on SDS-PAGE (Error! Reference source not found.B). Next to the production of rhCTSD-VHH7, also fusion proteins with VHH1, VHH5, VHH6 and VHH8 were recombinantly produced by GlycoDelete HEK293 cells and purified. Generally, the chromatograms obtained after the purification of these constructs were comparable to what was observed for rhCTSD-VHH7 in Error! Reference source notfound. However, their yield was lower compared to the 16 mg per liter cell culture that we obtained for proCTSD-His₆, being only a few milligrams per liter (Error! Reference source not found.E), probably due to aggregation.

### Example 12. Investigation of the VHH-Cathepsin D Internalisation and In Vitro Activity

To be able to evaluate the intracellular activity of the fusion proteins containing CTSD and the anti-CI-M6PR VHHs in vitro upon cellular uptake, we generated CTSD loss of function (CTSD^{-/-}) clones with the CRISPR/Cas9 system. Therefore, a human cervical cancer cell line, i.e. HeLa, and a mouse myoblast C2C12 cell line, were transfected with a plasmid containing the coding sequences of Cas9 and GFP separated by the self-cleaving T2A peptide-coding sequence under the same CAG promotor, and a guide sequence targeting the CTSD in its second exon (Error! Reference source not found.A). Both HeLa and C2C12 cells were diploid for CTSD. Transfection efficiency of the cells was assessed with fluorescence microscopy 48 hours post transfection (i.e. 30% for HeLa cells and 2% for C2C12 cells) after which single GFP-positive and Cas9-expressing cells were single cell sorted by fluorescence assisted cell sorting. After three weeks of growth, the CTSD region of interest was PCR-amplified from genomic DNA of each growing HeLa and C2C12 cell clone and successfully sequenced. We analysed the sequences of these clones using the online ICE analysis tool of Synthego, which compares the sample sequences with a reference sequence, the wild-type CTSD, and predicts an indel distribution based on the sequencing data. Despite sorting of the transfected cells into separate clones, hence expecting the same mutation in all cells of one clone, this tool sometimes appeared to predict several different mutations within the same clone, potentially partly due to poor quality of the DNA (Error! Reference source not found.B and C). Therefore, it provides an indel mutation percentage, which indicates the percentage of sequences in the sample containing this indel. We assumed that indels with an indel percentage higher than 75% implies the presence of indel mutations in both alleles. In addition, we made sure that this indel caused a frame shift, hence a functional knock out of CTSD. According to this criterion, the single seeded transfected HeLa cells showed a biallelic knock out efficiency of 23% when only taking into account the sequenced clones (n=53). Similarly for the C2C12 cells, a knock out efficiency of 25% was observed (n=26). We selected four HeLa clones (i.e. clone 1F6, 1E11, 2D3, 3D5, named after their plate number and coordinate position) for further analysis, which contained a frame shift causing an indel with an indel percentage of 100%. As there were no C2C12 clones with an indel percentage of 100%, we selected four C2C12 clones (i.e. clone 1B2, 1B3, 1C2 and 1C11) with the highest indel percentage of maximally 80%. All clones grew easily and we proceeded in their selection by assessing their intracellular CTSD activity (Error! Reference source not found.D). The intracellular activity was assessed using a CTSD synthetic substrate. Compared to the wild-type HeLa and C2C12 cells, the mutated clones showed significantly less CTSD activity and C2C12 clone 1C11 and HeLa clone 3D5 were eventually selected as these showed the best result in multiple independent experiments (Error! Reference source not found.E). The C2C12 clone 1C11 and HeLa clone 3D5 have a total knockout score of 90 and 100% respectively. As shown in Error! Reference source not found.B, CTSD in the C2C12 CTSD^{-/-} line (i.e. clone 1C11) harbours multiple indels (e.g. either one, 10 and 14 base pair-deletions) while two base pair deletions were homogeneously observed over the CTSD alleles in the HeLa 3D5 clone (Error! Reference source not found.C).

We eventually used these cell lines to assess endocytosis and intracellular activity of our pro-hCTSD-VHH chimeric constructs. The CTSD^{-/-} mouse and human cells were incubated with pro-hCTSD or pro-hCTSD fused to a particular anti-CI-M6PR VHH, either containing GlycoDelete or wild-typed glycans. We chose to perform the experiment with the anti-CI-M6PR-specific VHH7 and VHH8, earlier identified as the two most potent VHHs in terms of cell binding, internalisation and lysosomal targeting. Moreover, human to mouse cross-reactive binding as shown for VHH8, and to a lesser extent for VHH7, is beneficial if we want to assess endocytosis by mouse myoblasts. First, we verified the binding affinity of the pro-hCTSD-VHH proteins in comparison to the non-fused anti-CI-M6PR VHHs on recombinant hCI-M6PR_{D1-D3} and C2C12 cells (FIG. 36). Only for our VHH7 and VHH8-constructs, minimal loss of binding was observed and VHH8-fusion construct also showed binding on mouse cells. For the uptake assay, the cells of interest were incubated in duplicate with the chimeric proteins, pro-hCTSD or cell medium, and lysates were prepared after 1 h, 3 h, 6 h, 8 h and 24 h. Afterwards, the fluorescence corresponding to the amount of peptide cleaved by pro-hCTSD in the lysates was monitored and was set out in function time in Error! Reference source not found.

As reflected by increased activity, up to 10-fold amounts of the pro-hCTSD-VHH fusion proteins could be internalised by HeLa and C2C12, as compared to pro-hCTSD. Cells incubated with pro-hCTSD-VHH7 and -8 proteins already contained superior CTSD activity after one hour of incubation (Error! Reference source not found.) which was retained for at least eight hours. After 24 h, CTSD activity from the cells treated with the chimeric constructs fell back to levels comparable to endogenously expressed CTSD by HeLa or C2C12 cells. For both glycoforms of pro-hCTSD-VHH8, the highest intracellular proteolytic activity was observed in both cell lines. Moreover, the obtained plots are comparable for the human and mouse cell line, corresponding to VHH8's increased association to hCI-M6PR but also its capability to bind mCI-M6PR. Compared to VHH8, the intracellular CTSD activity is lower when fused to VHH7 in HeLa cells, but still up to three-fold higher compared to pro-hCTSD. Also, an increased pro-hCTSD-VHH7 activity was obtained from C2C12-lysates, although values are generally lower compared to the values obtained from pro-hCTSD-VHH7 treated HeLa lysates, even though the mouse cross-reactivity of VHH7 was only shown to a lower extent. Considering that pro-hCTSD has been reported to be both endocytosed through receptors in- and dependent from CI-M6PR, it is plausible that these mechanisms play an important role as well, although this would be predicted to be similar for non-fused pro-hCTSD.

Additionally, remaining M6P residues on the glycans, combined with the proteinaceous CI-M6PR binding may provide a synergistic effect in terms of internalisation. Also, this appears rather unlikely at first sight, because GlycoDelete pro-hCTSD had lost virtually all phosphorylated glycans and behaves rather similar as the wild-type HEK293-produced proteins. We have yet to perform N-glycan analysis on the fusion proteins to fully eliminate influences of N-glycans, but probably LRP1/LDLR-mediated process play a role.

To investigate the lysosomal targeting of pro-hCTSD and the pro-hCTSD-fusion proteins with anti-CI-M6PR VHHs -7 and -8, we labelled these with an AF488 fluorescent dye and incubated these for four hours on HeLa CTSD^{-/-} cells (clone 3D5) at 37° C. Subsequently, colocalisation with LAMP1 was assessed to investigate lysosomal delivery of rhCTSD.

In the first place, we identified the amount of AF488 signal that localised inside the cells by counting AF488 voxels. For pro-hCTSD-VHH7 and -8, increased internalisation was observed in HeLa cells compared to pro-hCTSD alone (Error! Reference source not found.A). Secondly, we further analysed the intracellular pro-hCTSD-VHH-AF488 signal together with LAMP1 and determined the colocalising fraction. For the chimeric proteins, percentages around 5% and 1.5-3% were observed for pro-hCTSD-VHH7 and -8 respectively while for pro-hCTSD alone, 5 to 12% AF488-colocalising signal was observed (Error! Reference source not found.B). Pro-hCTSD values are probably overestimated due to the poor internalisation levels (Error! Reference source not found.A) and consequently low signal to noise ratio for pro-hCTSD. Lysosomal targeting of pro-hCTSD, whether or not fused to any of the VHHs can also be observed in Error! Reference source not found.C, indicated by white arrows.

### Example 13. GlycoDelete-Produced Cathepsin D-VHH Proteins have a Circulatory Half-Life of Eight Hours

We investigated whether the GlycoDelete technology could provide an increased serum half-life for recombinant pro-hCTSD protein without any fusion partner, compared to pro-hCTSD expressed by wildtype HEK293 cells. Five mice injected with pro-hCTSD (n=5, 5 mg/kg) were therefore sacrificied for blood collection after 1, 3, 6, 16 and 48 hours. Using an ELISA assay, we detected recombinant protein in every serum sample (FIG. 36). However, serum-levels of both pro-hCTSD variants were low, partially due to the unsensitive ELISA assay and probably also as a result of pro-hCTSD's mannose-specific clearance routes (Error! Reference source not found.) and renal filtration (FIG. 37). In a follow-up experiment, we intraveneously injected pro-hCTSD, pro-hCTSD-VHH7 and PBS to C57BL/6J mice, after which blood sampling was performed at 30, 60, 180, 360 and 960 minutes post injection (Error! Reference source not found.A). Additionally we aimed at monitoring renal filtration by micturition at the same time points.

The half-life of pro-hCTSD-VHH7 from wild-type HEK293 cells was comparabe to non-fused pro-hCTSD but the half-life of the fusion protein with trimmed GlycoDelete glycans was prolonged to around 500 minutes (Error! Reference source not found.9, Error! Reference source not found.B). Although increased, this needs to be confirmed in further experiments with more sampling points later in the time course.

TABLE 9
Circulatory half-life (t_{1/2}) in minutes (min) for cathepsin D (pro-hCTSD) and pro-
hCTSD-VHH7, derived from wild-type HEK293 cells (S) and GlycoDelete cells (GD).

| | pro-hCTSD - S | pro-hCTSD - GD | pro-hCTSD-VHH7- S | pro-hCTSD-VHH7- GD |
|---|---|---|---|---|
| t_{1/2 replicate 1} (min) | 361.6 ± 16.0 | 364.4.0 ± 13.1 | 349.5 ± 14.5 | 520.1 ± 13.3 |

Technical replicates of the ELISA assay are shown, each assay measured serum samples of all five mice injected.

Important to note is that our molecules have an apparent molecular weight around 50 and 65 kDa, which is around the molecular weight cut-off for renal filtration (i.e. 60 kDa). This is certainly the case if glycoproteins are GlycoDelete-typed, as the trimmed N-glycans reduce the hydrodynamic radius. As renal clearance is likely a dominating factor in the pharmacokinetics of unfused pro-hCTSD that marks clearance by lectin-mediated mechanisms. Only upon molecular weight enhancement through fusion with a VHH, non-renal clearance mechanisms become more important to enhance half-life, and removal of the N-glycans of pro-hCTSD avoids lectin-mediated clearance. However, even the VHH-fused molecules are still close to the renal filtration limit, and further non-glycan hydrodynamic radius enhancements may still prolong half-life.

It is therefore worth mentioning that we have also investigated the potential to increase the molecular weight of pro-hCTSD by coupling it to derivatised polyethylene glycol, by oxidising terminal galactose or sialic acid residues of HEK293- or GlycoDelete produced N-glycan (GlyConnectTechnology). Because PEG increases the protein's molecular weight and radius, this strategy could be employed to prolong circulation times. In short, terminal Gal and Sia were oxidised by means of galactose oxidase or sodium periodate and coupled to a 20 kDa, two-arm branched aminoxy-derivatised PEG chain (Thooft et al., 2021; Org. Biomol. Chem., 2022, 20, 464-471). Because connecting PEG to oxidised Gal residues gave the most homogeneous sample as observed on western blot, we verified that not only the 20 kDa, two-arm branched PEG (20 kDa) but also a single-chain 20 kDa and 10 kDa PEG chain could be GlyConnected to wild-type and GlycoDelete CTSD. However, their serum half-life was not yet further investigated yet.

### Example 14. Recombinant Expression of rhGAA and rhGAA-VHH Chimeric Proteins

Besides the development of a next-generation pro-hCTSD enzyme replacement therapy molecule, we investigated this as well for rhGAA, another lysosomal enzyme used in the clinic for the treatment of Pompe disease. Just like most of the current ERT on the market or in development, rhGAA is usually produced in CHO cells and it is notoriously difficult to produce it. Most of the know-how on this is proprietary to Sanofi-Genzyme, which uses specialised perfusion bioreactors to enhance cell-densities and to limit residence time of the secreted protein in the production reactor. Even with this, yields are very much lower than for example monoclonal antibodies and large installations are required. As we aimed for expression in HEK293 and HEK293 GlycoDelete cells ultimately, we performed preliminary expression tests in HEK293 cells. These showed that only the non-codon optimised sequence coding for rhGAA could be expressed, though at very limited levels by HEK293 cells (Error! Reference source not found.A). To increase the yield, we designed other constructs containing rhGAA with different signal sequences (e.g. endogenous rhGAA, interleukin-2, Gaussia princeps^{60,61} and CD5), cloned in vectors other than the pcDNA3.3 vector that was originally used. In Error! Reference source not found.A, the results of the secreted (Error! Reference source not found.A left) and intracellular fraction (Error! Reference source not found.A right) of HEK293 cells transfected with these constructs are shown. As discussed, rhGAA undergoes extensive processing, but for biopharmaceutical development, the 110 kDa format is preferred. With this in mind, it is clear that using the endogenous secretion signal gives the best result. Although the CD5-secretion signal could also be used, if need to be. The lower molecular weight-bands correspond to the intermediate rhGAA forms (i.e. 95 kDa and 76 kDa). As we objected high expression of the full length, non-processed rhGAA (110 kDa), we chose to proceed with the pcDNA3.1 vector and investigated the use of additives in our culture media to boost expression levels. For this, we investigated M6P, sodium butyrate and N-acetyl cysteine. We chose M6P to prevent rhGAA re-uptake in the cells via CI-M6PR, sodium butyrate to enhance expression for SV40 early promoter-driven coding sequences and N-acetyl cysteine, an allosteric chaperone of rhGAA⁵⁶. However, none of these interventions could substantially increase expression levels (Error! Reference source not found.B). Subsequently we proceeded by designing rhGAA and VHH chimeric constructs, of which the VHH was either N- or C-terminally fused to rhGAA. Given the extensive N-terminal processing of rhGAA, only the rhGAA with C-terminally fused VHH could be efficiently expressed and secreted by HEK293 cells (Error! Reference source not found.C). In addition, we maximised the yields of the fusion proteins by analysing the optimal day of harvesting the constructs. Therefore, we transfected HEK293 and GlycoDelete cells for which we followed expression by analysing samples every day post transfection for six days in total. The highest amount of rhGAA-VHH proteins could be obtained after four days post transfection. In parallel, GlycoDelete EndoT processing of the chimeric rhGAA-VHH8 proteins was monitored but not complete, given that only the precursor rhGAA-VHH8 was reduced in molecular weight, while the intermediate rhGAA-VHH8 was not. For rhGAA we did observe a complete reduction in MW on western blot (Error! Reference source not found.C). By comparing the DSA-FACE profiles of untreated rhGAA (Myozyme®) with the CIP- and sialidase digested one, we identify a very small amount of phosphorylated glycans (Error! Reference source not found.D‘*’) and highly sialylated glycans. However, detailed N-glycan analysis of the chimeric proteins was not performed.

rhGAA-VHH fusion proteins were recombinantly expressed by HEK293 GlycoDelete cells and purified by IMAC and SEC. We show the obtained chromatograms of rhGAA-VHH8 in Error! Reference source not found.E, as it is representative for the other rhGAA chimerics that we purified. The very low expression yield of rhGAA-VHH8 is remarkable however, situating between 500-1,000 μg/L, also observed by the low absorbance at 280 nm during SEC and not unexpected, given the difficulty of rhGAA production (Error! Reference source not found.F).

### Example 15. Chimeric Fusion Proteins of Anti-CI-M6PR VHH and Lysosomal Enzymes can be Internalised by Diseased Cells and are Targeted Towards the Lysosome to Degrade Stored Glycogen

Comparable to the chimeric proteins of CTSD and anti-CI-M6PR VHHs, we analysed the in vitro internalisation, activity and lysosomal targeting potential for rhGAA and some of the rhGAA-VHH fusion proteins: rhGAA-VHH5, -7 and -8. Given that rhGAA is only slowly taken up by GAA^{-/-} fibroblasts and that at least 24 hours are needed to allow substantial processing and measurable activity⁶², the constructs were incubated for 16, 24 and 48 hours in triplicate (200 nM). We quantified the remaining amount of intracellular glycogen by digesting it to glucose and subsequent oxidation by which a colorimetric agent, Amplex red, could be reduced and measured. In parallel, we analysed the intracellular glucosidase activity using a synthetic substrate (Error! Reference source not found.). When analysing the amount of hydrolysed glucose in each of the treated samples, we could observe a reduction of at least 60% in glucose, and so accumulated glycogen, content for rhGAA and VHH-fusions thereof compared to the untreated wells (Error! Reference source not found.A). For rhGAA-VHH7 the most substantial reduction of resident glycogen was observed, being around 80%, for all time points investigated. This corresponds to the increased activity of rhGAA-VHH7 observed intracellularly compared to rhGAA-VHH5, -8 and non-fused rhGAA (Error! Reference source not found.B). Hence in this case, VHH7 appears to be the best choice, whereas this was VHH8 for pro-hCTSD (although we were not in the capacity to measure storage product, nor its clearance and the VHH7 construct appeared to end up in LAMP1-positive lysosomes more effectively than the VHH8 construct).

Finally, to obtain an idea of to what extent increased internalisation by means of a fused anti-CI-M6PR VHH would occur, we incubated the Pompe fibroblasts with rhGAA and rhGAA-VHH8 for four hours, bearing in mind the saturated endocytosis at that point. Afterwards, cells were prepared for microscopic examination in which nuclei and lysosomes were stained (Error! Reference source not found.). However, remarkable is the low absolute voxel counts for the AF488-signal of both proteins in general and certainly for rhGAA-VHH8 (Error! Reference source not found.A). This low intracellular AF488-signal may therefore not correlate well to percentages of intracellular lysosomal colocalisation as these may reflect higher and variable intralysosomal amounts of intracellular rhGAA compared to rhGAA fused to VHH8 (Error! Reference source not found.B). For one of the replicates, images are shown in Error! Reference source not found.C and these indeed show limited AF488 signal colocalising with LAMP1-stained lysosomes. Although not further investigated, it can be seen that both proteins have a differential cellular localisation. Whereas more rhGAA is present at perinuclear regions, signal from rhGAA-VHH8 is observed at the cell's periphery (Error! Reference source not found.C). Results for rhGAA-VHH7, which resulted in very substantially increased intracellular rhGAA-VHH7 activity versus rhGAA, are to be expected.

Conclusion on the application of fusion proteins of anti-CI-M6PR-VHH and Cathepsin D protease or acid α-glucosidase for use in ERT.

We have described the design and production of anti-CI-M6PR VHH fusions to the lysosomal enzyme pro-hCTSD and rhGAA. However, the extensive processing of both enzymes allowed only VHH-fusion at the C-terminus. The removal of the N-terminally positioned VHHs is probably the result from proteolytic processing at the N-terminus, which is typical for lysosomal enzymes and mainly occurring along the endolysosomal pathways (Error! Reference source not found.). It is possible that the secreted rhCTSD proteins are the result from limited proteolytic processing or VHH-rhCTSD upstream in the biosynthetic pathway or that these just escaped the endolysosomal path when more elaborate processing could have occurred. This is probably also the reason why more rigid or shorter linkers did not prevent this. Similar to the rhCTSD-VHH proteins, only chimeric rhGAA-constructs with C-terminally fused VHHs were efficiently expressed and secreted by HEK293 cells.

In general, both fusion proteins could be expressed by HEK293 and HEK293 GlycoDelete⁵² cells, yet with a yield that was overall very low. Moreover, both purification processes (i.e. SEC) showed that the fusion constructs express very poorly and tend to aggregate more easily, indicating that the VHH-fusion may affect its solubility. Further optimisation is thus required to obtain a higher yield of said secreted fusion products. Moreover, a detailed N-glycan analysis is required if distribution or efficacy is analyzed in vivo.

To analyse internalisation of rhCTSD, we incubated a purified VHH-containing pro-hCTSD on HeLa and C2C12 CTSD^{-/-} cells for one to 24 hours. Pro-hCTSD fused to VHH7 and -8 was rapidly taken up by HeLa cells up to 10-fold more than non-VHH fused pro-hCTSD. An increased intracellular activity was also observed in mouse C2C12 cells, although VHH7 is only to a lower extend mouse cross-reactive for its CI-M6PR.

When single doses of purified rhGAA and rhGAA-VHH fusions were incubated on GAA^{-/-} cells, we obtained glycogen reduction for all proteins investigated. By microscopic examination, we could not confirm increased lysosomal targeting of rhGAA-VHH8, instead, AF488 signal from the proteins investigated was observed at the cell's periphery, reminiscent of an early endosomal distribution. It is plausible that lysosomal colocalisation will be higher for rhGAA fused to VHH7, for which we earlier observed increased presence in the lysosomes.

Additional microscopic experiments using general endolysosomal markers or a specific early endosomal staining would allow us to pinpoint the precise location and traffic of the fusion proteins that we developed. Moreover, a dose-dependent cellular uptake for all molecules should verify the half-maximal dose at which internalisation is observed. This can be performed by monitoring GAA-mediated hydrolysis of 4methylumbelliferyl α-D-glucopyranoside or 4-nitrophenyl α-D-glucopyranoside in lysates or by measuring glycogen clearance. Additionally, competition with cognate VHHs, rhCI-M6PR_{D1-D3} and/or M6P can be considered as well to dissect the contributions in uptake of the VHH and the rhGAA-linked M6P-glycans. Ultimately, treatment of GAA^{-/-} mice and comparing remaining glycogen levels in tissues of interest (e.g. skeletal muscle, heart, lungs, etc.) to wild-type mice⁶⁹⁻⁷¹, is of paramount interest.

Additionally, because we have previously shown that it would take 10-12 days longer for the concentration of GlycoDelete anti-CD20 antibodies to fall below the therapeutic threshold concentration⁵², gaining an increase in circulatory residence would be highly desired for expensive biologics that need frequent dosing like ERT. In this context, we have investigated the potential to exploit the GlycoDelete technology as a superior feature to build next-generation ERT.

We injected pro-hCTSD and pro-hCTSD-VHH7 purified from wild-type and GlycoDelete HEK293 cells intravenously at 5 mg/kg and monitored the rhCTSD levels in serum for multiple time points. The obtained data showed an overall rapid clearance for pro-hCTSD; exhibiting a serum half-life around six hours for wild-type and GlycoDelete produced proteins. The time at which half of the chimeric GlycoDelete fusion protein with trimmed N-glycans was present was about eight hours. Although we showed a certain increase in serum half-life for the chimeric constructs, the high level of variation should be taken into account. Moreover, it needs to be further investigated whether this effect relates to increased tissue distribution, preferably for pro-hCTSD-VHH8, by radioactive labelling and gamma counting in tissues of interest⁷². Additionally, it would be interesting to analyse the circulation time and biodistribution or GlycoDelete rhGAA(-VHH) proteins as current half-life situates around 0.7 h to 1.2 hours for rhGAA or rhGAA variants⁷³⁻⁷⁶.

Recently, the group of Henrik Clausen developed a large set of glyco-engineered CHO cell lines to capture and investigate a large scope of the α-galactosidase glycans. They showed that α-galactosidase A harbouring three biantennary N-glycans with terminal (2,3-sialic acids had a half-life of 27.3 minutes, thereby more than doubling the half-life of the wild-type form that is used in the clinic (i.e. Fabrazyme®, t_{1/2}: 11.9 minutes). Moreover, the glyco-engineered variant was increasingly distributed in vivo and targeted to the tissues of interest. Interesting to mention is that α-galactosidase A containing hybrid M6P glycans, or high or low M6P residues, had similar serum half-lives of 8.3, 11.0 and 15.4 minutes respectively⁷⁷. However, Fabrazyme® is not the most ideal protein for such pharmacokinetic studies as it quickly denatures upon injection, resulting in highly variable results. Therefore, it is difficult to determine to what extend this approach would be beneficial for rhCTSD or rhGAA nevertheless probably only effective when fused to a lysosomal targeting moiety like an anti-CI-M6PR VHH.

Besides glyco-engineering, we have generated some proof-of-principle that pro-hCTSD can be GlyConnected to high-molecular weight PEG chains both on oxidised galactoses and sialic acids. Although controversial because of their immunogenicity after repeated dosing⁷⁸, it could be worthwhile to test whether this GlyConnection technology provides an increased hydrodynamic volume for rhCTSD and would lead to a longer circulation time in vivo.

Ultimately, a 24-hour serum half-life is aimed for using our GlycoPEGyated pro-hCTSD fusions and a subcutaneous route of administration to patients is preferred as this would further lead to prolonged exposure upon injection. For intrathecal administration (e.g. for treatment of neuronal ceroid lipofuscinosis 10, a CTSD-deficiency), efforts in prolonging serum half-life are less relevant and the 10-fold enhanced uptake of pro-hCTSD-VHH8 appears very attractive. To enhance the storage material clearance after the single or very few bolus doses that can be afforded in this challenging setting.

### Example 16. In Silico Design and Production of Humanized Variants of VHH7 and VHH8

Multiple humanized variants of the anti-CI-M6PR VHHs VHH7 and VHH8 were designed in silico and are depicted in the alignments in FIGS. 46 and 47, respectively (and as present in SEQ ID NOs: 93-96 for VHH7 humanized variants and SEQ ID NOs: 97-102 for VHH8 humanized variants). VHH7hWN and VHH8hWN were produced in HEK293S and purified through IMAC and SEC. The variants VHH7 h1-3 and VHH8 h1-5 were produced in E. coli and purification was performed through IMAC and desalting. An overview of their expression levels per 100 mL is shown in Table 10.

TABLE 10
Expression yield of humanized variants of VHH7 and VHH8.

| | | Total yield (mg) |
|---|---|---|
| VHH | Host | (100 ml culture) |
| VHH7h1 | E. coli | 1.12 |
| VHH7h2 | E. coli | 0.38 |
| VHH7h3 | E. coli | 0.72 |
| VHH7hWN | HEK293S | 4.04 |
| VHH8h1 | E. coli | 0.72 |
| VHH8h2 | E. coli | 0.71 |
| VHH8h3 | E. coli | 0.16 |
| VHH8h4 | E. coli | 1.32 |
| VHH8h5 | E. coli | 1.28 |
| VHH8hWN | HEK293S | 5.18 |

Example 17. pH-Dependent Dissociation of Humanized Variants of VHH7 and VHH8 from hDom₁₋₃ CI-M6PR.

For a selection of the humanized variants of VHH7 and VHH8, a biolayer interferometry (BLI) experiment was performed in which the human CI-M6PR domain₁₋₃His₆ was biotinylated and coupled to streptavidin biosensor tips. After loading, the tips were incubated with VHHs serially diluted in pH 7.4 kinetic buffer during the association phase and dissociation was performed at pH 7.4, pH 6.5, pH 6.0, pH 5.5 and pH 5.0. All biosensor tips were then regenerated before analysis of the subsequent VHH. Table 11 summarizes the kinetic parameters retrieved after processing and curve fitting of the BLI measurements. When both association and dissociation were performed at pH 7.4, a global fit was performed according to the 1:1 binding model of which the resulting affinity constants (K_{D}), association (kₒₙ) and dissociation rate constants (k_{off}) are shown. For measurements with association at pH 7.4 and dissociation at pH 6.5, 6.0, 5.5 and 5.0, the depicted dissociation rate constants are an average of the parameters determined by local curve fitting of the dissociation of 200, 100 and 50 nM VHH. Association-dissociation curves for VHH7 and its humanized variants are shown in FIG. 48 and those for VHH8 and its humanized variants are depicted in FIG. 49.

BLI revealed pH-dependent dissociation of the humanized variants VHH7 h1, VHH7 h2, VHH7 h3 and VHH7hWN, with dissociation profiles similar to their non-humanized counterpart VHH7, with the dissociation rate gradually but moderately increasing with decreasing pH (FIG. 48, Table 11). Furthermore, their affinity for the human CI-M6PR domain₁₋₃His6 at pH 7.4 remains almost unaltered upon humanization. Likewise, the pH-dependent dissociation profile of VHH8, where the dissociation rate only moderately increases between pH 7.4 and 6.0, but then demonstrates a rapid surge with close to one order of magnitude between pH 5.5 and 5.0, is unaltered for its humanized variants VHH8 h1, VHH8 h2, VHH8 h3 and VHH8hWN (FIG. 49, Table 11). Here also, the obtained K_{D}-value at pH 7.4 is comparable for all variants under evaluation.

TABLE 11
Overview of kinetics parameters determined through BLI of the binding of VHH7, VHH8
and a selection of their humanized variants to human CI-M6PR domain₁₋₃His₆.

| VHH | pH | K_{D} (M) | kₒₙ (M⁻¹s⁻¹) | k_{Off} (s⁻¹) | VHH | pH | K_{D} (M) | kₒₙ (M⁻¹s⁻¹) | k_{Off} (s⁻¹) |
|---|---|---|---|---|---|---|---|---|---|
| VHH7 | 7.4 | 1.01 × 10 [symbol] | 5.4 × 10 [symbol] | 5.5 × 10 [symbol] | VHH8 | 7.4 | 6.34 × 10 [symbol] | 6.1 × 10 [symbol] | 3.2 × 10 [symbol] |
| | 8.5 | | | 9.1 × 10 [symbol] | | 6.5 | | | 8.4 × 10 [symbol] |
| | 8.0 | | | 9.9 × 10 [symbol] | | 6.0 | | | 9.1 × 10 [symbol] |
| | 5.5 | | | 1.3 × 10 [symbol] | | 5.5 | | | 3.4 × 10 [symbol] |
| | 5.0 | | | 1.7 × 10 [symbol] | | 5.0 | | | 2.3 × 10 [symbol] |
| VHH7h1 | 7.4 | 1.41 × 10 [symbol] | 5.0 × 10 [symbol] | 7.1 × 10 [symbol] | VHH8h1 | 7.4 | 1.06 × 10 [symbol] | 4.5 × 10 [symbol] | 4.8 × 10 [symbol] |
| | 6.5 | | | 9.7 × 10 [symbol] | | 6.5 | | | 5.3 × 10 [symbol] |
| | 8.0 | | | 1.2 × 10 [symbol] | | 6.0 | | | 1.5 × 10 [symbol] |
| | 5.5 | | | 1.4 × 10 [symbol] | | 5.5 | | | 4.3 × 10 [symbol] |
| | 5.0 | | | 2.0 × 10 [symbol] | | 5.0 | | | 2.9 × 10 [symbol] |
| VHH7h2 | 7.4 | 1.36 × 10 [symbol] | 4.7 × 10 [symbol] | 6.4 × 10 [symbol] | VHH8h2 | 7.4 | 3.07 × 10 [symbol] | 2.2 × 10 [symbol] | 6.7 × 10 [symbol] |
| | 6.5 | | | 9.4 × 10 [symbol] | | 6.5 | | | 1.1 × 10 [symbol] |
| | 6.0 | | | 1.1 × 10 [symbol] | | 6.0 | | | 1.5 × 10 [symbol] |
| | 5.5 | | | 1.4 × 10 [symbol] | | 5.5 | | | 4.4 × 10 [symbol] |
| | 5.0 | | | 1 9 × 10 [symbol] | | 5.0 | | | 4.1 × 10 [symbol] |
| VHM7h3 | 7.4 | 1 40 × 10 [symbol] | 4.6 × 10 [symbol] | 6.4 × 10 [symbol] | VHH8h3 | 7.4 | 1.76 × 10 [symbol] | 2.9 × 10 [symbol] | 5.1 × 10 [symbol] |
| | 8.5 | | | 1.1 × 10 [symbol] | | 6.5 | | | 2.2 × 10 [symbol] |
| | 6.0 | | | 7.3 × 10 [symbol] | | 6.0 | | | 2.0 × 10 [symbol] |
| | 6.5 | | | 1.5 × 10 [symbol] | | 5.5 | | | 4.1 × 10 [symbol] |
| | 6.0 | | | 1 9 × 10 [symbol] | | 5.0 | | | 3.3 × 10 [symbol] |
| VHH7 | 7.4 | 1 44 × 10 [symbol] | 3.0 × 10 [symbol] | 4.4 × 10 [symbol] | VHH8 | 7.4 | 1.29 × 10 [symbol] | 4 4 × 10 [symbol] | 5.7 × 10 [symbol] |
| hWN | 6.5 | | | 8.6 × 10 [symbol] | hWN | 6.5 | | | 1.3 × 10 [symbol] |
| | 6.0 | | | 9.5 × 10 [symbol] | | 6.0 | | | 1.2 × 10 [symbol] |
| | 5.5 | | | 1 1 × 10 [symbol] | | 5.5 | | | 5.1 × 10 [symbol] |
| | 5.0 | | | 1.8 × 10 [symbol] | | 5.0 | | | 3.7 × 10 [symbol] |

[symbol]

indicates data missing or illegible when filed

Finally, a confirmatory in-tandem competitive BLI experiment of humanized anti-CI-M6PR VHHs purified from E. coli revealed that humanized VHH7 variants VHH7 h1, VHH7 h2 and VHH7 h3 competed with VHH7 for CI-M6PR hDom₁-3His₆ binding and not with VHH8, whereas humanized VHH8 variants VHH8 h1, VHH8 h2, VHH8 h3, VHH8 h4 and VHH8 h5 competed with VHH8 for CI-M6PR hDom₁-3His₆ binding and not with VHH7 (FIG. 55).

### Example 18. Repanning of the Original VHH Library for Identification of Novel Anti-CI-M6PR VHH CDR3-Families

In an attempt to identify novel anti-CI-M6PR VHHs that cannot be assigned to the previously identified CDR3-families of VHH1-VHH11, the original VHH-library of the llama that yielded an antigen-specific response to immunization with recombinant human CI-M6PR Dom₁-3His₆ was re-panned onto coated CI-M6PR hDom₁-3His₆. However, in comparison to the original panning efforts (as described in Example 2), in which phage elution was performed under acidic and basic conditions, the bound phages were now eluted either through addition of trypsin or through competition with both VHH7 and VHH8. In these panning experiments, the enrichment of the phage population for antigen-specific phages after the first round was calculated to be about 100-fold following elution with trypsin, while no enrichment was observed after competitive elution. Still, 96 colonies were randomly selected from both panning experiments and analyzed by ELISA for the presence of antigen-specific VHHs in their periplasmic extracts. For elution with trypsin, 83 colonies scored positive for binding of the coated CI-M6PR hDom₁₋₃ His₆ of which 55 different full-length VHHs were distinguished after sequence analysis. Following clustering of these VHH sequences into CDR3-families via the Cluster Database at High Identity with Tolerance¹⁰³, 13 different CDR3-groups were identified in this experiment. Out of those, 5 corresponded to CDR3-families already encountered in the original panning efforts. However, 11 new VHHs were identified belonging to 8 novel CDR3-families. In the same way, 54 different VHHs were identified in the screening of 96 colonies that resulted from the phage panning with competitive elution. Among these were 4 unique VHH sequences from 4 previously unknown CDR3-groups. Overall, 15 new VHHs belonging to 12 novel CDR3-groups (as depicted in SEQ ID NOs: 71-82) were thus identified in these panning efforts.

### Example 19. CI-M6PR-Specific VHH Families Competing for VHH7 and VHH8

In order to identify anti-CI-M6PR VHHs that bind an overlapping or identical epitope on CI-M6PR as VHH7 or VHH8 from a different ‘CDR3’-family, or VHH family, as defined herein, competitive ELISA experiments were performed on TG1 and WK6 E. coli periplasmic extracts containing one representative VHH of each of the newly identified CDR3-defined VHH families. The results of these experiments indicated binding site competition between a subset of these VHHs and VHH7 or VHH8 for binding of coated CI-M6PR hDom₁-3His₆, but were not entirely conclusive due to high background and poor quality of periplasmic extracts. For this reason, additional biolayer interferometry (BLI) experiments were performed for identification of competitors of VHH7 or VHH8 for the CI-M6PR binding site, wherein previously characterized VHH1 and VHH5 were also evaluated. Periplasmic extracts containing VHHs 1H06, 1H73 and 2H14 scored clearly negative for competition with VHH7 and VHH8 in the competitive ELISA-assays and were thus left out of subsequent experiments. In-tandem competitive BLI of anti-CI-M6PR VHHs purified from E. coli revealed that VHHs 1H11 and VHH1 competed with VHH7 for CI-M6PR hDom₁-3His₆ binding but not with VHH8; and 1H52 and VHH5 competed with VHH8 for CI-M6PR hDom₁-3His₆ binding but not with VHH7, whereas 1H21, 1H37, 2H74 and 2H79 did not compete for binding to CI-M6PR hDom₁₋₃ His₆ with VHH7 or VHH8. No saturating binding of CI-M6PR hDom₁-3His₆ was obtained for 1H74, 1H44 and 2H60 (FIG. 50, 51).

### Example 20. Analysis of Alternative CI-M6PR-Specific VHHs (as Disclosed by Houthoff et al.)

Verification of the novelty and uniqueness of the binding sites defined herein for VHH7, VHH 1H11 and VHH8 required to screen the panel of anti-CI-M6PR VHHs that were developed against the extracellular part of CI-M6PR by LinXis BV (as described in Houthoff et al. published as WO2020/185069A1). To make a representative selection providing evidence that those alternative VHHs reported in the state of the art clearly differ from the presently described VHHs was obtained by evaluation of those VHHs for binding to human CI-M6PR Dom₁₋₃His₆ and for binding to the CI-M6PR in competition with VHH7 or VHH8. An alignment of all 15 of the described LinXis VHH-sequences was made and clustering was performed based on CDR3 sequence identity (FIG. 52; SEQ ID NOs: 103-120). One representative VHH was produced for each of the 7 identified CDR3-based VHH families, since VHHs of the same family are known to bind the same binding site on their target. The LinXis VHHs were expressed in E. coli and purification was performed through IMAC and desalting. Quality control was performed through SDS-PAGE (FIG. 53).

In-tandem competitive biolayer interferometry of the purified alternative anti-CI-M6PR VHHs revealed that LinXis VHHs 13E8, as well as the VHH7 and VHH8, described herein, each specifically bound to non-overlapping epitopes on CI-M6PR hDom₁₋₃His₆, since no competition was observed. Moreover, at 400 nM, the LinXis VHHs 13B12, 13G10, 13A10, 13G12, 13A8 and 13F11 did not bind to immobilized biotinylated CI-M6PR hDom₁₋₃His₆ (FIG. 54).

### Example 21. Structural Analysis of VHH1, VHH5, VHH 1H11 and VHH 1H52 in Complexation with CI-M6PR hDom₁-3His₆

Since VHH1 and VHH 1H11 potentially bind the same epitope as VHH7, and VHH5 and VHH 1H52 the same epitope as VHH8, further characterization was aimed for by production in WK6 E. coli and purification via IMAC and desalting as described. An overview of their expression levels per 100 mL is shown in Table 12.

TABLE 12
The expression yield of anti-CI-M6PR VHHs.

| | | Total yield (mg) |
|---|---|---|
| VHH | Host organism | (100 ml culture) |
| VHH1 | E. coli | 0.18 |
| VHH5 | E. coli | 0.41 |
| 1H11 | E. coli | 0.18 |
| 1H52 | E. coli | 0.20 |

To prepare complexes for co-crystallization, hDom₁-3His₆ and anti-CI-M6PR proteins were incubated in a 1:2 (for VHH1, VHH5 and VHH 1H52) or 1:1.3 (for VHH 1H11) molar fashion in HBS buffer (50 mM HEPES, 150 mM NaCl, pH 7.5) and injected onto a HiLoad 16/600 Superdex 200 pg column for gel filtration (FIG. 56). Fractions containing VHH-antigen complex were collected and concentrated to 2.4-3.8 mg/mL using an Amicon Ultra-15 protein concentrator (UFC903024, Millipore). The presence of both CI-M6PR and VHH in the samples was confirmed via SDS-PAGE (FIG. 57) and intact protein mass spectrometry (data not shown). The masses of VHH1, VHH5, VHH1H11 and VHH1H52 were confirmed by intact mass spectrometry, taking the number of disulfide bridges into account (one for VHH1 and VHH1H11, and two for VHH5 and VHH1H52). A small fraction of VHH5, VHH1H11 and VHH1H52 carries an N-terminal pyroglutamate modification (26.17%, 9.07% and 3.47%, respectively).

Co-crystallization experiments were successful for the complexed VHH 1H11 as described in Example 9.

### Example 22. pH-Dependent Dissociation of Anti-CI-M6PR VHHs 1H11 and VHH1H52 from hDom₁₋₃ CI-M6PR

For VHH 1H11 and VHH 1H52, a BLI experiment was performed in which the human CI-M6PR domain₁₋₃His₆ was biotinylated and coupled to streptavidin biosensor tips. After loading, the tips were incubated with VHHs serially diluted in pH 7.4 kinetic buffer during the association phase and dissociation was performed at pH 7.4, pH 6.5, pH 6.0, pH 5.5 and pH 5.0. All biosensor tips were then regenerated before analysis of the subsequent VHH. Table 13 summarizes the kinetic parameters retrieved after processing and curve fitting of the BLI measurements. When both association and dissociation were performed at pH 7.4, a global fit was performed according to the 1:1 binding model of which the resulting affinity constants (K_{D}), association (kₒₙ) and dissociation rate constants (k_{off}) are shown. For measurements with association at pH 7.4 and dissociation at pH 6.5, 6.0, 5.5 and 5.0, the depicted dissociation rate constants are an average of the parameters determined by local curve fitting of the dissociation of 200, 100 and 50 nM VHH. Association-dissociation curves for VHH1H11 and VHH1H52 are shown in FIG. 58 and FIG. 59, respectively.

Analysis of the BLI data revealed that anti-CI-M6PR VHH 1H11, for which competition for binding of CI-M6PR hDom₁-3His₆ with VHH7 was demonstrated (in addition to VHH1) through BLI (Example 19), demonstrates a similar pH-dependent dissociation profile as VHH7 itself (FIG. 58). As is the case for VHH7, values for the dissociation rate constant gradually but moderately increase with decreasing pH from pH 7.4 down to pH 5.0 (Table 13). VHH 1H52, one of the anti-CI-M6PR VHHs that competed with VHH8 for binding of CI-M6PR hDom₁-3His₆ (next to VHH5) as shown through BLI (Example 19), also showed a similar pH-dependent dissociation profile as VHH8 (FIG. 59). Indeed, there is a rapid increase in the rate of dissociation between pH 5.5 and pH 5.0 (Table 13).

TABLE 13
Overview of binding data analysis as determined
by BLI for the binding of VHH 1H11 and VHH 1H52 to human
CI-M6PR domain₁₋₃His₆.

| VHH | pH | K_{D} (M) | k_{On} (M⁻¹s⁻¹) | koff (s⁻¹) |
|---|---|---|---|---|
| 1H11 | 7.4 | 4.90 × 10⁻⁹ | 5.1 × 10⁵ | 2.5 × 10⁻³ |
| | 6.5 | | | 4.8 × 10⁻³ |
| | 6 | | | 5.1 × 10⁻³ |
| | 5.5 | | | 6.3 × 10⁻³ |
| | 5 | | | 8.3 × 10⁻³ |
| 1H52 | 7.4 | 3.10 × 10⁻⁸ | 1.0 × 10⁶ | 3.2 × 10⁻² |
| | 6.5 | | | 6.7 × 10⁻² |
| | 6 | | | 3.0 × 10⁻² |
| | 5.5 | | | 5.1 × 10⁻² |
| | 5 | | | 2.0 × 10⁻¹ |

### Example 23. CI-M6PR Mutants and Binding of Different Anti-CI-M6PR VHHs in ELISA

Variants of the human CI-M6PR Dom₁-3His₆ with mutations in the epitopes of VHH7 or VHH8 were designed in silico based on the results of the PISA- and FastContact analysis of the corresponding crystal structures (see material and methods). To evaluate binding to the VHH7-epitope mutants as described herein and the VHH8-epitope mutants as described herein, the VHHs described herein were analyzed for their affinity to said different CI-M6PR mutants by ELISA. The experiment was performed using CI-M6PR_M85E, CI-M6PR_D87L, CI-M6PR_K89D and CI-M6PR_E148F mutants. Expression and purification of the CI-M6PR_F143R variant failed. ELISA binding profiles revealed a clear effect of M85E, D87L and E148F mutation on the binding ofVHH7, VHH7hWN, VHH 1H11 and VHH1. No effect was seen for binding of VHH8, VHH8hWN, VHH 1H52 and VHH5 to these mutants, with very similar ELISA binding profiles and deduced EC50 values (FIG. 60, 62).

Similarly, evaluation of the VHH8-epitope binders for the CI-M6PR mutants was analyzed by ELISA using CI-M6PR_L197D, CI-M6PR_D409F, CI-M6PR_E433R and CI-M6PR_F457E. Expression and purification of the M6PR_K191F variant failed. ELISA binding profiles revealed a clear effect of L197D, D409F and F457E on binding of VHH8 and VHH8hWN, D409F and E433R on binding of VHH 1H52 and F457E on binding of VHH5. No effectwas seen for binding ofVHH7, VHH7hWN, VHH 1H11 and VHH1 to these mutants (FIG. 61, 63).

### Materials and Methods

### 1. Design of Constructs

The hCI-M6PR_{D1-D3} (amino acids 1-467) sequence of the CI-M6PR (Genbank accession No J03528.1), containing a C-terminal His6 tag was codon optimized and ordered synthetically (IDT gBlocks) flanked by 5′-AAGAACAAGCCGCCACC 3′ (SEQ ID NO:12) and 5′GCTCTCCCTATTGTGAAGTCGC-3′(SEQ ID NO:13) oligos. Fragments obtained after PCR amplification (Phusion polymerase (NEB)) using 5′-AAGAACAAGCCGCCACCATG-3′ (SEQ ID NO:14) and 5-GTGCGACTTCACAATAGGGAGAGC-3′ (SEQ ID NO:15) primers were purified using DNA CleanNA beads (GC-Biotech, MB AC-60050). For cloning, fragments were incubated for 45′ at 37° C. with Klenow fragment (3′ to 5′ exo-) (NEB, M0212L), NEBuffer 2 (NEB), dATP (0.1 mM) and cloned using pcDNA™3.3-TOPO™ TA Cloning™ Kit (Thermo Fischer Scientific, K830001) according the provided protocol. The cloned plasmid was heat shock transformed (42° C., 90″) into MC1061 E. coli and sequence verified. The domain1-3His6 (aa 1-418 and aa 1-462) of the murine CI-M6PR (Genbank accession No NM_010515.2), was ordered as synthetic DNA fragment (not codon optimized) (IDTgBlocks) and cloned in a similar way.

From the 55 anti-CI-M6PR VHHs, 10 of the representative sequences corresponding to a particular CDR3 group were cloned into a Pichia pastoris expression vector (pAOX_LacZ module_AOXt_sh ble). The 10 sequences were amplified from the pMECs vector (forward primer: 5′ GGGCTCTTCAAGGCAGGTGCAGCTGCAGGAGTC 3′ (SEQ ID NO:16) and reverse primer: 5′ GGGCTCTTCAAGTCTAGTGATGGTGATGGTGGTGGCTGGAGACGGTGACCTGGG 3′ (SEQ ID NO:17)) containing Sapi restriction site overhangs. After purification with CleanNA beads (GC-Biotech, MB AC-60050), the insert (150 ng), vector backbone (75 ng), Sapi restriction enzyme (NEB, 0.5 μL), T4 ligase (NEB, 1 μL) and T4 ligase buffer were subjected to a PCR cycle (50×) of 2 minutes at 37° C. and 5 minutes at 16° C., followed by a final incubation for 10 minutes at 65° C.

Anti-CI-M6PR VHH6 was amplified by PCR using E. coli containing recombinant pMECS harbouring the VHH6 gene as template and primers A6E (5′ GATGTGCAGCTGCAGGAGTCTGGRGGAGG 3′ (SEQ ID NO:18)) and primer PMCF (5′ CTAGTGCGGCCGCTGAGGAGACGGTGACCTGGGT 3′ (SEQ ID NO:19)). After purification with CleanNA beads (GC-Biotech, MB AC-60050) the PCR product was digested overnight at 37° C. with PstI (NEB) and afterwards with BstEII (NEB) overnight at 60° C. In parallel, the pHEN6c vector was digested with PstI for 3 hours, purified and digested with BstEII for 3 hours. Ligation of the digested insert and vector was performed using T4 ligase and buffer (NEB) overnight at room temperature. Competent WK6 E. coli was transformed using a heat shock (90″ at 42° C.) with the ligated construct. All plasmids described have been sequence verified using Sanger sequencing.

### 2. Recombinant Production of the Human Domain1-3His6 Antigen

HEK293 suspension cells were cultivated in serum-free EX-CELL (Gibco 14571C-1000ML) and Freestyle 293 medium (Gibco) (1:1) supplemented with L-Glutamine (2 mM) and grown at 37° C. in 8% CO₂ while shaking at 125 rpm.

pcDNA™3.3-TOPO-hDom₁₋₃His₆ (675 μg) and SV40 Large T antigen DNA (1%) was used for transfection of HEK293 suspension cells (300 mL) with polyethylene imine (1:2) (PolyScience, linear, 25 kDa). The supernatant was harvested 3 days after transfection (200× g, 5′) and supplemented with MgCl₂ (2 mM), reduced L-Gluthation (100 mg/L, Sigma Aldrich, G4251-1G) and 1× cOmplete™Protease Inhibitor (Roche, 11697498001). After filtering (0.22 μm), the supernatant was loaded onto a HisTrap HP (5 mL) column (GE Healthcare, 17524801). After washing (5 CV of 20 mM imidazole, 0.5 M NaCl, 20 mM NaH₂PO₄/Na₂HPO₄, pH 7.5), the bound proteins were eluted (10 CV, 400 mM imidazole, 20 mM NaCl, 20 mM NaH₂PO₄/Na₂HPO₄, pH 7.5) and analysed on SDS-PAGE (4-20%, Genscript). Afterwards, the hDomain₁₋₃His₆ positive fractions were loaded on a HiLoad 16/600 Superdex 200 pg (GE Healthcare, 28989335) and eluted fractions were analysed on SDS-PAGE followed by staining with Coomassie B-Blue R250 and positive fractions were pooled and concentrated in MES buffer (50 mM MES, 150 mM NaCl, pH 6.5). The mDom1-3His6 was expressed and produced similarly to the human variant but only purified over a HisTrap (5 mL) column (GE Healthcare, 17524801). The eluted fractions were pooled, concentrated over a Amicon© Ultra-15 Centrifugal Filter Unit (Merck Millipore, UFC901008) and resuspended in MES buffer.

### 3. Immunization and Panning

Two llamas were subcutaneously injected (100 pg hDomain₁₋₃His₆, emulsified with Gerbu adjuvant P) on days 0, 7, 14, 21, 28 and 35. On day 40 peripheral blood lymphocytes were collected and from total isolated RNA, cDNA was synthesized with an oligo(dT) primer. Using this cDNA, the VHH encoding sequences were amplified by PCR, digested with PstI and NotI, and cloned into the PstI and NotI sites of the phagemid vector pMECS. This obtained two different VHH libraries consisting of 10⁸ independent transformants, with 81% and 85% of transformants resp. harbouring the vector with the right insert size.

In the original panning experiments (Example 1), both VHH libraries were separately panned on solid-phase coated antigen (100 μg/ml in PBS) for 3 rounds. The antigen used for panning was the same as the one used for immunization, using uncoated blocked wells as negative control. For each panning round, 2 wells were coated with antigen. The phages from one well were eluted under basic conditions (pH=11), the other one under acidic conditions (pH=2.5). The output of both conditions was mixed and used as input for the following round of panning. The enrichment for antigen-specific phages was assessed after each round of panning by comparing the number of phagemid particles eluted from antigen-coated wells with the number of phagemid particles eluted from negative control (uncoated blocked) wells.

At a later instance, the original VHH library that resulted in the successful identification of antigen-specific VHHs was re-panned in an effort to identify VHHs from CDR3-families besides those of VHH1-VHH11. Panning experiments were performed similarly, but here the bound phages were eluted through two other methods. On the one hand, elution was performed through the addition of 1 mg/ml of trypsin in PBS and the reaction stopped by adding 5 μl/well of 4 mg/ml AEBSF trypsin inhibitor. On the other hand, the bound phages were competitively eluted by the addition of purified VHH7 and VHH8 at 108 nM at 4.5 nM respectively. After one round of enrichment, the periplasmic extracts of 96 randomly picked colonies resulting from each of these panning experiments were assessed for specific binding of coated human CI-M6PR Dom₁-3His₆ through ELISA.

### 4. Production and Purification of Anti-CI-M6PR VHHs

The plasmid (1000 ng) was linearized using Pmel (1U, NEB) and used to transform electrocompetent⁹⁸ Pichia pastoris NRRL-Y-11430 by electroporation. Subsequently, Buffered Glycerol Complex Medium for Yeast (pH 6) was used for inoculation of a single clone transformant and growth for 48 h at 28° C. while shaking at 225 rpm. A buffer switch was performed to Buffered Complex Medium for Yeast (pH 6) and cultures were grown for another 48 h at 28° C. while shaking at 225 rpm. Every 12 h, the growing cultures were spiked with methanol (1%). Finally, the supernatant was harvested by centrifugation (1250 rpm, 15′) and adjusted to pH 7.

VHH1, VHH5, VHH6, VHH 1H11 and VHH 1H52 were expressed in E. coli by transforming competent WK6 E. coli cells with the pHEN6c vector containing the VHH open reading frames, the Lac operon, the PelB secretion signal, the ampicillin selection marker and an origin of replication. Transformed E. coli cells were inoculated in LB medium containing ampicillin (100 μg/mL) and incubated overnight at 37° C., while shaking at 200-250 rpm. Of this preculture 1 ml was added to 330 mL TB containing ampicillin (100 μg/mL), MgCl2 (2 mM) and glucose (0.1%) and incubated at 37° C. while shaking until OD600 was 0.6-0.9.

When reached the desired OD600, the expression was induced by addition of IPTG (Immunosource Cat° 102A) (1 mM) and the culture was incubated at 28° C., while shaking for 16-18 h. To extract the proteins the overnight-induced cultures were centrifuged for 8 minutes at 8000 rpm and the cell pellet was resuspended from 1 L culture in 12 ml TES by pipetting up and down, followed by shaking for 1 hour at 4° C. Per 12 ml TES, 18 ml TES (1:4 diluted in MQ) was added and further incubated on ice for an additional 20 hour, while shaking. The whole was centrifuged for 30 min at 8000 rpm at 4° C. and the supernatant was used for further purification.

For all VHHs, the clarified supernatant was supplemented with supplemented with MgCl₂ (2 mM), reduced L-Gluthation (100 mg/L, Sigma Aldrich, G4251-1G). After filtration (0.22 μm) the supernatant was loaded onto a HisTrap HP (5 mL) column (GE Healthcare, 17524801) after which the bound proteins were washed (5 CV of 20 mM imidazole, 0.5 M NaCl, 20 mM NaH₂PO₄/Na₂HPO₄, pH 7.5) and gradually eluted (10 CV, 400 mM imidazole, 20 mM NaCl, 20 mM NaH₂PO₄/Na₂HPO₄, pH 7.5). Analysis of selected peak fractions was performed on SDS-PAGE (4-20%, Genscript). Fractions containing the protein of interest were pooled and ran on a HiLoad16/10 desalting column, equilibrated with HBS buffer (50 mM HEPES, 150 mM NaCl, pH 7).

### 5. Cloning Production and Purification of Humanized Variants of VHH7 and VHH8 and of the Alternative M6PR-Specific VHHs

The human codon optimized coding sequences for VHH7hWN and VHH8hWN containing the IgG C_{H} signal peptide and a His₆-tag were ordered synthetically and incubated for 45 minutes at 37° C. with Klenow fragment (3′ to 5′ exo-) (NEB, M0212L), NEBuffer 2 (NEB), dATP (0.1 mM) and cloned using pcDNA™3.3-TOPO™ TA Cloning™ Kit (Thermo Fischer Scientific, K830001) according to the provided protocol. Codon optimized sequences of the humanized variants VHH7 h1-3 and VHH8 h1-5 and of the alternative CI-M6PR-specific VHHs (as disclosed in WO2020/185069A1 by Houthoff et al.) were cloned into the pVDS100 vector using the GenBuilder™ cloning kit (GenScript®; cat. no.: L00701) according to the manufacturer's instructions. The cloned plasmids were heat shock transformed (42° C., 90 seconds) into chemically competent E. coli and sequence verified. For recombinant protein production of VHH7hWN and VHH8hWN, the corresponding expression vectors were transfected into HEK293 suspension cells through PEI-transfection. The medium was harvested for purification on day 4 after transfection. VHH7 h1-3 and VHH8 h1-5 and the alternative M6PR-specific VHHs were expressed in E. coli by transforming competent cells with the pVDS100 vector containing the VHH open reading frames. Transformed E. coli cells were inoculated in selective LB medium and incubated overnight at 37° C., while shaking at 250 rpm. The preculture was diluted 1:50 in selective TB-medium supplemented with glucose and lactose for auto-induction of protein expression. The culture was incubated for 2 h at 37° C. while shaking at 250 rpm, after which the temperature was reduced to 30° C. and the culture was incubated for an additional 26 h. To extract the proteins the overnight-induced cultures were centrifuged for 20 minutes at 4000 rpm and the cell pellet was resuspended in D-PBS (1/12.5^{th} of the expression volume) by pipetting up and down, followed by shaking for 1 hour at 4° C. The whole was centrifuged for 20 min at 8500 rpm at 4° C. and the supernatant was used for further purification. All supernatants were filtrated (0.22 μm) before purification. Supernatant for VHH7hWN and VHH8hWN was loaded onto a HisTrap HP (5 mL) column (GE Healthcare, 17524801) after which the bound proteins were washed (5 CV of 20 mM imidazole, 0.5 M NaCl, 20 mM NaH₂PO₄/Na₂HPO₄, pH 7.5) and gradually eluted (10 CV, 400 mM imidazole, 20 mM NaCl, 20 mM NaH₂PO₄/Na₂HPO₄, pH 7.5). The VHH-positive fractions were loaded on a HiLoad 16/600 Superdex 75 pg (GE Healthcare) and eluted fractions were analysed on SDS-PAGE. Positive fractions were pooled and concentrated in HBS-buffer (50 mM HEPES, 150 mM NaCl, pH 7). For VHH7 h1-3, VHH8 h1-5 and the alternative M6PR-specific VHHs, the IMAC purification was performed on a Janus BioTx system (Perkin Elmer) according to standard procedures. Fractions containing the protein of interest were pooled and buffer exchanged to PBS prior to protein concentration.

### 6. Thermal Stability Assay

For VHH1-VHH11, 10 μM (20 μL) protein sample and 20×SYPRO Orange dye (20 IL) (Thermo Fischer Scientific, S-6650) was diluted in HBS buffer and triplicates were sampled in white Opaque 96-well microplate (Perkin Elmer, 6005290). The reaction was initiated at room temperature after which 25° C. was reached at 4.8° C. per second without acquisition. Afterwards, samples were continuously acquiesced (Ex/Em: 498 nm/610 nm) while the temperature was increased to 95° C. at 0.01° C. per second with 40 acquisitions per ° C. (Roche LightCycler480). The melting point is determined by truncating the data points to the melting point, and normalizing the data and performing a non-linear regression analysis employing a Boltzmann sigmoidal equation.

### 7. Enzyme Linked Immunosorbent Assay (ELISA)

The binding specificity of the anti-CI-M6PR VHH's VHH1-VHH11 and an anti-GFP VHH were assessed by ELISA. Wells of a microtiter plate (Maxisorp™ 96-well plates (Nunc, 442404)) were coated overnight at 4° C. with human domain₁₋₃His₆ (100 ng/well, diluted in 50 mM NaHCO₃/Na₂CO₃₂, pH 9.6. The next day, 200 μL of blocking buffer (1% probumin in PBST (0.05% (v/v) Tween 20 in PBS)) were added and the microtiter plate was incubated for 2 hours at room temperature, after washing with PBST. Different concentrations (0-7.5 μM or 0-1 μM, in 1% probumin in PBST) of the anti-CI-M6PR VHHs were added, and incubation was continued for 2 hours. The wells were washed three times with PBST and then incubated with anti-Camelid VHH HRP conjugated antibody (1:3000, Genscript, A01860). After 1 h incubation and washing with PBST, 3,3′,5,5′-tetramethylbenzidine (TMB) was added as substrate and absorbance of the solution in the wells was measured at 450 nm with an ELISA reader after adding H₂SO₄ (2N).

### 8. Flow Cytometry Analysis of Anti-CI-M6PR VHH Binding to the Native Receptor

To analyse binding of VHH1-VHH11 on the native CI-M6PR receptor, HEK293, Michigan Cancer Foundation-7 (MCF7) and L-D9 cells overexpressing a mouse-bovine chimeric CI-M6PR¹⁰² were used in a flow cytometry experiment. HEK293 and MCF7 cells were both cultivated in DMEM:F12 medium supplemented with FCS (10%) and L-glutamine (2 mM). The L-D9 cells were cultivated in DMEM modified to contain L-glutamine (4 mM), glucose (4500 mg/L), sodium pyruvate (1 mM), and sodium bicarbonate (1500 mg/L).

For every condition, 10E5 cells were taken and strained (40 μm cell strainer) and supplemented in a 96-well plate. After centrifugation (200×g, 5′, 4° C.), cells were resuspended in PBS (1% BSA) and incubated for 20′ on ice and afterwards incubated with serially diluted VHH (starting from 200 μg/ml in PBS containing 0.5% BSA) for 2 h at 4° C. The cells were washed three times in PBS (0.5% BSA) and incubated with anti-His-PE antibody (AD1.1.10, Novus Biologicals—NB100-64151) for 1 h at 4° C. After three washing steps, in PBS diluted (1/200) eFluor780 L/D staining (BD Biosciences) was incubated for 20′ on the cells and washed away afterwards. For every condition, 3E4 cells were analysed on the LSR HTS device (BD). As negative and positive control GFP-binding VHH (in house) and an anti-CI-M6PR antibody (1/400, Novus Biologicals NB100-64151) was included.

To assess internalisation of the VHHs, a comparable experiment was conducted however, incubation with VHHs was performed for 1 h at 37° C. on MCF7 cells after which cells were washed with PBS and trypsinized. After transferring to a FACS plate, cells were incubated with L/D staining (eFluor 780, 1/200) (BD Biosciences) for 20′ on ice. After washing the cells in PBS, cells were fixed using fixation/permeabilization concentrate (Cat° 00-513-43, eBioscience) that was diluted 4 times in fixation/permeabilization diluent (Cat° 00-5223-56, eBioscience) according the provided protocol. Subsequently, permeabilization of the cells was achieved (according provided protocol) by incubation and washing with permeabilization buffer (Cat° 00-8333-56, eBioscience), diluted in PBS. Finally, anti-His-PE antibody (AD1.1.10, Novus Biologicals— NB100-64151) was diluted in permeabilization buffer and incubated for 1 h at 4° C. After three times washing, 3E4 cells per condition were analysed on the LSR HTS device (BD).

### 9. Biolayer Interferometry (BLI) Kinetic Measurements

Conditions as used in Experiment 1 as shown in Table 3:

Analysis of the binding kinetics was performed using an Octet Red96 (FortéBio) device with Streptavidin SA Biosensor tips (VWR 733-2145). Therefore, 1 mL of human domain₁₋₃His₆ (0.5 mg/mL in 50 mM MES, 150 mM NaCl, pH 6.5) was incubated at room temperature for 30 minutes with EZ-Link™ NHS-PEG4-Biotin (1 mg) (Thermo Fischer A39259) and NaHCO₃ (100 mM). Biotinylated human domain₁₋₃His₆ was purified using a Zeba spin desalting Column™ (7K MWCO, 2 mL, Thermo Fischer 89890) and 1 μg/mL was used for immobilization on the Streptavidin SA Biosensor tips. The loaded tips were dipped into a particular anti-CI-M6PR VHH solution, serially diluted in kinetics buffer (0.2M NaH₂PO₄, 0.1M Na⁺ citrate, 0.01% BSA, 0.002% Tween20, pH 7.4). This was followed by dissociation of the particular anti-CI-M6PR VHH in kinetics buffer of pH 7.4, followed by regeneration in Glycine (10 mM, pH 3) and neutralisation. Same cycle was repeated but the dissociation was performed at pH 7.0, 6.0, 5.0, 4.5 and 4.0. A reference well and reference sensors were subtracted from ligand sensors afterwards and Savitsky-Golay filtering was applied on the data. Fitting of the data was performed using a 1:1 model, grouped per pH of dissociation.

Conditions as used in Experiment 2 as shown in Table 3:

Analysis of the binding kinetics was performed using an Octet Red96 (FortéBio) device with Streptavidin SA Biosensor tips (VWR, 733-2145). To this end, 1 mL of hCI-M6PR_{D1-D3} (0.5 mg/mL in 50 mM MES, 150 mM NaCl, pH 6.5) was incubated at room temperature for 30 minutes with a two-fold molar excesses of EZ-Link™ NHS-PEG₄-Biotin (Thermo Fischer, A39259) and NaHCO₃ (100 mM, pH 8.3). Biotinylated hCIMPR_{D1-D3} was purified using a Zeba spin desalting column (Thermo Fischer, 89890) and protein concentration was calculated by measuring the absorbance at 280 nm (extinction coefficient 50320 M¹ cm⁻¹). Two-fold biotin-labelled antigen was loaded on the tips (2.5 μg/mL) and subsequently dipped into a particular anti-CI-M6PR VHH-containing solution (ranging from 0-100 nM). Serial VHH dilutions were made in kinetics buffer (0.2 M Na₂HPO₄, 0.1 M Na⁺ citrate, 0.01% BSA, 0.002% Tween20, pH 7.4). After association (100 sec), dissociation was conducted (500 sec) for each anti-CI-M6PR VHH in the kinetics buffer, followed by biosensor regeneration in glycine (10 mM, pH 3) and neutralisation. The same cycle was repeated with dissociation performed in kinetics buffer of pH 7.0, 6.5, 6.0, 5.5, and 5.0.

A reference well and reference sensors were subtracted from ligand sensors afterwards and Savitsky-Golay filtering was applied on the data. Fitting of the data was performed using a 1:1 model, grouped per dissociation pH.

10. Labelling of Anti-CI-M6PR VHHs with Amine Reactive Alexa Fluor 488

Every anti-CI-M6PR VHH (1 mg) was diluted in HEPES (50 mM), NaCl (150 mM) and NaHCO₃ (100 mM), pH 8.3 and incubated with 1 mg Alexa Fluor 488 (AF488) NHS ester (Jena Biosciences, APC-002-5), resuspended in DMSO, for 1 h at room temperature. Afterwards, free AF488 NHS ester was removed using size exclusion chromatography (HiLoad 16/600 Superdex75 pg, GE Healthcare). Eluted fractions were pooled and degree of labelling (DOL) and functional binding to hDom1-3His6 was assessed (FIG. 18).

### 11. Microscopic Analysis of Lysosomal Targeting of the Anti-CI-M6PR VHHs

Michigan Cancer Foundation-7 (MCF7) cells were cultivated as previously described and seeded in 8-well chambers (iBidi, 80841) at 2×10⁴ cells/well in OptiMEM medium one day before the experiment. The next day, MCF7 cells were incubated with 7.5 μM anti-CI-M6PR VHHs and LysoTracker Deep Red DND-99 (100 nM, Thermo Fischer, L12492) for 45 minutes at 37° C., 5% CO₂ and washed with PBS afterwards. After fixation with paraformaldehyde (4%, 20′ at room temperature), the cells were washed with PBST and wells were blocked using goat serum (in PBST) for 1 h at room temperature. Finally, cells were stained with Hoechst (in PBS, 30′ at room temperature) and mounted in n-propyl gallate after washing with PBST.

Imaging was performed on the LSM880 Airyscan confocal microscope (Zeiss, Jena) used in FAST Airyscan SR-mode with a Plan-Apochromat 63×/1.4 Oil DIC M27 objective. For every VHH, optimal Z-stacks—to capture the entire cell—were taken at three different positions per well of three fluorescent compounds: LTR (λ_{EX}: 633 nm and λ_{Em}: >650 nm), AF488 (λ_{Ex}: 488 nm I_{Em}: 495-550 nm) and Hoechst/DAPI (λ_{Ex}: 405 nm and λ_{Em}: 420-480 nm).

For live-cell imaging, 2×10⁴ MCF7 cells/well were seeded in OptiMEM medium and incubated the next day with LysoTracker Deep Red DND-99 (50 nM, Thermo Fischer, L12492) for 30 minutes at 37° C., 5% CO₂ and washed with OptiMEM after which 7.5 μM AF488 labelled anti-CI-M6PR VHHs (in OptiMEM) were incubated on the cells.

For every well (i.e. anti-CI-M6PR VHH), Z-stacks were taken at three different positions every six minutes, for three hours in total. Z-slices (12) were acquired per position at a step size of 1.5 μm and XY pixel size was 275 nm by 275 nm. Excitation and emission wavelengths of the fluorescent compounds used were LTR (λ_{Ex}: 633 nm and λ_{Em}: 665-715 nm), AF488 (λ_{Ex}: 488 nm and A_{E}m: 520±35 nm), Hoechst/DAPI (λ_{Ex}: 405 nm and A_{Em}: 420-470 nm).

### 12. Microscopic Analysis the Lysosomal Targeting of Anti-CI-M6PR VHHs

HeLa CTSD^{-/-} (clone 3D5) cells were cultivated as previously described and seeded in 8-well chambers (iBidi, 80841) at 2.5×10⁴ cells/well in Ham F-12 medium (supplemented with penicillin and streptomycin) respectively. AF488-labelled VHH7 and VHH8 (5 μM) were incubated for four hours on the cells and washed three times with PBS afterwards. Cells were fixed in prewarmed PFA: first in 2% PFA in PBS for 5 minutes at 37° C., and then with 4% PFA for 10 minutes at room temperature. Washing with PBS was performed three times for 5 minutes before and after cell permeabilisation (0.2% Triton X-100) for 10 minutes at room temperature. Cells were then blocked for 30 minutes with normal goat serum diluted (1/100) in PBT buffer. Primary mouse anti-LAMP1 monoclonal antibody (Abcam, Ab25630, 1/500) was diluted in blocking buffer and incubated overnight at 4° C. After washing, 5 minutes each in PBS, the secondary goat anti mouse antibody, coupled to DyLight594 (1/1,000 in PBT) was incubated for two hours at room temperature. The cells were counterstained with DAPI (1/1,000 in PBS) for 15 minutes at 16° C. after washing three times with PBS. Lastly, washed and stained cells were stored at 4° C. after mounting in polyvinyl alcohol. Imaging was performed on the LSM880 Airyscan confocal microscope (Zeiss, Jena) used in FAST Airyscan SR-mode with a Plan-Apochromat 63x/1.4 Oil DIC M27 objective. For every VHH, optimal Z-stacks—to capture the entire cell volume—were taken at three different positions per well of three fluorescent compounds: LAMP1 (λ_{Ex}: 633 nm and λ_{Em}: >650 nm), AF488 (λ_{Ex}: 488 nm λ_{Em}: 495-550 nm) and Hoechst/DAPI (λ_{Ex}: 405 nm and λ_{Em}: 420-480 nm).

### 13. Image Processing and Analysis

Images acquired with the Airyscan detector were processed using ZEN software (Zeiss, Jena). The processing included pixel reassignment and default Wiener filtering. The processed images were then imported into Volocity (Quorum Technologies, Ontario) for further analysis. Both images acquired on the LSM880 and the Spinning Disk microscope were analyzed with Volocity software. In both cases, thresholds were determined for intensity values and size of segmented objects in the channel of (endo)lysosomal staining and in the channel of labelled VHH. In this way, two populations were created, one containing (endo)lysosomes, one containing the labelled VHHs. Applying the ‘intersect’ command allowed us to determine the fraction of VHH that localized inside the (endo)lysosomes, and the fraction of lysosomes that contains VHH. The total volume of analyzed cells was also measured to correct for. The calculation of the fractions was done based on the segmented volumes and volumes were expressed in ‘voxels’. A voxel is the 3D version of a pixel, so a volumetric unit in the image stack. For the live-cell imaging results, uptake per cell volume was calculated by dividing the sum of voxel count for each fluorescent VHH time point by the sum of voxel count per cell (representing the cell volume) at that time point. The percentages of VHH colocalising with lysosomes and the percentage of the entire endolysosomal pool containing the particular VHH were calculated by taking the ratio of the voxel counts of VHH-signal colocalising with LTR and of the total intracellular VHH signal. The percentage of lysosomes with VHHs was determined by the voxel count ratio of the VHH-signal colocalising with LTR and the total LTR signal. The last graph shows the absolute voxel counts of the intracellular VHH signal and the VHH-LTR colocalising signal.

### 14. Size-Exclusion Chromatography Coupled to Multi-Angle Light Scattering

To estimate the molecular mass and stoichiometry of the hCI-M6PR_{D1-D3} and anti-CI-M6PR VHH8 protein complex, we incubated both proteins in a 1:1 and 1:3 molar fashion in HBS buffer (50 mM HEPES, 150 mM NaCl, pH7.5, 0.1 μm filtered) containing sodium azide (0.02%). The total concentration of both samples was 0.81 and 1.08 mg/ml. After SEC (Superdex 200 increase HR 10/30), eluted proteins were detected with an online UV detector (Generic UV), a mini DAWN 8 (Wyatt) multi-angle laser light scattering (MALLS) detector and an Optilab refractive index (RI) instrument (Wyatt) at 298 K. The RI increment value (dn/dc value) at 298 K and 658 nm was calculated for the determination of the protein concentration and molecular mass (dn/dc: 0.1850 ml/g). Eluted fractions between 14-40 minutes (at 0.5 mL/min) were collected for analysis on SDS-PAGE. Data analyses and reporting was performed using the ASTRA 7.3.2 software.

### 15. Intact Mass Spectrometry of Anti-CI-M6PR VHH:Receptor Complexes

Intact proteins were separated on an Ultimate 3000 HPLC system (Thermo Fisher Scientific, Bremen, Germany) online connected to an LTQ Orbitrap XL mass spectrometer (Thermo Fischer Scientific). Briefly, approximately 4 μg of protein was injected on a Zorbax Poroshell 300SB-C8 column (5 μm, 300 Å, 1×75 mm IDxL; Agilent Technologies) and separated using a 15 min gradient from 5% to 80% solvent B at a flow rate of 100 μl/min (solvent A: 0.1% formic acid and 0.05% trifluoroacetic acid in water; solvent B: 0.1% formic acid and 0.05% trifluoroacetic acid in acetonitrile). The column temperature was maintained at 60° C. Eluting proteins were directly sprayed in the mass spectrometer with an ESI source using the following parameters: spray voltage of 4.2 kV, surface-induced dissociation of 30 V, capillary temperature of 325° C., capillary voltage of 35 V and a sheath gas flow rate of 7 (arbitrary units). The mass spectrometer was operated in MS1 mode using the orbitrap analyzer at a resolution of 100,000 (at m/z 400) and a mass range of 600-4000 m/z, in profile mode. The resulting MS spectra were deconvoluted with the BioPharma Finder 3.0 software (Thermo Fisher Scientific) using the Xtract deconvolution algorithm (isotopically resolved spectra), after which the deconvoluted spectra were annotated automatically using the BioPharma Finder protein sequence manager and protein identification tool.

### 16. Co-Crystallisation of the VHH-hCI-M6PRD1-D3 Complex

Complexes of hCI-M6PR_{D1-D3} with either VHH7, -8 or 1H11 were formed and polished on SEC in HBS buffer (20 mM HEPES, 150 mM NaCl, pH 7.5). To this end, solutions containing hCI-M6PR_{D1-D3} and a 1.25 molar excess of either anti-CI-M6PR VHH were injected onto a Superdex 200. The fractions containing VHH complexes were collected, supplemented with mannose-6-phosphate (1 mM, M3655-100MG, Sigma) and concentrated up to 3.5 mg/mL and 7 mg/mL respectively using an Amicon Ultra-15 protein concentrator (UFC903024, Millipore). VHH 1H11 complexes where concentrated to 3.7 mg/ml without the addition of mannose-6-phosphate. For crystalization of the complexes in general, nanolitre-scale sitting drop vapour diffusion crystallization experiments were set up at 287 K using commercially available sparse matrix crystals screens (Molecular Dimensions, Hampton Research) and a Mosquito crystallization robot (TTP Labtech). Promising hits were further optimized using gradient optimization in 96-well.

Two crystal forms of VHH7:hCI-M6PR_{D1-D3} were identified: a rhombohedral crystal, diffracting to 2.2 Å, crystallised from 0.3 M KBr, 0.1 M NaCacodylate pH 6.5, 8% w/v γ-PGA (Na+ form, LM) (PGA screen condition C9; Hu et al. Acta Crystallogr D Biol Crystallogr. 2008; 64: 957-63) and a tetragonal crystal form, diffracting to 3.0 Å, crystallised from 0.2 M NH₄NO₃, 0.1 M Bis-Tris propane pH 8.5, 18% v/v PEG Smear High (BCS screen condition F6). A single crystal form of hCI-M6PR_{D1-D3}:VHH8 was identified growing from sodium acetate trihydrate (0.08 M), sodium chloride (0.15 M), Tris (0.1 M), PEG Smear (0.015% v/v), pH 8) (BCS screen condition F3) which diffracted to 2.75 Å. Two crystal forms of VHH 1H11: hCI-M6PR_{D1-D3} were identified: a poorly diffracting rhombohedral crystal form crystallized from a few conditions amongst which 0.2 M (NH4)2SO4 0.1 M Sodium acetate 4.6 25% v/v PEG Smear Broad (BCS screen condition C10) and a tetragonal crystal form, diffracting to 2.7 Å, crystallized from a few conditions amongst which 0.1 M Ammonium sulfate, 0.1 M Tris pH 7.5, 20% w/v PEG 1500 (Proplex screen condition A7).

The crystals containing complexes of VHH7 and VHH8 grown from BCS conditions were cryoprotected in mother liquor supplemented with ZW221 cryosolution (17.5% v/v) (Sanchez, et al. Biochemistry 54, no. 21 (2015): 3360-3369) consisting of DMSO (40%), ethylene glycol (20%) and glycerol (40%). The crystal grown from the PGA condition was cryoprotected in mother liquor supplemented with glycerol (17.5% v/v) and the crystal containing the VHH 1H11 complex was cryoprotected in mother liquor supplemented with 17.5% (v/v) ethylene glycol prior to vitrification in liquid nitrogen. Final X-ray diffraction measurements of VHH8-hCI-M6PR_{D1-D3} crystals were performed at EMBL P14 beamline (Petra 3 synchrotron, Germany), Proxima PX1 beamline (Soleil synchrotron, France) for the VHH7-hCI-M6PR_{D1-D3} crystal and ESRF ID30A3 for the VHH 1H11-hCI-M6PR_{D1-D3} crystal. All datasets originate from individual crystals. Diffraction data integration and scaling was performed in XDS¹². Dataset statistics are reported in Table X. Initial phases were recovered by maximum-likelihood based molecular replacement as implemented in Phaser using CIMPR_{D1D2}, CI-M6PR_{D3} based on the bovine CIMPR structure (PDB: 1sz0¹) and a VHH. Structures were iteratively built and refined in Coot, Isolde (Croll, Tristan Ian. 2018. “ISOLDE: A Physically Realistic Environment for Model Building into Low-Resolution Electron-Density Maps.” Acta Crystallographica Section D: Structural Biology 74 (6):519-30) implemented in ChimeraX and Phenix refine.

TABLE 14
Crystallographic data collection and refinement statistics.
Values in parenthesis refer to highest resolution shell.

| | CIMPR VHH7 | CIMPR VHH7 | CIMPR VHH1H11 | CIMPR VHH8 |
|---|---|---|---|---|
| Collection Date | 15 Dec. 2020 | 15 Dec. 2020 | 30 Jan. 2022 | 11 Sep. 2020 |
| Synchrotron | EMBL P14 | EMBL P14 | ESRF ID30A3 | Soleil PX1 |
| Condition | PGA C9 (0.3M KBr, | BCS F6 (0.2 M | Proplex A7 (0.1 M | BCS F3 (75 mM Na |
| | 0.1M NaCaco pH | Ammonium | Ammonium sulfate, | acetate, 0.15M NaCl, |
| | 6.5, 8% w/v γ- | nitrate, 0.1 M | 0.1M Tris pH 7.5, 20 | 0.1M Tris pH 8.0, 15% |
| | PGA (Na+ form, | Bis-Tris propane | % w/v PEG 1500) | (v/v) PEG Smear |
| | LM)) | pH 8.5, 18% v/v | | Medium) |
| | | PEG Smear High) | | |
| Cryo | 17.5% Glycerol | 17% ZW221 | 17.5% Ethglyc | 17% ZW221 |
| Unit Cell | 129.09 129.09 | 135.63 135.63 | 126.18 126.18 105.07 | 170.17 182.89 110.32 |
| Parameters | 569.16 | 95.57 | | |
| | 90 90 120 | 90 90 90 | 90 90 90 | 90 90 90 |
| Space group | R 3 2 (n°155) | P 41 2 2 (n°91) | P 41 21 2 (n°92) | C 2 2 21 (n°20) |
| Wavelength (Å) | 0.9763 | 0.9763 | 0.9677 | 0.978565 |
| Resolution (Å) | 189.72-2.2 (2.33- | 95.91-3.00 | 56.43 -2.70 (2.86- | 124.58-2.75 (2.91-2.75) |
| | 2.20) | (3.18-3.00) | 2.70) | |
| Reflection observed | 1015492 (123223) | 492251 (78908) | 72465 (11789) | 552471 (88164) |
| unique | 92391 (14175) | 18434 (2910) | 23537 (3745) | 45143(7153) |
| mulitplicity | 10.99 (8.69) | 6.24 (6.33) | 3.08 (3.15) | 12.24 (12.33) |
| completeness | 99.2 (95.0) | 100.0 (99.9) | 98.3 (99.4) | 99.8 (98.7) |
| I/σ(I) | 15.98 (1.09) | 10.94 (1.02) | 4.96 (0.99) | 12.58 (1.29) |
| R-meas (%) | 9.7 (177.1) | 39.1 (382.3) | 21.1 (134.5) | 18.9 (183.9) |
| CC(½) | 99.9 (36.9) | 99.8 (41.0) | 98.2 (45.9) | 99.8 (53.3) |
| Wilson B (Å²) | 59.48 | 72.8 | 53.4 | 64.5 |
| Reflections used in | 92080 (8194) | 18401 (1790) | 23484 (2337) | 45041 (4463) |
| refinement | | | | |
| for R-free | 1996 (180) | 1841 (179) | 1996 (197) | 2238 (206) |
| R-work | 0.2058 (0.3039) | 0.2187 (0.3333) | 0.2379 (0.3915) | 0.2134 (0.3254) |
| R-free | 0.2325 (0.3094) | 0.2578 (0.3762) | 0.2813 (0.4425) | 0.2563 (0.3446) |
| Number of non- | 9315 | 4225 | 4202 | 8671 |
| hydrogen atoms | | | | |
| macromolecules | 8856 | 4155 | 4107 | 8452 |
| ligands | 222 | 63 | 81 | 202 |
| solvent | 237 | 7 | 14 | 17 |
| Protein residues | 1112 | 532 | 525 | 1086 |
| RMS(bonds Å) | 0.002 | 0.012 | 0.003 | 0.003 |
| RMS(angles °) | 0,55 | 1.82 | 0.6 | 0.61 |
| Ramachandran | 96.08 | 95.04 | 95.36 | 94.69 |
| favored (%) | | | | |
| allowed (%) | 3.55 | 4.96 | 4.64 | 5.12 |
| outliers (%) | 0.36 | 0 | 0 | 0.19 |
| Rotamer outliers | 1.33 | 1.73 | 1.1 | 1.07 |
| (%) | | | | |
| Clashscore | 2.2 | 0.12 | 4.04 | 0.71 |
| Average B-factor | 82 | 92.55 | 67.83 | 79.23 |
| macromolecules | 81.36 | 92.79 | 67.47 | 79.13 |
| ligands | 128.24 | 80.81 | 90.53 | 85.37 |
| solvent | 62.67 | 57.43 | 42.09 | 55.47 |
| Number of TLS | 8 | 9 | 4 | 8 |
| groups | | | | |

### 17. Production of Site-Directed Mutated Variants of Human CI-M6PR Dom1-3His6 and Evaluation of CI-M6PR VHH Binding

Variants of the human CI-M6PR Dom1-3His6 with mutations in the epitopes of VHH7 or VHH8 were designed in silico based on the results of the PISA- and FastContact analysis of the corresponding crystal structures. Expression plasmids encoding CI-M6PR variants CI-M6PR_M85E, CI-M6PR_D87L, CI-M6PR_K89D, CI-M6PR_F143R and CI-M6PR_E148F (in the VHH7 epitope; SEQ ID NOs:83-87) and CI-M6PR_K191F, CI-M6PR_L197D, CI-M6PR_D409F, CI-M6PR_E433R and CI-M6PR_F457E (in the VHH8 epitope; SEQ ID NOs:88-92) were generated via site directed mutagenesis (QuikChange Lightning kit, Agilent) of pcDNA™3.3-TOPO-hDom₁₋₃His₆ using primers listed in Table 15. Mutations were verified by bidirections Sanger sequencing.

TABLE 15
Primers used for site-directed mutagenesis

| | | SEQ ID |
|---|---|---|
| Name | sequence | NO: |
| SDM01_CIMPR-M85E_Fwd | GCGCCGTGTGTgaGCACGACCTGAAAACCCGGACC | 51 |
| SDM02_CIMPR-M85E_Rev | CAGGTCGTGCtcACACACGGCGCTAGAAGGTCCAC | 52 |
| | ACTGCACG | |
| SDM03_CIMPR-D87L_Fwd | GTGTATGCACctCCTGAAAACCCGGACCTACCACAGCG | 53 |
| SDM04_CIMPR-D87L_Rev | GGGTTTTCAGGagGTGCATACACACGGCGCTAGAAGG | 54 |
| SDM05_CIMPR-K89D_Fwd | GCACGACCTGgAcACCCGGACCTACCACAGCGTGGGC | 55 |
| SDM06_CIMPR-K89D_Rev | GGTCCGGGTgTcCAGGTCGTGCATACACACGGCGC | 56 |
| SDM07_CIMPR-F143R_Fwd | CACCCCTGAGcgTGTGACCGCCACAGAGTGTGTGC | 57 |
| SDM08_CIMPR-F143R_Rev | GGCGGTCACAcgCTCAGGGGTGCCCAGTGTCTTGCC | 58 |
| SDM09_CIMPR-E148F_Fwd | CCGCCACAttcTGTGTGCACTACTTCGAGTGGCGG | 59 |
| SDM10_CIMPR-E148F_Rev | GTAGTGCACACAgaaTGTGGCGGTCACAAACTCAGGGG | 60 |
| SDM11_CIMPR-K191F_Fwd | CCCTCTGATCttcCTGAGCGGCGCCTACCTGGTGGAC | 61 |
| | GACAGCG | |
| SDM12_CIMPR-K191F_Rev | GGCGCCGCTCAGgaaGATCAGAGGGTTCAGATCGTGC | 62 |
| | TTCCGC | |
| SDM13_CIMPR-L197D_Fwd | GCGGCGCCTACgacGTGGACGACAGCGATCCTGATAC | 63 |
| | CAGCC | |
| SDM14_CIMPR-L197D_Rev | GCTGTCGTCCACgtcGTAGGCGCCGCTCAGCTTGATC | 64 |
| | AGAGGG | |
| SDM15_CIMPR-D409F_Fwd | CAGCGACGGCttTCTGACCCTGATCTACTTTGGCGGC | 65 |
| | G | |
| SDM16_CIMPR-D409F_Rev | CAGGGTCAGAaaGCCGTCGCTGTATCTCAGGGTCTG | 66 |
| SDM17_CIMPR-E433R_Fwd | GATCAATTTCcgGTGTAACAAGACCGCCGGCAACGA | 67 |
| | CGGC | |
| SDM18_CIMPR-E433R_Rev | GGTCTTGTTACACcgGAAATTGATCACGCTCATTCT | 68 |
| | CTGG | |
| SDM19_CIMPR-F457E_Fwd | GGACTGCACCTACgagTTCACCTGGGACACAGAGTAT | 69 |
| | GCCTGCG | |
| SDM20_CIMPR-F457E_Rev | CCCAGGTGAActcGTAGGTGCAGTCCACCTCGCC | 70 |

Mutated His-tagged CI-M6PR variants were produced in HEK293S cells and purified by IMAC and Superdex200 gel filtration (yield see Table 16), switching to a final buffer containing 50 mM MES, 150 mM NaCl, pH 6.5.

To determine the effect of site-directed mutations, an ELISA was performed. To this end, 100 ng of each successfully purified mutant was coated per well on Nunc Maxisorp plates in 50 mM MES, 150 mM NaCl, pH 6.5 buffer overnight at 4° C. Plates were blocked with 4% milk in PBS, and a dilution series of selected VHHs (VHH7 and its humanized form VHH7hWN, VHH8 and its humanized form VHH8hWN, as well as VHH1, VHH5, VHH 1H11 and VHH 1H52) were incubated on the plates. Coating-only controls were included for background correction. Binding of VHHs was revealed using a secondary HRP-linked antibody (MonoRab anti-camelid VHH HRP linked antibody, Genscript), followed by detection using TMB substrate (TMB substrate set, BD Opteia). Reactions were stopped after five minutes using a 2N H₂SO₄ solution and plates were read at 450 nm, with a 655 nm background correction. Data were analyzed using the GraphPad Prism 9 software.

TABLE 16
Yields of site-directed mutated human CI-M6PR
Dom1-3His6 variants after purification.

| | |
|---|---|
| VHH7 epitope mutants | VHH8 epitope mutants |

| Mutant | Yield (μg) | Mutant | Yield (μg) |
|---|---|---|---|
| M85E | 92.13 | K191F | 1 |
| D87L | 115.16 | L197D | 380.04 |
| K89D | 416.51 | D409F | 549.47 |
| F143R | / | E433R | 720.46 |
| E148F | 92.22 | F457E | 817.66 |

### 18. Reagents Enzyme Assays

As source of enzyme for the in vitro assays, we used the residual amounts of the reconstituted recombinant enzyme rhGAA (algucosidase alfa, Myozyme® Genzyme Cambridge MA) prepared for the treatment of Pompe disease patients at the AZ-Sint Lucas hospital pharmacy.

### 19. Cultivation of Cell Lines

HEK293 suspension cells were cultivated in FREX medium composed of EX-CELL (Gibco 14571C) and Freestyle 293 medium (Gibco) supplemented L-glutamine (Lonza, 2 mM). HeLa (human) and C2C12 myoblast (mouse) cells were cultured in EMEM (Non-essential amino acids (NEAA), 1.5 g/L NaHCO₃, 2 mM L-glutamine, 1 mM sodium pyruvate, 10% fetal calf serum (FCS)) and DMEM (glucose, L-glutamine, NaHCO₃, sodium pyruvate 1 mM, 10% fetal calf serum), respectively and incubated with 5% CO₂ at 37° C. GAA^{-/-} fibroblasts (GM00248, Coriell Cell Repository) were grown as essentially described by Reuser et al. (1984)s3. Cells were seeded and grown to confluence in Minimum Essential Medium (MEM, Invitrogen) containing Earle's salts and non-essential amino acids supplemented with 15% FCS and 2 mM L-glutamine.

### 20. Generation of the ERT (Fusion) Constructs

Expression Plasmids for rhCTSD and rhGAA

The human CTSD and GAA coding sequences (AAA51922.1, NM_000152), containing a 5′-AAGAACAAGCCGCCACC-3′ sequence (SEQ ID NO: 36) and a His₆ tag and 3′-GCTCTCCCTATTGTGAAGTCGCAC-5′ (SEQ ID NO: 37), were ordered synthetically (IDT gBlocks). The gBlocks were PCR amplified (Phusion polymerase (NEB)) using 5′-AAGAACAAGCCGCCACCATG-3′ (SEQ ID NO:38) as forward and 5′-GTGCGACTTCACAATAGGGAGAGC-3′ (SEQ ID NO:39) as reverse primer. The PCR reaction was purified using DNA CleanNA beads (GC-Biotech, MB AC-60050) and incubated for 45 minutes at 37° C. with Klenow fragment (3′->5′ exo-) (NEB, M0212L), NEBuffer 2 (NEB), dATP (0.1 mM) and cloned using pcDNA™3.3-TOPO™ TA Cloning™ Kit (Thermo Fischer Scientific, K830001) according to the provided protocol. The cloned plasmid was heat shock transformed (42° C., 90 seconds) into chemically competent MC1061 E. coli and sequence verified.

### Expression Plasmids for Lysosomal Enzymes-VHH Fusion Proteins

The chimeric constructs containing both coding sequences for lysosomal enzymes and the anti-CI-M6PR VHHs of interest were cloned via Golden Gateway technology. Every coding sequence was cloned into an entry vector in between two type II restriction sites to be compatible for further assembly with the parts of interest. The insert (150 ng), MP-G-BB vector (with corresponding overhangs) (50 ng), Bsal HF (10U, NEB) and CutSmart buffer were incubated for one hour at 37° C. and 20 minutes at 80° C. Afterwards, ligase buffer and T4 DNA ligase (Thermo Fischer, 5 U) were added to enable ligation for 1 hour at room temperature. Competent DH5a E. coli were transformed with 15 μL of the reaction mixture and plated on selective LB plates. After sequence verification, the entry vectors of interest (“parts”) were combined with a compatible backbone (Error! Reference source not found. 17), other parts of interest, ATP (10 mM), T4 ligase, Bsal HF and CutSmart buffer and subjected to 30 cycles of 3 minutes at 37° C. and 3 minutes at 16° C. The reaction was finalised with an incubation at 50° C. (5 minutes) and 80° C. (5 minutes). Competent DH10B E. coli were heat-shock transformed and plated on LB plates with kanamycin.

TABLE 17
Entry vectors used for the modular cloning of
CTSD/GAA-VHH, VHH-CTSD/GAA expression vectors

| | | | |
|---|---|---|---|
| VHH-CTSD-FLAG₃His₆ | CTSD-VHH- | VHH-GAA-FLAG₃His₆ | GAA-VHH- |
| | FLAG₃His₆ | | FLAG₃His₆ |
| pEN-L4-AG-R1 | pEN-L4-AG-R1 | pEN-L4-AG-R1 | pEN-L4-AG-R1 |
| pGGA-pCMV-B | pGGA-pCMV-B | pGGA-pCMV-B | pGGA-pCMV-B |
| pGGB-Kozak-ATG-C | pGGB-Kozak-ATG- | pGGB-Kozak-ATG-C | pGGB-Kozak-ATG-C |
| | C | | |
| pGGC-IgG C_{H} signal peptide- | pGGC-CTSD(1- | pGGC-IgG C_{H} signal peptide- | pGGC-GAA(1-952)- |
| VHH-D | 412)-D | VHH-D | D |
| pGGD-G4S linker-E | pGGD-G4S linker-E | pGGD-G4S linker-E | pGGD-G4S linker-E |
| pGGE−CTSD(21- | pGGE−VHH- | pGGE−GAA(65- | pGGE−VHH- |
| 412)FLAG₃His₆-F | FLAG₃His₆-F | 952) FLAG₃His₆-F | FLAG₃His₆-F |
| pGGF-bGH pA-G | pGGF-bGH pA-G | pGGF-bGH pA-G | pGGF-bGH pA-G |

Cloning Guide RNA in pSpCas9(BB)-2A-GFP (PX458) Vector

The CTSD gene was knocked out in the HeLa and C2C12 cell lines by the CRISPR/Cas9 editing system. The oligos for the human and mouse guide RNA were designed by using the ‘Knock out Guide Designer Tool’ of Synthego (Synthego Performance Analysis, ICE Analysis. 2019. v2.0. Synthego) and were ordered as DNA oligos (IDT) (Error! Reference source not found.). Cloning into the pSpCas9(BB)-2A-GFP(PX458) vector was performed as previously described⁵⁴. The resulting ligated vector was transformed to E. coli MC1061 cells by heat shock and plated on selective LB agar plates. The vector construction was confirmed by colony PCR with GoTaq polymerase (Promega) using the reversed guide-oligo and U6 primer. Clones with a construct of the correct length of 346 bp, verified on 2% agarose gel, were inoculated overnight in 5 ml liquid LB medium with ampicillin (100 μg/ml) at 37° C. Afterwards, prepared plasmids were sequence verified by Sanger sequencing using the U6 primer (Table 18).

TABLE 18
GuideRNA sequences for generation of CTSD^{-/-}
Hela and C2C12 cells

| | | SEQ |
|---|---|---|
| | | ID |
| Primer name | sequence (5′ → 3′) | NO: |
| CATD_048_ | CACCGATGGACGTGAACTTGTGCAG | 40 |
| Guide_Fw | | |
| CATD_049_ | AAACCTGCACAAGTTCACGTCCATC | 41 |
| Guide_Re | | |
| CATD_055_ | CACCGGCAAGTTCACATCTATCCGT | 42 |
| GuideMEF_Fw | | |
| CATD_056_ | AAACACGGATAGATGTGAACTTGCC | 43 |
| GuideMEF_Re | | |
| U6 primer | AGCCTATGGAAAAACGCCAGCAACGC | 44 |

### 21. Single Cell GFP Sorting and Clone Analysis

The successfully transfected cells were selected by GFP single cell sorting with the BD FACSAria™ III sorter in 96-well plates, 72 hours after transfection. The single clones were incubated 3-4 weeks at 37° C. and 5% CO₂ until reasonably grown.

Genomic DNA of growing single clones was recovered by cell digestion with QuickExtract™ DNA extraction solution (Lucigen), according to the manufacturer's instructions. The DNA region of interest was amplified in a PCR reaction with the high fidelity Kapa hifi DNA polymerase (HotStart ReadyMix, Roche), and primers for either the human and the mouse gDNA (Error! Reference source not found.). The PCR product was purified using magnetic CleanNA beads (CleanNA, MB AC-60050) according to the manufacturer's protocol. Finally, Sanger sequencing of the PCR product was performed using nested primers (Error! Reference source not found.19) to detect the CTSD knock-out and the sequencing results were analysed with the online Synthego Performance Analysis, ICE Analysis. 2019. v2.0. Synthego.

TABLE 19
Primer sequences for sequence verification
of CTSD^{-/-} in Hela and C2C12 cells

| Primer name | sequence (5′ → 3′) | SEQ ID NO: |
|---|---|---|
| CATD_050_Fw_1 | GGCAATGGGTTGCCATTCAC | 45 |
| | TG | |
| CATD_051_ | GACTCTGAGATTCCCCAGGG | 46 |
| Nested_Fw_2 | GC | |
| CATD_052_Re_1 | GTCCATGTAGTTCTTGAGCA | 47 |
| | CCTCGG | |
| CATD_mouse_Fw_1 | CAGGAGTTTGTGACATGTTG | 48 |
| | TGGC | |
| CATD_mouse_ | CTAGAGGCACCTAGGTGCAT | 49 |
| Nested_Fw_2 | G | |
| CATD_mouse_Re_1 | GCACTCACATCCAGGTAGTT | 50 |
| | TTTGAGTAAC | |

### 22. Transfection

HeLa and C2C12 cells were transfected by FuGENE® HD transfection reagent (Promega), according the provided protocol with a FuGENE:DNA ratio of 3:1. The transfected cells were checked 48 h post transfection for GFP expression by fluorescence microscopy. For small scale HEK293 cell transfections, also FuGENE® HD transfection reagent was used, for rhGAA expression, medium was supplemented with M6P (10 mM), sodium butyrate (3 mM)⁵⁵ or N-acetyl cysteine (10 mM)⁵⁶. Large-scale HEK293 and HEK293 GlycoDelete suspension cell transfection and recombinant protein production, was performed with PEI-mediated transfection.

23. Two-Step Purification of the CTSD-VHH and rhGAA-VHH Fusion Proteins

The fusion constructs, once produced, were purified by immobilised metal ion chromatography (IMAC) using a 5 ml HisTrap HP column on the ÄKTA Pure system (GE Healthcare), after adding reduced glutathione (100 mg/L) and filtering through a 0.22 μm filter (Millipore Steritop™). The 300-400 mL sample was loaded onto the column via a buffer inlet. The column was washed with lipopolysaccharide free (LPS⁻) binding buffer (20 mM NaH₂PO₄, 0.5 M NaCl, 20 mM imidazole) to remove unbound material. The flow through was collected separately while the bound proteins were eluted with LPS⁻ elution buffer (20 mM NaH₂PO₄, 20 mM NaCl, 400 mM imidazole) and collected in fractions of 1.5 ml. The eluted fractions associated with a peak in UV absorbance at 280 nm were analysed by SDS-PAGE with Coomassie Blue staining. If necessary, the presence of the fusion protein was analysed by western blotting, using monoclonal mouse anti-His DyLight800 antibody (Rockland). Protein concentrations determined by measuring the absorbance at 280 nm by the Eppendorf BioSpectrometer© (estimated extinction coefficients), and purified proteins were stored at −20° C.

The fractions containing the desired fusion protein were concentrated with 30 kDa molecular weight cut-off (only for rhCTSD-VHH) Amicon® centrifuge concentrators (Millipore) and purified afterwards in a second purification step by gel filtration (SEC), using a Superdex 200 column on the AKTA Pure system (GE Healthcare), equilibrated with PBS and glycerol (10%). Every 5 ml of (concentrated) sample was manually loaded onto the column from a syringe through a 15 ml superloop. The purity of the desired protein in the fractions corresponding to the UV absorbance peak was confirmed with SDS-PAGE or with western blot, if necessary. Desired fractions were pooled and concentrated using 30 kDa (for rhCTSD-VHH) and 50 kDa (for rhGAA-VHH) molecular weight cut-off centrifuge concentrators (Amicon®). Protein concentrations determined by measuring the absorbance at 280 nm by the Eppendorf BioSpectrometer® (estimated extinction coefficients), and purified proteins were stored at −80° C.

### 24. N-glycan Analysis

The isolation and DSA-FACE analysis of N-glycans were done on as described previously. Single exoglycosidase or phosphatase treatments and combinations thereof used in the experiments were incubated overnight at 37° C. with APTS-labelled N-glycans in a NH₄OAc (20 mM) of pH 5.2. The used enzymes include: 1) Arthrobacter ureafaciens a2,3/6/8-sialidase (in-house produced, 40 mU/reaction), 2) Glyko© @31-4-galactosidase from Streptococcus pneumoniae (Prozyme, 0.4 mU), 3) @3-N-acetyl-hexosaminidase from Jack Beans (Prozyme, 10 mU), and 4) calf intestinal phosphatase (CIP, Promega, M1821), which was conducted in CIP buffer (50 mM Tris-HCl (pH 9.3), 1 mM MgCl₂, 0.1 mM ZnCl₂ and 1 mM spermidine) for three hours at 37° C. and analysed after an additional SEC step.

The data were visualised with the Genemapper software (v6, Applied Biosystems), and relative abundances of each peak were calculated based on the peak heights (in RFU). The obtained electropherograms were aligned and processed in Inkscape 0.91.

### 25. Activity Assay Cathepsin D

We used a CTSD and CTSE cleavable, quenched fluorigenic peptide: 7-methoxycoumarin-4-acetyl (Mca)-Gly-Lys-Pro-Ile-Leu-Phe-Phe-Arg-Leu-Lys(dinitrophenyl)-D-Arg-NH₂ (λ_{Ex}: 320-340 nm λ_{Em}: 393-420 nm) (Enzo Life Sciences, BML-P145-0001). Active rCTSD (100 ng) cleaves the peptide in between the two Phe of the peptide and enables the release of the quencher and emission of the fluorescent signal. Proteolytic activity was measured by the Fluostar plate reader at defined time intervals at 37° C. with 200 nM substrate (10 μL, 0.5-200 μM) in sodium acetate (50 mM, pH 4, 80 μL) for 120 minutes. Linear phase velocities were plotted against the substrate concentrations; K_{M} and Vₘₐₓ were calculated by nonlinear regression using the Michaelis-Menten equation V₀=Vₘₐₓ×[S]₀/(K_{M}+[S]₀) in Graphpad Prism 9.0.0, where V₀ is linear phase initial velocity, Vₘₐₓ is maximal enzyme velocity, [S]₀ is the substrate concentration, and K_{M} is the Michaelis-Menten constant. The experiment was performed in triplicate, with the mean values plotted.

### 26. Intracellular Activity in HeLa and C2C12 Cells

HeLa and C2C12 CTSD^{-/-} cells were seeded at 1×10⁵ cells per 24-well one day before treatment. Recombinant proteins were incubated in Ham F12 medium (200 nM) for one to 24 hours and harvested after washing (three times with PBS). Lysates were prepared in RIPA buffer (50 mM NaOAc (pH 4), 150 mM NaCl, 1% NP-40, 1% sodium deoxycholate, 0.1% SDS) on ice for 30 minutes at 4° C. After centrifugation for 30 minutes at 14,000×g, the supernatant was collected. For every sample, proteins were quantified by colorimetric detection of Cu⁺ by bicinchoninic acid (BCA Protein Assay Kit, Pierce™ 23225). Intracellular activity was monitored according to section Error! Reference source not found. using 2.5 μg protein in RIPA buffer (10 μL) in duplicate.

### 27. Pharmacokinetics Study

Female wild-type C57B16/N mice of 10 weeks old were injected with 5 mg/kg recombinant protein (n=5). After every time point (1 h, 3 h, 6 h, 16 h and 48 h) mice from each group were sacrificed and blood was sampled by severing the right atrium of the heart. The blood from the thoracic cavity was transferred to SST microtainers (BD, 63931) and centrifuged (14,000×g for 5 minutes). The serum was collected and aliquot for storage at −20° C. until further investigation. We quantified the amount of CTSD in the serum via ELISA. Mouse anti-hCTSD (Thermo Fischer, CTD-19, 1/500) was coated overnight at 4° C. (Maxisorp™ 96-well plates (Nunc, 442404) and blocked (1% albumin). Serum samples (1/5 in PBS) were incubated for one hour, washed and incubated with a primary rabbit anti-His antibody (Thermo Fischer, PA1-983B, 1/500). Detection was performed using a secondary goat anti-rabbit HRP antibody. Absorbance at 450 nm was measured after incubation with 3,3′,5,5′-tetramethylbenzidine (TMB) substrate and H₂SO₄ (2N). The ELISA assay was performed in duplicate and the mean absorbance from five mice per time point, per injected protein is set out in function of time.

A similar pharmacokinetics study was performed for the rhCTSD and rhCTSD-VHH proteins, but blood sampling was performed at 30 minutes, 1 h, 3 h, 6 h and 16 h, and urine was collected by encouraging micturition of the mice while holding them over a disposable Petri dish. The blood from the thoracic cavity was processed as described above and the quantification of rhCTSD and rhCTSD-VHH proteins was performed using the DuoSet ELISA for hCTSD (R&D Systems, DY1014-05).

Animals were supplied by Charles River Jackson and treated according to the European guidelines for animal experimentation. The experiments were approved by the local ethical committee of Ghent University (EC2019-052).

28. In Vitro rhGAA Efficacy Assay

GAA^{-/-} cells were seeded at 5×10⁴ cells per 24-well one day before treatment. Recombinant proteins were incubated in Ham F12 medium (200 nM) for multiple hours and harvested after washing (three times with PBS). Lysates were prepared in RIPA buffer on ice for 30 minutes at 4° C. After centrifugation for 30 minutes at 14,000×g, the supernatant was collected. The glycogen content in these supernatants obtained was quantified with an enzymatic assay: Aspergillus niger amyloglucosidase (Sigma, 11202332001, 0.05 mg/ml) and Bacillus α-amylase (Sigma, A6380, 0.0125 mg/ml) were added. Digestion of glycogen to glucose was allowed for one hour at 37° C. and later measured using a method that involves glucose-oxidation and Amplex red reduction (Life Technologies-Molecular Probes, A22189), performed according to the manufacturer's protocol. The amount of glucose measured directly correlated to the remaining amount of intracellular glycogen in GAA^{-/-} cells, that were or were not treated with (chimeric) rhGAA.

TABLE 20
CDR sequences of CI-M6PR VHHs binding to the
epitope of VHH7 or VHH8, wherein CDRs are
annotated according to AbM

| | SEQ | | SEQ | | SEQ | | SEQ |
|---|---|---|---|---|---|---|---|
| | ID | | ID | | ID | | ID |
| VHH | NO: | CDR1 | NO: | CDR2 | NO: | CDR3 | NO: |
| VHH7 | 7 | GIIFSDN | 103 | TLASY | 110 | SSPVLNDI | 117 |
| | | RMD | | GWKT | | | |
| VHH1 | 1 | GFTFDRYW | 104 | TINTG | 111 | GATYYRGN | 118 |
| | | MN | | GTGTY | | SAI | |
| VHH | 71 | GIIFSDN | 105 | TLASY | 112 | GQY | 119 |
| 1H11 | | RMD | | GWKT | | | |
| VHH | 72 | GGAFSTYH | 106 | AITRG | 113 | SPQGKWNALV | 120 |
| 2H79* | | MG | | GSSST | | GGTPDY | |
| | | | | K | | | |
| VHH8 | 8 | GRTFNTYN | 107 | AIRWS | 114 | SIVDFTTNPS | 121 |
| | | WG | | SSKTS | | HFGS | |
| VHH5 | 5 | GRTFSRLA | 108 | AISEN | 115 | DRAAYYCSGSG | 122 |
| | | MG | | GDSIH | | CYPSRAPAAAS | |
| | | | | | | YDY | |
| VHH | 73 | GFTWDSYV | 109 | CLDVD | 116 | VNRASMRFRRC | 123 |
| 1H52 | | IG | | DGSIY | | LQVLRYDY | |

*this VHH could not be confirmed as a binder of said epitopes.

SEQ ID NO: 124-138: CI-M6PR-specific VHHs as used in Houthoff et al. (WO2020/185069A1)

### REFERENCES

1. Kishnani, P. S. et al. Immune response to enzyme replacement therapies in lysosomal storage diseases and the role of immune tolerance induction. Mol Genet Metab 117, 66-83 (2016).
2. Nicolino, M. et al. Clinical outcomes after long-term treatment with alglucosidase alfa in infants and children with advanced Pompe disease. Genetics in Medicine 11, 210-219 (2009).
3. Kishnani, P. S. et al. Early Treatment with Alglucosidase Alfa Prolongs Long Term Survival of Infants with Pompe Disease. Pediatr Res 66, 329-335 (2009).
4. HGMD® home page. http://www.hgmd.cf.ac.uk/ac/index.php.
5. Peruzzo, P., Pavan, E. & Dardis, A. Molecular genetics of Pompe disease: a comprehensive overview. Ann Transl Med 7, (2019).
6. Kroos, M. et al. Update of the Pompe disease mutation database with 107 sequence variants and a format for severity rating. Hum Mutat 29, E13-26 (2008).
7. Martiniuk, F., Tzall, S. & Chen, A. Recombinant Human Acid α-Glucosidase Generated in Bacteria: Antigenic, but Enzymatically Inactive. DNA and Cell Biology 11, 701-706 (1992).
8. Hermans, M. M., Wisselaar, H. A., Kroos, M. A., Oostra, B. A. & Reuser, A. J. Human lysosomal alpha-glucosidase: functional characterization of the glycosylation sites. Biochem J 289, 681-686 (1993).
9. Roig-Zamboni, V. et al. Structure of human lysosomal acid α-glucosidase—a guide for the treatment of Pompe disease. Nat Commun 8, 1111 (2017).
10. Park, H. et al. Four unreported types of glycans containing mannose-6-phosphate are heterogeneously attached at three sites (including newly found Asn 233) to recombinant human acid alpha-glucosidase that is the only approved treatment for Pompe disease. Biochem Biophys Res Commun 495, 2418-2424 (2018).
11. van der Horst, G. T., Hoefsloot, E. H., Kroos, M. A. & Reuser, A. J. Cell-free translation of human lysosomal alpha-glucosidase: evidence for reduced precursor synthesis in an adult patient with glycogenosis type II. Biochim Biophys Acta 910, 123-129 (1987).
12. Hasilik, A. & Neufeld, E. F. Biosynthesis of lysosomal enzymes in fibroblasts. Synthesis as precursors of higher molecular weight. J Biol Chem 255, 4937-4945 (1980).
13. Hasilik, A. & Neufeld, E. F. Biosynthesis of lysosomal enzymes in fibroblasts. Phosphorylation of mannose residues. J Biol Chem 255, 4946-4950 (1980).
14. Hasilik, A., Waheed, A. & von Figura, K. Enzymatic phosphorylation of lysosomal enzymes in the presence of UDP-N-acetylglucosamine. Absence of the activity in I-cell fibroblasts. Biochemical and Biophysical Research Communications 98, 761-767 (1981).
15. Reuser, A. J., Kroos, M., Oude Elferink, R. P. & Tager, J. M. Defects in synthesis, phosphorylation, and maturation of acid alpha-glucosidase in glycogenosis type II. J Biol Chem 260, 8336-8341 (1985).
16. Moreland, R. J. et al. Lysosomal Acid α-Glucosidase Consists of Four Different Peptides Processed from a Single Chain Precursor. J. Biol. Chem. 280, 6780-6791 (2005).
17. Van Hove, J. L., Yang, H. W., Wu, J. Y., Brady, R. O. & Chen, Y. T. High-level production of recombinant human lysosomal acid alpha-glucosidase in Chinese hamster ovary cells which targets to heart muscle and corrects glycogen accumulation in fibroblasts from patients with Pompe disease. Proc Natl Acad Sci USA 93, 65-70 (1996).
18. Needham, M. et al. Prevalence of sporadic inclusion body myositis and factors contributing to delayed diagnosis. J Clin Neurosci 15, 1350-1353 (2008).
19. Askanas, V., Engel, W. K. & Nogalska, A. Pathogenic Considerations in Sporadic Inclusion-Body Myositis, a Degenerative Muscle Disease Associated With Aging and Abnormalities of Myoproteostasis. Journal of Neuropathology & Experimental Neurology 71, 680-693 (2012).
20. Nogalska, A., D'Agostino, C., Terracciano, C., Engel, W. K. & Askanas, V. Impaired Autophagy in Sporadic Inclusion-Body Myositis and in Endoplasmic Reticulum Stress-Provoked Cultured Human Muscle Fibers. The American Journal of Pathology 177, 1377-1387 (2010).
21. Kalmar, B. et al. The effect of treatment with BRX-220, a co-inducer of heat shock proteins, on sensory fibers of the rat following peripheral nerve injury. Exp Neurol 184, 636-647 (2003).
22. Kalmar, B. & Greensmith, L. Activation of the heat shock response in a primary cellular model of motoneuron neurodegeneration-evidence for neuroprotective and neurotoxic effects. Cell Mol Biol Lett 14, 319-335 (2009).
23. Ahmed, M. et al. Targeting protein homeostasis in sporadic inclusion body myositis. Science Translational Medicine 8, 331ra41-331ra41 (2016).
24. Fog, C. K. et al. The heat shock protein amplifier arimoclomol improves refolding, maturation and lysosomal activity of glucocerebrosidase. EBioMedicine 38, 142-153 (2018).
25. Kieran, D. et al. Treatment with arimoclomol, a coinducer of heat shock proteins, delays disease progression in ALS mice. Nature Medicine 10, 402-405 (2004).
26. Needham, M. & Mastaglia, F. L. Sporadic inclusion body myositis: A review of recent clinical advances and current approaches to diagnosis and treatment. Clin Neurophysiol 127, 1764-1773 (2016).
27. Mastaglia, F. L. & Needham, M. Inclusion body myositis: a review of clinical and genetic aspects, diagnostic criteria and therapeutic approaches. J Clin Neurosci 22, 6-13 (2015).
28. Greenberg, S. A. Inclusion body myositis: clinical features and pathogenesis. Nature Reviews Rheumatology 15, 257-272 (2019).
29. Lee, A. Y., Gulnik, S. V. & Erickson, J. W. Conformational switching in an aspartic proteinase. Nat Struct Mol Biol 5, 866-871 (1998).
30. Laurent-Matha, V., Derocq, D., Prebois, C., Katunuma, N. & Liaudet-Coopman, E. Processing of human cathepsin D is independent of its catalytic function and auto-activation: involvement of cathepsins Land B. J Biochem 139, 363-371 (2006).
31. Hasilik, A., von FIGURA, K., Conzelmann, E., Nehrkorn, H. & Sandhoff, K. Lysosomal Enzyme Precursors in Human Fibroblasts. European Journal of Biochemistry 125, 317-321 (1982).
32. Metcalf, P. & Fusek, M. Two crystal structures for cathepsin D: the lysosomal targeting signal and active site. EMBO J 12, 1293-1302 (1993).
33. Woessner, J. F. & Shamberger, R. J. Purification and Properties of Cathepsin D from Bovine Uterus. J. Biol. Chem. 246, 1951-1960 (1971).
34. Banay-Schwartz, M. et al. The pH dependence of breakdown of various purified brain proteins by cathepsin D preparations. Neurochemistry International 7, 607-614 (1985).
35. Cunningham, M. & Tang, J. Purification and properties of cathepsin D from porcine spleen. J. Biol.

### Chem. 251, 4528-4536 (1976).

36. Derocq, D. et al. Cathepsin D is partly endocytosed by the LRP1 receptor and inhibits LRP1-regulated intramembrane proteolysis. Oncogene 31, 3202-3212 (2012).
37. Markmann, S. et al. Lrp1/LDL Receptor Play Critical Roles in Mannose 6-Phosphate-Independent Lysosomal Enzyme Targeting. Traffic 16, 743-759 (2015).
38. Canuel, M., Korkidakis, A., Konnyu, K. & Morales, C. R. Sortilin mediates the lysosomal targeting of cathepsins D and H. Biochem Biophys Res Commun 373, 292-297 (2008).
39. Boonen, M. et al. Cathepsin D and its newly identified transport receptor SEZ6L2 can modulate neurite outgrowth. J Cell Sci 129, 557-568 (2016).
40. Peters, P. J., Neefjes, J. J., Oorschot, V., Ploegh, H. L. & Geuze, H. J. Segregation of MHC class II molecules from MHC class I molecules in the Golgi complex for transport to lysosomal compartments. Nature 349, 669-676 (1991).
41. Arnold, D. et al. Substrate specificity of cathepsins D and E determined by N-terminal and C-terminal sequencing of peptide pools. Eur J Biochem 249, 171-179 (1997).
42. Sun, H. et al. Proteolytic Characteristics of Cathepsin D Related to the Recognition and Cleavage of Its Target Proteins. PLOS ONE 8, e65733 (2013).
43. Barrett, A. J., Woessner, J. F. & Rawlings, N. D. Handbook of Proteolytic Enzymes. (Elsevier, 2012).
44. Dreyer, R. N. et al. Processing of the pre-beta-amyloid protein by cathepsin D is enhanced by a familial Alzheimer's disease mutation. Eur J Biochem 224, 265-271 (1994).
45. Ladror, U. S., Snyder, S. W., Wang, G. T., Holzman, T. F. & Krafft, G. A. Cleavage at the amino and carboxyl termini of Alzheimer's amyloid-beta by cathepsin D. Journal of Biological Chemistry 269, 18422-18428 (1994).
46. Garcia, M. et al. Biological and clinical significance of cathepsin D in breast cancer metastasis. Stem Cells 14, 642-650 (1996).
47. Steinfeld, R. et al. Cathepsin D Deficiency Is Associated with a Human Neurodegenerative Disorder. Am J Hum Genet 78, 988-998 (2006).
48. Kousi, M., Lehesjoki, A.-E. & Mole, S. E. Update of the mutation spectrum and clinical correlations of over 360 mutations in eight genes that underlie the neuronal ceroid lipofuscinoses. Hum Mutat 33, 42-63 (2012).
49. Marques, A. R. A. et al. Enzyme replacement therapy with recombinant pro-CTSD (cathepsin D) corrects defective proteolysis and autophagy in neuronal ceroid lipofuscinosis. Autophagy 16, 811-825 (2020).
50. Rossi, M. et al. Long-term enzyme replacement therapy for pompe disease with recombinant human alpha-glucosidase derived from chinese hamster ovary cells. J Child Neurol 22, 565-573 (2007).
51. Van den Hout, H. et al. Recombinant human α-glucosidase from rabbit milk in Pompe patients. The Lancet 356, 397-398 (2000).
52. Meuris, L. et al. GlycoDelete engineering of mammalian cells simplifies N-glycosylation of recombinant proteins. Nat Biotech 32, 485-489 (2014).
53. Reuser, A. J. et al. Uptake and stability of human and bovine acid alpha-glucosidase in cultured fibroblasts and skeletal muscle cells from glycogenosis type II patients. Exp Cell Res 155, 178-189 (1984).
54. Ran, F. A. et al. Genome engineering using the CRISPR-Cas9 system. Nature protocols 8, 2281-2308 (2013).
55. Backliwal, G. et al. Valproic acid: A viable alternative to sodium butyrate for enhancing protein expression in mammalian cell cultures. Biotechnology and Bioengineering 101, 182-189 (2008).
56. Porto, C. et al. Pharmacological Enhancement of α-Glucosidase by the Allosteric Chaperone N-acetylcysteine. Molecular Therapy 20, 2201-2211 (2012).
57. Chen, R. et al. Glycoproteomics analysis of human liver tissue by combination of multiple enzyme digestion and hydrazide chemistry. J Proteome Res 8, 651-661 (2009).
58. Baldwin, E. T. et al. Crystal structures of native and inhibited forms of human cathepsin D: implications for lysosomal targeting and drug design. Proc. Natl. Acad. Sci. USA 90, 6796-6800 (1993).
59. Lewandrowski, U., Moebius, J., Walter, U. & Sickmann, A. Elucidation of N-glycosylation sites on human platelet proteins: a glycoproteomic approach. Mol Cell Proteomics 5, 226-233 (2006).
60. Knappskog, S. et al. The level of synthesis and secretion of Gaussia princeps luciferase in transfected CHO cells is heavily dependent on the choice of signal peptide. Journal of Biotechnology 128, 705-715 (2007).
61. Yarimizu, T., Nakamura, M., Hoshida, H. & Akada, R. Synthetic signal sequences that enable efficient secretory protein production in the yeast Kluyveromyces marxianus. Microbial Cell Factories 14, 20 (2015).
62. Cardone, M. et al. Abnormal mannose-6-phosphate receptor trafficking impairs recombinant alpha-glucosidase uptake in Pompe disease fibroblasts. PathoGenetics 1, 6 (2008).
63. Bosch-Morató, M. et al. Increased amyloid β-peptide uptake in skeletal muscle is induced by hyposialylation and may account for apoptosis in GNE myopathy. Oncotarget 7, 13354 (2016).
64. Britson, K. A., Black, A. D., Wagner, K. R. & Lloyd, T. E. Performing Human Skeletal Muscle Xenografts in Immunodeficient Mice. J Vis Exp (2019) doi:10.3791/59966.
65. Britson, K. A. et al. Loss of TDP-43 function and rimmed vacuoles persist after T cell depletion in a xenograft model of sporadic inclusion body myositis. bioRxiv 2021.04.09.439185 (2021) doi:10.1101/2021.04.09.439185.
66. Badadani, M. et al. VCP Associated Inclusion Body Myopathy and Paget Disease of Bone Knock-In Mouse Model Exhibits Tissue Pathology Typical of Human Disease. PLoS One 5, (2010).
67. Custer, S. K., Neumann, M., Lu, H., Wright, A. C. & Taylor, J. P. Transgenic mice expressing mutant forms VCP/p97 recapitulate the full spectrum of IBMPFD including degeneration in muscle, brain and bone. Hum. Mol. Genet. 19, 1741-1755 (2010).
68. Saftig, P. et al. Mice deficient for the lysosomal proteinase cathepsin D exhibit progressive atrophy of the intestinal mucosa and profound destruction of lymphoid cells. EMBO J 14, 3599-3608 (1995).
69. Bijvoet, A. G. et al. Generalized glycogen storage and cardiomegaly in a knockout mouse model of Pompe disease. Hum. Mol. Genet. 7, 53-62 (1998).
70. Bijvoet, A. G. A. et al. Human Acid α-Glucosidase from Rabbit Milk HasTherapeutic Effect in Mice with Glycogen Storage Disease Type II. Hum. Mol. Genet. 8, 2145-2153 (1999).
71. Tiels, P. et al. A bacterial glycosidase enables mannose-6-phosphate modification and improved cellular uptake of yeast-produced recombinant human lysosomal enzymes. Nature Biotechnology 30, 1225-1231 (2012).
72. Xavier, C. et al. Site-specific labeling of his-tagged Nanobodies with ⁹⁹mTc: a practical guide. Methods Mol Biol 911, 485-490 (2012).
73. Khanna, R. et al. The Pharmacological Chaperone AT2220 Increases the Specific Activity and Lysosomal Delivery of Mutant Acid Alphα-Glucosidase, and Promotes Glycogen Reduction in a Transgenic Mouse Model of Pompe Disease. PLoS One 9, (2014).
74. Byrne, B. J. et al. A study on the safety and efficacy of reveglucosidase alfa in patients with late-onset Pompe disease. Orphanet Journal of Rare Diseases 12, 144 (2017).
75. Xu, S. et al. Improved efficacy of a next-generation ERT in murine Pompe disease. JCIInsight 4, (2019).
76. Pena, L. D. M. et al. Safety, tolerability, pharmacokinetics, pharmacodynamics, and exploratory efficacy of the novel enzyme replacement therapy avalglucosidase alfa (neoGAA) in treatment-naïve and alglucosidase alfa-treated patients with late-onset Pompe disease: A phase 1, open-label, multicenter, multinational, ascending dose study. Neuromuscul Disord 29, 167-186 (2019).
77. Tian, W. et al. The glycosylation design space for recombinant lysosomal replacement enzymes produced in CHO cells. Nat Commun 10, 1-13 (2019).
78. Zhang, P., Sun, F., Liu, S. & Jiang, S. Anti-PEG antibodies in the clinic: Current issues and beyond PEGylation. J Control Release 244, 184-193 (2016).
79. Deduve C: FROM CYTASES TO LYSOSOMES. Fed Proc 1964, 23:1045-1049.
80. De Duve C, Pressman B C, Gianetto R, Wattiaux R, Appelmans F: Tissue fractionation studies. 6. Intracellular distribution patterns of enzymes in rat-liver tissue. Biochem J 1955, 60:604-617.
81. Hers H G: α-Glucosidase deficiency in generalized glycogen-storage disease (Pompe's disease). Biochem J 1963, 86:11-16.
82. Barton N W, Brady R O, Dambrosia J M, Di Bisceglie A M, Doppelt S H, Hill S C, Mankin H J, Murray G J, Parker R I, Argoff C E: Replacement therapy for inherited enzyme deficiency—macrophage-targeted glucocerebrosidase for Gaucher's disease. N Engl J Med 1991, 324:1464-1470.
83. Maga J A, Zhou J, Kambampati R, Peng S, Wang X, Bohnsack R N, Thomm A, Golata S, Tom P, Dahms N M, et al.: Glycosylation-independent Lysosomal Targeting of Acid α-Glucosidase Enhances Muscle Glycogen Clearance in Pompe Mice. J Biol Chem 2013, 288:1428-1438.
84. Zhu Y, Li X, Mcvie-Wylie A, Jiang C, Thurberg B L, Raben N, Mattaliano R J, Cheng S H: Carbohydrate-remodelled acid α-glucosidase with higher affinity for the cation-independent mannose 6-phosphate receptor demonstrates iM6PRoved delivery to muscles of Pompe mice. Biochem J 2005, 389:619-628.
85. Zhu Y, Jiang J-L, Gumlaw N K, Zhang J, Bercury S D, Ziegler R J, Lee K, Kudo M, Canfield W M, Edmunds T, et al.: Glycoengineered acid alpha-glucosidase with iM6PRoved efficacy at correcting the metabolic aberrations and motor function deficits in a mouse model of Pompe disease. Mol Ther 2009, 17:954-963.
86. Basile I, Da Silva A, El Cheikh K, GodefroyA, Daurat M, Harmois A, Perez M, Caillaud C, Charbonne H-V, Pau B, et al.: Efficient therapy for refractory Pompe disease by mannose 6-phosphate analogue grafting on acid α-glucosidase. J Control Release 2018, 269:15-23.
87. El Cheikh K, Basile I, Da Silva A, Bernon C, Cerutti P, Salgues F, Perez M, Maynadier M, Gary-Bobo M, Caillaud C, et al.: Design of Potent Mannose 6-Phosphate Analogues for the Functionalization of Lysosomal Enzymes To IM6PRove the Treatment of Pompe Disease. Angew Chem Int Ed Engl 2016, 55:14774-14777.
88. Deduve C: FROM CYTASES TO LYSOSOMES. Fed Proc 1964, 23:1045-1049.
89. De Duve C, Pressman B C, Gianetto R, Wattiaux R, Appelmans F: Tissue fractionation studies. 6. Intracellular distribution patterns of enzymes in rat-liver tissue. Biochem J 1955, 60:604-617.
90. Hers H G: α-Glucosidase deficiency in generalized glycogen-storage disease (Pompe's disease). Biochem J 1963, 86:11-16.
91. Barton N W, Brady R O, Dambrosia J M, Di Bisceglie A M, Doppelt S H, Hill S C, Mankin H J, Murray G J, Parker R I, Argoff C E: Replacement therapy for inherited enzyme deficiency—macrophage-targeted glucocerebrosidase for Gaucher's disease. N Engl J Med 1991, 324:1464-1470.
92. Maga J A, Zhou J, Kambampati R, Peng S, Wang X, Bohnsack R N, Thomm A, Golata S, Tom P, Dahms N M, et al.: Glycosylation-independent Lysosomal Targeting of Acid α-Glucosidase Enhances Muscle Glycogen Clearance in Pompe Mice. J Biol Chem 2013, 288:1428-1438.
93. Zhu Y, Li X, Mcvie-Wylie A, Jiang C, Thurberg B L, Raben N, Mattaliano R J, Cheng S H: Carbohydrate-remodelled acid α-glucosidase with higher affinity for the cation-independent mannose 6-phosphate receptor demonstrates iM6PRoved delivery to muscles of Pompe mice. Biochem J 2005, 389:619-628.
94. Zhu Y, Jiang J-L, Gumlaw N K, Zhang J, Bercury S D, Ziegler R J, Lee K, Kudo M, Canfield W M, Edmunds T, et al.: Glycoengineered acid alpha-glucosidase with iM6PRoved efficacy at correcting the metabolic aberrations and motor function deficits in a mouse model of Pompe disease. Mol Ther 2009, 17:954-963.
95. Basile I, Da Silva A, El Cheikh K, GodefroyA, Daurat M, Harmois A, Perez M, Caillaud C, Charbonne H-V, Pau B, et al.: Efficient therapy for refractory Pompe disease by mannose 6-phosphate analogue grafting on acid α-glucosidase. J Control Release 2018, 269:15-23.
96. El Cheikh K, Basile I, Da Silva A, Bernon C, Cerutti P, Salgues F, Perez M, Maynadier M, Gary-Bobo M, Caillaud C, et al.: Design of Potent Mannose 6-Phosphate Analogues for the Functionalization of Lysosomal Enzymes To IM6PRove the Treatment of Pompe Disease. Angew Chem Int Ed Engl 2016, 55:14774-14777.
97. Banik S M, Pedram K, Wisnovsky S, Ahn G, Riley N M, Bertozzi C R: Lysosome-targeting chimaeras for degradation of extracellular proteins. Nature 2020, 584:291-297.
98. Lin-Cereghino J, Wong W W, Xiong S, Giang W, Luong L T, Vu J, Johnson S D, Lin-Cereghino G P: Condensed protocol for competent cell preparation and transformation of the methylotrophic yeast Pichia pastoris. Biotechniques 2005, 38:44-48.
99. Roy A, Kucukural A, Zhang Y: I-TASSER: a unified platform for automated protein structure and function prediction. Nature protocols 2010, 5:725-738.
100. Yang J, Zhang Y: I-TASSER server: New development for protein structure and function predictions. Nucleic Acids Research 2015, 43:W174-W181.
101. Schrödinger, LLC: The {PyMOL}Molecular Graphics System, Version˜1.8. 2015.
102. Chen H J, Remmler J, Delaney J C, Messner D J, Lobel P: Mutational analysis of the cation-independent mannose 6-phosphate/insulin-like growth factor II receptor. A consensus casein kinase II site followed by 2 leucines near the carboxyl terminus is important for intracellular targeting of lysosomal enzymes. J Biol Chem 1993, 268:22338-22346.
103. Li W, Godzik A: Cd-hit: a fast program for clustering and comparing large sets of protein or nucleotide sequences. Bioinformatics 2006, 22:1658-1659.
104. Olson L J, Dahms N M, Kim J-J P: The N-terminal Carbohydrate Recognition Site of the Cation-independent Mannose 6-Phosphate Receptor. J Biol Chem 2004, 279:34000-34009.
105. Olson L J, Misra S K, Ishihara M, Battaile K P, Grant O C, Sood A, Woods R J, Kim J-J P, Tiemeyer M, Ren G, et al.: Allosteric regulation of lysosomal enzyme recognition by the cation-independent mannose 6-phosphate receptor. Communications Biology 2020, 3:1-15.
106. Pardon, et al. A general protocol for the generation of Nanobodies for structural biology. Nature Protocols 2014. 9: 674-693.
107. Borden, L. A., Einstein, R., Gabel, C. A. & Maxfield, F. R. J. Biol. Chem. 1990. 265, 8497-8504.
108. Epenetos, A. A., Snook, D., Durbin, H., Johnson, P. M. & Taylor-Papadimitriou, J. Cancer Res. 1986. 46, 3183-3191.
109. Schröter, C. et al. mAbs 7, 138-151 (2014).

1.-27. (canceled)

28. A means for specifically binding the extracellular N-terminal domains 1, 2 and/or 3 of the human cation-independent mannose-6-phosphate receptor (CI-M6PR), wherein the means shows internalization upon binding CI-M6PR-expressing cells.

29. The means according to claim 28, wherein the means is an immunoglobulin-single-variable domain (ISVD).

30. The means of claim 28, wherein the means specifically binds an epitope comprising the amino acid residues Lys191, Gly194, Ala195, Tyr196, Leu197, Phe208, Arg219, Gln224, Leu225, Ile297, Lys357, Gly408, Asp409, Asn431, Glu433, and Phe457 as set forth in SEQ ID NO:20.

31. The means of claim 28, wherein the means specifically binds an epitope comprising the amino acid residues Lys59, Asn60, Met85, Asp87, Lys89, Ala146, Thr147, and Glu148, and Asp118 or Gln119, as set forth in SEQ ID NO:20.

32. The means of claim 28, wherein the means specifically binds to CI-M6PR via the paratope comprising residues 32, 52-57, 100-103, 108 as set forth in SEQ ID NO:8.

33. The means of claim 28, wherein the means specifically binds to CI-M6PR via the paratope comprising residues 31, 33, 35, 53, 54, 56, 57, 96, 104 as set forth in SEQ ID NO:7, or the paratope comprising residues 31-35, 50, 52-57, 96-98 as set forth in SEQ ID NO:71.

34. The means of claim 28, wherein the ISVD comprises 4 framework regions (FR) and 3 complementarity-determining regions (CDR) according to the following formula (1): FR1-CDR1-FR2-CDR2-FR3-CDR3-FR4 (1), and the CDR1, CDR2 and CDR3 regions are selected from those CDR1, CDR2 and CDR3 regions of a sequence selected from the group of sequences of SEQ ID NO: 1, 5, 7, 8, 71, or 73, wherein the CDR regions are annotated according to Kabat, MacCallum, IMGT, AbM, or Chothia.

35. The ISVD of claim 34, wherein said ISVD comprises a CDR1 sequence selected from SEQ ID NO:103-105, or 107-109, a CDR2 sequence selected from SEQ ID NO:110-112, or 114-116, and a CDR3 sequence selected from SEQ ID NO:117-119, or 121-123.

36. The means of claim 28, wherein means comprises a sequence selected from the group of sequences of SEQ ID NO: 1, 5, 7, 8, 71, or 73, or a sequence with at least 85% amino acid identity thereof, or a humanized variant thereof, such as any one of SEQ ID NO: 93-102.

37. The means of claim 28, wherein the means is comprised in a multi-specific or a multivalent binding agent.

38. The means of claim 37, further comprising a binding agent specifically binding a cell surface or extracellular molecule.

39. The means of claim 28, wherein the means is fused directly or via a linker to an enzyme.

40. The means of claim 39, wherein the enzyme is a lysosome localized enzyme.

41. The means of claim 39, wherein the enzyme is acid alfa-glucosidase or a functional homologue thereof, or is Cathepsin D or a functional homologue thereof.

42. The means of claim 41, wherein the fusion protein comprises a sequence selected from the group of sequences of SEQ ID NO:26-33 or a functional homologue with at least 90% identity thereof.

43. The means of claim 1, where the means further comprises a detectable label or a tag.

44. A nucleic acid molecule encoding the means of claim 29.

45. A method of producing the means of claim 29, the method comprising the steps of:
a. Introducing a nucleic acid molecule them means into a host cell, and
b. Isolating the CI-M6PR-specific binding agent from the medium.

46. The method according claim 45, wherein the host cell is a Glycodelete cell.

47. The means of claim 28, wherein the means comprises N-glycan structures wherein one or more glycans are present is selected from the group of a single GlcNAc, a GalGlcNAc and a SiaGalGlcNAc.

48. A method to treat a lysosomal storage disease in a subject in need thereof, the method comprising: administering to a subject suspected of having a lysosomal disease the means of claim 39.


SEQUENCE LISTING (139 sequences)

SEQ ID NO: 1 - protein, 120 aa, Artificial Sequence
  VHH1
QVQLQESGGGLVQPGGSLRLSCVASGFTFDRYWMNWLRQIPGKEIEWVSTINTGGTGTYYDDSVKGRFSISRDDAKNTLYLTMNSLKTEDTAVYYCARGATYYRGNSAIRGQGTQVTVSS

SEQ ID NO: 2 - protein, 118 aa, Artificial Sequence
  VHH2
QVQLQESGGGLVQPGGSLRLSCVFSGRTFMSYDVGWFRQAPGKERELLSSITSSSSSPYYAESVKGRFAVSRDGAKKTVYLRMNSLEPEDTAVYYCAARENWRNDWQYWGQTQVTVSS

SEQ ID NO: 3 - protein, 119 aa, Artificial Sequence
  VHH3
QVQLQESGGGLVQPGGSLRLSCAAFGLNFKNFDMIWFRQAPGEERDIVAAISSSSSTIWYEDSVKGRFTISRDNAKNTVYLQMNSLKPEDTAVYYCAAALTSWRRPDYWGQGTQVTVSS

SEQ ID NO: 4 - protein, 123 aa, Artificial Sequence
  VHH4
QVQLQESGGGLVQAGGSLRLSCAASGRTFSNYVMGWFRQAPGKEREFVGSISQGGKMTYYPDSVKGRFTISRDNAKNTMYLQMDGLKPEDTAVYYCAVSRGYPMVRVSEVDYWGQGTQVTVSS

SEQ ID NO: 5 - protein, 134 aa, Artificial Sequence
  VHH5
QVQLQESGGGLVQAGGSLKLSCAAAGRTFSRLAMGWFRQAPGKEREFVAAISENGDSIHYSDSVKGRFAVSRDNAKNTVYLQMNSLKPEDTAIYYCAADRAAYYCSGSGCYPSRAPAAASYDYWGQGTQVTVSS

SEQ ID NO: 6 - protein, 112 aa, Artificial Sequence
  VHH6
QVQLQESGGGLVQAGGSLRLSCAVSGSISSIGGMGWFRQAPGKQREFVAVILNNSGMTSYADSVKGRFTISRGNSKNTMYLQMNSLKPEDTAVYYCNYSGYWGQGTQVTVSS

SEQ ID NO: 7 - protein, 116 aa, Artificial Sequence
  VHH7
QVQLQESGGGLVQPGGSLRLSCAASGIIFSDNRMDWYRQAPGKQREWVATLASYGWKTYADSVKGRFTISRDNTKNTVYLRMNSLKPEDTAVYYCKASSPVLNDIWGQGTQVTVSS

SEQ ID NO: 8 - protein, 123 aa, Artificial Sequence
  VHH8
QVQLQESGGGLVQAGGSLRLSCEASGRTFNTYNWGWFRQAPGKEREFVAAIRWSSSKTSYADSVKGRFTISRDNAKNTIYLQMNSLKPEDTAVYYCAASIVDFTTNPSHFGSWGQGTQVTVSS

SEQ ID NO: 9 - protein, 113 aa, Artificial Sequence
  VHH9
QVQLQESGGGLVQAGGSLRLSCAASGTILSIKTMGWYRQAPGKEREWVATITSSSIPMYADSVKGRFTISRDDAKNTVYLQMNSLKPEDTGVYYCNAPWTDYWGQGTQVTVSS

SEQ ID NO: 10 - protein, 127 aa, Artificial Sequence
  VHH10
QVQLQESGGGSVQAGGSLRLSCAPAGRTFSRIRMGWFRQAPGKEREFVAAISSGGLSYYADSMKGRFTISRDNAKNTVLLQMNSLEPDDTAVYYCAAVLNDYIDDYVARNPTLYDYWGQGTQVTVSS

SEQ ID NO: 11 - protein, 124 aa, Artificial Sequence
  VHH11
QVQLQESGGGLVQAGDSLKLSCTASGPTFSEITGGWFRQVPGKEREFVASISSTGITTYYTDSVKGRFTVSRDNAKNTMYLQMNSLKPEDTGVYYCAARSTSRSIAITVGYDYWGQGTQVTVSS

SEQ ID NO: 12 - DNA, 17 bp, Artificial Sequence
  primer
aagaacaagccgccacc

SEQ ID NO: 13 - DNA, 22 bp, Artificial Sequence
  primer
gctctccctattgtgaagtcgc

SEQ ID NO: 14 - DNA, 20 bp, Artificial Sequence
  Primer
aagaacaagccgccaccatg

SEQ ID NO: 15 - DNA, 24 bp, Artificial Sequence
  primer
gtgcgacttcacaatagggagagc

SEQ ID NO: 16 - DNA, 33 bp, Artificial Sequence
  primer
gggctcttcaaggcaggtgcagctgcaggagtc

SEQ ID NO: 17 - DNA, 54 bp, Artificial Sequence
  primer
gggctcttcaagtctagtgatggtgatggtggtggctggagacggtgacctggg

SEQ ID NO: 18 - DNA, 29 bp, Artificial Sequence
  primer
gatgtgcagctgcaggagtctggrggagg

SEQ ID NO: 19 - DNA, 34 bp, Artificial Sequence
  primer
ctagtgcggccgctgaggagacggtgacctgggt

SEQ ID NO: 20 - protein, 2491 aa, Homo sapiens
MGAAAGRSPHLGPAPARRPQRSLLLLQLLLLVAAPGSTQAQAAPFPELCSYTWEAVDTKNNVLYKINICGSVDIVQCGPSSAVCMHDLKTRTYHSVGDSVLRSATRSLLEFNTTVSCDQQGTNHRVQSSIAFLCGKTLGTPEFVTATECVHYFEWRTTAACKKDIFKANKEVPCYVFDEELRKHDLNPLIKLSGAYLVDDSDPDTSLFINVCRDIDTLRDPGSQLRACPPGTAACLVRGHQAFDVGQPRDGLKLVRKDRLVLSYVREEAGKLDFCDGHSPAVTITFVCPSERREGTIPKLTAKSNCRYEIEWITEYACHRDYLESKTCSLSGEQQDVSIDLTPLAQSGGSSYISDGKEYLFYLNVCGETEIQFCNKKQAAVCQVKKSDTSQVKAAGRYHNQTLRYSDGDLTLIYFGGDECSSGFQRMSVINFECNKTAGNDGKGTPVFTGEVDCTYFFTWDTEYACVKEKEDLLCGATDGKKRYDLSALVRHAEPEQNWEAVDGSQTETEKKHFFINICHRVLQEGKARGCPEDAAVCAVDKNGSKNLGKFISSPMKEKGNIQLSYSDGDDCGHGKKIKTNITLVCKPGDLESAPVLRTSGEGGCFYEFEWHTAAACVLSKTEGENCTVFDSQAGFSFDLSPLTKKNGAYKVETKKYDFYINVCGPVSVSPCQPDSGACQVAKSDEKTWNLGLSNAKLSYYDGMIQLNYRGGTPYNNERHTPRATLITFLCDRDAGVGFPEYQEEDNSTYNFRWYTSYACPEEPLECVVTDPSTLEQYDLSSLAKSEGGLGGNWYAMDNSGEHVTWRKYYINVCRPLNPVPGCNRYASACQMKYEKDQGSFTEVVSISNLGMAKTGPVVEDSGSLLLEYVNGSACTTSDGRQTTYTTRIHLVCSRGRLNSHPIFSLNWECVVSFLWNTEAACPIQTTTDTDQACSIRDPNSGFVFNLNPLNSSQGYNVSGIGKIFMFNVCGTMPVCGTILGKPASGCEAETQTEELKNWKPARPVGIEKSLQLSTEGFITLTYKGPLSAKGTADAFIVRFVCNDDVYSGPLKFLHQDIDSGQGIRNTYFEFETALACVPSPVDCQVTDLAGNEYDLTGLSTVRKPWTAVDTSVDGRKRTFYLSVCNPLPYIPGCQGSAVGSCLVSEGNSWNLGVVQMSPQAAANGSLSIMYVNGDKCGNQRFSTRITFECAQISGSPAFQLQDGCEYVFIWRTVEACPVVRVEGDNCEVKDPRHGNLYDLKPLGLNDTIVSAGEYTYYFRVCGKLSSDVCPTSDKSKVVSSCQEKREPQGFHKVAGLLTQKLTYENGLLKMNFTGGDTCHKVYQRSTAIFFYCDRGTQRPVFLKETSDCSYLFEWRTQYACPPFDLTECSFKDGAGNSFDLSSLSRYSDNWEAITGTGDPEHYLINVCKSLAPQAGTEPCPPEAAACLLGGSKPVNLGRVRDGPQWRDGIIVLKYVDGDLCPDGIRKKSTTIRFTCSESQVNSRPMFISAVEDCEYTFAWPTATACPMKSNEHDDCQVTNPSTGHLFDLSSLSGRAGFTAAYSEKGLVYMSICGENENCPPGVGACFGQTRISVGKANKRLRYVDQVLQLVYKDGSPCPSKSGLSYKSVISFVCRPEAGPTNRPMLISLDKQTCTLFFSWHTPLACEQATECSVRNGSSIVDLSPLIHRTGGYEAYDESEDDASDTNPDFYINICQPLNPMHGVPCPAGAAVCKVPIDGPPIDIGRVAGPPILNPIANEIYLNFESSTPCLADKHFNYTSLIAFHCKRGVSMGTPKLLRTSECDFVFEWETPVVCPDEVRMDGCTLTDEQLLYSFNLSSLSTSTFKVTRDSRTYSVGVCTFAVGPEQGGCKDGGVCLLSGTKGASFGRLQSMKLDYRHQDEAVVLSYVNGDRCPPETDDGVPCVFPFIFNGKSYEECIIESRAKLWCSTTADYDRDHEWGFCRHSNSYRTSSIIFKCDEDEDIGRPQVFSEVRGCDVTFEWKTKVVCPPKKLECKFVQKHKTYDLRLLSSLTGSWSLVHNGVSYYINLCQKIYKGPLGCSERASICRRTTTGDVQVLGLVHTQKLGVIGDKVVVTYSKGYPCGGNKTASSVIELTCTKTVGRPAFKRFDIDSCTYYFSWDSRAACAVKPQEVQMVNGTITNPINGKSFSLGDIYFKLFRASGDMRTNGDNYLYEIQLSSITSSRNPACSGANICQVKPNDQHFSRKVGTSDKTKYYLQDGDLDVVFASSSKCGKDKTKSVSSTIFFHCDPLVEDGIPEFSHETADCQYLFSWYTSAVCPLGVGFDSENPGDDGQMHKGLSERSQAVGAVLSLLLVALTCCLLALLLYKKERRETVISKLTTCCRRSSNVSYKYSKVNKEEETDENETEWLMEEIQLPPPRQGKEGQENGHITTKSVKALSSLHGDDQDSEDEVLTIPEVKVHSGRGAGAESSHPVRNAQSNALQEREDDRVGLVRGEKARKGKSSSAQQKTVSSTKLVSFHDDSDEDLLHI

SEQ ID NO: 21 - protein, 2483 aa, Mus musculus
MRAVQLGPVPSGPRVALLPPLLLLLLLAAAGSAQAQAVDLDALCSYTWEAVDSKNNAVYKINVCGNVGISSCGPTSAICMCDLKTENCRSVGDSLLRSSARSLLEFNTTMGCQPSDSQHRIQTSITFLCGKTLGTPEFVTATDCVHYFEWRTTAACKKDIFKADKEVPCYAFDDKLQKHDLNPLIKLNGGYLVDDSDPDTSLFINVCRDIDSLRDPSTQLRVCPAGTAACLLKGNQAFDVGRPKEGLKLLSKDRLVLTYVKEEGEKPDFCNGHSPAVTVTFVCPSERREGTIPKLTAKSNCRYEVEWITEYACHRDYLQSESCSLSSEQHDITIDLSPLAQYGGSPYVSDGREYTFFINVCGDTKVSLCNNKEAAVCQEKKADSTQVKIAGRHQNQTLRYSDGDLTLIYSGGDECSSGFQRMSVINFECNKTAGKDGRGEPVFTGEVDCTYFFTWDTKYACIKEKEDLLCGAINGKKRYDLSVLARHSESEQNWEAVDGSQAESEKYFFINVCHRVLQEGKARNCPEDAAVCAVDKNGSKNLGKFVSSPTKEKGHIQLSYTDGDDCGSDKKISTNITLVCKPGDLESAPVLRAARSDGCFYEFEWHTAAACVLSKTEGENCTVLDAQAGFSFDLSLLTKKNGAYKVETEKYDFYINVCGPVSMDPCQSNSGACQVAKSGKSWNLGLSSTKLTYYDGMIQLSYRNGTPYNNEKHTPRATLITFLCDRDAGVGFPEYQEEDNSTYNFRWYTSYACPEEPLECMVTDPSMMEQYDLSSLVKSEGGSGGNWYAMENSREHVTRRKYYLNVCRPLNPVPGCDRYASACQMKYENHEGSLAETVSISNLGVAKIGPVVEESGSLLLEYVNGSACTTSDGQLTTYSTRIHLVCGRGFMNSHPIFTFNWECVVSFLWNTEAACPIQTITETDQACSIRDPSSGFVFNLSPLNDSAQGHVVLGIGKTFVFNICGAMPACGTVAGKPAYGCEAETQIEDIKDLRPQRPVGMERSLQLSAEGFLTLTYKGSSPSDRGTAFIIRFICNDDIYPGAPKFLHQDIDSTRGIRNTYFEFETALACTPSLVDCQVTDPAGNEYDLSALSMVRKPWTAVDTSAYGKRRHFYLSVCNPLPYIPGCHGIALGSCMVSEDNSFNLGVVQISPQATGNGSLSILYVNGDRCGDQRFSTRIVFECAQTSGSPMFQFVNNCEYVFVWRTVEACPVIREEGDNCQVKDPRHGNLYDLKPLGLNDTIVSVGEYTYYLRVCGKLSSDVCSAHDGSKAVSSCQEKKGPQGFQKVAGLLSQKLTFENGLLKMNYTGGDTCHKVYQRSTTIYFYCDRTTQKPVFLKETSDCSYMFEWRTQYACPPFNVTECSVQDAAGNSIDLSSLSRYSDNWEAVTRTGATEHYLINVCKSLSPHAGTEPCPPEAAVCLLNGSKPVNLGKVRDGPQWTDGVTVLQYVDGDLCPDKIRRRSTIIRFTCSDNQVNSRPLFISAVQDCEYTFSWPTPSACPVKSNTHDDCQVTNPSTGHLFDLSSLSGRAGINASYSEKGLVFMSICEENENCGPGVGACFGQTRISVGKASKRLSYKDQVLQLVYENGSPCPSLSDLRYKSVISFVCRPEAGPTNRPMLISLDKQTCTLFFSWHTPLACEQATECTVRNGSSIIDLSPLIHRTGGYEAYDESEDDTSDTTPDFYINICQPLNPMHGVPCPAGASVCKVPVDGPPIDIGRVTGPPIFNPVANEVYLNFESSTHCLADRYMNYTSLITFHCKRGVSMGTPKLIRTNDCDFVFEWETPIVCPDEVKTQGCAVTDEQLLYSFNLTSLSTSTFKVTRDARTYSIGVCTAAAGLGQEGCKDGGVCLLSGNKGASFGRLASMQLDYRHQDEAVILSYVNGDPCPPETDDGEPCVFPFIYKGKSYDECVLEGRAKLWCSKTANYDRDHEWGFCRQTNSYRMSAIIFTCDESEDIGRPQVFSEDRGCEVTFEWKTKVVCPPKKMECKFVQKHKTYDLRLLSSLTGSWDFVHEGNSYFINLCQRVYKGPLDCSERASICKKSATGQVQVLGLVHTQKLEVIDETVIVTYSKGYPCGGNKTASSVIELTCAKTVGRPAFKRFDSVSCTYYFYWYSRAACAVRPQEVTMVNGTLTNPVTGKSFSLGEIYFKLFSASGDMRTNGDNYLYEIQLSSITSSSYPACAGANICQVKPNDQHFSRKVGTSDMTKYYVQDGDLDVVFTSSSKCGKDKTKSVSSTIFFHCDPLVKDGIPEFSHETADCQYLFSWYTSAVCPLGVDFEDESAGPEYKGLSERSQAVGAVLSLLLVALTGCLLALLLHKKERRETVINKLTSCCRRSSGVSYKYSKVSKEEETDENETEWLMEEIQVPAPRLGKDGQENGHITTKAVKAEALSSLHGDDQDSEDEVLTVPEVKVHSGRGAEVESSQPLRNPQRKVLKEREGERLGLVRGEKARKGKFRPGQRKPTAPAKLVSFHDDSDEDLLHI

SEQ ID NO: 22 - protein, 2499 aa, Bos taurus
MEAAAGRSSHLGPAPAGRPPRCPLLLQLQLLLLLLLLPPGWVPGAAGTQGAEFPELCSYTWEAVDTKNNMLYKINICGNMGVAQCGPSSAVCMHDLKTDSFHSVGDSLLKTASRSLLEFNTTVNCKQQNHKIQSSITFLCGKTLGTPEFVTATDCVHYFEWRTTAACKKNIFKANKEVPCYAFDRELKKHDLNPLIKTSGAYLVDDSDPDTSLFINVCRDIEVLRASSPQVRVCPTGAAACLVRGDRAFDVGRPQEGLKLVSNDRLVLSYVKEGAGQPDFCDGHSPAVTITFVCPSERREGTIPKLTAKSNCRFEIEWVTEYACHRDYLESRSCSLSSAQHDVAVDLQPLSRVEASDSLFYTSEADEYTYYLSICGGSQAPICNKKDAAVCQVKKADSTQVKVAGRPQNLTLRYSDGDLTLIYFGGEECSSGFQRMSVINFECNQTAGNNGRGAPVFTGEVDCTYFFTWDTKYACVHEKEALLCGVSDGKQRFDLSALARHSELEQNWEAVDGSQREAEKKHFFINICHRVLQTGQARGCPEDAAVCAVDKNGSKNLGRFISSPTREKGNIQLSYSDGDECGGGQKIITNITLMCKPGDLESAPVLTTSRADGCFYEFEWRTAAACVLSRTEGDNCTVFDSQAGFSFDLTPLTKKDAYKVETDKYEFHINVCGPVSVGACPPDSGACQVSRSDRKSWNLGRSNAKLSYYDGMIQLTYRDGTPYNNEKRTPRATLITFLCDRDAGVGFPEYQEEDNSTYNFRWYTSYACPEEPLECIVTDPVTLDQYDLSRLAKSEGGPGGNWYSLDNGGARSTWRKYYINVCRPLNPVPGCDRYASACQMKYQGEQGSYSETVSISNLGVAKTGPMVEDSGSLLLEYVNGSACTTSDQRRTTYTTRIHLVCSTGSLYTHPIFSLNWECVVSFLWNTAAACPIRITTDIDQVCSIKDPNSGYVFDLNPLNNSRGYVVLGIGKTFLFNVCGDMPACGTLDGKPASGCEAEVQMDDMKTLKPGRLVGLEKSLQLSTEGFITLNYTGLPSHPNGRADAFIIRFVCNDDVYPGTPKFLHQDIDSSLGIRDTFFEFETALACVPSPVDCQVTDPAGNEYDLSGLSKARKPWTAVDTFDEGKKRTFYLSVCTPLPYIPGCHGTAVGCCLVTEDSKLNLGVVQISPQVGANGSLSLVYVNGDKCKNQRFSTRINLECAHTTGSPTFQLQNDCEYVFLWRTVEACPVVRAEGDYCEVRDPRHGNLYNLIPLGLNDTVVRAGEYTYYFRVCGELTSGVCPTSDKSKVISSCQEKRGPQGFQKVAGLFNQKLTYENGVLKMNYTGGDTCHKVYQRSTTIFFYCDRSTQAPVFLQETSDCSYLFEWRTQYACPPYDLTECSFKNEAGETYDLSSLSRYSDNWEAVTGTGSTEHYLINVCKSLSPQAGSDPCPPEAAVCLLGGPKPVNLGRVRDSPQWSQGLTLLKYVDGDLCPDQIRKKSTTIRFTCSESHVNSRPMFISAVEDCEYTFSWPTAAACAVKSNVHDDCQVTNPATGHLFDLSSLSGRAGFTAAYSEKGLVYLSVCGDNENCANGVGACFGQTRISVGKASKRLTYVDQVLQLVYEGGSPCPSKTGLSYKSVISFVCRPEVGPTNRPMLISLDKRTCTLFFSWHTPLACEQTTECSVRNGSSLIDLSPLIHRTGGYEAYDESEDDGSDTSPDFYINICQPLNPMHGLACPAGTAVCKVPVDGPPIDIGRVAGPPILNPIANEVYLNFESSTPCLADRHFNYTSLITFHCKRGVSMGTPKLLRTSVCDFVFEWETPLVCPDEVKTDGCSLTDEQLYYSFNLSSLSKSTFKVTRGPHTYSVGVCTAAAGLDEGGCKDGAVCLLSGSKGASFGRLASMKLDYRHQDEAVILSYANGDTCPPETEDGEPCVFPFVFNGKSYEECVVESRARLWCATTANYDRDHEWGFCKHSTSHRTSVIIFKCDEDADVGRPQVFSEVRGCEVTFEWKTKVVCPPKKMECKFVQKHRTYDLRLLSSLTGSWSFVHNGASYYINLCQKIYKGPQDCSERASVCKKSTSGEVQVLGLVHTQKLDVVDDRVIVTYSKGHYCGDNKTASAVIELTCAKTVGRPSFTRFDVDSCTYHFSWDSRAACAVKPQEVQMVNGTITNPANGRSFSLGDIYFKRFSASGDVRTNGDRYIYEIQLSSITGSSSPACSGASICQRKANDQHFSRKVGTSNQTRYYVQDGDLDVVFTSSSKCGKDKTKSVSSTIFFHCDPLVKDGIPEFSHETADCQYLFSWHTSAVCPLGAGFDEEIAGDDAQEHKGLSERSQAVGAVLSLLLVALTACLLTLLLYKKERREMVMSRLTNCCRRSANVSYKYSKVNKEEEADENETEWLMEEIQPPAPRPGKEGQENGHVAAKSVRAADTLSALHGDEQDSEDEVLTLPEVKVRPPGRAPGAEGGPPLRPLPRKAPPPLRADDRVGLVRGEPARRGRPRAAATPISTFHDDSDEDLLHV

SEQ ID NO: 23 - protein, 476 aa, Bos taurus
MEAAAGRSSHLGPAPAGRPPRCPLLLQLQLLLLLLLLPPGWVPGAAGTQGAEFPELCSYTWEAVDTKNNMLYKINICGNMGVAQCGPSSAVCMHDLKTDSFHSVGDSLLKTASRSLLEFNTTVNCKQQNHKIQSSITFLCGKTLGTPEFVTATDCVHYFEWRTTAACKKNIFKANKEVPCYAFDRELKKHDLNPLIKTSGAYLVDDSDPDTSLFINVCRDIEVLRASSPQVRVCPTGAAACLVRGDRAFDVGRPQEGLKLVSNDRLVLSYVKEGAGQPDFCDGHSPAVTITFVCPSERREGTIPKLTAKSNCRFEIEWVTEYACHRDYLESRSCSLSSAQHDVAVDLQPLSRVEASDSLFYTSEADEYTYYLSICGGSQAPICNKKDAAVCQVKKADSTQVKVAGRPQNLTLRYSDGDLTLIYFGGEECSSGFQRMSVINFECNQTAGNNGRGAPVFTGEVDCTYFFTWDTKYACV

SEQ ID NO: 24 - protein, 467 aa, Homo sapiens
MGAAAGRSPHLGPAPARRPQRSLLLLQLLLLVAAPGSTQAQAAPFPELCSYTWEAVDTKNNVLYKINICGSVDIVQCGPSSAVCMHDLKTRTYHSVGDSVLRSATRSLLEFNTTVSCDQQGTNHRVQSSIAFLCGKTLGTPEFVTATECVHYFEWRTTAACKKDIFKANKEVPCYVFDEELRKHDLNPLIKLSGAYLVDDSDPDTSLFINVCRDIDTLRDPGSQLRACPPGTAACLVRGHQAFDVGQPRDGLKLVRKDRLVLSYVREEAGKLDFCDGHSPAVTITFVCPSERREGTIPKLTAKSNCRYEIEWITEYACHRDYLESKTCSLSGEQQDVSIDLTPLAQSGGSSYISDGKEYLFYLNVCGETEIQFCNKKQAAVCQVKKSDTSQVKAAGRYHNQTLRYSDGDLTLIYFGGDECSSGFQRMSVINFECNKTAGNDGKGTPVFTGEVDCTYFFTWDTEYACV

SEQ ID NO: 25 - protein, 462 aa, Mus musculus
MRAVQLGPVPSGPRVALLPPLLLLLLLAAAGSAQAQAVDLDALCSYTWEAVDSKNNAVYKINVCGNVGISSCGPTSAICMCDLKTENCRSVGDSLLRSSARSLLEFNTTMGCQPSDSQHRIQTSITFLCGKTLGTPEFVTATDCVHYFEWRTTAACKKDIFKADKEVPCYAFDDKLQKHDLNPLIKLNGGYLVDDSDPDTSLFINVCRDIDSLRDPSTQLRVCPAGTAACLLKGNQAFDVGRPKEGLKLLSKDRLVLTYVKEEGEKPDFCNGHSPAVTVTFVCPSERREGTIPKLTAKSNCRYEVEWITEYACHRDYLQSESCSLSSEQHDITIDLSPLAQYGGSPYVSDGREYTFFINVCGDTKVSLCNNKEAAVCQEKKADSTQVKIAGRHQNQTLRYSDGDLTLIYSGGDECSSGFQRMSVINFECNKTAGKDGRGEPVFTGEVDCTYFFTWDTKYACI

SEQ ID NO: 26 - protein, 1094 aa, Artificial Sequence
  human acid alfa-glucosidase (hGAA) fused to CI-M6PR-VHH1 amino acid sequence (hGAA-G4S(3)-VHH1)
MGSMGVRHPPCSHRLLAVCALVSLATAALLGHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWCSGGGGGSGGGGSGGGGSLLQVQLQESGGGLVQPGGSLRLSCVASGFTFDRYWMNWLRQIPGKEIEWVSTINTGGTGTYYDDSVKGRFSISRDDAKNTLYLTMNSLKTEDTAVYYCARGATYYRGNSAIRGQGTQVTVSS

SEQ ID NO: 27 - protein, 1108 aa, Artificial Sequence
  human acid alfa-glucosidase (hGAA) fused to CI-M6PR-VHH5 amino acid sequence (hGAA-G4S(3)-VHH5)
MGSMGVRHPPCSHRLLAVCALVSLATAALLGHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWCSGGGGGSGGGGSGGGGSLLQVQLQESGGGLVQAGGSLKLSCAAAGRTFSRLAMGWFRQAPGKEREFVAAISENGDSIHYSDSVKGRFAVSRDNAKNTVYLQMNSLKPEDTAIYYCAADRAAYYCSGSGCYPSRAPAAASYDYWGQGTQVTVSS

SEQ ID NO: 28 - protein, 1090 aa, Artificial Sequence
  human acid alfa-glucosidase (hGAA) fused to CI-M6PR-VHH7 amino acid sequence (hGAA-G4S(3)-VHH7)
MGSMGVRHPPCSHRLLAVCALVSLATAALLGHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWCSGGGGGSGGGGSGGGGSLLQVQLQESGGGLVQPGGSLRLSCAASGIIFSDNRMDWYRQAPGKQREWVATLASYGWKTYADSVKGRFTISRDNTKNTVYLRMNSLKPEDTAVYYCKASSPVLNDIWGQGTQVTVSS

SEQ ID NO: 29 - protein, 1097 aa, Artificial Sequence
  human acid alfa-glucosidase (hGAA) fused to CI-M6PR-VHH8 amino acid sequence (hGAA-G4S(3)-VHH8)
MGSMGVRHPPCSHRLLAVCALVSLATAALLGHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWCSGGGGGSGGGGSGGGGSLLQVQLQESGGGLVQAGGSLRLSCEASGRTFNTYNWGWFRQAPGKEREFVAAIRWSSSKTSYADSVKGRFTISRDNAKNTIYLQMNSLKPEDTAVYYCAASIVDFTTNPSHFGSWGQGTQVTVSS

SEQ ID NO: 30 - protein, 553 aa, Artificial Sequence
  human Cathespin D (hCTSD) fused to CI-M6PR-VHH1 amino acid sequence (hCTSD-G4S(3)-VHH1)
MGSQPSSLLPLALCLLAAPASALVRIPLHKFTSIRRTMSEVGGSVEDLIAKGPVSKYSQAVPAVTEGPIPEVLKNYMDAQYYGEIGIGTPPQCFTVVFDTGSSNLWVPSIHCKLLDIACWIHHKYNSDKSSTYVKNGTSFDIHYGSGSLSGYLSQDTVSVPCQSASSASALGGVKVERQVFGEATKQPGITFIAAKFDGILGMAYPRISVNNVLPVFDNLMQQKLVDQNIFSFYLSRDPDAQPGGELMLGGTDSKYYKGSLSYLNVTRKAYWQVHLDQVEVASGLTLCKEGCEAIVDTGTSLMVGPVDEVRELQKAIGAVPLIQGEYMIPCEKVSTLPAITLKLGGKGYKLSPEDYTLKVSQAGKTLCLSGFMGMDIPPPSGPLWILGDVFIGRYYTVFDRDNNRVGFAEAARLSGGGGGSGGGGSGGGGSLLQVQLQESGGGLVQPGGSLRLSCVASGFTFDRYWMNWLRQIPGKEIEWVSTINTGGTGTYYDDSVKGRFSISRDDAKNTLYLTMNSLKTEDTAVYYCARGATYYRGNSAIRGQGTQVTVSS

SEQ ID NO: 31 - protein, 567 aa, Artificial Sequence
  human Cathespin D (hCTSD) fused to CI-M6PR-VHH5 amino acid sequence (hCTSD-G4S(3)-VHH5)
MGSQPSSLLPLALCLLAAPASALVRIPLHKFTSIRRTMSEVGGSVEDLIAKGPVSKYSQAVPAVTEGPIPEVLKNYMDAQYYGEIGIGTPPQCFTVVFDTGSSNLWVPSIHCKLLDIACWIHHKYNSDKSSTYVKNGTSFDIHYGSGSLSGYLSQDTVSVPCQSASSASALGGVKVERQVFGEATKQPGITFIAAKFDGILGMAYPRISVNNVLPVFDNLMQQKLVDQNIFSFYLSRDPDAQPGGELMLGGTDSKYYKGSLSYLNVTRKAYWQVHLDQVEVASGLTLCKEGCEAIVDTGTSLMVGPVDEVRELQKAIGAVPLIQGEYMIPCEKVSTLPAITLKLGGKGYKLSPEDYTLKVSQAGKTLCLSGFMGMDIPPPSGPLWILGDVFIGRYYTVFDRDNNRVGFAEAARLSGGGGGSGGGGSGGGGSLLQVQLQESGGGLVQAGGSLKLSCAAAGRTFSRLAMGWFRQAPGKEREFVAAISENGDSIHYSDSVKGRFAVSRDNAKNTVYLQMNSLKPEDTAIYYCAADRAAYYCSGSGCYPSRAPAAASYDYWGQGTQVTVSS

SEQ ID NO: 32 - protein, 549 aa, Artificial Sequence
  human Cathespin D (hCTSD) fused to CI-M6PR-VHH7 amino acid sequence (hCTSD-G4S(3)-VHH7)
MGSQPSSLLPLALCLLAAPASALVRIPLHKFTSIRRTMSEVGGSVEDLIAKGPVSKYSQAVPAVTEGPIPEVLKNYMDAQYYGEIGIGTPPQCFTVVFDTGSSNLWVPSIHCKLLDIACWIHHKYNSDKSSTYVKNGTSFDIHYGSGSLSGYLSQDTVSVPCQSASSASALGGVKVERQVFGEATKQPGITFIAAKFDGILGMAYPRISVNNVLPVFDNLMQQKLVDQNIFSFYLSRDPDAQPGGELMLGGTDSKYYKGSLSYLNVTRKAYWQVHLDQVEVASGLTLCKEGCEAIVDTGTSLMVGPVDEVRELQKAIGAVPLIQGEYMIPCEKVSTLPAITLKLGGKGYKLSPEDYTLKVSQAGKTLCLSGFMGMDIPPPSGPLWILGDVFIGRYYTVFDRDNNRVGFAEAARLSGGGGGSGGGGSGGGGSLLQVQLQESGGGLVQPGGSLRLSCAASGIIFSDNRMDWYRQAPGKQREWVATLASYGWKTYADSVKGRFTISRDNTKNTVYLRMNSLKPEDTAVYYCKASSPVLNDIWGQGTQVTVSS

SEQ ID NO: 33 - protein, 556 aa, Artificial Sequence
  human Cathespin D (hCTSD) fused to CI-M6PR-VHH8 amino acid sequence (hCTSD-G4S(3)-VHH8)
MGSQPSSLLPLALCLLAAPASALVRIPLHKFTSIRRTMSEVGGSVEDLIAKGPVSKYSQAVPAVTEGPIPEVLKNYMDAQYYGEIGIGTPPQCFTVVFDTGSSNLWVPSIHCKLLDIACWIHHKYNSDKSSTYVKNGTSFDIHYGSGSLSGYLSQDTVSVPCQSASSASALGGVKVERQVFGEATKQPGITFIAAKFDGILGMAYPRISVNNVLPVFDNLMQQKLVDQNIFSFYLSRDPDAQPGGELMLGGTDSKYYKGSLSYLNVTRKAYWQVHLDQVEVASGLTLCKEGCEAIVDTGTSLMVGPVDEVRELQKAIGAVPLIQGEYMIPCEKVSTLPAITLKLGGKGYKLSPEDYTLKVSQAGKTLCLSGFMGMDIPPPSGPLWILGDVFIGRYYTVFDRDNNRVGFAEAARLSGGGGGSGGGGSGGGGSLLQVQLQESGGGLVQAGGSLRLSCEASGRTFNTYNWGWFRQAPGKEREFVAAIRWSSSKTSYADSVKGRFTISRDNAKNTIYLQMNSLKPEDTAVYYCAASIVDFTTNPSHFGSWGQGTQVTVSS

SEQ ID NO: 34 - protein, 43 aa, Artificial Sequence
  FLAG(3)HIS(6) tag
AAAYPYDVPDYGSDYKDDDDKDYKDDDDKDYKDDDDKHHHHHH

SEQ ID NO: 35 - protein, 11 aa, Artificial Sequence
  amino acid sequence from synthetic substrate Mca-GKPILFFRL(dinitrophenyl)DR-NH2
GKPILFFRLDR

SEQ ID NO: 36 - DNA, 17 bp, Artificial Sequence
  primer
aagaacaagccgccacc

SEQ ID NO: 37 - DNA, 24 bp, Artificial Sequence
  primer
gctctccctattgtgaagtcgcac

SEQ ID NO: 38 - DNA, 20 bp, Artificial Sequence
  Primer
aagaacaagccgccaccatg

SEQ ID NO: 39 - DNA, 24 bp, Artificial Sequence
  primer
gtgcgacttcacaatagggagagc

SEQ ID NO: 40 - DNA, 25 bp, Artificial Sequence
  primer
caccgatggacgtgaacttgtgcag

SEQ ID NO: 41 - DNA, 25 bp, Artificial Sequence
  primer
aaacctgcacaagttcacgtccatc

SEQ ID NO: 42 - DNA, 25 bp, Artificial Sequence
  primer
caccggcaagttcacatctatccgt

SEQ ID NO: 43 - DNA, 25 bp, Artificial Sequence
  primer
aaacacggatagatgtgaacttgcc

SEQ ID NO: 44 - DNA, 26 bp, Artificial Sequence
  primer
agcctatggaaaaacgccagcaacgc

SEQ ID NO: 45 - DNA, 22 bp, Artificial Sequence
  primer
ggcaatgggttgccattcactg

SEQ ID NO: 46 - DNA, 22 bp, Artificial Sequence
  primer
gactctgagattccccaggggc

SEQ ID NO: 47 - DNA, 26 bp, Artificial Sequence
  primer
gtccatgtagttcttgagcacctcgg

SEQ ID NO: 48 - DNA, 24 bp, Artificial Sequence
  primer
caggagtttgtgacatgttgtggc

SEQ ID NO: 49 - DNA, 21 bp, Artificial Sequence
  primer
ctagaggcacctaggtgcatg

SEQ ID NO: 50 - DNA, 30 bp, Artificial Sequence
  primer
gcactcacatccaggtagtttttgagtaac

SEQ ID NO: 51 - DNA, 35 bp, Artificial Sequence
  primer
gcgccgtgtgtgagcacgacctgaaaacccggacc

SEQ ID NO: 52 - DNA, 43 bp, Artificial Sequence
  primer
caggtcgtgctcacacacggcgctagaaggtccacactgcacg

SEQ ID NO: 53 - DNA, 38 bp, Artificial Sequence
  primer
gtgtatgcacctcctgaaaacccggacctaccacagcg

SEQ ID NO: 54 - DNA, 37 bp, Artificial Sequence
  primer
gggttttcaggaggtgcatacacacggcgctagaagg

SEQ ID NO: 55 - DNA, 37 bp, Artificial Sequence
  primer
gcacgacctggacacccggacctaccacagcgtgggc

SEQ ID NO: 56 - DNA, 35 bp, Artificial Sequence
  primer
ggtccgggtgtccaggtcgtgcatacacacggcgc

SEQ ID NO: 57 - DNA, 35 bp, Artificial Sequence
  primer
cacccctgagcgtgtgaccgccacagagtgtgtgc

SEQ ID NO: 58 - DNA, 36 bp, Artificial Sequence
  primer
ggcggtcacacgctcaggggtgcccagtgtcttgcc

SEQ ID NO: 59 - DNA, 35 bp, Artificial Sequence
  primer
ccgccacattctgtgtgcactacttcgagtggcgg

SEQ ID NO: 60 - DNA, 38 bp, Artificial Sequence
  primer
gtagtgcacacagaatgtggcggtcacaaactcagggg

SEQ ID NO: 61 - DNA, 44 bp, Artificial Sequence
  primer
ccctctgatcttcctgagcggcgcctacctggtggacgacagcg

SEQ ID NO: 62 - DNA, 43 bp, Artificial Sequence
  primer
ggcgccgctcaggaagatcagagggttcagatcgtgcttccgc

SEQ ID NO: 63 - DNA, 42 bp, Artificial Sequence
  primer
gcggcgcctacgacgtggacgacagcgatcctgataccagcc

SEQ ID NO: 64 - DNA, 43 bp, Artificial Sequence
  primer
gctgtcgtccacgtcgtaggcgccgctcagcttgatcagaggg

SEQ ID NO: 65 - DNA, 38 bp, Artificial Sequence
  primer
cagcgacggctttctgaccctgatctactttggcggcg

SEQ ID NO: 66 - DNA, 36 bp, Artificial Sequence
  primer
cagggtcagaaagccgtcgctgtatctcagggtctg

SEQ ID NO: 67 - DNA, 40 bp, Artificial Sequence
  primer
gatcaatttccggtgtaacaagaccgccggcaacgacggc

SEQ ID NO: 68 - DNA, 40 bp, Artificial Sequence
  primer
ggtcttgttacaccggaaattgatcacgctcattctctgg

SEQ ID NO: 69 - DNA, 44 bp, Artificial Sequence
  primer
ggactgcacctacgagttcacctgggacacagagtatgcctgcg

SEQ ID NO: 70 - DNA, 34 bp, Artificial Sequence
  primer
cccaggtgaactcgtaggtgcagtccacctcgcc

SEQ ID NO: 71 - protein, 115 aa, Artificial Sequence
  VHH1H11
QVQLQESGGGLVQPGGSLRLSCAASGIIFSDNRMDWYRQAPGKQREWVATLASYGWKTYADSVKDRFTISRDNAKNTVNLYMNSLQPEDTAVVHHHYYCNSGQYWGQGTQVTVSS

SEQ ID NO: 72 - protein, 126 aa, Artificial Sequence
  VHH2H79
QVQLQESGGGQVQAGDSLGLSCAASGGAFSTYHMGWFRQAPGKQRELVAAITRGGSSSTKYVDSVKGRFTISRDNAKNTMYLQMNSLQPDDTAVYYCAASPQGKWNALVGGTPDYWGQGTQVTVSS

SEQ ID NO: 73 - protein, 128 aa, Artificial Sequence
  VHH1H52
QVQLQESGGGLVQPGGSLRLSCTASGFTWDSYVIGWFRQAPGKGREGVSCLDVDDGSIYYHDSAKGRFSISRDNAKNTVYLQMNSLKPEDTAVYYCAAVNRASMRFRRCLQVLRYDYWGQGTQVTVSS

SEQ ID NO: 74 - protein, 124 aa, Artificial Sequence
  VHH1H21
QVQLQESGGGLVQAGGSLRLSCVASGRTFSTLHMGWFHQAPGKERELVAAITRSGDTYYADFVKGRFTISRDNAKNTVYLQMISLKPEDTAVYYCAATGPGGWARLRAHEYDYWGQGTQVTVSS

SEQ ID NO: 75 - protein, 118 aa, Artificial Sequence
  VHH1H37
QVQLQESGGGLVQPGGSLRLTCAPSGRTYSTYWMAWFRQAPGKEREIVARINWSDGSTFYTESVKGRFTISGDNAKNTVYLQMTNLKPEDTAVYYCLARTVVSLDDYWGQGTQVTVSS

SEQ ID NO: 76 - protein, 118 aa, Artificial Sequence
  VHH2H74
QVQLQESGGGLVQAGGSLRLTCAPSGRTYSTYWMAWFRQAPGKEREIVARINWSDGSTFYTESVKGRFTISGDNAKNTVYLQMTNLKPEDTAVYYCLARTVVSLDDYWGQGTQVTVSS

SEQ ID NO: 77 - protein, 126 aa, Artificial Sequence
  VHH1H74
QVQLQESGGGLVQPGGSLRLACAASGFTLEDYSIGWFRQAPGKEREGVSCISSSDHSTYYADSVKGRFTISRDNAQNTVYLHMNSLKPEDTAVYYCAGRYSSLARIETNWHDYNYWGQGTQVTVSS

SEQ ID NO: 78 - protein, 128 aa, Artificial Sequence
  VHH1H44
QVQLQESGGGLVQAGGSLRLSCAASGRTFNNYAMGWFRQAPGKEREFVGVIDWSGGVTYYDNSVKGRFAISRDNAKNTVYLQMNSLKPEDTAVYYCAAGVSPRSGIWYYSRRNAYDYWGQGTQVTVSS

SEQ ID NO: 79 - protein, 113 aa, Artificial Sequence
  VHH2H60
QVQLQESGGGLVQPGGSLRLSCTASGMSSSTDTMTWYRQAPGKQRDLVAYITTTGVTNYADSVRGRFTISRDNAKNTVYLQMNSLKPEDTAVYYCRSAWNNYWGQGTQVTVSS

SEQ ID NO: 80 - protein, 124 aa, Artificial Sequence
  VHH1H73
QVQLQESGGGLVQAGGSLRLSCAASGRTFSNYDMGWFRQAPGAEREFVAYLSWSGGTTYYADSVKGRVTISRDNAKNTVYLQINSLKPEDTAVYYCAAGSAIVRITTTRDYHYWGQGTQVTVSS

SEQ ID NO: 81 - protein, 124 aa, Artificial Sequence
  VHH2H14
QVQLQESGGGLVQAGGSLRLSCAASGRTFSNYDMGWFRQAPGAEREFVAYVSWSGGTTYYADSVKGRVTISRDNAKNTVYLQMNSLRPEDTAVYYCAAGSQIVRITTTRDYHYWGQGTQVTVSS

SEQ ID NO: 82 - protein, 125 aa, Artificial Sequence
  VHH1H06
QVQLQESGGGLVQAGESLRLSCAASGNIGSIAIMGWYRQAPGKQRELVATIDGRSTNYADSVKGRFTISRDNAKNTLYLQMNSLKSDDTAVYYCAAARYYISLYYYRMQNNYDYWGQGTQVTVSS

SEQ ID NO: 83 - protein, 433 aa, Artificial Sequence
  CI-M6PR_M85E mutant
QAAPFPELCSYTWEAVDTKNNVLYKINICGSVDIVQCGPSSAVCEHDLKTRTYHSVGDSVLRSATRSLLEFNTTVSCDQQGTNHRVQSSIAFLCGKTLGTPEFVTATECVHYFEWRTTAACKKDIFKANKEVPCYVFDEELRKHDLNPLIKLSGAYLVDDSDPDTSLFINVCRDIDTLRDPGSQLRACPPGTAACLVRGHQAFDVGQPRDGLKVVRKDRLVLSYVREEAGKLDFCDGHSPAVTITFVCPSERREGTIPKLTAKSNCRYEIEWITEYACHRDYLESKTCSLSGEQQDVSIDLTPLAQSGGSSYISDGKEYLFYLNVCGETEIQFCNKKQAAVCQVKKSDTSQVKAAGRYHNQTLRYSDGDLTLIYFGGDECSSGFQRMSVINFECNKTAGNDGKGTPVFTGEVDCTYFFTWDTEYACVHHHHHH

SEQ ID NO: 84 - protein, 433 aa, Artificial Sequence
  CI-M6PR_D87L mutant
QAAPFPELCSYTWEAVDTKNNVLYKINICGSVDIVQCGPSSAVCMHDLKTRTYHSVGDSVLRSATRSLLEFNTTVSCDQQGTNHRVQSSIAFLCGKTLGTPEFVTATECVHYFEWRTTAACKKDIFKANKEVPCYVFDEELRKHDLNPLIKLSGAYLVDDSDPDTSLFINVCRDIDTLRDPGSQLRACPPGTAACLVRGHQAFDVGQPRDGLKVVRKDRLVLSYVREEAGKLDFCDGHSPAVTITFVCPSERREGTIPKLTAKSNCRYEIEWITEYACHRDYLESKTCSLSGEQQDVSIDLTPLAQSGGSSYISDGKEYLFYLNVCGETEIQFCNKKQAAVCQVKKSDTSQVKAAGRYHNQTLRYSDGDLTLIYFGGDECSSGFQRMSVINFECNKTAGNDGKGTPVFTGEVDCTYFFTWDTEYACVHHHHHH

SEQ ID NO: 85 - protein, 433 aa, Artificial Sequence
  CI-M6PR_K89D mutant
QAAPFPELCSYTWEAVDTKNNVLYKINICGSVDIVQCGPSSAVCMHDLDTRTYHSVGDSVLRSATRSLLEFNTTVSCDQQGTNHRVQSSIAFLCGKTLGTPEFVTATECVHYFEWRTTAACKKDIFKANKEVPCYVFDEELRKHDLNPLIKLSGAYLVDDSDPDTSLFINVCRDIDTLRDPGSQLRACPPGTAACLVRGHQAFDVGQPRDGLKVVRKDRLVLSYVREEAGKLDFCDGHSPAVTITFVCPSERREGTIPKLTAKSNCRYEIEWITEYACHRDYLESKTCSLSGEQQDVSIDLTPLAQSGGSSYISDGKEYLFYLNVCGETEIQFCNKKQAAVCQVKKSDTSQVKAAGRYHNQTLRYSDGDLTLIYFGGDECSSGFQRMSVINFECNKTAGNDGKGTPVFTGEVDCTYFFTWDTEYACVHHHHHH

SEQ ID NO: 86 - protein, 433 aa, Artificial Sequence
  CI-M6PR_F143R mutant
QAAPFPELCSYTWEAVDTKNNVLYKINICGSVDIVQCGPSSAVCMHDLKTRTYHSVGDSVLRSATRSLLEFNTTVSCDQQGTNHRVQSSIAFLCGKTLGTPERVTATECVHYFEWRTTAACKKDIFKANKEVPCYVFDEELRKHDLNPLIKLSGAYLVDDSDPDTSLFINVCRDIDTLRDPGSQLRACPPGTAACLVRGHQAFDVGQPRDGLKVVRKDRLVLSYVREEAGKLDFCDGHSPAVTITFVCPSERREGTIPKLTAKSNCRYEIEWITEYACHRDYLESKTCSLSGEQQDVSIDLTPLAQSGGSSYISDGKEYLFYLNVCGETEIQFCNKKQAAVCQVKKSDTSQVKAAGRYHNQTLRYSDGDLTLIYFGGDECSSGFQRMSVINFECNKTAGNDGKGTPVFTGEVDCTYFFTWDTEYACVHHHHHH

SEQ ID NO: 87 - protein, 433 aa, Artificial Sequence
  CI-M6PR_E148F mutant
QAAPFPELCSYTWEAVDTKNNVLYKINICGSVDIVQCGPSSAVCMHDLKTRTYHSVGDSVLRSATRSLLEFNTTVSCDQQGTNHRVQSSIAFLCGKTLGTPEFVTATFCVHYFEWRTTAACKKDIFKANKEVPCYVFDEELRKHDLNPLIKLSGAYLVDDSDPDTSLFINVCRDIDTLRDPGSQLRACPPGTAACLVRGHQAFDVGQPRDGLKVVRKDRLVLSYVREEAGKLDFCDGHSPAVTITFVCPSERREGTIPKLTAKSNCRYEIEWITEYACHRDYLESKTCSLSGEQQDVSIDLTPLAQSGGSSYISDGKEYLFYLNVCGETEIQFCNKKQAAVCQVKKSDTSQVKAAGRYHNQTLRYSDGDLTLIYFGGDECSSGFQRMSVINFECNKTAGNDGKGTPVFTGEVDCTYFFTWDTEYACVHHHHHH

SEQ ID NO: 88 - protein, 433 aa, Artificial Sequence
  CI-M6PR_K191F mutant
QAAPFPELCSYTWEAVDTKNNVLYKINICGSVDIVQCGPSSAVCMHDLKTRTYHSVGDSVLRSATRSLLEFNTTVSCDQQGTNHRVQSSIAFLCGKTLGTPEFVTATECVHYFEWRTTAACKKDIFKANKEVPCYVFDEELRKHDLNPLIFLSGAYLVDDSDPDTSLFINVCRDIDTLRDPGSQLRACPPGTAACLVRGHQAFDVGQPRDGLKVVRKDRLVLSYVREEAGKLDFCDGHSPAVTITFVCPSERREGTIPKLTAKSNCRYEIEWITEYACHRDYLESKTCSLSGEQQDVSIDLTPLAQSGGSSYISDGKEYLFYLNVCGETEIQFCNKKQAAVCQVKKSDTSQVKAAGRYHNQTLRYSDGDLTLIYFGGDECSSGFQRMSVINFECNKTAGNDGKGTPVFTGEVDCTYFFTWDTEYACVHHHHHH

SEQ ID NO: 89 - protein, 433 aa, Artificial Sequence
  CI-M6PR_L197D mutant
QAAPFPELCSYTWEAVDTKNNVLYKINICGSVDIVQCGPSSAVCMHDLKTRTYHSVGDSVLRSATRSLLEFNTTVSCDQQGTNHRVQSSIAFLCGKTLGTPEFVTATECVHYFEWRTTAACKKDIFKANKEVPCYVFDEELRKHDLNPLIKLSGAYDVDDSDPDTSLFINVCRDIDTLRDPGSQLRACPPGTAACLVRGHQAFDVGQPRDGLKVVRKDRLVLSYVREEAGKLDFCDGHSPAVTITFVCPSERREGTIPKLTAKSNCRYEIEWITEYACHRDYLESKTCSLSGEQQDVSIDLTPLAQSGGSSYISDGKEYLFYLNVCGETEIQFCNKKQAAVCQVKKSDTSQVKAAGRYHNQTLRYSDGDLTLIYFGGDECSSGFQRMSVINFECNKTAGNDGKGTPVFTGEVDCTYFFTWDTEYACVHHHHHH

SEQ ID NO: 90 - protein, 433 aa, Artificial Sequence
  CI-M6PR_D409F mutant
QAAPFPELCSYTWEAVDTKNNVLYKINICGSVDIVQCGPSSAVCMHDLKTRTYHSVGDSVLRSATRSLLEFNTTVSCDQQGTNHRVQSSIAFLCGKTLGTPEFVTATECVHYFEWRTTAACKKDIFKANKEVPCYVFDEELRKHDLNPLIKLSGAYLVDDSDPDTSLFINVCRDIDTLRDPGSQLRACPPGTAACLVRGHQAFDVGQPRDGLKVVRKDRLVLSYVREEAGKLDFCDGHSPAVTITFVCPSERREGTIPKLTAKSNCRYEIEWITEYACHRDYLESKTCSLSGEQQDVSIDLTPLAQSGGSSYISDGKEYLFYLNVCGETEIQFCNKKQAAVCQVKKSDTSQVKAAGRYHNQTLRYSDGFLTLIYFGGDECSSGFQRMSVINFECNKTAGNDGKGTPVFTGEVDCTYFFTWDTEYACVHHHHHH

SEQ ID NO: 91 - protein, 433 aa, Artificial Sequence
  CI-M6PR_E433R mutant
QAAPFPELCSYTWEAVDTKNNVLYKINICGSVDIVQCGPSSAVCMHDLKTRTYHSVGDSVLRSATRSLLEFNTTVSCDQQGTNHRVQSSIAFLCGKTLGTPEFVTATECVHYFEWRTTAACKKDIFKANKEVPCYVFDEELRKHDLNPLIKLSGAYLVDDSDPDTSLFINVCRDIDTLRDPGSQLRACPPGTAACLVRGHQAFDVGQPRDGLKVVRKDRLVLSYVREEAGKLDFCDGHSPAVTITFVCPSERREGTIPKLTAKSNCRYEIEWITEYACHRDYLESKTCSLSGEQQDVSIDLTPLAQSGGSSYISDGKEYLFYLNVCGETEIQFCNKKQAAVCQVKKSDTSQVKAAGRYHNQTLRYSDGDLTLIYFGGDECSSGFQRMSVINFRCNKTAGNDGKGTPVFTGEVDCTYFFTWDTEYACVHHHHHH

SEQ ID NO: 92 - protein, 433 aa, Artificial Sequence
  CI-M6PR_F457E mutant
QAAPFPELCSYTWEAVDTKNNVLYKINICGSVDIVQCGPSSAVCMHDLKTRTYHSVGDSVLRSATRSLLEFNTTVSCDQQGTNHRVQSSIAFLCGKTLGTPEFVTATECVHYFEWRTTAACKKDIFKANKEVPCYVFDEELRKHDLNPLIKLSGAYLVDDSDPDTSLFINVCRDIDTLRDPGSQLRACPPGTAACLVRGHQAFDVGQPRDGLKVVRKDRLVLSYVREEAGKLDFCDGHSPAVTITFVCPSERREGTIPKLTAKSNCRYEIEWITEYACHRDYLESKTCSLSGEQQDVSIDLTPLAQSGGSSYISDGKEYLFYLNVCGETEIQFCNKKQAAVCQVKKSDTSQVKAAGRYHNQTLRYSDGDLTLIYFGGDECSSGFQRMSVINFECNKTAGNDGKGTPVFTGEVDCTYEFTWDTEYACVHHHHHH

SEQ ID NO: 93 - protein, 116 aa, Artificial Sequence
  VHH7h1
EVQLVESGGGLVQPGGSLRLSCAASGIIFSDNRMDWYRQAPGKQREWVATLASYGWKTYADSVKGRFTISRDNSKNTVYLQMNSLRPEDTAVYYCKASSPVLNDIWGQGTLVTVSS

SEQ ID NO: 94 - protein, 116 aa, Artificial Sequence
  VHH7h2
DVQLVESGGGLVQPGGSLRLSCAASGIIFSDNRMDWYRQAPGKQREWVATLASYGWKTYADSVKGRFTISRDNSKNTVYLQMNSLRPEDTAVYYCKASSPVLNDIWGQGTLVTVSS

SEQ ID NO: 95 - protein, 116 aa, Artificial Sequence
  VHH7h3
DVQLVESGGGLVQPGGSLRLSCAASGIIFSDNRMDWYRQAPGKQREWVATLASYGWKTYADSVKGRFTISRDNTKNTVYLQMNSLRPEDTAVYYCKASSPVLNDIWGQGTLVTVSS

SEQ ID NO: 96 - protein, 116 aa, Artificial Sequence
  VHH7hWN
DVQLVESGGGLVQPGGSLRLSCAASGIIFSDNRMDWYRQAPGKQREWVATLASYGWKTYADSVKGRFTISRDNAKNTVYLQMNSLRPEDTAVYYCKASSPVLNDIWGQGTLVTVSS

SEQ ID NO: 97 - protein, 123 aa, Artificial Sequence
  VHH8h1
EVQLVESGGGLVQPGGSLRLSCAASGRTFNTYNWGWFRQAPGKEREFVAAIRWSSSKTSYADSVKGRFTISRDNSKNTVYLQMNSLRPEDTAVYYCAASIVDFTTNPSHFGSWGQGTLVTVSS

SEQ ID NO: 98 - protein, 123 aa, Artificial Sequence
  VHH8h2
DVQLVESGGGLVQPGGSLRLSCAASGRTFNTYNWGWFRQAPGKEREFVAAIRWSSSKTSYADSVKGRFTISRDNSKNTVYLQMNSLRPEDTAVYYCAASIVDFTTNPSHFGSWGQGTLVTVSS

SEQ ID NO: 99 - protein, 123 aa, Artificial Sequence
  VHH8h3
DVQLVESGGGLVQAGGSLRLSCAASGRTFNTYNWGWFRQAPGKEREFVAAIRWSSSKTSYADSVKGRFTISRDNSKNTVYLQMNSLRPEDTAVYYCAASIVDFTTNPSHFGSWGQGTLVTVSS

SEQ ID NO: 100 - protein, 123 aa, Artificial Sequence
  VHH8h4
DVQLVESGGGLVQPGGSLRLSCEASGRTFNTYNWGWFRQAPGKEREFVAAIRWSSSKTSYADSVKGRFTISRDNSKNTVYLQMNSLRPEDTAVYYCAASIVDFTTNPSHFGSWGQGTLVTVSS

SEQ ID NO: 101 - protein, 123 aa, Artificial Sequence
  VHH8h5
DVQLVESGGGLVQAGGSLRLSCEASGRTFNTYNWGWFRQAPGKEREFVAAIRWSSSKTSYADSVKGRFTISRDNSKNTVYLQMNSLRPEDTAVYYCAASIVDFTTNPSHFGSWGQGTLVTVSS

SEQ ID NO: 102 - protein, 123 aa, Artificial Sequence
  VHH8hWN
DVQLVESGGGLVQPGGSLRLSCAASGRTFNTYNWGWFRQAPGKEREFVAAIRWSSSKTSYADSVKGRFTISRDNAKNTIYLQMNSLRPEDTAVYYCAASIVDFTTNPSHFGSWGQGTLVTVSS

SEQ ID NO: 103 - protein, 10 aa, Artificial Sequence
  CDR sequence of CI-M6PR VHHs binding to the epitope of VHH7 or VHH8
GIIFSDNRMD

SEQ ID NO: 104 - protein, 10 aa, Artificial Sequence
  CDR sequence of CI-M6PR VHHs binding to the epitope of VHH7 or VHH8
GFTFDRYWMN

SEQ ID NO: 105 - protein, 10 aa, Artificial Sequence
  CDR sequence of CI-M6PR VHHs binding to the epitope of VHH7 or VHH8
GIIFSDNRMD

SEQ ID NO: 106 - protein, 10 aa, Artificial Sequence
  CDR sequence of CI-M6PR VHHs binding to the epitope of VHH7 or VHH8
GGAFSTYHMG

SEQ ID NO: 107 - protein, 10 aa, Artificial Sequence
  CDR sequence of CI-M6PR VHHs binding to the epitope of VHH7 or VHH8
GRTFNTYNWG

SEQ ID NO: 108 - protein, 10 aa, Artificial Sequence
  CDR sequence of CI-M6PR VHHs binding to the epitope of VHH7 or VHH8
GRTFSRLAMG

SEQ ID NO: 109 - protein, 10 aa, Artificial Sequence
  CDR sequence of CI-M6PR VHHs binding to the epitope of VHH7 or VHH8
GFTWDSYVIG

SEQ ID NO: 110 - protein, 9 aa, Artificial Sequence
  CDR sequence of CI-M6PR VHHs binding to the epitope of VHH7 or VHH8
TLASYGWKT

SEQ ID NO: 111 - protein, 10 aa, Artificial Sequence
  CDR sequence of CI-M6PR VHHs binding to the epitope of VHH7 or VHH8
TINTGGTGTY

SEQ ID NO: 112 - protein, 9 aa, Artificial Sequence
  CDR sequence of CI-M6PR VHHs binding to the epitope of VHH7 or VHH8
TLASYGWKT

SEQ ID NO: 113 - protein, 11 aa, Artificial Sequence
  CDR sequence of CI-M6PR VHHs binding to the epitope of VHH7 or VHH8
AITRGGSSSTK

SEQ ID NO: 114 - protein, 10 aa, Artificial Sequence
  CDR sequence of CI-M6PR VHHs binding to the epitope of VHH7 or VHH8
AIRWSSSKTS

SEQ ID NO: 115 - protein, 10 aa, Artificial Sequence
  CDR sequence of CI-M6PR VHHs binding to the epitope of VHH7 or VHH8
AISENGDSIH

SEQ ID NO: 116 - protein, 10 aa, Artificial Sequence
  CDR sequence of CI-M6PR VHHs binding to the epitope of VHH7 or VHH8
CLDVDDGSIY

SEQ ID NO: 117 - protein, 8 aa, Artificial Sequence
  CDR sequence of CI-M6PR VHHs binding to the epitope of VHH7 or VHH8
SSPVLNDI

SEQ ID NO: 118 - protein, 11 aa, Artificial Sequence
  CDR sequence of CI-M6PR VHHs binding to the epitope of VHH7 or VHH8
GATYYRGNSAI

SEQ ID NO: 119 - protein, 5 aa, Artificial Sequence
  CDR sequence of CI-M6PR VHHs binding to the epitope of VHH7 or VHH8
NSGQY

SEQ ID NO: 120 - protein, 16 aa, Artificial Sequence
  CDR sequence of CI-M6PR VHHs binding to the epitope of VHH7 or VHH8
SPQGKWNALVGGTPDY

SEQ ID NO: 121 - protein, 14 aa, Artificial Sequence
  CDR sequence of CI-M6PR VHHs binding to the epitope of VHH7 or VHH8
SIVDFTTNPSHFGS

SEQ ID NO: 122 - protein, 25 aa, Artificial Sequence
  CDR sequence of CI-M6PR VHHs binding to the epitope of VHH7 or VHH8
DRAAYYCSGSGCYPSRAPAAASYDY

SEQ ID NO: 123 - protein, 19 aa, Artificial Sequence
  CDR sequence of CI-M6PR VHHs binding to the epitope of VHH7 or VHH8
VNRASMRFRRCLQVLRYDY

SEQ ID NO: 124 - protein, 114 aa, Artificial Sequence
  Fig. 52 13A12
EVQLVESGGGLVQAGGSLRLSCAASGIIFGANTVGWYRQVPGKERDVVASITSGDSTYYGDSVRGRFTISRDNAKNTMYLQMNSLKPEDTAIYYCRARTTDGSYWGQGTQVTVS

SEQ ID NO: 125 - protein, 115 aa, Artificial Sequence
  Fig. 52 13B12
EVQLVESGGGLVQAGGSLRLSCAASGIIFGANTVGWYRQVPGKERDVVASITSVDSTYYGDSVRGRFTISRDNAKNTVYLQMNSLKPEDTAIYYCRARTTDGSYWGQGTQVTVSS

SEQ ID NO: 126 - protein, 115 aa, Artificial Sequence
  Fig. 52 13A2
EVQLVESGGGLVQAGGSLRLSCAASGIIFSANTVGWYRQVPGKERDVVASITSGDSTYYGDSVRDRFTISRDNAKNTVYLQMNSLKPDDTAIYYCRARTTDGSYWGQGTQVTVSS

SEQ ID NO: 127 - protein, 114 aa, Artificial Sequence
  Fig 52. 13C11
EVQLVESGGGLVQAGGSLRLSCAASGLTFSSYTMAWYRQAPGKEREVVAAISSGDSIYHEGSVQGRFIISRDNAKNTVYLQMNSLKAEDTAVYYCRANGPGTYWGQGTQVTVSS

SEQ ID NO: 128 - protein, 114 aa, Artificial Sequence
  Fig. 52 13G10
EVQLVESGGGLVQAGGSLRLSCAASGLTFSSYTMAWYRQAPGKEREVVAAISSGDSIYHEDSVQGRFIISRDNAKNTVYLQMNSLKAEDTAVYYCRANGPGTYWGQGTQVTVSS

SEQ ID NO: 129 - protein, 114 aa, Artificial Sequence
  Fig. 52 13E5
EVQLVESGGGLVQAGGSLRLSCAASGLTFSRYTMSWYRQAPGKEREVVAAISSGDSTYHEDSVKGRFIISRDNAKNTVYLQMNSLKTEDTAVYYCRANGPGTYWGQGTQVTVSS

SEQ ID NO: 130 - protein, 112 aa, Artificial Sequence
  Fig. 52 13A10
EVQLVESGGGLVQAGGSLRLSCSASGSIFSINYMAWYRQEPGKEREVVASMSWRGDSTYLADSVQGRFTISRNNAKNTMYLQMNSLKPEDTALYYCKANNYWGQGTQVTVSS

SEQ ID NO: 131 - protein, 112 aa, Artificial Sequence
  Fig. 52 13D11
EVQLVESGGGLVQAGGSLRLSCAASGSIFSINYMAWYRQTPGKEREVVASMSWRRDSTYLADSVQGRFTISRNNAKNTMYLQMNSLKPEDTALYYCKANDYWGQGTQVTVSS

SEQ ID NO: 132 - protein, 112 aa, Artificial Sequence
  Fig. 52 13A3
EVQLVESGGGLVQAGGSLRLSCAASGSIFRLNYMAWYRQAPGKEREVVASMSRRSDSTYLADAVQGRFTISMNNAGNTMYLQMNSLKPEDTALYYCKANDYWGQGTQVTVSS

SEQ ID NO: 133 - protein, 112 aa, Artificial Sequence
  Fig. 52 13F4
EVQLVESGGGLVQAGGSLRLSCVASGSIFSINYMAWYRQTPGKEREVVASMSRRSDSTYLADAVQGRFTISRNNAGNTMYLQMNSLKPEDTALYYCKANDYWGQGTQVTVSS

SEQ ID NO: 134 - protein, 119 aa, Artificial Sequence
  Fig. 52 13G12
EVQLVESGGGLVQAGGSLRLSCAASGRAFSISSMSWFRQAPGKERVVVASIAWSGDSTYYADSVQGRFTISKDNAKNTLSLQMNSLKPEDTAVYYCSAYTQVSVNNRYWGQGTQVTVSS

SEQ ID NO: 135 - protein, 109 aa, Artificial Sequence
  Fig. 52 13E8
EVQLVESGGGLVQAGGSLRLSCAASRSISPMGWYRQAPGKQRELVAIMPSSGPPIYADSVQGRFTISRDNAKNTVYLQMNSLKPEDTAVYYCNVGGGYWGQGTQVTVSS

SEQ ID NO: 136 - protein, 109 aa, Artificial Sequence
  Fig. 52 13B7
EVQLVESGGGLVQAGGSLRLSCAASRSISPMGWYRQAPGKQRELVAIMPSSGTPIYADSVQGRFTISRDNAKNTVYLQMNSLKPEDTAVYYCNVGGGYWGQGTQVTVSS

SEQ ID NO: 137 - protein, 123 aa, Artificial Sequence
  Fig. 52 13A8
EVQLVESGGGLVPAGGSLRLSCAASGRTFSNYAMGWFRQAPGKERKFVATISWSGGVTYYADSVKGRFTISRDNAKNTVYLLMNSLKPEDTAVYYCAAKRDSSSYDHRRYDYWGQGTQVTVSS

SEQ ID NO: 138 - protein, 124 aa, Artificial Sequence
  Fig. 52 13F11
EVQLVESGGRLVQPGDSLRLSCAASGRTFSDNAMGWFRQAPGKERRFVAGISWAGGSTYYSDSVKGRFTISRDNAKNTVYLQMNSLKPEDTAVYYCAAGLRAWVQRMPKDYNYWGQGTQVTVSS

SEQ ID NO: 139 - protein, 9 aa, Artificial Sequence
  synthetic substrate (Mca-GKPILFFRL(dinitrophenyl)DR-NH2)
GKPILFFRL